(19) United States
(12) Patent Application Publication
(10) Pub. No.: US 2003/0064377 A1
(43) Pub. Date: Apr. 3, 2003

(54) Compositions and methods relating to prostate specific genes and proteins

(76) Inventors: Yongming Sun, San Jose, CA (US); Herve E. Recipon, San Francisco, CA (US); Sei-Yu Chen, Foster City, CA (US); Chenghua Liu, San Jose, CA (US)

Correspondence Address: Licata & Tyrrell P.C., 66 East Main Street, Marlton, NJ, 08053, US

(21) Appl. No.: 10/012,600

(22) Filed: Nov. 6, 2001

Related U.S. Application Data
(60) Provisional application No. 60/246,109, filed on Nov. 6, 2000.

(51) Int. Cl. C12Q 1/68; G01N 33/574; C07H 21/04; C12N 9/00; C12P 21/02; C12N 5/06

(52) U.S. Cl. 435/6; 435/7.23; 435/69.1; 435/183; 435/320.1; 435/325; 536/23.2

(57) ABSTRACT

The present invention relates to newly identified nucleic acids and polypeptides present in normal and neoplastic prostate cells, including fragments, variants and derivatives of the nucleic acids and polypeptides. The present invention also relates to antibodies to the polypeptides of the invention, as well as agonists and antagonists of the polypeptides of the invention. The invention also relates to compositions comprising the nucleic acids, polypeptides, antibodies, variants, derivatives, agonists and antagonists of the invention and methods for the use of these compositions. These uses include identifying, diagnosing, monitoring, staging, imaging and treating prostate cancer and non-cancerous disease states in prostate tissue, identifying prostate tissue, monitoring and identifying and/or designing agonists and antagonists of polypeptides of the invention. The uses also include gene therapy, production of transgenic animals and cells, and production of engineered prostate tissue for treatment and research.

This application claims the benefit of priority from U.S. Provisional Application Serial No. 60/246,109 filed Nov. 6, 2000, which is herein incorporated by reference in its entirety.

### FIELD OF THE INVENTION

The present invention relates to newly identified nucleic acid molecules and polypeptides present in normal and neoplastic prostate cells, including fragments, variants and derivatives of the nucleic acids and polypeptides. The present invention also relates to antibodies to the polypeptides of the invention, as well as agonists and antagonists of the polypeptides of the invention. The invention also relates to compositions comprising the nucleic acids, polypeptides, antibodies, variants, derivatives, agonists and antagonists of the invention and methods for the use of these compositions. These uses include identifying, diagnosing, monitoring, staging, imaging and treating prostate cancer and non-cancerous disease states in prostate tissue, identifying prostate tissue and monitoring and identifying and/or designing agonists and antagonists of polypeptides of the invention. The uses also include gene therapy, production of transgenic animals and cells, and production of engineered prostate tissue for treatment and research.

### BACKGROUND OF THE INVENTION

Prostate cancer is the most prevalent cancer in men and is the second leading cause of death from cancer among males in the United States. AJCC Cancer Staging Handbook 203 (Irvin D. Fleming et al. eds., 5^{th} ed. 1998); Walter J. Burdette, Cancer: Etiology, Diagnosis, and Treatment 147 (1998). In 1999, it was estimated that 37,000 men in the United States would die as result of prostate cancer. Elizabeth A. Platz et al., & Edward Giovannucci, Epidemiology of and Risk Factors for Prostate Cancer, in Management of Prostate Cancer 21 (Eric A Klein, ed. 2000). Cancer of the prostate typically occurs in older males, with a median age of 74 years for clinical diagnosis. Burdette, supra at 147. A man's risk of being diagnosed with invasive prostate cancer in his lifetime is one in six. Platz et al., supra at 21.

Although our understanding of the etiology of prostate cancer is incomplete, the results of extensive research in this area point to a combination of age, genetic and environmental/dietary factors. Platz et al., supra at 19; Burdette, supra at 147; Steven K. Clinton, Diet and Nutrition in Prostate Cancer Prevention and Therapy, in Prostate Cancer: A Multidisciplinary Guide 246-269 (Philip W. Kantoff et al. eds. 1997). Broadly speaking, genetic risk factors predisposing one to prostate cancer include race and a family history of the disease. Platz et al., supra at 19, 28-29, 32-34. Aside from these generalities, a deeper understanding of the genetic basis of prostate cancer has remained elusive. Considerable research has been directed to studying the link between prostate cancer, androgens, and androgen regulation, as androgens play a crucial role in prostate growth and differentiation. Meena Augustus et al., Molecular Genetics and Markers of Progression, in Management of Prostate Cancer 59 (Eric A Klein ed. 2000). While a number of studies have concluded that prostate tumor development is linked to elevated levels of circulating androgen (e.g., testosterone and dihydrotestosterone), the genetic determinants of these levels remain unknown. Platz et al., supra at 29-30.

Several studies have explored a possible link between prostate cancer and the androgen receptor (AR) gene, the gene product of which mediates the molecular and cellular effects of testosterone and dihydrotestosterone in tissues responsive to androgens. Id. at 30. Differences in the number of certain trinucleotide repeats in exon 1, the region involved in transactivational control, have been of particular interest. Augustus et al., supra at 60. For example, these studies have revealed that as the number of CAG repeats decreases the transactivation ability of the gene product increases, as does the risk of prostate cancer. Platz et al., supra at 30-31. Other research has focused on the α-reductase Type 2 gene, the gene which codes for the enzyme that converts testosterone into dihydrotestosterone. Id. at 30. Dihydrotestosterone has greater affinity for the AR than testosterone, resulting in increased transactivation of genes responsive to androgens. Id. While studies have reported differences among the races in the length of a TA dinucleotide repeat in the 3′ untranslated region, no link has been established between the length of that repeat and prostate cancer. Id. Interestingly, while ras gene mutations are implicated in numerous other cancers, such mutations appear not to play a significant role in prostate cancer, at least among Caucasian males. Augustus, supra at 52.

Environmental/dietary risk factors which may increase the risk of prostate cancer include intake of saturated fat and calcium. Platz et al., supra at 19, 25-26. Conversely, intake of selenium, vitamin E and tomato products (which contain the carotenoid lycopene) apparently decrease that risk. Id. at 19, 26-28 The impact of physical activity, cigarette smoking, and alcohol consumption on prostate cancer is unclear. Platz et al., supra at 23-25.

Periodic screening for prostate cancer is most effectively performed by digital rectal examination (DRE) of the prostate, in conjunction with determination of the serum level of prostate-specific antigen (PSA). Burdette, supra at 148. While the merits of such screening are the subject of considerable debate, Jerome P. Richie & Irving D. Kaplan, Screening for Prostate Cancer: The Horns of a Dilemma, in Prostate Cancer: A Multidisciplinary Guide 1-10 (Philip W. Kantoff et al. eds. 1997), the American Cancer Society and American Urological Association recommend that both of these tests be performed annually on men 50 years or older with a life expectancy of at least 10 years, and younger men at high risk for prostate cancer. Ian M. Thompson & John Foley, Screening for Prostate Cancer, in Management of Prostate Cancer 71 (Eric A Klein ed. 2000). If necessary, these screening methods may be followed by additional tests, including biopsy, ultrasonic imaging, computerized tomography, and magnetic resonance imaging. Christopher A. Haas & Martin I. Resnick, Trends in Diagnosis, Biopsy, and Imaging, in Management of Prostate Cancer 89-98 (Eric A Klein ed. 2000); Burdette, supra at 148.

Once the diagnosis of prostate cancer has been made, treatment decisions for the individual are typically linked to the stage of prostate cancer present in that individual, as well as his age and overall health. Burdette, supra at 151. One preferred classification system for staging prostate cancer was developed by the American Urological Association (AUA). Id. at 148. The AUA classification system divides prostate tumors into four broad stages, A to D, which are in turn accompanied by a number of smaller substages. Burdette, supra at 152-153; Anthony V. D'Amico et al., The Staging of Prostate Cancer, in Prostate Cancer: A Multidisciplinary Guide 41 (Philip W. Kantoff et al. eds. 1997).

Stage A prostate cancer refers to the presence of microscopic cancer within the prostate gland. D'Amico, supra at 41. This stage is comprised of two substages: A1, which involves less than four well-differentiated cancer foci within the prostate, and A2, which involves greater than three well-differentiated cancer foci or alternatively, moderately to poorly differentiated foci within the prostate. Burdette, supra at 152; D'Amico, supra at 41. Treatment for stage A1 preferentially involves following PSA levels and periodic DRE. Burdette, supra at 151. Should PSA levels rise, preferred treatments include radical prostatectomy in patients 70 years of age and younger, external beam radiotherapy for patients between 70 and 80 years of age, and hormone therapy for those over 80 years of age. Id.

Stage B prostate cancer is characterized by the presence of a palpable lump within the prostate. Burdette, supra at 152-53; D'Amico, supra at 41. This stage is comprised of three substages: B1, in which the lump is less than 2 cm and is contained in one lobe of the prostate; B2, in which the lump is greater than 2 cm yet is still contained within one lobe; and B3, in which the lump has spread to both lobes. Burdette, supra, at 152-53. For stages B 1 and B2, the treatment again involves radical prostatectomy in patients 70 years of age and younger, external beam radiotherapy for patients between 70 and 80 years of age, and hormone therapy for those over 80 years of age. Id. at 151. In stage B3, radical prostatectomy is employed if the cancer is well-differentiated and PSA levels are below 15 ng/mL; otherwise, external beam radiation is the chosen treatment option. Id.

Stage C prostate cancer involves a substantial cancer mass accompanied by extraprostatic extension. Burdette, supra at 153; D'Amico, supra at 41. Like stage A prostate cancer, Stage C is comprised of two substages: substage C1, in which the tumor is relatively minimal, with minor prostatic extension, and substage C2, in which the tumor is large and bulky, with major prostatic extension. Id. The treatment of choice for both substages is external beam radiation. Burdette, supra at 151.

The fourth and final stage of prostate cancer, Stage D, describes the extent to which the cancer has metastasized. Burdette, supra at 153; D'Amico, supra at 41. This stage is comprised of four substages: (1) D0, in which acid phophatase levels are persistently high, (2) D1, in which only the pelvic lymph nodes have been invaded, (3) D2, in which the lymph nodes above the aortic bifurcation have been invaded, with or without distant metastasis, and (4) D3, in which the metastasis progresses despite intense hormonal therapy. Id. Treatment at this stage may involve hormonal therapy, chemotherapy, and removal of one or both testes. Burdette, supra at 151.

Despite the need for accurate staging of prostate cancer, current staging methodology is limited. The wide variety of biological behavior displayed by neoplasms of the prostate has resulted in considerable difficulty in predicting and assessing the course of prostate cancer. Augustus et al., supra at 47. Indeed, despite the fact that most prostate cancer patients have carcinomas that are of intermediate grade and stage, prognosis for these types of carcinomas is highly variable. Andrew A Renshaw & Christopher L. Corless, Prognostic Features in the Pathology of Prostate Cancer, in Prostate Cancer: A Multidisciplinary Guide 26 (Philip W. Kantoff et al. eds. 1997). Techniques such as transrectal ultrasound, abdominal and pelvic computerized tomography, and MRI have not been particularly useful in predicting local tumor extension. D'Amico, supra at 53 (editors' comment). While the use of serum PSA in combination with the Gleason score is currently the most effective method of staging prostate cancer, id., PSA is of limited predictive value, Augustus et al., supra at 47; Renshaw et al., supra at 26, and the Gleason score is prone to variability and error, King, C. R. & Long, J. P., Int'l. J Cancer 90(6): 326-30 (2000). As such, the current focus of prostate cancer research has been to obtain biomarkers to help better assess the progression of the disease. Augustus et al., supra at 47; Renshaw et al., supra at 26; Pettaway, C. A., Tech. Urol. 4(1): 35-42 (1998).

Accordingly, there is a great need for more sensitive and accurate methods for predicting whether a person is likely to develop prostate cancer, for diagnosing prostate cancer, for monitoring the progression of the disease, for staging the prostate cancer, for determining whether the prostate cancer has metastasized and for imaging the prostate cancer. There is also a need for better treatment of prostate cancer.

### SUMMARY OF THE INVENTION

The present invention solves these and other needs in the art by providing nucleic acid molecules and polypeptides as well as antibodies, agonists and antagonists, thereto that may be used to identify, diagnose, monitor, stage, image and treat prostate cancer and non-cancerous disease states in prostate; identify and monitor prostate tissue; and identify and design agonists and antagonists of polypeptides of the invention. The invention also provides gene therapy, methods for producing transgenic animals and cells, and methods for producing engineered prostate tissue for treatment and research.

Accordingly, one object of the invention is to provide nucleic acid molecules that are specific to prostate cells and/or prostate tissue. These prostate specific nucleic acids (PSNAs) may be a naturally-occurring cDNA, genomic DNA, RNA, or a fragment of one of these nucleic acids, or may be a non-naturally-occurring nucleic acid molecule. If the PSNA is genomic DNA, then the PSNA is a prostate specific gene (PSG). In a preferred embodiment, the nucleic acid molecule encodes a polypeptide that is specific to prostate. In a more preferred embodiment, the nucleic acid molecule encodes a polypeptide that comprises an amino acid sequence of SEQ ID NO: 136 through 240. In another highly preferred embodiment, the nucleic acid molecule comprises a nucleic acid sequence of SEQ ID NO: 1 through 135. By nucleic acid molecule, it is also meant to be inclusive of sequences that selectively hybridize or exhibit substantial sequence similarity to a nucleic acid molecule encoding a PSP, or that selectively hybridize or exhibit substantial sequence similarity to a PSNA, as well as allelic variants of a nucleic acid molecule encoding a PSP, and allelic variants of a PSNA. Nucleic acid molecules comprising a part of a nucleic acid sequence that encodes a PSP or that comprises a part of a nucleic acid sequence of a PSNA are also provided.

A related object of the present invention is to provide a nucleic acid molecule comprising one or more expression control sequences controlling the transcription and/or translation of all or a part of a PSNA. In a preferred embodiment, the nucleic acid molecule comprises one or more expression control sequences controlling the transcription and/or translation of a nucleic acid molecule that encodes all or a fragment of a PSP.

Another object of the invention is to provide vectors and/or host cells comprising a nucleic acid molecule of the instant invention. In a preferred embodiment, the nucleic acid molecule encodes all or a fragment of a PSP. In another preferred embodiment, the nucleic acid molecule comprises all or a part of a PSNA.

Another object of the invention is to provided methods for using the vectors and host cells comprising a nucleic acid molecule of the instant invention to recombinantly produce polypeptides of the invention.

Another object of the invention is to provide a polypeptide encoded by a nucleic acid molecule of the invention. In a preferred embodiment, the polypeptide is a PSP. The polypeptide may comprise either a fragment or a full-length protein as well as a mutant protein (mutein), fusion protein, homologous protein or a polypeptide encoded by an allelic variant of a PSP.

Another object of the invention is to provide an antibody that specifically binds to a polypeptide of the instant invention.

Another object of the invention is to provide agonists and antagonists of the nucleic acid molecules and polypeptides of the instant invention.

Another object of the invention is to provide methods for using the nucleic acid molecules to detect or amplify nucleic acid molecules that have similar or identical nucleic acid sequences compared to the nucleic acid molecules described herein. In a preferred embodiment, the invention provides methods of using the nucleic acid molecules of the invention for identifying, diagnosing, monitoring, staging, imaging and treating prostate cancer and non-cancerous disease states in prostate. In another preferred embodiment, the invention provides methods of using the nucleic acid molecules of the invention for identifying and/or monitoring prostate tissue. The nucleic acid molecules of the instant invention may also be used in gene therapy, for producing transgenic animals and cells, and for producing engineered prostate tissue for treatment and research.

The polypeptides and/or antibodies of the instant invention may also be used to identify, diagnose, monitor, stage, image and treat prostate cancer and non-cancerous disease states in prostate. The invention provides methods of using the polypeptides of the invention to identify and/or monitor prostate tissue, and to produce engineered prostate tissue.

The agonists and antagonists of the instant invention may be used to treat prostate cancer and non-cancerous disease states in prostate and to produce engineered prostate tissue.

Yet another object of the invention is to provide a computer readable means of storing the nucleic acid and amino acid sequences of the invention. The records of the computer readable means can be accessed for reading and displaying of sequences for comparison, alignment and ordering of the sequences of the invention to other sequences.

### DETAILED DESCRIPTION OF THE INVENTION

Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well-known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press (1989) and Sambrook et al., Molecular Cloning: A Laboratory Manual, 3d ed., Cold Spring Harbor Press (2001); Ausubel et al., Current Protocols in Molecular Biology, Greene Publishing Associates (1992, and Supplements to 2000); Ausubel et al., Short Protocols in Molecular Biology: A Compendium of Methods from Current Protocols in Molecular Biology—4^{th} Ed., Wiley & Sons (1999); Harlow and Lane, Antibodies: A Laboratory Manual, Cold Spring Harbor Laboratory Press (1990); and Harlow and Lane, Using Antibodies: A Laboratory Manual, Cold Spring Harbor Laboratory Press (1999); each of which is incorporated herein by reference in its entirety.

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

A “nucleic acid molecule” of this invention refers to a polymeric form of nucleotides and includes both sense and antisense strands of RNA, cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” The term “nucleic acid molecule” usually refers to a molecule of at least 10 bases in length, unless otherwise specified. The term includes single- and double-stranded forms of DNA. In addition, a polynucleotide may include either or both naturally-occurring and modified nucleotides linked together by naturally-occurring and/or non-naturally occurring nucleotide linkages.

The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, intemucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) The term “nucleic acid molecule” also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

A “gene” is defined as a nucleic acid molecule that comprises a nucleic acid sequence that encodes a polypeptide and the expression control sequences that surround the nucleic acid sequence that encodes the polypeptide. For instance, a gene may comprise a promoter, one or more enhancers, a nucleic acid sequence that encodes a polypeptide, downstream regulatory sequences and, possibly, other nucleic acid sequences involved in regulation of the expression of an RNA. As is well-known in the art, eukaryotic genes usually contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed to not contribute to a mature mRNA transcript, but rather to be “spliced out” during processing of the transcript.

A nucleic acid molecule or polypeptide is “derived” from a particular species if the nucleic acid molecule or polypeptide has been isolated from the particular species, or if the nucleic acid molecule or polypeptide is homologous to a nucleic acid molecule or polypeptide isolated from a particular species.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases, or genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, (4) does not occur in nature as part of a larger sequence or (5) includes nucleotides or internucleoside bonds that are not found in nature. The term “isolated” or “substantially pure” also can be used in reference to recombinant or cloned DNA isolates, chemically synthesized polynucleotide analogs, or polynucleotide analogs that are biologically synthesized by heterologous systems. The term “isolated nucleic acid molecule” includes nucleic acid molecules that are integrated into a host cell chromosome at a heterologous site, recombinant fusions of a native fragment to a heterologous sequence, recombinant vectors present as episomes or as integrated into a host cell chromosome.

A “part” of a nucleic acid molecule refers to a nucleic acid molecule that comprises a partial contiguous sequence of at least 10 bases of the reference nucleic acid molecule. Preferably, a part comprises at least 15 to 20 bases of a reference nucleic acid molecule. In theory, a nucleic acid sequence of 17 nucleotides is of sufficient length to occur at random less frequently than once in the three gigabase human genome, and thus to provide a nucleic acid probe that can uniquely identify the reference sequence in a nucleic acid mixture of genomic complexity. A preferred part is one that comprises a nucleic acid sequence that can encode at least 6 contiguous amino acid sequences (fragments of at least 18 nucleotides) because they are useful in directing the expression or synthesis of peptides that are useful in mapping the epitopes of the polypeptide encoded by the reference nucleic acid. See, e.g., Geysen et al., Proc. Natl. Acad. Sci. USA 81:3998-4002 (1984); and U.S. Pat. Nos. 4,708,871 and 5,595,915, the disclosures of which are incorporated herein by reference in their entireties. A part may also comprise at least 25, 30, 35 or 40 nucleotides of a reference nucleic acid molecule, or at least 50, 60, 70, 80, 90, 100, 150, 200, 250, 300, 350, 400 or 500 nucleotides of a reference nucleic acid molecule. A part of a nucleic acid molecule may comprise no other nucleic acid sequences. Alternatively, a part of a nucleic acid may comprise other nucleic acid sequences from other nucleic acid molecules.

The term “oligonucleotide” refers to a nucleic acid molecule generally comprising a length of 200 bases or fewer. The term often refers to single-stranded deoxyribonucleotides, but it can refer as well to single- or double-stranded ribonucleotides, RNA:DNA hybrids and double-stranded DNAs, among others. Preferably, oligonucleotides are 10 to 60 bases in length and most preferably 12, 13, 14, 15, 16, 17, 18, 19 or 20 bases in length. Other preferred oligonucleotides are 25, 30, 35, 40, 45, 50, 55 or 60 bases in length. Oligonucleotides may be single-stranded, e.g. for use as probes or primers, or may be double-stranded, e.g. for use in the construction of a mutant gene. Oligonucleotides of the invention can be either sense or antisense oligonucleotides. An oligonucleotide can be derivatized or modified as discussed above for nucleic acid molecules.

Oligonucleotides, such as single-stranded DNA probe oligonucleotides, often are synthesized by chemical methods, such as those implemented on automated oligonucleotide synthesizers. However, oligonucleotides can be made by a variety of other methods, including in vitro recombinant DNA-mediated techniques and by expression of DNAs in cells and organisms. Initially, chemically synthesized DNAs typically are obtained without a 5′ phosphate. The 5′ ends of such oligonucleotides are not substrates for phosphodiester bond formation by ligation reactions that employ DNA ligases typically used to form recombinant DNA molecules. Where ligation of such oligonucleotides is desired, a phosphate can be added by standard techniques, such as those that employ a kinase and ATP. The 3′ end of a chemically synthesized oligonucleotide generally has a free hydroxyl group and, in the presence of a ligase, such as T4 DNA ligase, readily will form a phosphodiester bond with a 5′ phosphate of another polynucleotide, such as another oligonucleotide. As is well-known, this reaction can be prevented selectively, where desired, by removing the 5′ phosphates of the other polynucleotide(s) prior to ligation.

The term “naturally-occurring nucleotide” referred to herein includes naturally-occurring deoxyribonucleotides and ribonucleotides. The term “modified nucleotides” referred to herein includes nucleotides with modified or substituted sugar groups and the like. The term “nucleotide linkages” referred to herein includes nucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081-9093 (1986); Stein et al. Nucl. Acids Res. 16:3209-3221 (1988); Zon et al. Anti-Cancer Drug Design 6:539-568 (1991); Zon et al., in Eckstein (ed.) Oligonucleotides and Analogues: A Practical Approach, pp. 87-108, Oxford University Press (1991); U.S. Pat. No. 5,151,510; Uhlmann and Peyman Chemical Reviews 90:543 (1990), the disclosures of which are hereby incorporated by reference.

Unless specified otherwise, the left hand end of a polynucleotide sequence in sense orientation is the 5′ end and the right hand end of the sequence is the 3′ end. In addition, the left hand direction of a polynucleotide sequence in sense orientation is referred to as the 5′ direction, while the right hand direction of the polynucleotide sequence is referred to as the 3′ direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

The term “allelic variant” refers to one of two or more alternative naturally-occurring forms of a gene, wherein each gene possesses a unique nucleotide sequence. In a preferred embodiment, different alleles of a given gene have similar or identical biological properties.

The term “percent sequence identity” in the context of nucleic acid sequences refers to the residues in two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183: 63-98 (1990); Pearson, Methods Mol. Biol. 132: 185-219 (2000); Pearson, Methods Enzymol. 266: 227-258 (1996); Pearson, J. Mol. Biol. 276: 71-84 (1998); herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleic acid sequence encompasses its complement unless otherwise specified. Thus, a reference to a nucleic acid molecule having a particular sequence should be understood to encompass its complementary strand, with its complementary sequence. The complementary strand is also useful, e.g., for antisense therapy, hybridization probes and PCR primers.

In the molecular biology art, researchers use the terms “percent sequence identity”, “percent sequence similarity” and “percent sequence homology” interchangeably. In this application, these terms shall have the same meaning with respect to nucleic acid sequences only.

The term “substantial similarity” or “substantial sequence similarity,” when referring to a nucleic acid or fragment thereof, indicates that, when optimally aligned with appropriate nucleotide insertions or deletions with another nucleic acid (or its complementary strand), there is nucleotide sequence identity in at least about 50%, more preferably 60% of the nucleotide bases, usually at least about 70%, more usually at least about 80%, preferably at least about 90%, and more preferably at least about 95-98% of the nucleotide bases, as measured by any well-known algorithm of sequence identity, such as FASTA, BLAST or Gap, as discussed above.

Alternatively, substantial similarity exists when a nucleic acid or fragment thereof hybridizes to another nucleic acid, to a strand of another nucleic acid, or to the complementary strand thereof, under selective hybridization conditions. Typically, selective hybridization will occur when there is at least about 55% sequence identity, preferably at least about 65%, more preferably at least about 75%, and most preferably at least about 90% sequence identity, over a stretch of at least about 14 nucleotides, more preferably at least 17 nucleotides, even more preferably at least 20, 25, 30, 35, 40, 50, 60, 70, 80, 90 or 100 nucleotides.

Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. “Stringent hybridization conditions” and “stringent wash conditions” in the context of nucleic acid hybridization experiments depend upon a number of different physical parameters. The most important parameters include temperature of hybridization, base composition of the nucleic acids, salt concentration and length of the nucleic acid. One having ordinary skill in the art knows how to vary these parameters to achieve a particular stringency of hybridization. In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tm is the temperature at which 50% of the target sequence hybridizes to a perfectly matched probe. See Sambrook (1989), supra, p. 9.51, hereby incorporated by reference.

The Tₘ for a particular DNA-DNA hybrid can be estimated by the formula:

Tₘ=81.5° C.+16.6 (log₁₀[Na⁺])+0.41(fraction G+C)−0.63(% formamide)−(600/1)

where 1 is the length of the hybrid in base pairs.

The Tₘ for a particular RNA—RNA hybrid can be estimated by the formula:

Tₘ=79.8° C.+18.5(log₁₀[Na⁺])+0.58(fraction G+C)+11.8(fraction G+C)²−0.35(% formamide)−(820/1).

The Tₘ for a particular RNA-DNA hybrid can be estimated by the formula:

Tₘ=79.8° C.+18.5(log₁₀[Na⁺])+0.58(fraction G+C)+11.8(fraction G+C)²−0.50(% formamide)−(820/1).

In general, the Tm decreases by 1-1.5° C. for each 1% of mismatch between two nucleic acid sequences. Thus, one having ordinary skill in the art can alter hybridization and/or washing conditions to obtain sequences that have higher or lower degrees of sequence identity to the target nucleic acid. For instance, to obtain hybridizing nucleic acids that contain up to 10% mismatch from the target nucleic acid sequence, 10-15° C. would be subtracted from the calculated Tm of a perfectly matched hybrid, and then the hybridization and washing temperatures adjusted accordingly. Probe sequences may also hybridize specifically to duplex DNA under certain conditions to form triplex or other higher order DNA complexes. The preparation of such probes and suitable hybridization conditions are well-known in the art.

An example of stringent hybridization conditions for hybridization of complementary nucleic acid sequences having more than 100 complementary residues on a filter in a Southern or Northern blot or for screening a library is 50% formamide/6× SSC at 42° C. for at least ten hours and preferably overnight (approximately 16 hours). Another example of stringent hybridization conditions is 6× SSC at 68° C. without formamide for at least ten hours and preferably overnight. An example of moderate stringency hybridization conditions is 6× SSC at 55° C. without formamide for at least ten hours and preferably overnight. An example of low stringency hybridization conditions for hybridization of complementary nucleic acid sequences having more than 100 complementary residues on a filter in a Southern or Northern blot or for screening a library is 6× SSC at 42° C. for at least ten hours. Hybridization conditions to identify nucleic acid sequences that are similar but not identical can be identified by experimentally changing the hybridization temperature from 68° C. to 42° C. while keeping the salt concentration constant (6× SSC), or keeping the hybridization temperature and salt concentration constant (e.g. 42° C. and 6× SSC) and varying the formamide concentration from 50% to 0%. Hybridization buffers may also include blocking agents to lower background. These agents are well-known in the art. See Sambrook et al. (1989), supra, pages 8.46 and 9.46-9.58, herein incorporated by reference. See also Ausubel (1992), supra, Ausubel (1999), supra, and Sambrook (2001), supra.

Wash conditions also can be altered to change stringency conditions. An example of stringent wash conditions is a 0.2× SSC wash at 65° C. for 15 minutes (see Sambrook (1989), supra, for SSC buffer). Often the high stringency wash is preceded by a low stringency wash to remove excess probe. An exemplary medium stringency wash for duplex DNA of more than 100 base pairs is 1× SSC at 45° C. for 15 minutes. An exemplary low stringency wash for such a duplex is 4× SSC at 40° C. for 15 minutes. In general, signal-to-noise ratio of 2× or higher than that observed for an unrelated probe in the particular hybridization assay indicates detection of a specific hybridization.

As defined herein, nucleic acid molecules that do not hybridize to each other under stringent conditions are still substantially similar to one another if they encode polypeptides that are substantially identical to each other. This occurs, for example, when a nucleic acid molecule is created synthetically or recombinantly using high codon degeneracy as permitted by the redundancy of the genetic code.

Hybridization conditions for nucleic acid molecules that are shorter than 100 nucleotides in length (e.g., for oligonucleotide probes) may be calculated by the formula:

Tₘ=81.5° C.+16.6(log₁₀[Na⁺])+0.41(fraction G+C)−(600/N),

wherein N is change length and the [Na+] is 1 M or less. See Sambrook (1989), supra, p. 11.46. For hybridization of probes shorter than 100 nucleotides, hybridization is usually performed under stringent conditions (5-10° C. below the Tₘ) using high concentrations (0.1-1.0 pmol/ml) of probe. Id. at p. 11.45. Determination of hybridization using mismatched probes, pools of degenerate probes or “guessmers,” as well as hybridization solutions and methods for empirically determining hybridization conditions are well-known in the art. See, e.g., Ausubel (1999), supra; Sambrook (1989), supra, pp. 11.45-11.57.

The term “digestion” or “digestion of DNA” refers to catalytic cleavage of the DNA with a restriction enzyme that acts only at certain sequences in the DNA. The various restriction enzymes referred to herein are commercially available and their reaction conditions, cofactors and other requirements for use are known and routine to the skilled artisan. For analytical purposes, typically, 1 μg of plasmid or DNA fragment is digested with about 2 units of enzyme in about 20 μg of reaction buffer. For the purpose of isolating DNA fragments for plasmid construction, typically 5 to 50 μg of DNA are digested with 20 to 250 units of enzyme in proportionately larger volumes. Appropriate buffers and substrate amounts for particular restriction enzymes are described in standard laboratory manuals, such as those referenced below, and they are specified by commercial suppliers. Incubation times of about 1 hour at 37° C. are ordinarily used, but conditions may vary in accordance with standard procedures, the supplier's instructions and the particulars of the reaction. After digestion, reactions may be analyzed, and fragments may be purified by electrophoresis through an agarose or polyacrylamide gel, using well-known methods that are routine for those skilled in the art.

The term “ligation” refers to the process of forming phosphodiester bonds between two or more polynucleotides, which most often are double-stranded DNAS. Techniques for ligation are well-known to the art and protocols for ligation are described in standard laboratory manuals and references, such as, e.g., Sambrook (1989), supra.

Genome-derived “single exon probes,” are probes that comprise at least part of an exon (“reference exon”) and can hybridize detectably under high stringency conditions to transcript-derived nucleic acids that include the reference exon but do not hybridize detectably under high stringency conditions to nucleic acids that lack the reference exon. Single exon probes typically further comprise, contiguous to a first end of the exon portion, a first intronic and/or intergenic sequence that is identically contiguous to the exon in the genome, and may contain a second intronic and/or intergenic sequence that is identically contiguous to the exon in the genome. The minimum length of genome-derived single exon probes is defined by the requirement that the exonic portion be of sufficient length to hybridize under high stringency conditions to transcript-derived nucleic acids, as discussed above. The maximum length of genome-derived single exon probes is defined by the requirement that the probes contain portions of no more than one exon. The single exon probes may contain priming sequences not found in contiguity with the rest of the probe sequence in the genome, which priming sequences are useful for PCR and other amplification-based technologies.

The term “microarray” or “nucleic acid microarray” refers to a substrate-bound collection of plural nucleic acids, hybridization to each of the plurality of bound nucleic acids being separately detectable. The substrate can be solid or porous, planar or non-planar, unitary or distributed. Microarrays or nucleic acid microarrays include all the devices so called in Schena (ed.), DNA Microarrays: A Practical Approach (Practical Approach Series), Oxford University Press (1999); Nature Genet. 21(1)(suppl.):1-60 (1999); Schena (ed.), Microarray Biochip: Tools and Technology, Eaton Publishing Company/BioTechniques Books Division (2000). These microarrays include substrate-bound collections of plural nucleic acids in which the plurality of nucleic acids are disposed on a plurality of beads, rather than on a unitary planar substrate, as is described, inter alia, in Brenner et al., Proc. Natl. Acad. Sci. USA 97(4):1665-1670 (2000).

The term “mutated” when applied to nucleic acid molecules means that nucleotides in the nucleic acid sequence of the nucleic acid molecule may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. In a preferred embodiment, the nucleic acid molecule comprises the wild type nucleic acid sequence encoding a PSP or is a PSNA. The nucleic acid molecule may be mutated by any method known in the art including those mutagenesis techniques described infra.

The term “error-prone PCR” refers to a process for performing PCR under conditions where the copying fidelity of the DNA polymerase is low, such that a high rate of point mutations is obtained along the entire length of the PCR product. See, e.g., Leung et al., Technique 1: 11-15 (1989) and Caldwell et al., PCR Methods Applic. 2: 28-33 (1992).

The term “oligonucleotide-directed mutagenesis” refers to a process which enables the generation of site-specific mutations in any cloned DNA segment of interest. See, e.g., Reidhaar-Olson et al., Science 241: 53-57 (1988).

The term “assembly PCR” refers to a process which involves the assembly of a PCR product from a mixture of small DNA fragments. A large number of different PCR reactions occur in parallel in the same vial, with the products of one reaction priming the products of another reaction.

The term “sexual PCR mutagenesis” or “DNA shuffling” refers to a method of error-prone PCR coupled with forced homologous recombination between DNA molecules of different but highly related DNA sequence in vitro, caused by random fragmentation of the DNA molecule based on sequence similarity, followed by fixation of the crossover by primer extension in an error-prone PCR reaction. See, e.g., Stemmer, Proc. Natl. Acad. Sci. U.S.A. 91: 10747-10751 (1994). DNA shuffling can be carried out between several related genes (“Family shuffling”).

The term “in vivo mutagenesis” refers to a process of generating random mutations in any cloned DNA of interest which involves the propagation of the DNA in a strain of bacteria such as E. coli that carries mutations in one or more of the DNA repair pathways. These “mutator” strains have a higher random mutation rate than that of a wild-type parent. Propagating the DNA in a mutator strain will eventually generate random mutations within the DNA.

The term “cassette mutagenesis” refers to any process for replacing a small region of a double-stranded DNA molecule with a synthetic oligonucleotide “cassette” that differs from the native sequence. The oligonucleotide often contains completely and/or partially randomized native sequence.

The term “recursive ensemble mutagenesis” refers to an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. See, e.g., Arkin et al, Proc. Natl. Acad. Sci. U.S.A. 89: 7811-7815 (1992).

The term “exponential ensemble mutagenesis” refers to a process for generating combinatorial libraries with a high percentage of unique and functional mutants, wherein small groups of residues are randomized in parallel to identify, at each altered position, amino acids which lead to functional proteins. See, e.g., Delegrave et al., Biotechnology Research 11: 1548-1552 (1993); Arnold, Current Opinion in Biotechnology 4: 450-455 (1993). Each of the references mentioned above are hereby incorporated by reference in its entirety.

“Operatively linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include the promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “vector,” as used herein, is intended to refer to a nucleic acid molecule capable of transporting another nucleic acid to which it has been linked. One type of vector is a “plasmid”, which refers to a circular double-stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome. Viral vectors that infect bacterial cells are referred to as bacteriophages. Certain vectors are capable of autonomous replication in a host cell into which they are introduced (e.g., bacterial vectors having a bacterial origin of replication). Other vectors can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include other forms of expression vectors that serve equivalent functions.

The term “recombinant host cell” (or simply “host cell”), as used herein, is intended to refer to a cell into which an expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the phrase “open reading frame” and the equivalent acronym “ORF” refer to that portion of a transcript-derived nucleic acid that can be translated in its entirety into a sequence of contiguous amino acids. As so defined, an ORF has length, measured in nucleotides, exactly divisible by 3. As so defined, an ORF need not encode the entirety of a natural protein.

As used herein, the phrase “ORF-encoded peptide” refers to the predicted or actual translation of an ORF.

As used herein, the phrase “degenerate variant” of a reference nucleic acid sequence intends all nucleic acid sequences that can be directly translated, using the standard genetic code, to provide an amino acid sequence identical to that translated from the reference nucleic acid sequence.

The term “polypeptide” encompasses both naturally-occurring and non-naturally-occurring proteins and polypeptides, polypeptide fragments and polypeptide mutants, derivatives and analogs. A polypeptide may be monomeric or polymeric. Further, a polypeptide may comprise a number of different modules within a single polypeptide each of which has one or more distinct activities. A preferred polypeptide in accordance with the invention comprises a PSP encoded by a nucleic acid molecule of the instant invention, as well as a fragment, mutant, analog and derivative thereof.

The term “isolated protein” or “isolated polypeptide” is a protein or polypeptide that by virtue of its origin or source of derivation (1) is not associated with naturally associated components that accompany it in its native state, (2) is free of other proteins from the same species (3) is expressed by a cell from a different species, or (4) does not occur in nature. Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well-known in the art.

A protein or polypeptide is “substantially pure,” “substantially homogeneous” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well-known in the art, such as polyacrylamide gel electrophoresis of a protein sample, followed by visualizing a single polypeptide band upon staining the gel with a stain well-known in the art. For certain purposes, higher resolution may be provided by using HPLC or other means well-known in the art for purification.

The term “polypeptide fragment” as used herein refers to a polypeptide of the instant invention that has an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “derivative” refers to polypeptides or fragments thereof that are substantially similar in primary structural sequence but which include, e.g., in vivo or in vitro chemical and biochemical modifications that are not found in the native polypeptide. Such modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Other modification include, e.g., labeling with radionuclides, and various enzymatic modifications, as will be readily appreciated by those skilled in the art. A variety of methods for labeling polypeptides and of substituents or labels useful for such purposes are well-known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well-known in the art. See Ausubel (1992), supra; Ausubel (1999), supra, herein incorporated by reference. The term “fusion protein” refers to polypeptides of the instant invention comprising polypeptides or fragments coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

The term “analog” refers to both polypeptide analogs and non-peptide analogs. The term “polypeptide analog” as used herein refers to a polypeptide of the instant invention that is comprised of a segment of at least 25 amino acids that has substantial identity to a portion of an amino acid sequence but which contains non-natural amino acids or non-natural inter-residue bonds. In a preferred embodiment, the analog has the same or similar biological activity as the native polypeptide. Typically, polypeptide analogs comprise a conservative amino acid substitution (or insertion or deletion) with respect to the naturally-occurring sequence. Analogs typically are at least 20 amino acids long, preferably at least 50 amino acids long or longer, and can often be as long as a full-length naturally-occurring polypeptide.

The term “non-peptide analog” refers to a compound with properties that are analogous to those of a reference polypeptide of the instant invention. A non-peptide compound may also be termed a “peptide mimetic” or a “peptidomimetic.” Such compounds are often developed with the aid of computerized molecular modeling. Peptide mimetics that are structurally similar to useful peptides may be used to produce an equivalent effect. Generally, peptidomimetics are structurally similar to a paradigm polypeptide (i.e., a polypeptide that has a desired biochemical property or pharmacological activity), but have one or more peptide linkages optionally replaced by a linkage selected from the group consisting of:—CH₂NH—, —CH₂S—, —CH₂—CH₂—, —CH═CH—(cis and trans), —COCH₂—, —CH(OH)CH₂—, and —CH₂SO—, by methods well-known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo et al., Ann. Rev. Biochem. 61:387-418 (1992), incorporated herein by reference). For example, one may add internal cysteine residues capable of forming intramolecular disulfide bridges which cyclize the peptide.

A “polypeptide mutant” or “mutein” refers to a polypeptide of the instant invention whose sequence contains substitutions, insertions or deletions of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. Further, a mutein may have the same or different biological activity as the naturally-occurring protein. For instance, a mutein may have an increased or decreased biological activity. A mutein has at least 50% sequence similarity to the wild type protein, preferred is 60% sequence similarity, more preferred is 70% sequence similarity. Even more preferred are muteins having 80%, 85% or 90% sequence similarity to the wild type protein. In an even more preferred embodiment, a mutein exhibits 95% sequence identity, even more preferably 97%, even more preferably 98% and even more preferably 99%. Sequence similarity may be measured by any common sequence analysis algorithm, such as Gap or Bestfit.

Preferred amino acid substitutions are those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs. For example, single or multiple amino acid substitutions (preferably conservative amino acid substitutions) may be made in the naturally-occurring sequence (preferably in the portion of the polypeptide outside the domain(s) forming intermolecular contacts. In a preferred embodiment, the amino acid substitutions are moderately conservative substitutions or conservative substitutions. In a more preferred embodiment, the amino acid substitutions are conservative substitutions. A conservative amino acid substitution should not substantially change the structural characteristics of the parent sequence (e.g., a replacement amino acid should not tend to disrupt a helix that occurs in the parent sequence, or disrupt other types of secondary structure that characterizes the parent sequence). Examples of art-recognized polypeptide secondary and tertiary structures are described in Creighton (ed.), Proteins Structures and Molecular Principles, W. H. Freeman and Company (1984); Branden et al. (ed.), Introduction to Protein Structure, Garland Publishing (1991); Thornton et al., Nature 354:105-106 (1991), each of which are incorporated herein by reference.

As used herein, the twenty conventional amino acids and their abbreviations follow conventional usage. See Golub et al. (eds.), Immunology—A Synthesis 2^{nd} Ed., Sinauer Associates (1991), which is incorporated herein by reference. Stereoisomers (e.g., D-amino acids) of the twenty conventional amino acids, unnatural amino acids such as —, -disubstituted amino acids, N-alkyl amino acids, and other unconventional amino acids may also be suitable components for polypeptides of the present invention. Examples of unconventional amino acids include: 4-hydroxyproline, γ-carboxyglutamate, -N,N,N-trimethyllysine, -N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the polypeptide notation used herein, the lefthand direction is the amino terminal direction and the right hand direction is the carboxy-terminal direction, in accordance with standard usage and convention.

A protein has “homology” or is “homologous” to a protein from another organism if the encoded amino acid sequence of the protein has a similar sequence to the encoded amino acid sequence of a protein of a different organism and has a similar biological activity or function. Alternatively, a protein may have homology or be homologous to another protein if the two proteins have similar amino acid sequences and have similar biological activities or functions. Although two proteins are said to be “homologous,” this does not imply that there is necessarily an evolutionary relationship between the proteins. Instead, the term “homologous” is defined to mean that the two proteins have similar amino acid sequences and similar biological activities or functions. In a preferred embodiment, a homologous protein is one that exhibits 50% sequence similarity to the wild type protein, preferred is 60% sequence similarity, more preferred is 70% sequence similarity. Even more preferred are homologous proteins that exhibit 80%, 85% or 90% sequence similarity to the wild type protein. In a yet more preferred embodiment, a homologous protein exhibits 95%, 97%, 98% or 99% sequence similarity.

When “sequence similarity” is used in reference to proteins or peptides, it is recognized that residue positions that are not identical often differ by conservative amino acid substitutions. In a preferred embodiment, a polypeptide that has “sequence similarity” comprises conservative or moderately conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol. 24: 307-31 (1994), herein incorporated by reference.

For instance, the following six groups each contain amino acids that are conservative substitutions for one another:

1) Serine (S), Threonine (T);

2) Aspartic Acid (D), Glutamic Acid (E);

3) Asparagine (N), Glutamine (O);

4) Arginine (R), Lysine (K);

5) Isoleucine (I), Leucine (L), Methionine (M), Alanine (A), Valine (V), and

6) Phenylalanine (F), Tyrosine (Y), Tryptophan (W).

Alternatively, a conservative replacement is any change having a positive value in the PAM250 log-likelihood matrix disclosed in Gonnet et al., Science 256: 1443-45 (1992), herein incorporated by reference. A “moderately conservative” replacement is any change having a normegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Other programs include FASTA, discussed supra.

A preferred algorithm when comparing a sequence of the invention to a database containing a large number of sequences from different organisms is the computer program BLAST, especially blastp or tblastn. See, e.g., Altschul et al., J. Mol. Biol. 215: 403-410 (1990); Altschul et al., Nucleic Acids Res. 25:3389-402 (1997); herein incorporated by reference. Preferred parameters for blastp are:

| | |
|---|---|
| Expectation value: | 10 (default) |
| Filter: | seg (default) |
| Cost to open a gap: | 11 (default) |
| Cost to extend a gap: | 1 (default |
| Max. alignments: | 100 (default) |
| Word size: | 11 (default) |
| No. of descriptions: | 100 (default) |
| Penalty Matrix: | BLOSUM62 |

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

Database searching using amino acid sequences can be measured by algorithms other than blastp are known in the art. For instance, polypeptide sequences can be compared using FASTA, a program in GCG Version 6.1. FASTA (e.g., FASTA2 and FASTA3) provides alignments and percent sequence identity of the regions of the best overlap between the query and search sequences (Pearson (1990), supra; Pearson (2000), supra. For example, percent sequence identity between amino acid sequences can be determined using FASTA with its default or recommended parameters (a word size of 2 and the PAM250 scoring matrix), as provided in GCG Version 6.1, herein incorporated by reference.

An “antibody” refers to an intact immunoglobulin, or to an antigen-binding portion thereof that competes with the intact antibody for specific binding to a molecular species, e.g., a polypeptide of the instant invention. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. An Fab fragment is a monovalent fragment consisting of the VL, VH, CL and CH1 domains; an F(ab′)₂ fragment is a bivalent fragment comprising two Fab fragments linked by a disulfide bridge at the hinge region; an Fd fragment consists of the VH and CH1 domains; an Fv fragment consists of the VL and VH domains of a single arm of an antibody; and a dAb fragment consists of a VH domain. See, e.g., Ward et al., Nature 341: 544-546 (1989).

By “bind specifically” and “specific binding” is here intended the ability of the antibody to bind to a first molecular species in preference to binding to other molecular species with which the antibody and first molecular species are admixed. An antibody is said specifically to “recognize” a first molecular species when it can bind specifically to that first molecular species.

A single-chain antibody (scFv) is an antibody in which a VL and VH region are paired to form a monovalent molecule via a synthetic linker that enables them to be made as a single protein chain. See, e.g., Bird et al., Science 242: 423-426 (1988); Huston et al., Proc. Natl. Acad. Sci. USA 85: 5879-5883 (1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites. See e.g., Holliger et al., Proc. Natl. Acad. Sci. USA 90: 6444-6448 (1993); Poljak et al., Structure 2: 1121-1123 (1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest. A chimeric antibody is an antibody that contains one or more regions from one antibody and one or more regions from one or more other antibodies.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature. It is known that purified proteins, including purified antibodies, may be stabilized with non-naturally-associated components. The non-naturally-associated component may be a protein, such as albumin (e.g., BSA) or a chemical such as polyethylene glycol (PEG).

A “neutralizing antibody” or “an inhibitory antibody” is an antibody that inhibits the activity of a polypeptide or blocks the binding of a polypeptide to a ligand that normally binds to it. An “activating antibody” is an antibody that increases the activity of a polypeptide.

The term “epitope” includes any protein determinant capable of specifically binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is less than 1M, preferably less than 100 nM and most preferably less than 10 nM.

The term “patient” as used herein includes human and veterinary subjects.

Throughout this specification and claims, the word “comprise,” or variations such as “comprises” or “comprising,” will be understood to imply the inclusion of a stated integer or group of integers but not the exclusion of any other integer or group of integers.

The term “prostate specific” refers to a nucleic acid molecule or polypeptide that is expressed predominantly in the prostate as compared to other tissues in the body. In a preferred embodiment, a “prostate specific” nucleic acid molecule or polypeptide is expressed at a level that is 5-fold higher than any other tissue in the body. In a more preferred embodiment, the “prostate specific” nucleic acid molecule or polypeptide is expressed at a level that is 10-fold higher than any other tissue in the body, more preferably at least 15-fold, 20-fold, 25-fold, 50-fold or 100-fold higher than any other tissue in the body. Nucleic acid molecule levels may be measured by nucleic acid hybridization, such as Northern blot hybridization, or quantitative PCR. Polypeptide levels may be measured by any method known to accurately quantitate protein levels, such as Western blot analysis.

Nucleic Acid Molecules Regulatory Sequences, Vectors, Host Cells and Recombinant Methods of Making Polypeptides

Nucleic Acid Molecules

One aspect of the invention provides isolated nucleic acid molecules that are specific to the prostate or to prostate cells or tissue or that are derived from such nucleic acid molecules. These isolated prostate specific nucleic acids (PSNAs) may comprise a cDNA, a genomic DNA, RNA, or a fragment of one of these nucleic acids, or may be a non-naturally-occurring nucleic acid molecule. In a preferred embodiment, the nucleic acid molecule encodes a polypeptide that is specific to prostate, a prostate-specific polypeptide (PSP). In a more preferred embodiment, the nucleic acid molecule encodes a polypeptide that comprises an amino acid sequence of SEQ ID NO: 136 through 240. In another highly preferred embodiment, the nucleic acid molecule comprises a nucleic acid sequence of SEQ ID NO: 1 through 135.

A PSNA may be derived from a human or from another animal. In a preferred embodiment, the PSNA is derived from a human or other mammal. In a more preferred embodiment, the PSNA is derived from a human or other primate. In an even more preferred embodiment, the PSNA is derived from a human.

By “nucleic acid molecule” for purposes of the present invention, it is also meant to be inclusive of nucleic acid sequences that selectively hybridize to a nucleic acid molecule encoding a PSNA or a complement thereof. The hybridizing nucleic acid molecule may or may not encode a polypeptide or may not encode a PSP. However, in a preferred embodiment, the hybridizing nucleic acid molecule encodes a PSP. In a more preferred embodiment, the invention provides a nucleic acid molecule that selectively hybridizes to a nucleic acid molecule that encodes a polypeptide comprising an amino acid sequence of SEQ ID NO: 136 through 240. In an even more preferred embodiment, the invention provides a nucleic acid molecule that selectively hybridizes to a nucleic acid molecule comprising the nucleic acid sequence of SEQ ID NO: 1 through 135.

In a preferred embodiment, the nucleic acid molecule selectively hybridizes to a nucleic acid molecule encoding a PSP under low stringency conditions. In a more preferred embodiment, the nucleic acid molecule selectively hybridizes to a nucleic acid molecule encoding a PSP under moderate stringency conditions. In a more preferred embodiment, the nucleic acid molecule selectively hybridizes to a nucleic acid molecule encoding a PSP under high stringency conditions. In an even more preferred embodiment, the nucleic acid molecule hybridizes under low, moderate or high stringency conditions to a nucleic acid molecule encoding a polypeptide comprising an amino acid sequence of SEQ ID NO: 136 through 240. In a yet more preferred embodiment, the nucleic acid molecule hybridizes under low, moderate or high stringency conditions to a nucleic acid molecule comprising a nucleic acid sequence selected from SEQ ID NO: 1 through 135. In a preferred embodiment of the invention, the hybridizing nucleic acid molecule may be used to express recombinantly a polypeptide of the invention.

By “nucleic acid molecule” as used herein it is also meant to be inclusive of sequences that exhibits substantial sequence similarity to a nucleic acid encoding a PSP or a complement of the encoding nucleic acid molecule. In a preferred embodiment, the nucleic acid molecule exhibits substantial sequence similarity to a nucleic acid molecule encoding human PSP. In a more preferred embodiment, the nucleic acid molecule exhibits substantial sequence similarity to a nucleic acid molecule encoding a polypeptide having an amino acid sequence of SEQ ID NO: 136 through 240. In a preferred embodiment, the similar nucleic acid molecule is one that has at least 60% sequence identity with a nucleic acid molecule encoding a PSP, such as a polypeptide having an amino acid sequence of SEQ ID NO: 136 through 240, more preferably at least 70%, even more preferably at least 80% and even more preferably at least 85%. In a more preferred embodiment, the similar nucleic acid molecule is one that has at least 90% sequence identity with a nucleic acid molecule encoding a PSP, more preferably at least 95%, more preferably at least 97%, even more preferably at least 98%, and still more preferably at least 99%. In another highly preferred embodiment, the nucleic acid molecule is one that has at least 99.5%, 99.6%, 99.7%, 99.8% or 99.9% sequence identity with a nucleic acid molecule encoding a PSP.

In another preferred embodiment, the nucleic acid molecule exhibits substantial sequence similarity to a PSNA or its complement. In a more preferred embodiment, the nucleic acid molecule exhibits substantial sequence similarity to a nucleic acid molecule comprising a nucleic acid sequence of SEQ ID NO: 1 through 135. In a preferred embodiment, the nucleic acid molecule is one that has at least 60% sequence identity with a PSNA, such as one having a nucleic acid sequence of SEQ ID NO: 1 through 135, more preferably at least 70%, even more preferably at least 80% and even more preferably at least 85%. In a more preferred embodiment, the nucleic acid molecule is one that has at least 90% sequence identity with a PSNA, more preferably at least 95%, more preferably at least 97%, even more preferably at least 98%, and still more preferably at least 99%. In another highly preferred embodiment, the nucleic acid molecule is one that has at least 99.5%, 99.6%, 99.7%, 99.8% or 99.9% sequence identity with a PSNA.

A nucleic acid molecule that exhibits substantial sequence similarity may be one that exhibits sequence identity over its entire length to a PSNA or to a nucleic acid molecule encoding a PSP, or may be one that is similar over only a part of its length. In this case, the part is at least 50 nucleotides of the PSNA or the nucleic acid molecule encoding a PSP, preferably at least 100 nucleotides, more preferably at least 150 or 200 nucleotides, even more preferably at least 250 or 300 nucleotides, still more preferably at least 400 or 500 nucleotides.

The substantially similar nucleic acid molecule may be a naturally-occurring one that is derived from another species, especially one derived from another primate, wherein the similar nucleic acid molecule encodes an amino acid sequence that exhibits significant sequence identity to that of SEQ ID NO: 136 through 240 or demonstrates significant sequence identity to the nucleotide sequence of SEQ ID NO: 1 through 135. The similar nucleic acid molecule may also be a naturally-occurring nucleic acid molecule from a human, when the PSNA is a member of a gene family. The similar nucleic acid molecule may also be a naturally-occurring nucleic acid molecule derived from a non-primate, mammalian species, including without limitation, domesticated species, e.g., dog, cat, mouse, rat, rabbit, hamster, cow, horse and pig; and wild animals, e.g., monkey, fox, lions, tigers, bears, giraffes, zebras, etc. The substantially similar nucleic acid molecule may also be a naturally-occurring nucleic acid molecule derived from a non-mammalian species, such as birds or reptiles. The naturally-occurring substantially similar nucleic acid molecule may be isolated directly from humans or other species. In another embodiment, the substantially similar nucleic acid molecule may be one that is experimentally produced by random mutation of a nucleic acid molecule. In another embodiment, the substantially similar nucleic acid molecule may be one that is experimentally produced by directed mutation of a PSNA. Further, the substantially similar nucleic acid molecule may or may not be a PSNA. However, in a preferred embodiment, the substantially similar nucleic acid molecule is a PSNA.

By “nucleic acid molecule” it is also meant to be inclusive of allelic variants of a PSNA or a nucleic acid encoding a PSP. For instance, single nucleotide polymorphisms (SNPs) occur frequently in eukaryotic genomes. In fact, more than 1.4 million SNPs have already identified in the human genome, International Human Genome Sequencing Consortium, Nature 409: 860-921 (2001). Thus, the sequence determined from one individual of a species may differ from other allelic forms present within the population. Additionally, small deletions and insertions, rather than single nucleotide polymorphisms, are not uncommon in the general population, and often do not alter the function of the protein. Further, amino acid substitutions occur frequently among natural allelic variants, and often do not substantially change protein function.

In a preferred embodiment, the nucleic acid molecule comprising an allelic variant is a variant of a gene, wherein the gene is transcribed into an mRNA that encodes a PSP. In a more preferred embodiment, the gene is transcribed into an mRNA that encodes a PSP comprising an amino acid sequence of SEQ ID NO: 136 through 240. In another preferred embodiment, the allelic variant is a variant of a gene, wherein the gene is transcribed into an mRNA that is a PSNA. In a more preferred embodiment, the gene is transcribed into an mRNA that comprises the nucleic acid sequence of SEQ ID NO: 1 through 135. In a preferred embodiment, the allelic variant is a naturally-occurring allelic variant in the species of interest. In a more preferred embodiment, the species of interest is human.

By “nucleic acid molecule” it is also meant to be inclusive of a part of a nucleic acid sequence of the instant invention. The part may or may not encode a polypeptide, and may or may not encode a polypeptide that is a PSP. However, in a preferred embodiment, the part encodes a PSP. In one aspect, the invention comprises a part of a PSNA. In a second aspect, the invention comprises a part of a nucleic acid molecule that hybridizes or exhibits substantial sequence similarity to a PSNA. In a third aspect, the invention comprises a part of a nucleic acid molecule that is an allelic variant of a PSNA. In a fourth aspect, the invention comprises a part of a nucleic acid molecule that encodes a PSP. A part comprises at least 10 nucleotides, more preferably at least 15, 17, 18, 20, 25, 30, 35, 40, 50, 60, 70, 80, 90, 100, 150, 200, 250, 300, 350, 400 or 500 nucleotides. The maximum size of a nucleic acid part is one nucleotide shorter than the sequence of the nucleic acid molecule encoding the full-length protein.

By “nucleic acid molecule” it is also meant to be inclusive of sequence that encoding a fusion protein, a homologous protein, a polypeptide fragment, a mutein or a polypeptide analog, as described below.

Nucleotide sequences of the instantly-described nucleic acids were determined by sequencing a DNA molecule that had resulted, directly or indirectly, from at least one enzymatic polymerization reaction (e.g., reverse transcription and/or polymerase chain reaction) using an automated sequencer (such as the MegaBACE™ 1000, Molecular Dynamics, Sunnyvale, Calif., USA). Further, all amino acid sequences of the polypeptides of the present invention were predicted by translation from the nucleic acid sequences so determined, unless otherwise specified.

In a preferred embodiment of the invention, the nucleic acid molecule contains modifications of the native nucleic acid molecule. These modifications include normative internucleoside bonds, post-synthetic modifications or altered nucleotide analogues. One having ordinary skill in the art would recognize that the type of modification that can be made will depend upon the intended use of the nucleic acid molecule. For instance, when the nucleic acid molecule is used as a hybridization probe, the range of such modifications will be limited to those that permit sequence-discriminating base pairing of the resulting nucleic acid. When used to direct expression of RNA or protein in vitro or in vivo, the range of such modifications will be limited to those that permit the nucleic acid to function properly as a polymerization substrate. When the isolated nucleic acid is used as a therapeutic agent, the modifications will be limited to those that do not confer toxicity upon the isolated nucleic acid.

In a preferred embodiment, isolated nucleic acid molecules can include nucleotide analogues that incorporate labels that are directly detectable, such as radiolabels or fluorophores, or nucleotide analogues that incorporate labels that can be visualized in a subsequent reaction, such as biotin or various haptens. In a more preferred embodiment, the labeled nucleic acid molecule may be used as a hybridization probe.

Common radiolabeled analogues include those labeled with ³³P, ³²P, and ³⁵S, such as -³² P-dATP, -³²P-dCTP, -³²P-dGTP, -³²P-dTTP, -³²P-3'dATP, -³²P-ATP, -³²P-CTP, -³²P-GTP, -³²P-UTP, -³⁵S-dATP, α-³⁵S-GTP, α-³³P-dATP, and the like.

Commercially available fluorescent nucleotide analogues readily incorporated into the nucleic acids of the present invention include Cy3-dCTP, Cy3-dUTP, Cy5-dCTP, Cy3-dUTP (Amersham Pharmacia Biotech, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY(G TMR-14-dUTP, BODIPY® TR-14-dUTP, Rhodamine Green™-5-dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor® 546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodamine Green™-5-UTP, Alexa Fluor® 488-5-UTP, Alexa Fluor® 546-14-UTP (Molecular Probes, Inc. Eugene, Oreg., USA). One may also custom synthesize nucleotides having other fluorophores. See Henegariu et al., Nature Biotechnol. 18: 345-348 (2000), the disclosure of which is incorporated herein by reference in its entirety.

Haptens that are commonly conjugated to nucleotides for subsequent labeling include biotin (biotin-1 1-dUTP, Molecular Probes, Inc., Eugene, Oreg., USA; biotin-21-UTP, biotin-21-dUTP, Clontech Laboratories, Inc., Palo Alto, Calif., USA), digoxigenin (DIG-11-dUTP, alkali labile, DIG-11-UTP, Roche Diagnostics Corp., Indianapolis, Ind., USA), and dinitrophenyl (dinitrophenyl-11-dUTP, Molecular Probes, Inc., Eugene, Oreg., USA).

Nucleic acid molecules can be labeled by incorporation of labeled nucleotide analogues into the nucleic acid. Such analogues can be incorporated by enzymatic polymerization, such as by nick translation, random priming, polymerase chain reaction (PCR), terminal transferase tailing, and end-filling of overhangs, for DNA molecules, and in vitro transcription driven, e.g., from phage promoters, such as T7, T3, and SP6, for RNA molecules. Commercial kits are readily available for each such labeling approach. Analogues can also be incorporated during automated solid phase chemical synthesis. Labels can also be incorporated after nucleic acid synthesis, with the 5′ phosphate and 3′ hydroxyl providing convenient sites for post-synthetic covalent attachment of detectable labels.

Other post-synthetic approaches also permit internal labeling of nucleic acids. For example, fluorophores can be attached using a cisplatin reagent that reacts with the N7 of guanine residues (and, to a lesser extent, adenine bases) in DNA, RNA, and PNA to provide a stable coordination complex between the nucleic acid and fluorophore label (Universal Linkage System) (available from Molecular Probes, Inc., Eugene, Oreg., USA and Amersham Pharmacia Biotech, Piscataway, N.J., USA); see Alers et al., Genes, Chromosomes & Cancer 25: 301-305 (1999); Jelsma et al., J. NIH Res. 5: 82 (1994); Van Belkum et al., BioTechniques 16: 148-153 (1994), incorporated herein by reference. As another example, nucleic acids can be labeled using a disulfide-containing linker (FastTag™ Reagent, Vector Laboratories, Inc., Burlingame, Calif., USA) that is photo- or thermally-coupled to the target nucleic acid using aryl azide chemistry; after reduction, a free thiol is available for coupling to a hapten, fluorophore, sugar, affinity ligand, or other marker.

One or more independent or interacting labels can be incorporated into the nucleic acid molecules of the present invention. For example, both a fluorophore and a moiety that in proximity thereto acts to quench fluorescence can be included to report specific hybridization through release of fluorescence quenching or to report exonucleotidic excision. See, e.g., Tyagi et al., Nature Biotechnol. 14: 303-308 (1996); Tyagi et al., Nature Biotechnol. 16: 49-53 (1998); Sokol et al., Proc. Natl. Acad. Sci. USA 95: 11538-11543 (1998); Kostrikis et al., Science 279: 1228-1229 (1998); Marras et al., Genet. Anal. 14: 151-156 (1999); U.S. Pat. No. 5,846,726; 5,925,517; 5,925,517; 5,723,591 and 5,538,848; Holland et al., Proc. Natl. Acad. Sci. USA 88: 7276-7280 (1991); Heid et al., Genome Res. 6(10): 986-94 (1996); Kuimelis et al., Nucleic Acids Symp. Ser. (37): 255-6 (1997); the disclosures of which are incorporated herein by reference in their entireties.

Nucleic acid molecules of the invention may be modified by altering one or more native phosphodiester internucleoside bonds to more nuclease-resistant, internucleoside bonds. See Hartmann et al. (eds.), Manual of Antisense Methodology: Perspectives in Antisense Science, Kluwer Law International (1999); Stein et al. (eds.), Applied Antisense Oligonucleotide Technology, Wiley-Liss (1998); Chadwick et al. (eds.), Oligonucleotides as Therapeutic Agents—Symposium No. 209, John Wiley & Son Ltd (1997); the disclosures of which are incorporated herein by reference in their entireties. Such altered intemucleoside bonds are often desired for antisense techniques or for targeted gene correction. See Gamper et al., Nucl. Acids Res. 28(21): 4332-4339 (2000), the disclosure of which is incorporated herein by reference in its entirety.

Modified oligonucleotide backbones include, without limitation, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties. In a preferred embodiment, the modified intemucleoside linkages may be used for antisense techniques.

Other modified oligonucleotide backbones do not include a phosphorus atom, but have backbones that are formed by short chain alkyl or cycloalkyl intemucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. No. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437 and 5,677,439; the disclosures of which are incorporated herein by reference in their entireties.

In other preferred oligonucleotide mimetics, both the sugar and the intemucleoside linkage are replaced with novel groups, such as peptide nucleic acids (PNA). In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza nitrogen atoms of the amide portion of the backbone, typically by methylene carbonyl linkages. PNA can be synthesized using a modified peptide synthesis protocol. PNA oligomers can be synthesized by both Fmoc and tBoc methods. Representative U.S. patents that teach the preparation of PNA compounds include, but are not limited to, U.S. Pat. Nos. 5,539,082; 5,714,33 1; and 5,719,262, each of which is herein incorporated by reference. Automated PNA synthesis is readily achievable on commercial synthesizers (see, e.g., “PNA User's Guide,” Rev. 2, February 1998, Perseptive Biosystems Part No. 60138, Applied Biosystems, Inc., Foster City, Calif.).

PNA molecules are advantageous for a number of reasons. First, because the PNA backbone is uncharged, PNA/DNA and PNA/RNA duplexes have a higher thermal stability than is found in DNA/DNA and DNA/RNA duplexes. The Tm of a PNA/DNA or PNA/RNA duplex is generally 1° C. higher per base pair than the Tm of the corresponding DNA/DNA or DNA/RNA duplex (in 100 mM NaCl). Second, PNA molecules can also form stable PNA/DNA complexes at low ionic strength, under conditions in which DNA/DNA duplex formation does not occur. Third, PNA also demonstrates greater specificity in binding to complementary DNA because a PNA/DNA mismatch is more destabilizing than DNA/DNA mismatch. A single mismatch in mixed a PNA/DNA 15-mer lowers the Tm by 8-20° C. (15° C. on average). In the corresponding DNA/DNA duplexes, a single mismatch lowers the Tm by 4-16° C. (11° C. on average). Because PNA probes can be significantly shorter than DNA probes, their specificity is greater. Fourth, PNA oligomers are resistant to degradation by enzymes, and the lifetime of these compounds is extended both in vivo and in vitro because nucleases and proteases do not recognize the PNA polyamide backbone with nucleobase sidechains. See, e.g., Ray et al., FASEB J 14(9): 1041-60 (2000); Nielsen et al., Pharmacol Toxicol. 86(1): 3-7 (2000); Larsen et al., Biochim Biophys Acta. 1489(1): 159-66 (1999); Nielsen, Curr. Opin. Struct. Biol. 9(3): 353-7 (1999), and Nielsen, Curr. Opin. Biotechnol. 10(1): 71-5 (1999), the disclosures of which are incorporated herein by reference in their entireties.

Nucleic acid molecules may be modified compared to their native structure throughout the length of the nucleic acid molecule or can be localized to discrete portions thereof. As an example of the latter, chimeric nucleic acids can be synthesized that have discrete DNA and RNA domains and that can be used for targeted gene repair and modified PCR reactions, as further described in U.S. Pat. Nos. 5,760,012 and 5,731,181, Misra et al., Biochem. 37: 1917-1925 (1998); and Finn et al., Nucl. Acids Res. 24: 3357-3363 (1996), the disclosures of which are incorporated herein by reference in their entireties.

Unless otherwise specified, nucleic acids of the present invention can include any topological conformation appropriate to the desired use; the term thus explicitly comprehends, among others, single-stranded, double-stranded, triplexed, quadruplexed, partially double-stranded, partially-triplexed, partially-quadruplexed, branched, hairpinned, circular, and padlocked conformations. Padlock conformations and their utilities are further described in Banér et al., Curr. Opin. Biotechnol. 12: 11-15 (2001); Escude et al., Proc. Natl. Acad. Sci. USA 14: 96(19):10603-7 (1999); Nilsson et al., Science 265(5181): 2085-8 (1994), the disclosures of which are incorporated herein by reference in their entireties. Triplex and quadruplex conformations, and their utilities, are reviewed in Praseuth et al., Biochim. Biophys. Acta. 1489(1): 181-206 (1999); Fox, Curr. Med. Chem. 7(1): 17-37 (2000); Kochetkova et al., Methods Mol. Biol. 130: 189-201 (2000); Chan et al., J. Mol. Med. 75(4): 267-82 (1997), the disclosures of which are incorporated herein by reference in their entireties.

Methods for Using Nucleic Acid Molecules as Probes and Primers

The isolated nucleic acid molecules of the present invention can be used as hybridization probes to detect, characterize, and quantify hybridizing nucleic acids in, and isolate hybridizing nucleic acids from, both genomic and transcript-derived nucleic acid samples. When free in solution, such probes are typically, but not invariably, detectably labeled; bound to a substrate, as in a microarray, such probes are typically, but not invariably unlabeled.

In one embodiment, the isolated nucleic acids of the present invention can be used as probes to detect and characterize gross alterations in the gene of a PSNA, such as deletions, insertions, translocations, and duplications of the PSNA genomic locus through fluorescence in situ hybridization (FISH) to chromosome spreads. See, e.g., Andreeff et al. (eds.), Introduction to Fluorescence In Situ Hybridization: Principles and Clinical Applications, John Wiley & Sons (1999), the disclosure of which is incorporated herein by reference in its entirety. The isolated nucleic acids of the present invention can be used as probes to assess smaller genomic alterations using, e.g., Southern blot detection of restriction fragment length polymorphisms. The isolated nucleic acid molecules of the present invention can be used as probes to isolate genomic clones that include the nucleic acid molecules of the present invention, which thereafter can be restriction mapped and sequenced to identify deletions, insertions, translocations, and substitutions (single nucleotide polymorphisms, SNPs) at the sequence level.

In another embodiment, the isolated nucleic acid molecules of the present invention can be used as probes to detect, characterize, and quantify PSNA in, and isolate PSNA from, transcript-derived nucleic acid samples. In one aspect, the isolated nucleic acid molecules of the present invention can be used as hybridization probes to detect, characterize by length, and quantify mRNA by Northern blot of total or poly-A⁺-selected RNA samples. In another aspect, the isolated nucleic acid molecules of the present invention can be used as hybridization probes to detect, characterize by location, and quantify mRNA by in situ hybridization to tissue sections. See, e.g., Schwarchzacher et al., In Situ Hybridization, Springer-Verlag New York (2000), the disclosure of which is incorporated herein by reference in its entirety. In another preferred embodiment, the isolated nucleic acid molecules of the present invention can be used as hybridization probes to measure the representation of clones in a cDNA library or to isolate hybridizing nucleic acid molecules acids from cDNA libraries, permitting sequence level characterization of mRNAs that hybridize to PSNAs, including, without limitations, identification of deletions, insertions, substitutions, truncations, alternatively spliced forms and single nucleotide polymorphisms. In yet another preferred embodiment, the nucleic acid molecules of the instant invention may be used in microarrays.

All of the aforementioned probe techniques are well within the skill in the art, and are described at greater length in standard texts such as Sambrook (2001), supra; Ausubel (1999), supra; and Walker et al. (eds.), The Nucleic Acids Protocols Handbook, Humana Press (2000), the disclosures of which are incorporated herein by reference in their entirety.

Thus, in one embodiment, a nucleic acid molecule of the invention may be used as a probe or primer to identify or amplify a second nucleic acid molecule that selectively hybridizes to the nucleic acid molecule of the invention. In a preferred embodiment, the probe or primer is derived from a nucleic acid molecule encoding a PSP. In a more preferred embodiment, the probe or primer is derived from a nucleic acid molecule encoding a polypeptide having an amino acid sequence of SEQ ID NO: 136 through 240. In another preferred embodiment, the probe or primer is derived from a PSNA. In a more preferred embodiment, the probe or primer is derived from a nucleic acid molecule having a nucleotide sequence of SEQ ID NO: 1 through 135.

In general, a probe or primer is at least 10 nucleotides in length, more preferably at least 12, more preferably at least 14 and even more preferably at least 16 or 17 nucleotides in length. In an even more preferred embodiment, the probe or primer is at least 18 nucleotides in length, even more preferably at least 20 nucleotides and even more preferably at least 22 nucleotides in length. Primers and probes may also be longer in length. For instance, a probe or primer may be 25 nucleotides in length, or may be 30, 40 or 50 nucleotides in length. Methods of performing nucleic acid hybridization using oligonucleotide probes are well-known in the art. See, e.g., Sambrook et al., 1989, supra, Chapter 11 and pp. 11.31-11.32 and 11.40-11.44, which describes radiolabeling of short probes, and pp. 11.45-11.53, which describe hybridization conditions for oligonucleotide probes, including specific conditions for probe hybridization (pp. 11.50-11.51).

Methods of performing primer-directed amplification are also well-known in the art. Methods for performing the polymerase chain reaction (PCR) are compiled, inter alia, in McPherson, PCR Basics: From Background to Bench, Springer Verlag (2000); Innis et al. (eds.), PCR Applications: Protocols for Functional Genomics, Academic Press (1999); Gelfand et al. (eds.), PCR Strategies, Academic Press (1998); Newton et al., PCR, Springer-Verlag New York (1997); Burke (ed.), PCR: Essential Techniques, John Wiley & Son Ltd (1996); White (ed.), PCR Cloning Protocols: From Molecular Cloning to Genetic Engineering, Vol. 67, Humana Press (1996); McPherson et al. (eds.), PCR 2: A Practical Approach, Oxford University Press, Inc. (1995); the disclosures of which are incorporated herein by reference in their entireties. Methods for performing RT-PCR are collected, e.g., in Siebert et al. (eds.), Gene Cloning and Analysis by RT-PCR, Eaton Publishing Company/Bio Techniques Books Division, 1998; Siebert (ed.), PCR Technique:RT-PCR, Eaton Publishing Company/BioTechniques Books (1995); the disclosure of which is incorporated herein by reference in its entirety.

PCR and hybridization methods may be used to identify and/or isolate allelic variants, homologous nucleic acid molecules and fragments of the nucleic acid molecules of the invention. PCR and hybridization methods may also be used to identify, amplify and/or isolate nucleic acid molecules that encode homologous proteins, analogs, fusion protein or muteins of the invention. The nucleic acid primers of the present invention can be used to prime amplification of nucleic acid molecules of the invention, using transcript-derived or genomic DNA as template.

The nucleic acid primers of the present invention can also be used, for example, to prime single base extension (SBE) for SNP detection (See, e.g., U.S. Pat. No. 6,004,744, the disclosure of which is incorporated herein by reference in its entirety).

Isothermal amplification approaches, such as rolling circle amplification, are also now well-described. See, e.g., Schweitzer et al., Curr. Opin. Biotechnol. 12(1): 21-7 (2001); U.S. Pat. Nos. 5,854,033 and 5,714,320; and international patent publications WO 97/19193 and WO 00/15779, the disclosures of which are incorporated herein by reference in their entireties. Rolling circle amplification can be combined with other techniques to facilitate SNP detection. See, e.g., Lizardi et al., Nature Genet. 19(3): 225-32 (1998).

Nucleic acid molecules of the present invention may be bound to a substrate either covalently or noncovalently. The substrate can be porous or solid, planar or non-planar, unitary or distributed. The bound nucleic acid molecules may be used as hybridization probes, and may be labeled or unlabeled. In a preferred embodiment, the bound nucleic acid molecules are unlabeled.

In one embodiment, the nucleic acid molecule of the present invention is bound to a porous substrate, e.g., a membrane, typically comprising nitrocellulose, nylon, or positively-charged derivatized nylon. The nucleic acid molecule of the present invention can be used to detect a hybridizing nucleic acid molecule that is present within a labeled nucleic acid sample, e.g., a sample of transcript-derived nucleic acids. In another embodiment, the nucleic acid molecule is bound to a solid substrate, including, without limitation, glass, amorphous silicon, crystalline silicon or plastics. Examples of plastics include, without limitation, polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof. The solid substrate may be any shape, including rectangular, disk-like and spherical. In a preferred embodiment, the solid substrate is a microscope slide or slide-shaped substrate.

The nucleic acid molecule of the present invention can be attached covalently to a surface of the support substrate or applied to a derivatized surface in a chaotropic agent that facilitates denaturation and adherence by presumed noncovalent interactions, or some combination thereof. The nucleic acid molecule of the present invention can be bound to a substrate to which a plurality of other nucleic acids are concurrently bound, hybridization to each of the plurality of bound nucleic acids being separately detectable. At low density, e.g. on a porous membrane, these substrate-bound collections are typically denominated macroarrays; at higher density, typically on a solid support, such as glass, these substrate bound collections of plural nucleic acids are colloquially termed microarrays. As used herein, the term microarray includes arrays of all densities. It is, therefore, another aspect of the invention to provide microarrays that include the nucleic acids of the present invention.

Expression Vectors, Host Cells and Recombinant Methods of Producing Polypeptides

Another aspect of the present invention relates to vectors that comprise one or more of the isolated nucleic acid molecules of the present invention, and host cells in which such vectors have been introduced.

The vectors can be used, inter alia, for propagating the nucleic acids of the present invention in host cells (cloning vectors), for shuttling the nucleic acids of the present invention between host cells derived from disparate organisms (shuttle vectors), for inserting the nucleic acids of the present invention into host cell chromosomes (insertion vectors), for expressing sense or antisense RNA transcripts of the nucleic acids of the present invention in vitro or within a host cell, and for expressing polypeptides encoded by the nucleic acids of the present invention, alone or as fusions to heterologous polypeptides (expression vectors). Vectors of the present invention will often be suitable for several such uses.

Vectors are by now well-known in the art, and are described, inter alia, in Jones et al. (eds.), Vectors: Cloning Applications: Essential Techniques (Essential Techniques Series), John Wiley & Son Ltd. (1998); Jones et al. (eds.), Vectors: Expression Systems: Essential Techniques (Essential Techniques Series), John Wiley & Son Ltd. (1998); Gacesa et al., Vectors: Essential Data, John Wiley & Sons Ltd. (1995); Cid-Arregui (eds.), Viral Vectors: Basic Science and Gene Therapy, Eaton Publishing Co. (2000); Sambrook (2001), supra; Ausubel (1999), supra; the disclosures of which are incorporated herein by reference in their entireties. Furthermore, an enormous variety of vectors are available commercially. Use of existing vectors and modifications thereof being well within the skill in the art, only basic features need be described here.

Nucleic acid sequences may be expressed by operatively linking them to an expression control sequence in an appropriate expression vector and employing that expression vector to transform an appropriate unicellular host. Expression control sequences are sequences which control the transcription, post-transcriptional events and translation of nucleic acid sequences. Such operative linking of a nucleic sequence of this invention to an expression control sequence, of course, includes, if not already part of the nucleic acid sequence, the provision of a translation initiation codon, ATG or GTG, in the correct reading frame upstream of the nucleic acid sequence.

A wide variety of host/expression vector combinations may be employed in expressing the nucleic acid sequences of this invention. Useful expression vectors, for example, may consist of segments of chromosomal, non-chromosomal and synthetic nucleic acid sequences.

In one embodiment, prokaryotic cells may be used with an appropriate vector. Prokaryotic host cells are often used for cloning and expression. In a preferred embodiment, prokaryotic host cells include E. coli, Pseudomonas, Bacillus and Streptomyces. In a preferred embodiment, bacterial host cells are used to express the nucleic acid molecules of the instant invention. Useful expression vectors for bacterial hosts include bacterial plasmids, such as those from E. coli, Bacillus or Streptomyces, including pBluescript, pGEX-2T, pUC vectors, col E1, pCR1, pBR322, pMB9 and their derivatives, wider host range plasmids, such as RP4, phage DNAs, e.g., the numerous derivatives of phage lambda, e.g., NM989, λGT10 and λGT11, and other phages, e.g., M13 and filamentous single-stranded phage DNA. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin and zeocin; auxotrophic markers can also be used.

In other embodiments, eukaryotic host cells, such as yeast, insect, mammalian or plant cells, may be used. Yeast cells, typically S. cerevisiae, are useful for eukaryotic genetic studies, due to the ease of targeting genetic changes by homologous recombination and the ability to easily complement genetic defects using recombinantly expressed proteins. Yeast cells are useful for identifying interacting protein components, e.g. through use of a two-hybrid system. In a preferred embodiment, yeast cells are useful for protein expression. Vectors of the present invention for use in yeast will typically, but not invariably, contain an origin of replication suitable for use in yeast and a selectable marker that is functional in yeast. Yeast vectors include Yeast Integrating plasmids (e.g., YIp5) and Yeast Replicating plasmids (the YRp and YEp series plasmids), Yeast Centromere plasmids (the YCp series plasmids), Yeast Artificial Chromosomes (YACs) which are based on yeast linear plasmids, denoted YLp, pGPD-2, 2 μ plasmids and derivatives thereof, and improved shuttle vectors such as those described in Gietz et al., Gene, 74: 527-34 (1988) (YIplac, YEplac and YCplac). Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trp1-D1 and lys2-201.

Insect cells are often chosen for high efficiency protein expression. Where the host cells are from Spodoptera frugiperda, e.g., Sf9 and Sf21 cell lines, and expresSF™ cells (Protein Sciences Corp., Meriden, Conn., USA)), the vector replicative strategy is typically based upon the baculovirus life cycle. Typically, baculovirus transfer vectors are used to replace the wild-type AcMNPV polyhedrin gene with a heterologous gene of interest. Sequences that flank the polyhedrin gene in the wild-type genome are positioned 5′ and 3′ of the expression cassette on the transfer vectors. Following co-transfection with AcMNPV DNA, a homologous recombination event occurs between these sequences resulting in a recombinant virus carrying the gene of interest and the polyhedrin or p10 promoter. Selection can be based upon visual screening for lacZ fusion activity.

In another embodiment, the host cells may be mammalian cells, which are particularly useful for expression of proteins intended as pharmaceutical agents, and for screening of potential agonists and antagonists of a protein or a physiological pathway. Mammalian vectors intended for autonomous extrachromosomal replication will typically include a viral origin, such as the SV40 origin (for replication in cell lines expressing the large T-antigen, such as COS1 and COS7 cells), the papillomavirus origin, or the EBV origin for long term episomal replication (for use, e.g., in 293-EBNA cells, which constitutively express the EBV EBNA-1 gene product and adenovirus E1A). Vectors intended for integration, and thus replication as part of the mammalian chromosome, can, but need not, include an origin of replication functional in mammalian cells, such as the SV40 origin. Vectors based upon viruses, such as adenovirus, adeno-associated virus, vaccinia virus, and various mammalian retroviruses, will typically replicate according to the viral replicative strategy. Selectable markers for use in mammalian cells include resistance to neomycin (G418), blasticidin, hygromycin and to zeocin, and selection based upon the purine salvage pathway using HAT medium.

Expression in mammalian cells can be achieved using a variety of plasmids, including pSV2, pBC12BI, and p91023, as well as lytic virus vectors (e.g., vaccinia virus, adeno virus, and baculovirus), episomal virus vectors (e.g., bovine papillomavirus), and retroviral vectors (e.g., murine retroviruses). Useful vectors for insect cells include baculoviral vectors and pVL 941.

Plant cells can also be used for expression, with the vector replicon typically derived from a plant virus (e.g., cauliflower mosaic virus, CaMV; tobacco mosaic virus, TMV) and selectable markers chosen for suitability in plants.

It is known that codon usage of different host cells may be different. For example, a plant cell and a human cell may exhibit a difference in codon preference for encoding a particular amino acid. As a result, human mRNA may not be efficiently translated in a plant, bacteria or insect host cell. Therefore, another embodiment of this invention is directed to codon optimization. The codons of the nucleic acid molecules of the invention may be modified to resemble, as much as possible, genes naturally contained within the host cell without altering the amino acid sequence encoded by the nucleic acid molecule.

Any of a wide variety of expression control sequences may be used in these vectors to express the DNA sequences of this invention. Such useful expression control sequences include the expression control sequences associated with structural genes of the foregoing expression vectors. Expression control sequences that control transcription include, e.g., promoters, enhancers and transcription termination sites. Expression control sequences in eukaryotic cells that control post-transcriptional events include splice donor and acceptor sites and sequences that modify the half-life of the transcribed RNA, e.g., sequences that direct poly(A) addition or binding sites for RNA-binding proteins. Expression control sequences that control translation include ribosome binding sites, sequences which direct targeted expression of the polypeptide to or within particular cellular compartments, and sequences in the 5′ and 3′ untranslated regions that modify the rate or efficiency of translation.

Examples of useful expression control sequences for a prokaryote, e.g., E. coli, will include a promoter, often a phage promoter, such as phage lambda pL promoter, the trc promoter, a hybrid derived from the trp and lac promoters, the bacteriophage T7 promoter (in E. coli cells engineered to express the T7 polymerase), the TAC or TRC system, the major operator and promoter regions of phage lambda, the control regions of fd coat protein, or the araBAD operon. Prokaryotic expression vectors may further include transcription terminators, such as the aspA terminator, and elements that facilitate translation, such as a consensus ribosome binding site and translation termination codon, Schomer et al., Proc. Natl. Acad. Sci. USA 83: 8506-8510 (1986).

Expression control sequences for yeast cells, typically S. cerevisiae, will include a yeast promoter, such as the CYC1 promoter, the GAL1 promoter, the GAL10 promoter, ADH 1 promoter, the promoters of the yeast_-mating system, or the GPD promoter, and will typically have elements that facilitate transcription termination, such as the transcription termination signals from the CYC1 or ADH1 gene.

Expression vectors useful for expressing proteins in mammalian cells will include a promoter active in mammalian cells. These promoters include those derived from mammalian viruses, such as the enhancer-promoter sequences from the immediate early gene of the human cytomegalovirus (CMV), the enhancer-promoter sequences from the Rous sarcoma virus long terminal repeat (RSV LTR), the enhancer-promoter from SV40 or the early and late promoters of adenovirus. Other expression control sequences include the promoter for 3-phosphoglycerate kinase or other glycolytic enzymes, the promoters of acid phosphatase. Other expression control sequences include those from the gene comprising the PSNA of interest. Often, expression is enhanced by incorporation of polyadenylation sites, such as the late SV40 polyadenylation site and the polyadenylation signal and transcription termination sequences from the bovine growth hormone (BGH) gene, and ribosome binding sites. Furthermore, vectors can include introns, such as intron II of rabbit β-globin gene and the SV40 splice elements.

Preferred nucleic acid vectors also include a selectable or amplifiable marker gene and means for amplifying the copy number of the gene of interest. Such marker genes are well-known in the art. Nucleic acid vectors may also comprise stabilizing sequences (e.g., ori- or ARS-like sequences and telomere-like sequences), or may alternatively be designed to favor directed or non-directed integration into the host cell genome. In a preferred embodiment, nucleic acid sequences of this invention are inserted in frame into an expression vector that allows high level expression of an RNA which encodes a protein comprising the encoded nucleic acid sequence of interest. Nucleic acid cloning and sequencing methods are well-known to those of skill in the art and are described in an assortment of laboratory manuals, including Sambrook (1989), supra, Sambrook (2000), supra; and Ausubel (1992), supra, Ausubel (1999), supra. Product information from manufacturers of biological, chemical and immunological reagents also provide useful information.

Expression vectors may be either constitutive or inducible. Inducible vectors include either naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters. Examples of inducible synthetic promoters are the hybrid Plac/ara-1 promoter and the PLtetO-1 promoter. The PltetO-1 promoter takes advantage of the high expression levels from the PL promoter of phage lambda, but replaces the lambda repressor sites with two copies of operator 2 of the Tn10 tetracycline resistance operon, causing this promoter to be tightly repressed by the Tet repressor protein and induced in response to tetracycline (Tc) and Tc derivatives such as anhydrotetracycline. Vectors may also be inducible because they contain hormone response elements, such as the glucocorticoid response element (GRE) and the estrogen response element (ERE), which can confer hormone inducibility where vectors are used for expression in cells having the respective hormone receptors. To reduce background levels of expression, elements responsive to ecdysone, an insect hormone, can be used instead, with coexpression of the ecdysone receptor.

In one aspect of the invention, expression vectors can be designed to fuse the expressed polypeptide to small protein tags that facilitate purification and/or visualization. Tags that facilitate purification include a polyhistidine tag that facilitates purification of the fusion protein by immobilized metal affinity chromatography, for example using NiNTA resin (Qiagen Inc., Valencia, Calif., USA) or TALON™ resin (cobalt immobilized affinity chromatography medium, Clontech Labs, Palo Alto, Calif., USA). The fusion protein can include a chitin-binding tag and self-excising intein, permitting chitin-based purification with self-removal of the fused tag (IMPACT™ system, New England Biolabs, Inc., Beverley, Mass, USA). Alternatively, the fusion protein can include a calmodulin-binding peptide tag, permitting purification by calmodulin affinity resin (Stratagene, La Jolla, Calif., USA), or a specifically excisable fragment of the biotin carboxylase carrier protein, permitting purification of in vivo biotinylated protein using an avidin resin and subsequent tag removal (Promega, Madison, Wis., USA). As another useful alternative, the proteins of the present invention can be expressed as a fusion protein with glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione. Other tags include, for example, the Xpress epitope, detectable by anti-Xpress antibody (Invitrogen, Carlsbad, Calif., USA), a myc tag, detectable by anti-myc tag antibody, the V5 epitope, detectable by anti-V5 antibody (Invitrogen, Carlsbad, Calif., USA), FLAG® epitope, detectable by anti-FLAG® antibody (Stratagene, La Jolla, Calif., USA), and the HA epitope.

For secretion of expressed proteins, vectors can include appropriate sequences that encode secretion signals, such as leader peptides. For example, the pSecTag2 vectors (Invitrogen, Carlsbad, Calif., USA) are 5.2 kb mammalian expression vectors that carry the secretion signal from the V-J2-C region of the mouse Ig kappa-chain for efficient secretion of recombinant proteins from a variety of mammalian cell lines.

Expression vectors can also be designed to fuse proteins encoded by the heterologous nucleic acid insert to polypeptides that are larger than purification and/or identification tags. Useful fusion proteins include those that permit display of the encoded protein on the surface of a phage or cell, fusion to intrinsically fluorescent proteins, such as those that have a green fluorescent protein (GFP)-like chromophore, fusions to the IgG Fc region, and fusion proteins for use in two hybrid systems.

Vectors for phage display fuse the encoded polypeptide to, e.g., the gene III protein (pIII) or gene VIII protein (pVIII) for display on the surface of filamentous phage, such as M13. See Barbas et al, Phage Display: A Laboratory Manual, Cold Spring Harbor Laboratory Press (2001); Kay et al. (eds.), Phage Display of Peotides and Proteins: A Laboratory Manual, Academic Press, Inc., (1996); Abelson et al. (eds.), Combinatorial Chemistry (Methods in Enzymology, Vol. 267) Academic Press (1996). Vectors for yeast display, e.g. the pYD1 yeast display vector (Invitrogen, Carlsbad, Calif., USA), use the -agglutinin yeast adhesion receptor to display recombinant protein on the surface of S. cerevisiae. Vectors for mammalian display, e.g., the pDisplay™ vector (Invitrogen, Carlsbad, Calif., USA), target recombinant proteins using an N-terminal cell surface targeting signal and a C-terminal transmembrane anchoring domain of platelet derived growth factor receptor.

A wide variety of vectors now exist that fuse proteins encoded by heterologous nucleic acids to the chromophore of the substrate-independent, intrinsically fluorescent green fluorescent protein from Aequorea victoria (“GFP”) and its variants. The GFP-like chromophore can be selected from GFP-like chromophores found in naturally occurring proteins, such as A. victoria GFP (GenBank accession number AAA27721), Renilla reniformis GFP, FP583 (GenBank accession no. AF168419) (DsRed), FP593 (AF27271 1), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. See Li et al., J. Biol. Chem. 272: 28545-28549 (1997). Alternatively, the GFP-like chromophore can be selected from GFP-like chromophores modified from those found in nature. The methods for engineering such modified GFP-like chromophores and testing them for fluorescence activity, both alone and as part of protein fusions, are well-known in the art. See Heim et al., Curr. Biol. 6: 178-182 (1996) and Palm et al., Methods Enzymol. 302: 378-394 (1999), incorporated herein by reference in its entirety. A variety of such modified chromophores are now commercially available and can readily be used in the fusion proteins of the present invention. These include EGFP (“enhanced GFP”), EBFP (“enhanced blue fluorescent protein”), BFP2, EYFP (“enhanced yellow fluorescent protein”), ECFP (“enhanced cyan fluorescent protein”) or Citrine. EGFP (see, e.g, Cormack et al., Gene 173: 33-38 (1996); U.S. Pat. Nos. 6,090,919 and 5,804,387) is found on a variety of vectors, both plasmid and viral, which are available commercially (Clontech Labs, Palo Alto, Calif., USA); EBFP is optimized for expression in mammalian cells whereas BFP2, which retains the original jellyfish codons, can be expressed in bacteria (see, e.g,. Heim et al, Curr. Biol. 6: 178-182 (1996) and Cormack et al., Gene 173: 33-38 (1996)). Vectors containing these blue-shifted variants are available from Clontech Labs (Palo Alto, Calif., USA). Vectors containing EYFP, ECFP (see, e.g., Heim et al., Curr. Biol. 6: 178-182 (1996); Miyawaki et al., Nature 388: 882-887 (1997)) and Citrine (see, e.g., Heikal et al., Proc. Natl. Acad. Sci. USA 97: 11996-12001 (2000)) are also available from Clontech Labs. The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties. See also Conn (ed.), Green Fluorescent Protein (Methods in Enzymology, Vol. 302), Academic Press, Inc. (1999). The GFP-like chromophore of each of these GFP variants can usefully be included in the fusion proteins of the present invention.

Fusions to the IgG Fc region increase serum half life of protein pharmaceutical products through interaction with the FcRn receptor (also denominated the FcRp receptor and the Brambell receptor, FcRb), further described in International Patent Application Nos. WO 97/43316, WO 97/34631, WO 96/32478, WO 96/18412.

For long-term, high-yield recombinant production of the proteins, protein fusions, and protein fragments of the present invention, stable expression is preferred. Stable expression is readily achieved by integration into the host cell genome of vectors having selectable markers, followed by selection of these integrants. Vectors such as pUB6/V5-His A, B, and C (Invitrogen, Carlsbad, Calif., USA) are designed for high-level stable expression of heterologous proteins in a wide range of mammalian tissue types and cell lines. pUB6/V5-His uses the promoter/enhancer sequence from the human ubiquitin C gene to drive expression of recombinant proteins: expression levels in 293, CHO, and NIH3T3 cells are comparable to levels from the CMV and human EF-1a promoters. The bsd gene permits rapid selection of stably transfected mammalian cells with the potent antibiotic blasticidin.

Replication incompetent retroviral vectors, typically derived from Moloney murine leukemia virus, also are useful for creating stable transfectants having integrated provirus. The highly efficient transduction machinery of retroviruses, coupled with the availability of a variety of packaging cell lines such as RetroPack™ PT 67, EcoPack2™-293, AmphoPack-293, and GP2-293 cell lines (all available from Clontech Laboratories, Palo Alto, Calif., USA), allow a wide host range to be infected with high efficiency; varying the multiplicity of infection readily adjusts the copy number of the integrated provirus.

Of course, not all vectors and expression control sequences will function equally well to express the nucleic acid sequences of this invention. Neither will all hosts function equally well with the same expression system. However, one of skill in the art may make a selection among these vectors, expression control sequences and hosts without undue experimentation and without departing from the scope of this invention. For example, in selecting a vector, the host must be considered because the vector must be replicated in it. The vector's copy number, the ability to control that copy number, the ability to control integration, if any, and the expression of any other proteins encoded by the vector, such as antibiotic or other selection markers, should also be considered. The present invention further includes host cells comprising the vectors of the present invention, either present episomally within the cell or integrated, in whole or in part, into the host cell chromosome. Among other considerations, some of which are described above, a host cell strain may be chosen for its ability to process the expressed protein in the desired fashion. Such post-translational modifications of the polypeptide include, but are not limited to, acetylation, carboxylation, glycosylation, phosphorylation, lipidation, and acylation, and it is an aspect of the present invention to provide PSPs with such post-translational modifications.

Polypeptides of the invention may be post-translationally modified. Post-translational modifications include phosphorylation of amino acid residues serine, threonine and/or tyrosine, N-linked and/or O-linked glycosylation, methylation, acetylation, prenylation, methylation, acetylation, arginylation, ubiquination and racemization. One may determine whether a polypeptide of the invention is likely to be post-translationally modified by analyzing the sequence of the polypeptide to determine if there are peptide motifs indicative of sites for post-translational modification. There are a number of computer programs that permit prediction of post-translational modifications. See, e.g., www.expasy.org (accessed Aug. 31, 2001), which includes PSORT, for prediction of protein sorting signals and localization sites, SignalP, for prediction of signal peptide cleavage sites, MITOPROT and Predotar, for prediction of mitochondrial targeting sequences, NetOGlyc, for prediction of type O-glycosylation sites in mammalian proteins, big-PI Predictor and DGPI, for prediction of prenylation-anchor and cleavage sites, and NetPhos, for prediction of Ser, Thr and Tyr phosphorylation sites in eukaryotic proteins. Other computer programs, such as those included in GCG, also may be used to determine post-translational modification peptide motifs.

General examples of types of post-translational modifications may be found in web sites such as the Delta Mass database http://www.abrf.org/ABRF/Research Committees/deltamass/deltamass.html (accessed Oct. 19, 2001); “GlycoSuiteDB: a new curated relational database of glycoprotein glycan structures and their biological sources” Cooper et al. Nucleic Acids Res. 29; 332-335 (2001) and http://www.glycosuite.com/(accessed Oct. 19, 2001); “O-GLYCBASE version 4.0: a revised database of O-glycosylated proteins” Gupta et al. Nucleic Acids Research, 27: 370-372 (1999) and http://www.cbs.dtu.dk/databases/OGLYCBASE/(accessed Oct. 19, 2001); “PhosphoBase, a database of phosphorylation sites: release 2.0.”, Kreegipuu et al. Nucleic Acids Res 27(1):237-239 (1999) and http://www.cbs.dtu.dk/databases/PhosphoBase/(accessed Oct. 19, 2001); or http://pir.georgetown.edu/pirwww/search/textresid.html (accessed Oct. 19, 2001).

Tumorigenesis is often accompanied by alterations in the post-translational modifications of proteins. Thus, in another embodiment, the invention provides polypeptides from cancerous cells or tissues that have altered post-translational modifications compared to the post-translational modifications of polypeptides from normal cells or tissues. A number of altered post-translational modifications are known. One common alteration is a change in phosphorylation state, wherein the polypeptide from the cancerous cell or tissue is hyperphosphorylated or hypophosphorylated compared to the polypeptide from a normal tissue, or wherein the polypeptide is phosphorylated on different residues than the polypeptide from a normal cell. Another common alteration is a change in glycosylation state, wherein the polypeptide from the cancerous cell or tissue has more or less glycosylation than the polypeptide from a normal tissue, and/or wherein the polypeptide from the cancerous cell or tissue has a different type of glycosylation than the polypeptide from a noncancerous cell or tissue. Changes in glycosylation may be critical because carbohydrate-protein and carbohydrate-carbohydrate interactions are important in cancer cell progression, dissemination and invasion. See, e.g., Barchi, Curr. Pharm. Des. 6: 485-501 (2000), Verma, Cancer Biochem. Biophys. 14: 151-162 (1994) and Dennis et al., Bioessays 5: 412-421 (1999).

Another post-translational modification that may be altered in cancer cells is prenylation. Prenylation is the covalent attachment of a hydrophobic prenyl group (either famesyl or geranylgeranyl) to a polypeptide. Prenylation is required for localizing a protein to a cell membrane and is often required for polypeptide function. For instance, the Ras superfamily of GTPase signaling proteins must be prenylated for function in a cell. See, e.g., Prendergast et al., Semin. Cancer Biol. 10: 443-452 (2000) and Khwaja et al., Lancet 355: 741-744 (2000).

Other post-translation modifications that may be altered in cancer cells include, without limitation, polypeptide methylation, acetylation, arginylation or racemization of amino acid residues. In these cases, the polypeptide from the cancerous cell may exhibit either increased or decreased amounts of the post-translational modification compared to the corresponding polypeptides from noncancerous cells.

Other polypeptide alterations in cancer cells include abnormal polypeptide cleavage of proteins and aberrant protein-protein interactions. Abnormal polypeptide cleavage may be cleavage of a polypeptide in a cancerous cell that does not usually occur in a normal cell, or a lack of cleavage in a cancerous cell, wherein the polypeptide is cleaved in a normal cell. Aberrant protein-protein interactions may be either covalent cross-linking or non-covalent binding between proteins that do not normally bind to each other. Alternatively, in a cancerous cell, a protein may fail to bind to another protein to which it is bound in a noncancerous cell. Alterations in cleavage or in protein-protein interactions may be due to over- or underproduction of a polypeptide in a cancerous cell compared to that in a normal cell, or may be due to alterations in post-translational modifications (see above) of one or more proteins in the cancerous cell. See, e.g., Henschen-Edman, Ann. N.Y. Acad. Sci. 936: 580-593 (2001).

Alterations in polypeptide post-translational modifications, as well as changes in polypeptide cleavage and protein-protein interactions, may be determined by any method known in the art. For instance, alterations in phosphorylation may be determined by using anti-phosphoserine, anti-phosphothreonine or anti-phosphotyrosine antibodies or by amino acid analysis. Glycosylation alterations may be determined using antibodies specific for different sugar residues, by carbohydrate sequencing, or by alterations in the size of the glycoprotein, which can be determined by, e.g., SDS polyacrylamide gel electrophoresis (PAGE). Other alterations of post-translational modifications, such as prenylation, racemization, methylation, acetylation and arginylation, may be determined by chemical analysis, protein sequencing, amino acid analysis, or by using antibodies specific for the particular post-translational modifications. Changes in protein-protein interactions and in polypeptide cleavage may be analyzed by any method known in the art including, without limitation, non-denaturing PAGE (for non-covalent protein-protein interactions), SDS PAGE (for covalent protein-protein interactions and protein cleavage), chemical cleavage, protein sequencing or immunoassays.

In another embodiment, the invention provides polypeptides that have been post-translationally modified. In one embodiment, polypeptides may be modified enzymatically or chemically, by addition or removal of a post-translational modification. For example, a polypeptide may be glycosylated or deglycosylated enzymatically. Similarly, polypeptides may be phosphorylated using a purified kinase, such as a MAP kinase (e.g, p38, ERK, or JNK) or a tyrosine kinase (e.g., Src or erbB2). A polypeptide may also be modified through synthetic chemistry. Alternatively, one may isolate the polypeptide of interest from a cell or tissue that expresses the polypeptide with the desired post-translational modification. In another embodiment, a nucleic acid molecule encoding the polypeptide of interest is introduced into a host cell that is capable of post-translationally modifying the encoded polypeptide in the desired fashion. If the polypeptide does not contain a motif for a desired post-translational modification, one may alter the post-translational modification by mutating the nucleic acid sequence of a nucleic acid molecule encoding the polypeptide so that it contains a site for the desired post-translational modification. Amino acid sequences that may be post-translationally modified are known in the art. See, e.g., the programs described above on the website www.expasy.org. The nucleic acid molecule is then be introduced into a host cell that is capable of post-translationally modifying the encoded polypeptide. Similarly, one may delete sites that are post-translationally modified by either mutating the nucleic acid sequence so that the encoded polypeptide does not contain the post-translational modification motif, or by introducing the native nucleic acid molecule into a host cell that is not capable of post-translationally modifying the encoded polypeptide.

In selecting an expression control sequence, a variety of factors should also be considered. These include, for example, the relative strength of the sequence, its controllability, and its compatibility with the nucleic acid sequence of this invention, particularly with regard to potential secondary structures. Unicellular hosts should be selected by consideration of their compatibility with the chosen vector, the toxicity of the product coded for by the nucleic acid sequences of this invention, their secretion characteristics, their ability to fold the polypeptide correctly, their fermentation or culture requirements, and the ease of purification from them of the products coded for by the nucleic acid sequences of this invention.

The recombinant nucleic acid molecules and more particularly, the expression vectors of this invention may be used to express the polypeptides of this invention as recombinant polypeptides in a heterologous host cell. The polypeptides of this invention may be full-length or less than full-length polypeptide fragments recombinantly expressed from the nucleic acid sequences according to this invention. Such polypeptides include analogs, derivatives and muteins that may or may not have biological activity.

Vectors of the present invention will also often include elements that permit in vitro transcription of RNA from the inserted heterologous nucleic acid. Such vectors typically include a phage promoter, such as that from T7, T3, or SP6, flanking the nucleic acid insert. Often two different such promoters flank the inserted nucleic acid, permitting separate in vitro production of both sense and antisense strands.

Transformation and other methods of introducing nucleic acids into a host cell (e.g., conjugation, protoplast transformation or fusion, transfection, electroporation, liposome delivery, membrane fusion techniques, high velocity DNA-coated pellets, viral infection and protoplast fusion) can be accomplished by a variety of methods which are well-known in the art (See, for instance, Ausubel, supra, and Sambrook et al., supra). Bacterial, yeast, plant or mammalian cells are transformed or transfected with an expression vector, such as a plasmid, a cosmid, or the like, wherein the expression vector comprises the nucleic acid of interest. Alternatively, the cells may be infected by a viral expression vector comprising the nucleic acid of interest. Depending upon the host cell, vector, and method of transformation used, transient or stable expression of the polypeptide will be constitutive or inducible. One having ordinary skill in the art will be able to decide whether to express a polypeptide transiently or stably, and whether to express the protein constitutively or inducibly.

A wide variety of unicellular host cells are useful in expressing the DNA sequences of this invention. These hosts may include well-known eukaryotic and prokaryotic hosts, such as strains of, fungi, yeast, insect cells such as Spodoptera frugiperda (SF9), animal cells such as CHO, as well as plant cells in tissue culture. Representative examples of appropriate host cells include, but are not limited to, bacterial cells, such as E. coli, Caulobacter crescentus, Streptomyces species, and Salmonella typhimurium; yeast cells, such as Saccharomyces cerevisiae, Schizosaccharomyces pombe, Pichiapastoris, Pichia methanolica; insect cell lines, such as those from Spodoptera frugiperda, e.g., Sf9 and Sf21 cell lines, and expresSF™ cells (Protein Sciences Corp., Meriden, Conn., USA), Drosophila S2 cells, and Trichoplusia ni High Five®D Cells (Invitrogen, Carlsbad, Calif., USA); and mammalian cells. Typical mammalian cells include BHK cells, BSC 1 cells, BSC 40 cells, BMT 10 cells, VERO cells, COS1 cells, COS7 cells, Chinese hamster ovary (CHO) cells, 3T3 cells, NIH 3T3 cells, 293 cells, HEPG2 cells, HeLa cells, L cells, MDCK cells, HEK293 cells, WI38 cells, murine ES cell lines (e.g., from strains 129/SV, C57/BL6, DBA-1, 129/SVJ), K562 cells, Jurkat cells, and BW5147 cells. Other mammalian cell lines are well-known and readily available from the American Type Culture Collection (ATCC) (Manassas, Va., USA) and the National Institute of General Medical Sciences (NIGMS) Human Genetic Cell Repository at the Coriell Cell Repositories (Camden, N.J., USA). Cells or cell lines derived from prostate are particularly preferred because they may provide a more native post-translational processing. Particularly preferred are human prostate cells.

Particular details of the transfection, expression and purification of recombinant proteins are well documented and are understood by those of skill in the art. Further details on the various technical aspects of each of the steps used in recombinant production of foreign genes in bacterial cell expression systems can be found in a number of texts and laboratory manuals in the art. See, e.g., Ausubel (1992), supra, Ausubel (1999), supra, Sambrook (1989), supra, and Sambrook (2001), supra, herein incorporated by reference.

Methods for introducing the vectors and nucleic acids of the present invention into the host cells are well-known in the art; the choice of technique will depend primarily upon the specific vector to be introduced and the host cell chosen.

Nucleic acid molecules and vectors may be introduced into prokaryotes, such as E. coli, in a number of ways. For instance, phage lambda vectors will typically be packaged using a packaging extract (e.g., Gigapack® packaging extract, Stratagene, La Jolla, Calif., USA), and the packaged virus used to infect E. coli.

Plasmid vectors will typically be introduced into chemically competent or electrocompetent bacterial cells. E. coli cells can be rendered chemically competent by treatment, e.g., with CaCl₂, or a solution of Mg²⁺, Mn²⁺, Ca²⁺, Rb⁺ or K⁺, dimethyl sulfoxide, dithiothreitol, and hexamine cobalt (III), Hanahan, J. Mol. Biol. 166(4):557-80 (1983), and vectors introduced by heat shock. A wide variety of chemically competent strains are also available commercially (e.g., Epicurian coli® XL10-Gold® Ultracompetent Cells (Stratagene, La Jolla, Calif., USA); DH5 competent cells (Clontech Laboratories, Palo Alto, Calif., USA); and TOP10 Chemically Competent E. coli Kit (Invitrogen, Carlsbad, Calif., USA)). Bacterial cells can be rendered electrocompetent, that is, competent to take up exogenous DNA by electroporation, by various pre-pulse treatments; vectors are introduced by electroporation followed by subsequent outgrowth in selected media. An extensive series of protocols is provided online in Electroprotocols (BioRad, Richmond, Calif., USA) (http://www.biorad.com/LifeScience/pdf, New_Gene_Pulser.pdf).

Vectors can be introduced into yeast cells by spheroplasting, treatment with lithium salts, electroporation, or protoplast fusion. Spheroplasts are prepared by the action of hydrolytic enzymes such as snail-gut extract, usually denoted Glusulase, or Zymolyase, an enzyme from Arthrobacter luteus, to remove portions of the cell wall in the presence of osmotic stabilizers, typically 1 M sorbitol. DNA is added to the spheroplasts, and the mixture is co-precipitated with a solution of polyethylene glycol (PEG) and Ca²⁺. Subsequently, the cells are resuspended in a solution of sorbitol, mixed with molten agar and then layered on the surface of a selective plate containing sorbitol.

For lithium-mediated transformation, yeast cells are treated with lithium acetate, which apparently permeabilizes the cell wall, DNA is added and the cells are co-precipitated with PEG. The cells are exposed to a brief heat shock, washed free of PEG and lithium acetate, and subsequently spread on plates containing ordinary selective medium. Increased frequencies of transformation are obtained by using specially-prepared single-stranded carrier DNA and certain organic solvents. Schiestl et al., Curr. Genet. 16(5-6): 339-46 (1989).

For electroporation, freshly-grown yeast cultures are typically washed, suspended in an osmotic protectant, such as sorbitol, mixed with DNA, and the cell suspension pulsed in an electroporation device. Subsequently, the cells are spread on the surface of plates containing selective media. Becker et al., Methods Enzymol. 194: 182-187 (1991). The efficiency of transformation by electroporation can be increased over 100-fold by using PEG, single-stranded carrier DNA and cells that are in late log-phase of growth. Larger constructs, such as YACs, can be introduced by protoplast fusion.

Mammalian and insect cells can be directly infected by packaged viral vectors, or transfected by chemical or electrical means. For chemical transfection, DNA can be coprecipitated with CaPO₄ or introduced using liposomal and nonliposomal lipid-based agents. Commercial kits are available for CaPO₄ transfection (CalPhoS™ Mammalian Transfection Kit, Clontech Laboratories, Palo Alto, Calif., USA), and lipid-mediated transfection can be practiced using commercial reagents, such as LIPOFECTAMINE™ 2000, LIPOFECTAMINE™ Reagent, CELLFECTIN® Reagent, and LIPOFECTIN™ Reagent (Invitrogen, Carlsbad, Calif., USA), DOTAP Liposomal Transfection Reagent, FuGENE 6, X-tremeGENE Q2, DOSPER, (Roche Molecular Biochemicals, Indianapolis, Ind. USA), Effectene™, PolyFect®, Superfect® (Qiagen, Inc., Valencia, Calif., USA). Protocols for electroporating mammalian cells can be found online in Electroprotocols (Bio-Rad, Richmond, Calif., USA) (http://www.bio-rad.com/LifeScience/pdf/New_Gene_Pulser.pdf); Norton et al. (eds.), Gene Transfer Methods: Introducing DNA into Living Cells and Organisms, BioTechniques Books, Eaton Publishing Co. (2000); incorporated herein by reference in its entirety. Other transfection techniques include transfection by particle bombardment and microinjection. See, e.g., Cheng et al., Proc. Natl. Acad. Sci. USA 90(10): 4455-9 (1993); Yang et al., Proc. Natl. Acad. Sci. USA 87(24): 9568-72 (1990).

Production of the recombinantly produced proteins of the present invention can optionally be followed by purification.

Purification of recombinantly expressed proteins is now well by those skilled in the art. See, e.g., Thorner et al. (eds.), Applications of Chimeric Genes and Hybrid Proteins Part A: Gene Expression and Protein Purification (Methods in Enzymology, Vol. 326), Academic Press (2000); Harbin (ed.), Cloning, Gene Expression and Protein Purification: Experimental Procedures and Process Rationale, Oxford Univ. Press (2001); Marshak et al., Strategies for Protein Purification and Characterization: A Laboratory Course Manual, Cold Spring Harbor Laboratory Press (1996); and Roe (ed.), Protein Purification Applications, Oxford University Press (2001); the disclosures of which are incorporated herein by reference in their entireties, and thus need not be detailed here.

Briefly, however, if purification tags have been fused through use of an expression vector that appends such tags, purification can be effected, at least in part, by means appropriate to the tag, such as use of immobilized metal affinity chromatography for polyhistidine tags. Other techniques common in the art include ammonium sulfate fractionation, immunoprecipitation, fast protein liquid chromatography (FPLC), high performance liquid chromatography (HPLC), and preparative gel electrophoresis.

Polypeptides

Another object of the invention is to provide polypeptides encoded by the nucleic acid molecules of the instant invention. In a preferred embodiment, the polypeptide is a prostate specific polypeptide (PSP). In an even more preferred embodiment, the polypeptide is derived from a polypeptide comprising the amino acid sequence of SEQ ID NO: 136 through 240. A polypeptide as defined herein may be produced recombinantly, as discussed supra, may be isolated from a cell that naturally expresses the protein, or may be chemically synthesized following the teachings of the specification and using methods well-known to those having ordinary skill in the art.

In another aspect, the polypeptide may comprise a fragment of a polypeptide, wherein the fragment is as defined herein. In a preferred embodiment, the polypeptide fragment is a fragment of a PSP. In a more preferred embodiment, the fragment is derived from a polypeptide comprising the amino acid sequence of SEQ ID NO: 136 through 240. A polypeptide that comprises only a fragment of an entire PSP may or may not be a polypeptide that is also a PSP. For instance, a full-length polypeptide may be prostate-specific, while a fragment thereof may be found in other tissues as well as in prostate. A polypeptide that is not a PSP, whether it is a fragment, analog, mutein, homologous protein or derivative, is nevertheless useful, especially for immunizing animals to prepare anti-PSP antibodies. However, in a preferred embodiment, the part or fragment is a PSP. Methods of determining whether a polypeptide is a PSP are described infra.

Fragments of at least 6 contiguous amino acids are useful in mapping B cell and T cell epitopes of the reference protein. See, e.g., Geysen et al., Proc. Natl. Acad. Sci. USA 81: 3998-4002 (1984) and U.S. Pat. Nos. 4,708,871 and 5,595,915, the disclosures of which are incorporated herein by reference in their entireties. Because the fragment need not itself be immunogenic, part of an immunodominant epitope, nor even recognized by native antibody, to be useful in such epitope mapping, all fragments of at least 6 amino acids of the proteins of the present invention have utility in such a study.

Fragments of at least 8 contiguous amino acids, often at least 15 contiguous amino acids, are useful as immunogens for raising antibodies that recognize the proteins of the present invention. See, e.g., Lerner, Nature 299: 592-596 (1982); Shinnick et al., Annu. Rev. Microbiol. 37: 425-46 (1983); Sutcliffe et al., Science 219: 660-6 (1983), the disclosures of which are incorporated herein by reference in their entireties. As further described in the above-cited references, virtually all 8-mers, conjugated to a carrier, such as a protein, prove immunogenic, meaning that they are capable of eliciting antibody for the conjugated peptide; accordingly, all fragments of at least 8 amino acids of the proteins of the present invention have utility as immunogens.

Fragments of at least 8, 9, 10 or 12 contiguous amino acids are also useful as competitive inhibitors of binding of the entire protein, or a portion thereof, to antibodies (as in epitope mapping), and to natural binding partners, such as subunits in a multimeric complex or to receptors or ligands of the subject protein; this competitive inhibition permits identification and separation of molecules that bind specifically to the protein of interest, U.S. Pat. Nos. 5,539,084 and 5,783,674, incorporated herein by reference in their entireties.

The protein, or protein fragment, of the present invention is thus at least 6 amino acids in length, typically at least 8, 9, 10 or 12 amino acids in length, and often at least 15 amino acids in length. Often, the protein of the present invention, or fragment thereof, is at least 20 amino acids in length, even 25 amino acids, 30 amino acids, 35 amino acids, or 50 amino acids or more in length. Of course, larger fragments having at least 75 amino acids, 100 amino acids, or even 150 amino acids are also useful, and at times preferred.

One having ordinary skill in the art can produce fragments of a polypeptide by truncating the nucleic acid molecule, e.g., a PSNA, encoding the polypeptide and then expressing it recombinantly. Alternatively, one can produce a fragment by chemically synthesizing a portion of the full-length polypeptide. One may also produce a fragment by enzymatically cleaving either a recombinant polypeptide or an isolated naturally-occurring polypeptide. Methods of producing polypeptide fragments are well-known in the art. See, e.g., Sambrook (1989), supra; Sambrook (2001), supra; Ausubel (1992), supra; and Ausubel (1999), supra. In one embodiment, a polypeptide comprising only a fragment of polypeptide of the invention, preferably a PSP, may be produced by chemical or enzymatic cleavage of a polypeptide. In a preferred embodiment, a polypeptide fragment is produced by expressing a nucleic acid molecule encoding a fragment of the polypeptide, preferably a PSP, in a host cell.

By “polypeptides” as used herein it is also meant to be inclusive of mutants, fusion proteins, homologous proteins and allelic variants of the polypeptides specifically exemplified.

A mutant protein, or mutein, may have the same or different properties compared to a naturally-occurring polypeptide and comprises at least one amino acid insertion, duplication, deletion, rearrangement or substitution compared to the amino acid sequence of a native protein. Small deletions and insertions can often be found that do not alter the function of the protein. In one embodiment, the mutein may or may not be prostate-specific. In a preferred embodiment, the mutein is prostate-specific. In a preferred embodiment, the mutein is a polypeptide that comprises at least one amino acid insertion, duplication, deletion, rearrangement or substitution compared to the amino acid sequence of SEQ ID NO: 136 through 240. In a more preferred embodiment, the mutein is one that exhibits at least 50% sequence identity, more preferably at least 60% sequence identity, even more preferably at least 70%, yet more preferably at least 80% sequence identity to a PSP comprising an amino acid sequence of SEQ ID NO: 136 through 240. In yet a more preferred embodiment, the mutein exhibits at least 85%, more preferably 90%, even more preferably 95% or 96%, and yet more preferably at least 97%, 98%, 99% or 99.5% sequence identity to a PSP comprising an amino acid sequence of SEQ ID NO: 136 through 240.

A mutein may be produced by isolation from a naturally-occurring mutant cell, tissue or organism. A mutein may be produced by isolation from a cell, tissue or organism that has been experimentally mutagenized. Alternatively, a mutein may be produced by chemical manipulation of a polypeptide, such as by altering the amino acid residue to another amino acid residue using synthetic or semi-synthetic chemical techniques. In a preferred embodiment, a mutein may be produced from a host cell comprising an altered nucleic acid molecule compared to the naturally-occurring nucleic acid molecule. For instance, one may produce a mutein of a polypeptide by introducing one or more mutations into a nucleic acid sequence of the invention and then expressing it recombinantly. These mutations may be targeted, in which particular encoded amino acids are altered, or may be untargeted, in which random encoded amino acids within the polypeptide are altered. Muteins with random amino acid alterations can be screened for a particular biological activity or property, particularly whether the polypeptide is prostate-specific, as described below. Multiple random mutations can be introduced into the gene by methods well-known to the art, e.g., by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis and site-specific mutagenesis. Methods of producing muteins with targeted or random amino acid alterations are well-known in the art. See, e.g., Sambrook (1989), supra; Sambrook (2001), supra; Ausubel (1992), supra; and Ausubel (1999), U.S. Pat. No. 5,223,408, and the references discussed supra, each herein incorporated by reference.

By “polypeptide” as used herein it is also meant to be inclusive of polypeptides homologous to those polypeptides exemplified herein. In a preferred embodiment, the polypeptide is homologous to a PSP. In an even more preferred embodiment, the polypeptide is homologous to a PSP selected from the group having an amino acid sequence of SEQ ID NO: 136 through 240. In a preferred embodiment, the homologous polypeptide is one that exhibits significant sequence identity to a PSP. In a more preferred embodiment, the polypeptide is one that exhibits significant sequence identity to an comprising an amino acid sequence of SEQ ID NO: 136 through 240. In an even more preferred embodiment, the homologous polypeptide is one that exhibits at least 50% sequence identity, more preferably at least 60% sequence identity, even more preferably at least 70%, yet more preferably at least 80% sequence identity to a PSP comprising an amino acid sequence of SEQ ID NO: 136 through 240. In a yet more preferred embodiment, the homologous polypeptide is one that exhibits at least 85%, more preferably 90%, even more preferably 95% or 96%, and yet more preferably at least 97% or 98% sequence identity to a PSP comprising an amino acid sequence of SEQ ID NO: 136 through 240. In another preferred embodiment, the homologous polypeptide is one that exhibits at least 99%, more preferably 99.5%, even more preferably 99.6%, 99.7%, 99.8% or 99.9% sequence identity to a PSP comprising an amino acid sequence of SEQ ID NO: 136 through 240. In a preferred embodiment, the amino acid substitutions are conservative amino acid substitutions as discussed above.

In another embodiment, the homologous polypeptide is one that is encoded by a nucleic acid molecule that selectively hybridizes to a PSNA. In a preferred embodiment, the homologous polypeptide is encoded by a nucleic acid molecule that hybridizes to a PSNA under low stringency, moderate stringency or high stringency conditions, as defined herein. In a more preferred embodiment, the PSNA is selected from the group consisting of SEQ ID NO: 1 through 135. In another preferred embodiment, the homologous polypeptide is encoded by a nucleic acid molecule that hybridizes to a nucleic acid molecule that encodes a PSP under low stringency, moderate stringency or high stringency conditions, as defined herein. In a more preferred embodiment, the PSP is selected from the group consisting of SEQ ID NO: 136 through 240.

The homologous polypeptide may be a naturally-occurring one that is derived from another species, especially one derived from another primate, such as chimpanzee, gorilla, rhesus macaque, baboon or gorilla, wherein the homologous polypeptide comprises an amino acid sequence that exhibits significant sequence identity to that of SEQ ID NO: 136 through 240. The homologous polypeptide may also be a naturally-occurring polypeptide from a human, when the PSP is a member of a family of polypeptides. The homologous polypeptide may also be a naturally-occurring polypeptide derived from a non-primate, mammalian species, including without limitation, domesticated species, e.g., dog, cat, mouse, rat, rabbit, guinea pig, hamster, cow, horse, goat or pig. The homologous polypeptide may also be a naturally-occurring polypeptide derived from a non-mammalian species, such as birds or reptiles. The naturally-occurring homologous protein may be isolated directly from humans or other species. Alternatively, the nucleic acid molecule encoding the naturally-occurring homologous polypeptide may be isolated and used to express the homologous polypeptide recombinantly. In another embodiment, the homologous polypeptide may be one that is experimentally produced by random mutation of a nucleic acid molecule and subsequent expression of the nucleic acid molecule. In another embodiment, the homologous polypeptide may be one that is experimentally produced by directed mutation of one or more codons to alter the encoded amino acid of a PSP. Further, the homologous protein may or may not encode polypeptide that is a PSP. However, in a preferred embodiment, the homologous polypeptide encodes a polypeptide that is a PSP.

Relatedness of proteins can also be characterized using a second functional test, the ability of a first protein competitively to inhibit the binding of a second protein to an antibody. It is, therefore, another aspect of the present invention to provide isolated proteins not only identical in sequence to those described with particularity herein, but also to provide isolated proteins (“cross-reactive proteins”) that competitively inhibit the binding of antibodies to all or to a portion of various of the isolated polypeptides of the present invention. Such competitive inhibition can readily be determined using immunoassays well-known in the art.

As discussed above, single nucleotide polymorphisms (SNPs) occur frequently in eukaryotic genomes, and the sequence determined from one individual of a species may differ from other allelic forms present within the population. Thus, by “polypeptide” as used herein it is also meant to be inclusive of polypeptides encoded by an allelic variant of a nucleic acid molecule encoding a PSP. In a preferred embodiment, the polypeptide is encoded by an allelic variant of a gene that encodes a polypeptide having the amino acid sequence selected from the group consisting of SEQ ID NO: 136 through 240. In a yet more preferred embodiment, the polypeptide is encoded by an allelic variant of a gene that has the nucleic acid sequence selected from the group consisting of SEQ ID NO: 1 through 135.

In another embodiment, the invention provides polypeptides which comprise derivatives of a polypeptide encoded by a nucleic acid molecule according to the instant invention. In a preferred embodiment, the polypeptide is a PSP. In a preferred embodiment, the polypeptide has an amino acid sequence selected from the group consisting of SEQ ID NO: 136 through 240, or is a mutein, allelic variant, homologous protein or fragment thereof. In a preferred embodiment, the derivative has been acetylated, carboxylated, phosphorylated, glycosylated or ubiquitinated. In another preferred embodiment, the derivative has been labeled with, e.g., radioactive isotopes such as ¹²⁵i, ³²P, ³⁵S, and ³H. In another preferred embodiment, the derivative has been labeled with fluorophores, chemiluminescent agents, enzymes, and antiligands that can serve as specific binding pair members for a labeled ligand.

Polypeptide modifications are well-known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as, for instance Creighton, Protein Structure and Molecular Properties, 2nd ed., W. H. Freeman and Company (1993). Many detailed reviews are available on this subject, such as, for example, those provided by Wold, in Johnson (ed.), Posttranslational Covalent Modification of Proteins, pgs. 1-12, Academic Press (1983); Seifter et al., Meth. Enzymol. 182: 626-646 (1990) and Rattan et al., Ann. N. Y Acad. Sci. 663: 48-62 (1992).

It will be appreciated, as is well-known and as noted above, that polypeptides are not always entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine.

Useful post-synthetic (and post-translational) modifications include conjugation to detectable labels, such as fluorophores. A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X.

A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA).

The polypeptides of the present invention can also be conjugated to fluorophores, other proteins, and other macromolecules, using bifunctional linking reagents. Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SLAB, Sulfo-SMCC, Sulfo-SMPB, Sulfo-LC-SMPT, SVSB, TFCS (all available Pierce, Rockford, Ill., USA).

The polypeptides, fragments, and fusion proteins of the present invention can be conjugated, using such cross-linking reagents, to fluorophores that are not amine- or thiol-reactive. Other labels that usefully can be conjugated to the polypeptides, fragments, and fusion proteins of the present invention include radioactive labels, echosonographic contrast reagents, and MRI contrast agents.

The polypeptides, fragments, and fusion proteins of the present invention can also usefully be conjugated using cross-linking agents to carrier proteins, such as KLH, bovine thyroglobulin, and even bovine serum albumin (BSA), to increase immunogenicity for raising anti-PSP antibodies.

The polypeptides, fragments, and fusion proteins of the present invention can also usefully be conjugated to polyethylene glycol (PEG); PEGylation increases the serum half-life of proteins administered intravenously for replacement therapy. Delgado et al, Crit. Rev. Ther. Drug Carrier Syst. 9(3-4): 249-304 (1992); Scott et al., Curr. Pharm. Des. 4(6): 423-38 (1998); DeSantis et al., Curr. Opin. Biotechnol. 10(4): 324-30 (1999), incorporated herein by reference in their entireties. PEG monomers can be attached to the protein directly or through a linker, with PEGylation using PEG monomers activated with tresyl chloride (2,2,2-trifluoroethanesulphonyl chloride) permitting direct attachment under mild conditions.

In yet another embodiment, the invention provides analogs of a polypeptide encoded by a nucleic acid molecule according to the instant invention. In a preferred embodiment, the polypeptide is a PSP. In a more preferred embodiment, the analog is derived from a polypeptide having part or all of the amino acid sequence of SEQ ID NO: 136 through 240. In a preferred embodiment, the analog is one that comprises one or more substitutions of non-natural amino acids or non-native inter-residue bonds compared to the naturally-occurring polypeptide. In general, the non-peptide analog is structurally similar to a PSP, but one or more peptide linkages is replaced by a linkage selected from the group consisting of—CH₂NH—, —CH₂S—, —CH₂—CH₂—, —CH═CH—(cis and trans), —COCH₂—, —CH(OH)CH₂—and —CH₂SO—. In another embodiment, the non-peptide analog comprises substitution of one or more amino acids of a PSP with a D-amino acid of the same type or other non-natural amino acid in order to generate more stable peptides. D-amino acids can readily be incorporated during chemical peptide synthesis: peptides assembled from D-amino acids are more resistant to proteolytic attack; incorporation of D-amino acids can also be used to confer specific three-dimensional conformations on the peptide. Other amino acid analogues commonly added during chemical synthesis include omithine, norleucine, phosphorylated amino acids (typically phosphoserine, phosphothreonine, phosphotyrosine), L-malonyltyrosine, a non-hydrolyzable analog of phosphotyrosine (see, e.g., Kole et al., Biochem. Biophys. Res. Com. 209: 817-821 (1995)), and various halogenated phenylalanine derivatives.

Non-natural amino acids can be incorporated during solid phase chemical synthesis or by recombinant techniques, although the former is typically more common. Solid phase chemical synthesis of peptides is well established in the art. Procedures are described, inter alia, in Chan et al. (eds.), Fmoc Solid Phase Peptide Synthesis: A Practical Approach (Practical Approach Series), Oxford Univ. Press (March 2000); Jones, Amino Acid and Peptide Synthesis (Oxford Chemistry Primers, No 7), Oxford Univ. Press (1992); and Bodanszky, Principles of Peptide Synthesis (Springer Laboratory), Springer Verlag (1993); the disclosures of which are incorporated herein by reference in their entireties.

Amino acid analogues having detectable labels are also usefully incorporated during synthesis to provide derivatives and analogs. Biotin, for example can be added using biotinoyl-(9-fluorenylmethoxycarbonyl)-L-lysine (FMOC biocytin) (Molecular Probes, Eugene, Oreg., USA). Biotin can also be added enzymatically by incorporation into a fusion protein of a E. coli BirA substrate peptide. The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS-FMOC-L-glutamic acid or the corresponding tBOC derivative (both from Molecular Probes, Inc., Eugene, Oreg., USA). Tetramethylrhodamine fluorophores can be incorporated during automated FMOC synthesis of peptides using (FMOC)-TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides.

A large number of other FMOC-protected non-natural amino acid analogues capable of incorporation during chemical synthesis are available commercially, including, e.g., Fmoc-2-aminobicyclo[2.2.1]heptane-2-carboxylic acid, Fmoc-3-endo-aminobicyclo[2.2.1]heptane-2-endo-carboxylic acid, Fmoc-3-exo-aminobicyclo[2.2.1]heptane-2-exo-carboxylic acid, Fmoc-3-endo-amino-bicyclo[2.2.1]hept-5-ene-2-endo-carboxylic acid, Fmoc-3-exo-amino-bicyclo[2.2.1]hept-5-ene-2-exo-carboxylic acid, Fmoc-cis-2-amino-1-cyclohexanecarboxylic acid, Fmoc-trans-2-amino-1-cyclohexanecarboxylic acid, Fmoc-1-amino-1-cyclopentanecarboxylic acid, Fmoc-cis-2-amino-1-cyclopentanecarboxylic acid, Fmoc-1-amino-1-cyclopropanecarboxylic acid, Fmoc-D-2-amino-4-(ethylthio)butyric acid, Fmoc-L-2-amino-4-(ethylthio)butyric acid, Fmoc-L-buthionine, Fmoc-S-methyl-L-Cysteine, Fmoc-2-aminobenzoic acid (anthranillic acid), Fmoc-3-aminobenzoic acid, Fmoc-4-aminobenzoic acid, Fmoc-2-aminobenzophenone-2′-carboxylic acid, Fmoc-N-(4-aminobenzoyl)-β-alanine, Fmoc-2-amino-4,5-dimethoxybenzoic acid, Fmoc-4-aminohippuric acid, Fmoc-2-amino-3-hydroxybenzoic acid, Fmoc-2-amino-5-hydroxybenzoic acid, Fmoc-3-amino-4-hydroxybenzoic acid, Fmoc-4-amino-3-hydroxybenzoic acid, Fmoc-4-amino-2-hydroxybenzoic acid, Fmoc-5-amino-2-hydroxybenzoic acid, Fmoc-2-amino-3-methoxybenzoic acid, Fmoc-4-amino-3-methoxybenzoic acid, Fmoc-2-amino-3-methylbenzoic acid, Fmoc-2-amino-5-methylbenzoic acid, Fmoc-2-amino-6-methylbenzoic acid, Fmoc-3-amino-2-methylbenzoic acid, Fmoc-3-amino-4-methylbenzoic acid, Fmoc-4-amino-3-methylbenzoic acid, Fmoc-3-amino-2-naphtoic acid, Fmoc-D,L-3-amino-3-phenylpropionic acid, Fmoc-L-Methyldopa, Fmoc-2-amino-4,6-dimethyl-3-pyridinecarboxylic acid, Fmoc-D,L-amino-2-thiophenacetic acid, Fmoc-4-(carboxymethyl)piperazine, Fmoc-4-carboxypiperazine, Fmoc-4-(carboxymethyl)homopiperazine, Fmoc-4-phenyl-4-piperidinecarboxylic acid, Fmoc-L-1,2,3,4-tetrahydronorharman-3-carboxylic acid, Fmoc-L-thiazolidine-4-carboxylic acid, all available from The Peptide Laboratory (Richmond, CA, USA).

Non-natural residues can also be added biosynthetically by engineering a suppressor tRNA, typically one that recognizes the UAG stop codon, by chemical aminoacylation with the desired unnatural amino acid. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl. Acad. Sci. USA 96(9): 4780-5 (1999); Wang et al., Science 292(5516): 498-500 (2001).

Fusion Proteins

The present invention further provides fusions of each of the polypeptides and fragments of the present invention to heterologous polypeptides. In a preferred embodiment, the polypeptide is a PSP. In a more preferred embodiment, the polypeptide that is fused to the heterologous polypeptide comprises part or all of the amino acid sequence of SEQ ID NO: 136 through 240, or is a mutein, homologous polypeptide, analog or derivative thereof. In an even more preferred embodiment, the nucleic acid molecule encoding the fusion protein comprises all or part of the nucleic acid sequence of SEQ ID NO: 1 through 135, or comprises all or part of a nucleic acid sequence that selectively hybridizes or is homologous to a nucleic acid molecule comprising a nucleic acid sequence of SEQ ID NO: 1 through 135.

The fusion proteins of the present invention will include at least one fragment of the protein of the present invention, which fragment is at least 6, typically at least 8, often at least 15, and usefully at least 16, 17, 18, 19, or 20 amino acids long. The fragment of the protein of the present to be included in the fusion can usefully be at least 25 amino acids long, at least 50 amino acids long, and can be at least 75, 100, or even 150 amino acids long. Fusions that include the entirety of the proteins of the present invention have particular utility.

The heterologous polypeptide included within the fusion protein of the present invention is at least 6 amino acids in length, often at least 8 amino acids in length, and usefully at least 15, 20, and 25 amino acids in length. Fusions that include larger polypeptides, such as the IgG Fc region, and even entire proteins (such as GFP chromophore-containing proteins) are particular useful.

As described above in the description of vectors and expression vectors of the present invention, which discussion is incorporated here by reference in its entirety, heterologous polypeptides to be included in the fusion proteins of the present invention can usefully include those designed to facilitate purification and/or visualization of recombinantly-expressed proteins. See, e.g., Ausubel, Chapter 16, (1992), supra. Although purification tags can also be incorporated into fusions that are chemically synthesized, chemical synthesis typically provides sufficient purity that further purification by HPLC suffices; however, visualization tags as above described retain their utility even when the protein is produced by chemical synthesis, and when so included render the fusion proteins of the present invention useful as directly detectable markers of the presence of a polypeptide of the invention.

As also discussed above, heterologous polypeptides to be included in the fusion proteins of the present invention can usefully include those that facilitate secretion of recombinantly expressed proteins—into the periplasmic space or extracellular milieu for prokaryotic hosts, into the culture medium for eukaryotic cells -through incorporation of secretion signals and/or leader sequences. For example, a His⁶ tagged protein can be purified on a Ni affinity column and a GST fusion protein can be purified on a glutathione affinity column. Similarly, a fusion protein comprising the Fc domain of IgG can be purified on a Protein A or Protein G column and a fusion protein comprising an epitope tag such as myc can be purified using an immunoaffinity column containing an anti-c-myc antibody. It is preferable that the epitope tag be separated from the protein encoded by the essential gene by an enzymatic cleavage site that can be cleaved after purification. See also the discussion of nucleic acid molecules encoding fusion proteins that may be expressed on the surface of a cell.

Other useful protein fusions of the present invention include those that permit use of the protein of the present invention as bait in a yeast two-hybrid system. See Bartel et al. (eds.), The Yeast Two-Hybrid System, Oxford University Press (1997); Zhu et al., Yeast Hybrid Technologies, Eaton Publishing (2000); Fields et al., Trends Genet. 10(8): 286-92 (1994); Mendelsohn et al., Curr. Opin. Biotechnol. 5(5): 482-6 (1994); Luban et al., Curr. Opin. Biotechnol. 6(1): 59-64 (1995); Allen et al, Trends Biochem. Sci. 20(12): 511-6 (1995); Drees, Curr. Opin. Chem. Biol. 3(1): 64-70 (1999); Topcu et al., Pharm. Res. 17(9): 1049-55 (2000); Fashena et al., Gene 250(1-2): 1-14 (2000);; Colas et al., (1996) Genetic selection of peptide aptamers that recognize and inhibit cyclin-dependent kinase 2. Nature 380, 548-550; Norman, T. et al., (1999) Genetic selection of peptide inhibitors of biological pathways. Science 285, 591-595, Fabbrizio et al., (1999) Inhibition of mammalian cell proliferation by genetically selected peptide aptamers that functionally antagonize E2F activity. Oncogene 18, 4357-4363; Xu et al., (1997) Cells that register logical relationships among proteins. Proc Natl Acad Sci USA. 94, 12473-12478; Yang, et al., (1995) Protein-peptide interactions analyzed with the yeast two-hybrid system. Nuc. Acids Res. 23, 1152-1156; Kolonin et al., (1998) Targeting cyclin-dependent kinases in Drosophila with peptide aptamers. Proc Natl Acad Sci USA 95, 14266-14271; Cohen et al., (1998) An artificial cell-cycle inhibitor isolated from a combinatorial library. Proc Natl Acad Sci USA 95, 14272-14277; Uetz, P.; Giot, L.; al, e.; Fields, S.; Rothberg, J. M. (2000) A comprehensive analysis of protein-protein interactions in Saccharomyces cerevisiae. Nature 403, 623-627; Ito, et al., (2001) A comprehensive two-hybrid analysis to explore the yeast protein interactome. Proc Natl Acad Sci USA 98, 4569-4574, the disclosures of which are incorporated herein by reference in their entireties. Typically, such fusion is to either E. coli LexA or yeast GAL4 DNA binding domains. Related bait plasmids are available that express the bait fused to a nuclear localization signal.

Other useful fusion proteins include those that permit display of the encoded protein on the surface of a phage or cell, fusions to intrinsically fluorescent proteins, such as green fluorescent protein (GFP), and fusions to the IgG Fc region, as described above, which discussion is incorporated here by reference in its entirety.

The polypeptides and fragments of the present invention can also usefully be fused to protein toxins, such as Pseudomonas exotoxin A, diphtheria toxin, shiga toxin A, anthrax toxin lethal factor, ricin, in order to effect ablation of cells that bind or take up the proteins of the present invention.

Fusion partners include, inter alia, myc, hemagglutinin (HA), GST, immunoglobulins, β-galactosidase, biotin trpE, protein A, β-lactamase, -amylase, maltose binding protein, alcohol dehydrogenase, polyhistidine (for example, six histidine at the amino and/or carboxyl terminus of the polypeptide), lacZ, green fluorescent protein (GFP), yeast mating factor, GAL4 transcription activation or DNA binding domain, luciferase, and serum proteins such as ovalbumin, albumin and the constant domain of IgG. See, e.g., Ausubel (1992), supra and Ausubel (1999), supra. Fusion proteins may also contain sites for specific enzymatic cleavage, such as a site that is recognized by enzymes such as Factor XIII, trypsin, pepsin, or any other enzyme known in the art. Fusion proteins will typically be made by either recombinant nucleic acid methods, as described above, chemically synthesized using techniques well-known in the art (e.g., a Merrifield synthesis), or produced by chemical cross-linking.

Another advantage of fusion proteins is that the epitope tag can be used to bind the fusion protein to a plate or column through an affinity linkage for screening binding proteins or other molecules that bind to the PSP.

As further described below, the isolated polypeptides, muteins, fusion proteins, homologous proteins or allelic variants of the present invention can readily be used as specific immunogens to raise antibodies that specifically recognize PSPs, their allelic variants and homologues. The antibodies, in turn, can be used, inter alia, specifically to assay for the polypeptides of the present invention, particularly PSPs, e.g. by ELISA for detection of protein fluid samples, such as serum, by immunohistochemistry or laser scanning cytometry, for detection of protein in tissue samples, or by flow cytometry, for detection of intracellular protein in cell suspensions, for specific antibody-mediated isolation and/or purification of PSPs, as for example by immunoprecipitation, and for use as specific agonists or antagonists of PSPs.

One may determine whether polypeptides including muteins, fusion proteins, homologous proteins or allelic variants are functional by methods known in the art. For instance, residues that are tolerant of change while retaining function can be identified by altering the protein at known residues using methods known in the art, such as alanine scanning mutagenesis, Cunningham et al., Science 244(4908): 1081-5 (1989); transposon linker scanning mutagenesis, Chen et al., Gene 263(1-2): 39-48 (2001); combinations of homolog- and alanine-scanning mutagenesis, Jin et al., J. Mol. Biol. 226(3): 851-65 (1992); combinatorial alanine scanning, Weiss et al., Proc. Natl. Acad. Sci USA 97(16): 8950-4 (2000), followed by functional assay. Transposon linker scanning kits are available commercially (New England Biolabs, Beverly, Mass., USA, catalog. no. E7-102S; EZ::TN™ In-Frame Linker Insertion Kit, catalogue no. EZI04KN, Epicentre Technologies Corporation, Madison, Wis., USA).

Purification of the polypeptides including fragments, homologous polypeptides, muteins, analogs, derivatives and fusion proteins is well-known and within the skill of one having ordinary skill in the art. See, e.g., Scopes, Protein Purification, 2d ed. (1987). Purification of recombinantly expressed polypeptides is described above. Purification of chemically-synthesized peptides can readily be effected, e.g., by HPLC.

Accordingly, it is an aspect of the present invention to provide the isolated proteins of the present invention in pure or substantially pure form in the presence of absence of a stabilizing agent. Stabilizing agents include both proteinaceous or non-proteinaceous material and are well-known in the art. Stabilizing agents, such as albumin and polyethylene glycol (PEG) are known and are commercially available.

Although high levels of purity are preferred when the isolated proteins of the present invention are used as therapeutic agents, such as in vaccines and as replacement therapy, the isolated proteins of the present invention are also useful at lower purity. For example, partially purified proteins of the present invention can be used as immunogens to raise antibodies in laboratory animals.

In preferred embodiments, the purified and substantially purified proteins of the present invention are in compositions that lack detectable ampholytes, acrylamide monomers, bis-acrylamide monomers, and polyacrylamide.

The polypeptides, fragments, analogs, derivatives and fusions of the present invention can usefully be attached to a substrate. The substrate can be porous or solid, planar or non-planar; the bond can be covalent or noncovalent.

For example, the polypeptides, fragments, analogs, derivatives and fusions of the present invention can usefully be bound to a porous substrate, commonly a membrane, typically comprising nitrocellulose, polyvinylidene fluoride (PVDF), or cationically derivatized, hydrophilic PVDF; so bound, the proteins, fragments, and fusions of the present invention can be used to detect and quantify antibodies, e.g. in serum, that bind specifically to the immobilized protein of the present invention.

As another example, the polypeptides, fragments, analogs, derivatives and fusions of the present invention can usefully be bound to a substantially nonporous substrate, such as plastic, to detect and quantify antibodies, e.g. in serum, that bind specifically to the immobilized protein of the present invention. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof; when the assay is performed in a standard microtiter dish, the plastic is typically polystyrene.

The polypeptides, fragments, analogs, derivatives and fusions of the present invention can also be attached to a substrate suitable for use as a surface enhanced laser desorption ionization source; so attached, the protein, fragment, or fusion of the present invention is useful for binding and then detecting secondary proteins that bind with sufficient affinity or avidity to the surface-bound protein to indicate biologic interaction there between. The proteins, fragments, and fusions of the present invention can also be attached to a substrate suitable for use in surface plasmon resonance detection; so attached, the protein, fragment, or fusion of the present invention is useful for binding and then detecting secondary proteins that bind with sufficient affinity or avidity to the surface-bound protein to indicate biological interaction there between.

Antibodies

In another aspect, the invention provides antibodies, including fragments and derivatives thereof, that bind specifically to polypeptides encoded by the nucleic acid molecules of the invention, as well as antibodies that bind to fragments, muteins, derivatives and analogs of the polypeptides. In a preferred embodiment, the antibodies are specific for a polypeptide that is a PSP, or a fragment, mutein, derivative, analog or fusion protein thereof. In a more preferred embodiment, the antibodies are specific for a polypeptide that comprises SEQ ID NO: 136 through 240, or a fragment, mutein, derivative, analog or fusion protein thereof.

The antibodies of the present invention can be specific for linear epitopes, discontinuous epitopes, or conformational epitopes of such proteins or protein fragments, either as present on the protein in its native conformation or, in some cases, as present on the proteins as denatured, as, e.g., by solubilization in SDS. New epitopes may be also due to a difference in post translational modifications (PTMs) in disease versus normal tissue. For example, a particular site on a PSP may be glycosylated in cancerous cells, but not glycosylated in normal cells or visa versa. In addition, alternative splice forms of a PSP may be indicative of cancer. Differential degradation of the C or N-terminus of a PSP may also be a marker or target for anticancer therapy. For example, a PSP may be N-terminal degraded in cancer cells exposing new epitopes to which antibodies may selectively bind for diagnostic or therapeutic uses.

As is well-known in the art, the degree to which an antibody can discriminate as among molecular species in a mixture will depend, in part, upon the conformational relatedness of the species in the mixture; typically, the antibodies of the present invention will discriminate over adventitious binding to non-PSP polypeptides by at least 2-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the protein of the present invention in samples derived from human prostate.

Typically, the affinity or avidity of an antibody (or antibody multimer, as in the case of an IgM pentamer) of the present invention for a protein or protein fragment of the present invention will be at least about 1×10⁻⁶ molar (M), typically at least about 5×10⁻⁷ M, 1×10⁻⁷ M, with affinities and avidities of at least 1×10⁻⁸ M, 5×10⁻⁹ M, 1×10⁻¹⁰ M and up to 1×10⁻¹³ M proving especially useful.

The antibodies of the present invention can be naturally-occurring forms, such as IgG, IgM, IgD, IgE, IgY, and IgA, from any avian, reptilian, or mammalian species.

Human antibodies can, but will infrequently, be drawn directly from human donors or human cells. In this case, antibodies to the proteins of the present invention will typically have resulted from fortuitous immunization, such as autoimmune immunization, with the protein or protein fragments of the present invention. Such antibodies will typically, but will not invariably, be polyclonal. In addition, individual polyclonal antibodies may be isolated and cloned to generate monoclonals.

Human antibodies are more frequently obtained using transgenic animals that express human immunoglobulin genes, which transgenic animals can be affirmatively immunized with the protein immunogen of the present invention. Human Ig-transgenic mice capable of producing human antibodies and methods of producing human antibodies therefrom upon specific immunization are described, inter alia, in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE, IgY, and IgA antibodies of the present invention can also be obtained from other species, including mammals such as rodents (typically mouse, but also rat, guinea pig, and hamster) lagomorphs, typically rabbits, and also larger mammals, such as sheep, goats, cows, and horses, and other egg laying birds or reptiles such as chickens or alligators. For example, avian antibodies may be generated using techniques described in WO 00/29444, published May 25, 2000, the contents of which are hereby incorporated in their entirety. In such cases, as with the transgenic human-antibody-producing non-human mammals, fortuitous immunization is not required, and the non-human mammal is typically affirmatively immunized, according to standard immunization protocols, with the protein or protein fragment of the present invention.

As discussed above, virtually all fragments of 8 or more contiguous amino acids of the proteins of the present invention can be used effectively as immunogens when conjugated to a carrier, typically a protein such as bovine thyroglobulin, keyhole limpet hemocyanin, or bovine serum albumin, conveniently using a bifunctional linker such as those described elsewhere above, which discussion is incorporated by reference here.

Immunogenicity can also be conferred by fusion of the polypeptide and fragments of the present invention to other moieties. For example, peptides of the present invention can be produced by solid phase synthesis on a branched polylysine core matrix; these multiple antigenic peptides (MAPs) provide high purity, increased avidity, accurate chemical definition and improved safety in vaccine development. Tam et al., Proc. Natl. Acad. Sci. USA 85: 5409-5413 (1988); Posnett et al., J. Biol. Chem. 263: 1719-1725 (1988).

Protocols for immunizing non-human mammals or avian species are well-established in the art. See Harlow et al. (eds.), Using Antibodies: A Laboratory Manual, Cold Spring Harbor Laboratory (1998); Coligan et al. (eds.), Current Protocols in Immunology, John Wiley & Sons, Inc. (2001); Zola, Monoclonal Antibodies: Preparation and Use of Monoclonal Antibodies and Engineered Antibody Derivatives (Basics: From Background to Bench), Springer Verlag (2000); Gross M, Speck J. Dtsch. Tierarztl. Wochenschr. 103: 417-422 (1996), the disclosures of which are incorporated herein by reference. Immunization protocols often include multiple immunizations, either with or without adjuvants such as Freund's complete adjuvant and Freund's incomplete adjuvant, and may include naked DNA immunization (Moss, Semin. Immunol. 2: 317-327 (1990).

Antibodies from non-human mammals and avian species can be polyclonal or monoclonal, with polyclonal antibodies having certain advantages in immunohistochemical detection of the proteins of the present invention and monoclonal antibodies having advantages in identifying and distinguishing particular epitopes of the proteins of the present invention. Antibodies from avian species may have particular advantage in detection of the proteins of the present invention, in human serum or tissues (Vikinge et al., Biosens. Bioelectron. 13: 1257-1262 (1998).

Following immunization, the antibodies of the present invention can be produced using any art-accepted technique. Such techniques are well-known in the art, Coligan, supra; Zola, supra; Howard et al. (eds.), Basic Methods in Antibody Production and Characterization, CRC Press (2000); Harlow, supra; Davis (ed.), Monoclonal Antibody Protocols, Vol. 45, Humana Press (1995); Delves (ed.), Antibody Production: Essential Techniques, John Wiley & Son Ltd (1997); Kenney, Antibody Solution: An Antibody Methods Manual, Chapman & Hall (1997), incorporated herein by reference in their entireties, and thus need not be detailed here.

Briefly, however, such techniques include, inter alia, production of monoclonal antibodies by hybridomas and expression of antibodies or fragments or derivatives thereof from host cells engineered to express immunoglobulin genes or fragments thereof. These two methods of production are not mutually exclusive: genes encoding antibodies specific for the proteins or protein fragments of the present invention can be cloned from hybridomas and thereafter expressed in other host cells. Nor need the two necessarily be performed together: e.g., genes encoding antibodies specific for the proteins and protein fragments of the present invention can be cloned directly from B cells known to be specific for the desired protein, as further described in U.S. Pat. No. 5,627,052, the disclosure of which is incorporated herein by reference in its entirety, or from antibody-displaying phage.

Recombinant expression in host cells is particularly useful when fragments or derivatives of the antibodies of the present invention are desired.

Host cells for recombinant production of either whole antibodies, antibody fragments, or antibody derivatives can be prokaryotic or eukaryotic.

Prokaryotic hosts are particularly useful for producing phage displayed antibodies of the present invention.

The technology of phage-displayed antibodies, in which antibody variable region fragments are fused, for example, to the gene III protein (pIII) or gene VIII protein (pVIII) for display on the surface of filamentous phage, such as M13, is by now well-established. See, e.g., Sidhu, Curr. Opin. Biotechnol. 11(6): 610-6 (2000); Griffiths et al, Curr. Opin. Biotechnol. 9(1): 102-8 (1998); Hoogenboom et al., Immunotechnology, 4(1): 1-20 (1998); Rader et al, Current Opinion in Biotechnology 8: 503-508 (1997); Aujame et al, Human Antibodies 8: 155-168 (1997); Hoogenboom, Trends in Biotechnol. 15: 62-70 (1997); de Kruif et al, 17: 453-455 (1996); Barbas et al., Trends in Biotechnol. 14: 230-234 (1996); Winter et al, Ann. Rev. Immunol. 433-455 (1994). Techniques and protocols required to generate, propagate, screen (pan), and use the antibody fragments from such libraries have recently been compiled. See, e.g., Barbas (2001), supra; Kay, supra; Abelson, supra, the disclosures of which are incorporated herein by reference in their entireties.

Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell.

Eukaryotic cells are also useful for expression of the antibodies, antibody fragments, and antibody derivatives of the present invention.

For example, antibody fragments of the present invention can be produced in Pichia pastoris and in Saccharomyces cerevisiae. See, e.g., Takahashi et al., Biosci. Biotechnol. Biochem. 64(10): 2138-44 (2000); Freyre et al., J. Biotechnol. 76(2-3):1 57-63 (2000); Fischer et al., Biotechnol. Appl. Biochem. 30 (Pt 2): 117-20 (1999); Pennell et al., Res. Immunol. 149(6): 599-603 (1998); Eldin et al., J. Immunol. Methods. 201(1): 67-75 (1997);, Frenken et al., Res. Immunol. 149(6): 589-99 (1998); Shusta et al., Nature Biotechnol. 16(8): 773-7 (1998), the disclosures of which are incorporated herein by reference in their entireties.

Antibodies, including antibody fragments and derivatives, of the present invention can also be produced in insect cells. See, e.g., Li et al., Protein Expr. Purif. 21(1): 121-8 (2001); Ailor et al., Biotechnol. Bioeng. 58(2-3): 196-203 (1998); Hsu et al., Biotechnol. Prog. 13(1): 96-104 (1997); Edelman et al., Immunology 91(1): 13-9 (1997); and Nesbit et al., J. Immunol. Methods 151(1-2): 201-8 (1992), the disclosures of which are incorporated herein by reference in their entireties.

Antibodies and fragments and derivatives thereof of the present invention can also be produced in plant cells, particularly maize or tobacco, Giddings et al., Nature Biotechnol. 18(11): 1151-5 (2000); Gavilondo et al., Biotechniques 29(1): 128-38 (2000); Fischer et al., J. Biol. Regul. Homeost. Agents 14(2): 83-92 (2000); Fischer et al., Biotechnol. Appl. Biochem. 30 (Pt 2): 113-6 (1999); Fischer et al., Biol. Chem. 380(7-8): 825-39 (1999); Russell, Curr. Top. Microbiol. Immunol. 240: 119-38 (1999); and Ma et al., Plant Physiol. 109(2): 341-6 (1995), the disclosures of which are incorporated herein by reference in their entireties.

Antibodies, including antibody fragments and derivatives, of the present invention can also be produced in transgenic, non-human, mammalian milk. See, e.g. Pollock et al., J. Immunol Methods. 231: 147-57 (1999); Young et al., Res. Immunol. 149: 609-10 (1998); Limonta et al., Immunotechnology 1: 107-13 (1995), the disclosures of which are incorporated herein by reference in their entireties.

Mammalian cells useful for recombinant expression of antibodies, antibody fragments, and antibody derivatives of the present invention include CHO cells, COS cells, 293 cells, and myeloma cells.

Verma et al., J. Immunol. Methods 216(1-2):165-81 (1998), herein incorporated by reference, review and compare bacterial, yeast, insect and mammalian expression systems for expression of antibodies.

Antibodies of the present invention can also be prepared by cell free translation, as further described in Merk et al., J. Biochem. (Tokyo) 125(2): 328-33 (1999) and Ryabova et al., Nature Biotechnol. 15(1): 79-84 (1997), and in the milk of transgenic animals, as further described in Pollock et al., J. Immunol. Methods 231(1-2): 147-57 (1999), the disclosures of which are incorporated herein by reference in their entireties.

The invention further provides antibody fragments that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful fragments are Fab, Fab′, Fv, F(ab)′₂, and single chain Fv (scFv) fragments. Other useful fragments are described in Hudson, Curr. Opin. Biotechnol. 9(4): 395-402 (1998).

It is also an aspect of the present invention to provide antibody derivatives that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

Among such useful derivatives are chimeric, primatized, and humanized antibodies; such derivatives are less immunogenic in human beings, and thus more suitable for in vivo administration, than are unmodified antibodies from non-human mammalian species. Another useful derivative is PEGylation to increase the serum half life of the antibodies.

Chimeric antibodies typically include heavy and/or light chain variable regions (including both CDR and framework residues) of immunoglobulins of one species, typically mouse, fused to constant regions of another species, typically human. See, e.g., U.S. Pat. No. 5,807,715; Morrison et al., Proc. Natl. Acad. Sci USA. 81(21): 6851-5 (1984); Sharon et al., Nature 309(5966): 364-7 (1984); Takeda et al., Nature 314(6010): 452-4 (1985), the disclosures of which are incorporated herein by reference in their entireties. Primatized and humanized antibodies typically include heavy and/or light chain CDRs from a murine antibody grafted into a non-human primate or human antibody V region framework, usually further comprising a human constant region, Riechmann et al., Nature 332(6162): 323-7 (1988); Co et al., Nature 351(6326): 501-2 (1991); U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties.

Other useful antibody derivatives of the invention include heteromeric antibody complexes and antibody fusions, such as diabodies (bispecific antibodies), single-chain diabodies, and intrabodies.

It is contemplated that the nucleic acids encoding the antibodies of the present invention can be operably joined to other nucleic acids forming a recombinant vector for cloning or for expression of the antibodies of the invention. The present invention includes any recombinant vector containing the coding sequences, or part thereof, whether for eukaryotic transduction, transfection or gene therapy. Such vectors may be prepared using conventional molecular biology techniques, known to those with skill in the art, and would comprise DNA encoding sequences for the immunoglobulin V-regions including framework and CDRs or parts thereof, and a suitable promoter either with or without a signal sequence for intracellular transport. Such vectors may be transduced or transfected into eukaryotic cells or used for gene therapy (Marasco et al., Proc. Natl. Acad. Sci. (USA) 90: 7889-7893 (1993); Duan et al., Proc. Natl. Acad. Sci. (USA) 91: 5075-5079 (1994), by conventional techniques, known to those with skill in the art.

The antibodies of the present invention, including fragments and derivatives thereof, can usefully be labeled. It is, therefore, another aspect of the present invention to provide labeled antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

The choice of label depends, in part, upon the desired use.

For example, when the antibodies of the present invention are used for immunohistochemical staining of tissue samples, the label is preferably an enzyme that catalyzes production and local deposition of a detectable product.

Enzymes typically conjugated to antibodies to permit their immunohistochemical visualization are well-known, and include alkaline phosphatase, β-galactosidase, glucose oxidase, horseradish peroxidase (HRP), and urease. Typical substrates for production and deposition of visually detectable products include o-nitrophenyl-beta-D-galactopyranoside (ONPG); o-phenylenediamine dihydrochloride (OPD); p-nitrophenyl phosphate (PNPP); p-nitrophenyl-beta-D-galactopryanoside (PNPG); 3′,3′-diaminobenzidine (DAB); 3-amino-9-ethylcarbazole (AEC); 4-chloro-1-naphthol (CN); 5-bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Glucoside.

Other substrates can be used to produce products for local deposition that are luminescent. For example, in the presence of hydrogen peroxide (H₂O₂), horseradish peroxidase (HRP) can catalyze the oxidation of cyclic diacylhydrazides, such as luminol. Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133: 331-53 (1986); Kricka et al, J. Immunoassay 17(1): 67-83 (1996); and Lundqvist et al., J. Biolumin. Chemilumin. 10(6): 353-9 (1995), the disclosures of which are incorporated herein by reference in their entireties. Kits for such enhanced chemiluminescent detection (ECL) are available commercially.

The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores.

There are a wide variety of fluorophore labels that can usefully be attached to the antibodies of the present invention.

For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCP-Cy5.5, PE-Cy5, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention.

For secondary detection using labeled avidin, streptavidin, captavidin or neutravidin, the antibodies of the present invention can usefully be labeled with biotin.

When the antibodies of the present invention are used, e.g., for Western blotting applications, they can usefully be labeled with radioisotopes, such as ³³P, ³²P, ³⁵S, ³H, and ¹²⁵I.

As another example, when the antibodies of the present invention are used for radioimmunotherapy, the label can usefully be ²²⁸Th, ²²⁷Ac, ²²⁵Ac, ²²³Ra, ²¹³Bi, ²¹²Pb, ²¹²Bi, ²¹¹At, ²⁰³Pb, ¹⁹⁴OS, ¹⁸⁸Re, ¹⁸⁶Re, ¹⁵³Sm, ¹⁴⁹Tb, ¹³¹I, ¹²⁵I, ¹¹¹In, ¹⁰⁵Rh, ^{99m}Tc, ⁹⁷Ru, ⁹⁰Y, ⁹⁰Sr, ⁸⁸y, ⁷²Se, ⁶⁷CU, or ⁴⁷Sc.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2): 529-38 (1998), or by radioisotopic labeling.

As would be understood, use of the labels described above is not restricted to the application for which they are mentioned.

The antibodies of the present invention, including fragments and derivatives thereof, can also be conjugated to toxins, in order to target the toxin's ablative action to cells that display and/or express the proteins of the present invention. Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, vol. 166), Humana Press (2000); and Frankel et al. (eds.), Clinical Applications of Immunotoxins, Springer-Verlag (1998), the disclosures of which are incorporated herein by reference in their entireties.

The antibodies of the present invention can usefully be attached to a substrate, and it is, therefore, another aspect of the invention to provide antibodies that bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention, attached to a substrate.

Substrates can be porous or nonporous, planar or nonplanar.

For example, the antibodies of the present invention can usefully be conjugated to filtration media, such as NHS-activated Sepharose or CNBr-activated Sepharose for purposes of immunoaffinity chromatography.

For example, the antibodies of the present invention can usefully be attached to paramagnetic microspheres, typically by biotin-streptavidin interaction, which microspheres can then be used for isolation of cells that express or display the proteins of the present invention. As another example, the antibodies of the present invention can usefully be attached to the surface of a microtiter plate for ELISA.

As noted above, the antibodies of the present invention can be produced in prokaryotic and eukaryotic cells. It is, therefore, another aspect of the present invention to provide cells that express the antibodies of the present invention, including hybridoma cells, B cells, plasma cells, and host cells recombinantly modified to express the antibodies of the present invention.

In yet a further aspect, the present invention provides aptamers evolved to bind specifically to one or more of the proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated nucleic acids of the present invention.

In sum, one of skill in the art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

Transgenic Animals and Cells

In another aspect, the invention provides transgenic cells and non-human organisms comprising nucleic acid molecules of the invention. In a preferred embodiment, the transgenic cells and non-human organisms comprise a nucleic acid molecule encoding a PSP. In a preferred embodiment, the PSP comprises an amino acid sequence selected from SEQ ID NO: 136 through 240, or a fragment, mutein, homologous protein or allelic variant thereof. In another preferred embodiment, the transgenic cells and non-human organism comprise a PSNA of the invention, preferably a PSNA comprising a nucleotide sequence selected from the group consisting of SEQ ID NO: 1 through 135, or a part, substantially similar nucleic acid molecule, allelic variant or hybridizing nucleic acid molecule thereof.

In another embodiment, the transgenic cells and non-human organisms have a targeted disruption or replacement of the endogenous orthologue of the human PSG. The transgenic cells can be embryonic stem cells or somatic cells. The transgenic non-human organisms can be chimeric, nonchimeric heterozygotes, and nonchimeric homozygotes. Methods of producing transgenic animals are well-known in the art. See, e.g., Hogan et al., Manipulating the Mouse Embryo: A Laboratory Manual, 2d ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999).

Any technique known in the art may be used to introduce a nucleic acid molecule of the invention into an animal to produce the founder lines of transgenic animals. Such techniques include, but are not limited to, pronuclear microinjection. (see, e.g., Paterson et al., Appl. Microbiol Biotechnol. 40: 691-698 (1994); Carver et al., Biotechnology 11: 1263-1270 (1993); Wright et al., Biotechnology 9: 830-834 (1991); and U.S. Pat. No. 4,873,191 (1989 retrovirus-mediated gene transfer into germ lines, blastocysts or embryos (see, e.g., Van der Putten et al., Proc. Natl. Acad. Sci., USA 82: 6148-6152 (1985)); gene targeting in embryonic stem cells (see, e.g., Thompson et al., Cell 56: 313-321 (1989)); electroporation of cells or embryos (see, e.g., Lo, 1983, Mol. Cell. Biol. 3: 1803-1814 (1983)); introduction using a gene gun (see, e.g., Ulmer et al., Science 259: 1745-49 (1993); introducing nucleic acid constructs into embryonic pleuripotent stem cells and transferring the stem cells back into the blastocyst; and sperm-mediated gene transfer (see, e.g., Lavitrano et al., Cell 57: 717-723 (1989)).

Other techniques include, for example, nuclear transfer into enucleated oocytes of nuclei from cultured embryonic, fetal, or adult cells induced to quiescence (see, e.g., Campell et al., Nature 380: 64-66 (1996); Wilmut et al., Nature 385: 810-813 (1997)). The present invention provides for transgenic animals that carry the transgene (i.e., a nucleic acid molecule of the invention) in all their cells, as well as animals which carry the transgene in some, but not all their cells, i.e., mosaic animals or chimeric animals.

The transgene may be integrated as a single transgene or as multiple copies, such as in concatamers, e.g., head-to-head tandems or head-to-tail tandems. The transgene may also be selectively introduced into and activated in a particular cell type by following, e.g., the teaching of Lasko et al. et al., Proc. Natl. Acad. Sci. USA 89: 6232-6236 (1992). The regulatory sequences required for such a cell-type specific activation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (RT-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

Methods for creating a transgenic animal with a disruption of a targeted gene are also well-known in the art. In general, a vector is designed to comprise some nucleotide sequences homologous to the endogenous targeted gene. The vector is introduced into a cell so that it may integrate, via homologous recombination with chromosomal sequences, into the endogenous gene, thereby disrupting the function of the endogenous gene. The transgene may also be selectively introduced into a particular cell type, thus inactivating the endogenous gene in only that cell type. See, e.g., Gu et al., Science 265: 103-106 (1994). The regulatory sequences required for such a cell-type specific inactivation will depend upon the particular cell type of interest, and will be apparent to those of skill in the art. See, e.g., Smithies et al., Nature 317: 230-234 (1985); Thomas et al., Cell 51: 503-512 (1987); Thompson et al., Cell 5: 313-321 (1989).

In one embodiment, a mutant, non-functional nucleic acid molecule of the invention (or a completely unrelated DNA sequence) flanked by DNA homologous to the endogenous nucleic acid sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene. See, e.g., Thomas, supra and Thompson, supra. However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from an animal or patient or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc.

The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. See, e.g., U.S. Pat. Nos. 5,399,349 and 5,460,959, each of which is incorporated by reference herein in its entirety.

When the cells to be administered are non-autologous or non-MHC compatible cells, they can be administered using well-known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

Computer Readable Means

A further aspect of the invention relates to a computer readable means for storing the nucleic acid and amino acid sequences of the instant invention. In a preferred embodiment, the invention provides a computer readable means for storing SEQ ID NO: 1 through 135 and SEQ ID NO: 136 through 240 as described herein, as the complete set of sequences or in any combination. The records of the computer readable means can be accessed for reading and display and for interface with a computer system for the application of programs allowing for the location of data upon a query for data meeting certain criteria, the comparison of sequences, the alignment or ordering of sequences meeting a set of criteria, and the like.

The nucleic acid and amino acid sequences of the invention are particularly useful as components in databases useful for search analyses as well as in sequence analysis algorithms. As used herein, the terms “nucleic acid sequences of the invention” and “amino acid sequences of the invention” mean any detectable chemical or physical characteristic of a polynucleotide or polypeptide of the invention that is or may be reduced to or stored in a computer readable form. These include, without limitation, chromatographic scan data or peak data, photographic data or scan data therefrom, and mass spectrographic data.

This invention provides computer readable media having stored thereon sequences of the invention. A computer readable medium may comprise one or more of the following: a nucleic acid sequence comprising a sequence of a nucleic acid sequence of the invention; an amino acid sequence comprising an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of one or more nucleic acid sequences of the invention; a data set representing a nucleic acid sequence encoding an amino acid sequence comprising the sequence of an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of a nucleic acid sequence of the invention; a data set representing a nucleic acid sequence encoding an amino acid sequence comprising the sequence of an amino acid sequence of the invention. The computer readable medium can be any composition of matter used to store information or data, including, for example, commercially available floppy disks, tapes, hard drives, compact disks, and video disks.

Also provided by the invention are methods for the analysis of character sequences, particularly genetic sequences. Preferred methods of sequence analysis include, for example, methods of sequence homology analysis, such as identity and similarity analysis, RNA structure analysis, sequence assembly, cladistic analysis, sequence motif analysis, open reading frame determination, nucleic acid base calling, and sequencing chromatogram peak analysis.

A computer-based method is provided for performing nucleic acid sequence identity or similarity identification. This method comprises the steps of providing a nucleic acid sequence comprising the sequence of a nucleic acid of the invention in a computer readable medium; and comparing said nucleic acid sequence to at least one nucleic acid or amino acid sequence to identify sequence identity or similarity.

A computer-based method is also provided for performing amino acid homology identification, said method comprising the steps of: providing an amino acid sequence comprising the sequence of an amino acid of the invention in a computer readable medium; and comparing said an amino acid sequence to at least one nucleic acid or an amino acid sequence to identify homology.

A computer-based method is still further provided for assembly of overlapping nucleic acid sequences into a single nucleic acid sequence, said method comprising the steps of: providing a first nucleic acid sequence comprising the sequence of a nucleic acid of the invention in a computer readable medium; and screening for at least one overlapping region between said first nucleic acid sequence and a second nucleic acid sequence.

Diagnostic Methods for Prostate Cancer

The present invention also relates to quantitative and qualitative diagnostic assays and methods for detecting, diagnosing, monitoring, staging and predicting cancers by comparing expression of a PSNA or a PSP in a human patient that has or may have prostate cancer, or who is at risk of developing prostate cancer, with the expression of a PSNA or a PSP in a normal human control. For purposes of the present invention, “expression of a PSNA” or “PSNA expression” means the quantity of PSG mRNA that can be measured by any method known in the art or the level of transcription that can be measured by any method known in the art in a cell, tissue, organ or whole patient. Similarly, the term “expression of a PSP” or “PSP expression” means the amount of PSP that can be measured by any method known in the art or the level of translation of a PSG PSNA that can be measured by any method known in the art.

The present invention provides methods for diagnosing prostate cancer in a patient, in particular squamous cell carcinoma, by analyzing for changes in levels of PSNA or PSP in cells, tissues, organs or bodily fluids compared with levels of PSNA or PSP in cells, tissues, organs or bodily fluids of preferably the same type from a normal human control, wherein an increase, or decrease in certain cases, in levels of a PSNA or PSP in the patient versus the normal human control is associated with the presence of prostate cancer or with a predilection to the disease. In another preferred embodiment, the present invention provides methods for diagnosing prostate cancer in a patient by analyzing changes in the structure of the mRNA of a PSG compared to the mRNA from a normal control. These changes include, without limitation, aberrant splicing, alterations in polyadenylation and/or alterations in 5′ nucleotide capping. In yet another preferred embodiment, the present invention provides methods for diagnosing prostate cancer in a patient by analyzing changes in a PSP compared to a PSP from a normal control. These changes include, e.g., alterations in glycosylation and/or phosphorylation of the PSP or subcellular PSP localization.

In a preferred embodiment, the expression of a PSNA is measured by determining the amount of an mRNA that encodes an amino acid sequence selected from SEQ ID NO: 136 through 240, a homolog, an allelic variant, or a fragment thereof. In a more preferred embodiment, the PSNA expression that is measured is the level of expression of a PSNA mRNA selected from SEQ ID NO: 1 through 135, or a hybridizing nucleic acid, homologous nucleic acid or allelic variant thereof, or a part of any of these nucleic acids. PSNA expression may be measured by any method known in the art, such as those described supra, including measuring mRNA expression by Northern blot, quantitative or qualitative reverse transcriptase PCR (RT-PCR), microarray, dot or slot blots or in situ hybridization. See, e.g., Ausubel (1992), supra; Ausubel (1999), supra; Sambrook (1989), supra; and Sambrook (2001), supra. PSNA transcription maybe measured by any method known in the art including using a reporter gene hooked up to the promoter of a PSG of interest or doing nuclear run-off assays. Alterations in mRNA structure, e.g., aberrant splicing variants, may be determined by any method known in the art, including, RT-PCR followed by sequencing or restriction analysis. As necessary, PSNA expression may be compared to a known control, such as normal prostate nucleic acid, to detect a change in expression.

In another preferred embodiment, the expression of a PSP is measured by determining the level of a PSP having an amino acid sequence selected from the group consisting of SEQ ID NO: 136 through 240, a homolog, an allelic variant, or a fragment thereof. Such levels are preferably determined in at least one of cells, tissues, organs and/or bodily fluids, including determination of normal and abnormal levels. Thus, for instance, a diagnostic assay in accordance with the invention for diagnosing over- or underexpression of PSNA or PSP compared to normal control bodily fluids, cells, or tissue samples may be used to diagnose the presence of prostate cancer. The expression level of a PSP may be determined by any method known in the art, such as those described supra. In a preferred embodiment, the PSP expression level may be determined by radioimmunoassays, competitive-binding assays, ELISA, Western blot, FACS, immunohistochemistry, immunoprecipitation, proteomic approaches: two-dimensional gel electrophoresis (2D electrophoresis) and non-gel-based approaches such as mass spectrometry or protein interaction profiling. See, e.g, Harlow (1999), supra; Ausubel (1992), supra; and Ausubel (1999), supra. Alterations in the PSP structure may be determined by any method known in the art, including, e.g., using antibodies that specifically recognize phosphoserine, phosphothreonine or phosphotyrosine residues, two-dimensional polyacrylamide gel electrophoresis (2D PAGE) and/or chemical analysis of amino acid residues of the protein. Id.

In a preferred embodiment, a radioimmunoassay (RIA) or an ELISA is used. An antibody specific to a PSP is prepared if one is not already available. In a preferred embodiment, the antibody is a monoclonal antibody. The anti-PSP antibody is bound to a solid support and any free protein binding sites on the solid support are blocked with a protein such as bovine serum albumin. A sample of interest is incubated with the antibody on the solid support under conditions in which the PSP will bind to the anti-PSP antibody. The sample is removed, the solid support is washed to remove unbound material, and an anti-PSP antibody that is linked to a detectable reagent (a radioactive substance for RIA and an enzyme for ELISA) is added to the solid support and incubated under conditions in which binding of the PSP to the labeled antibody will occur. After binding, the unbound labeled antibody is removed by washing. For an ELISA, one or more substrates are added to produce a colored reaction product that is based upon the amount of a PSP in the sample. For an RIA, the solid support is counted for radioactive decay signals by any method known in the art. Quantitative results for both RIA and ELISA typically are obtained by reference to a standard curve.

Other methods to measure PSP levels are known in the art. For instance, a competition assay may be employed wherein an anti-PSP antibody is attached to a solid support and an allocated amount of a labeled PSP and a sample of interest are incubated with the solid support. The amount of labeled PSP detected which is attached to the solid support can be correlated to the quantity of a PSP in the sample.

Of the proteomic approaches, 2D PAGE is a well-known technique. Isolation of individual proteins from a sample such as serum is accomplished using sequential separation of proteins by isoelectric point and molecular weight. Typically, polypeptides are first separated by isoelectric point (the first dimension) and then separated by size using an electric current (the second dimension). In general, the second dimension is perpendicular to the first dimension. Because no two proteins with different sequences are identical on the basis of both size and charge, the result of 2D PAGE is a roughly square gel in which each protein occupies a unique spot. Analysis of the spots with chemical or antibody probes, or subsequent protein microsequencing can reveal the relative abundance of a given protein and the identity of the proteins in the sample.

Expression levels of a PSNA can be determined by any method known in the art, including PCR and other nucleic acid methods, such as ligase chain reaction (LCR) and nucleic acid sequence based amplification (NASBA), can be used to detect malignant cells for diagnosis and monitoring of various malignancies. For example, reverse-transcriptase PCR (RT-PCR) is a powerful technique which can be used to detect the presence of a specific mRNA population in a complex mixture of thousands of other mRNA species. In RT-PCR, an mRNA species is first reverse transcribed to complementary DNA (cDNA) with use of the enzyme reverse transcriptase; the cDNA is then amplified as in a standard PCR reaction.

Hybridization to specific DNA molecules (e.g., oligonucleotides) arrayed on a solid support can be used to both detect the expression of and quantitate the level of expression of one or more PSNAs of interest. In this approach, all or a portion of one or more PSNAs is fixed to a substrate. A sample of interest, which may comprise RNA, e.g., total RNA or poly A-selected mRNA, or a complementary DNA (cDNA) copy of the RNA is incubated with the solid support under conditions in which hybridization will occur between the DNA on the solid support and the nucleic acid molecules in the sample of interest. Hybridization between the substrate-bound DNA and the nucleic acid molecules in the sample can be detected and quantitated by several means, including, without limitation, radioactive labeling or fluorescent labeling of the nucleic acid molecule or a secondary molecule designed to detect the hybrid.

The above tests can be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva or any other bodily secretion or derivative thereof. By blood it is meant to include whole blood, plasma, serum or any derivative of blood. In a preferred embodiment, the specimen tested for expression of PSNA or PSP includes, without limitation, prostate tissue, fluid obtained by bronchial alveolar lavage (BAL), sputum, prostate cells grown in cell culture, blood, serum, lymph node tissue and lymphatic fluid. In another preferred embodiment, especially when metastasis of a primary prostate cancer is known or suspected, specimens include, without limitation, tissues from brain, bone, bone marrow, liver, adrenal glands and colon. In general, the tissues may be sampled by biopsy, including, without limitation, needle biopsy, e.g., transthoracic needle aspiration, cervical mediatinoscopy, endoscopic lymph node biopsy, video-assisted thoracoscopy, exploratory thoracotomy, bone marrow biopsy and bone marrow aspiration. See Scott, supra and Franklin, pp. 529-570, in Kane, supra. For early and inexpensive detection, assaying for changes in PSNAs or PSPs in cells in sputum samples may be particularly useful. Methods of obtaining and analyzing sputum samples is disclosed in Franklin, supra.

All the methods of the present invention may optionally include determining the expression levels of one or more other cancer markers in addition to determining the expression level of a PSNA or PSP. In many cases, the use of another cancer marker will decrease the likelihood of false positives or false negatives. In one embodiment, the one or more other cancer markers include other PSNA or PSPs as disclosed herein. Other cancer markers useful in the present invention will depend on the cancer being tested and are known to those of skill in the art. In a preferred embodiment, at least one other cancer marker in addition to a particular PSNA or PSP is measured. In a more preferred embodiment, at least two other additional cancer markers are used. In an even more preferred embodiment, at least three, more preferably at least five, even more preferably at least ten additional cancer markers are used.

Diagnosing

In one aspect, the invention provides a method for determining the expression levels and/or structural alterations of one or more PSNAs and/or PSPs in a sample from a patient suspected of having prostate cancer. In general, the method comprises the steps of obtaining the sample from the patient, determining the expression level or structural alterations of a PSNA and/or PSP and then ascertaining whether the patient has prostate cancer from the expression level of the PSNA or PSP. In general, if high expression relative to a control of a PSNA or PSP is indicative of prostate cancer, a diagnostic assay is considered positive if the level of expression of the PSNA or PSP is at least two times higher, and more preferably are at least five times higher, even more preferably at least ten times higher, than in preferably the same cells, tissues or bodily fluid of a normal human control. In contrast, if low expression relative to a control of a PSNA or PSP is indicative of prostate cancer, a diagnostic assay is considered positive if the level of expression of the PSNA or PSP is at least two times lower, more preferably are at least five times lower, even more preferably at least ten times lower than in preferably the same cells, tissues or bodily fluid of a normal human control. The normal human control may be from a different patient or from uninvolved tissue of the same patient.

The present invention also provides a method of determining whether prostate cancer has metastasized in a patient. One may identify whether the prostate cancer has metastasized by measuring the expression levels and/or structural alterations of one or more PSNAs and/or PSPs in a variety of tissues. The presence of a PSNA or PSP in a certain tissue at levels higher than that of corresponding noncancerous tissue (e.g., the same tissue from another individual) is indicative of metastasis if high level expression of a PSNA or PSP is associated with prostate cancer. Similarly, the presence of a PSNA or PSP in a tissue at levels lower than that of corresponding noncancerous tissue is indicative of metastasis if low level expression of a PSNA or PSP is associated with prostate cancer. Further, the presence of a structurally altered PSNA or PSP that is associated with prostate cancer is also indicative of metastasis.

In general, if high expression relative to a control of a PSNA or PSP is indicative of metastasis, an assay for metastasis is considered positive if the level of expression of the PSNA or PSP is at least two times higher, and more preferably are at least five times higher, even more preferably at least ten times higher, than in preferably the same cells, tissues or bodily fluid of a normal human control. In contrast, if low expression relative to a control of a PSNA or PSP is indicative of metastasis, an assay for metastasis is considered positive if the level of expression of the PSNA or PSP is at least two times lower, more preferably are at least five times lower, even more preferably at least ten times lower than in preferably the same cells, tissues or bodily fluid of a normal human control.

The PSNA or PSP of this invention may be used as element in an array or a multi-analyte test to recognize expression patterns associated with prostate cancers or other prostate related disorders. In addition, the sequences of either the nucleic acids or proteins may be used as elements in a computer program for pattern recognition of prostate disorders.

Staging

The invention also provides a method of staging prostate cancer in a human patient. The method comprises identifying a human patient having prostate cancer and analyzing cells, tissues or bodily fluids from such human patient for expression levels and/or structural alterations of one or more PSNAs or PSPs. First, one or more tumors from a variety of patients are staged according to procedures well-known in the art, and the expression level of one or more PSNAs or PSPs is determined for each stage to obtain a standard expression level for each PSNA and PSP. Then, the PSNA or PSP expression levels are determined in a biological sample from a patient whose stage of cancer is not known. The PSNA or PSP expression levels from the patient are then compared to the standard expression level. By comparing the expression level of the PSNAs and PSPs from the patient to the standard expression levels, one may determine the stage of the tumor. The same procedure may be followed using structural alterations of a PSNA or PSP to determine the stage of a prostate cancer.

Monitoring

Further provided is a method of monitoring prostate cancer in a human patient. One may monitor a human patient to determine whether there has been metastasis and, if there has been, when metastasis began to occur. One may also monitor a human patient to determine whether a preneoplastic lesion has become cancerous. One may also monitor a human patient to determine whether a therapy, e.g., chemotherapy, radiotherapy or surgery, has decreased or eliminated the prostate cancer. The method comprises identifying a human patient that one wants to monitor for prostate cancer, periodically analyzing cells, tissues or bodily fluids from such human patient for expression levels of one or more PSNAs or PSPs, and comparing the PSNA or PSP levels over time to those PSNA or PSP expression levels obtained previously. Patients may also be monitored by measuring one or more structural alterations in a PSNA or PSP that are associated with prostate cancer.

If increased expression of a PSNA or PSP is associated with metastasis, treatment failure, or conversion of a preneoplastic lesion to a cancerous lesion, then detecting an increase in the expression level of a PSNA or PSP indicates that the tumor is metastasizing, that treatment has failed or that the lesion is cancerous, respectively. One having ordinary skill in the art would recognize that if this were the case, then a decreased expression level would be indicative of no metastasis, effective therapy or failure to progress to a neoplastic lesion. If decreased expression of a PSNA or PSP is associated with metastasis, treatment failure, or conversion of a preneoplastic lesion to a cancerous lesion, then detecting an decrease in the expression level of a PSNA or PSP indicates that the tumor is metastasizing, that treatment has failed or that the lesion is cancerous, respectively. In a preferred embodiment, the levels of PSNAs or PSPs are determined from the same cell type, tissue or bodily fluid as prior patient samples. Monitoring a patient for onset of prostate cancer metastasis is periodic and preferably is done on a quarterly basis, but may be done more or less frequently.

The methods described herein can further be utilized as prognostic assays to identify subjects having or at risk of developing a disease or disorder associated with increased or decreased expression levels of a PSNA and/or PSP. The present invention provides a method in which a test sample is obtained from a human patient and one or more PSNAs and/or PSPs are detected. The presence of higher (or lower) PSNA or PSP levels as compared to normal human controls is diagnostic for the human patient being at risk for developing cancer, particularly prostate cancer. The effectiveness of therapeutic agents to decrease (or increase) expression or activity of one or more PSNAs and/or PSPs of the invention can also be monitored by analyzing levels of expression of the PSNAs and/or PSPs in a human patient in clinical trials or in in vitro screening assays such as in human cells. In this way, the gene expression pattern can serve as a marker, indicative of the physiological response of the human patient or cells, as the case may be, to the agent being tested.

Detection of Genetic Lesions or Mutations

The methods of the present invention can also be used to detect genetic lesions or mutations in a PSG, thereby determining if a human with the genetic lesion is susceptible to developing prostate cancer or to determine what genetic lesions are responsible, or are partly responsible, for a person's existing prostate cancer. Genetic lesions can be detected, for example, by ascertaining the existence of a deletion, insertion and/or substitution of one or more nucleotides from the PSGs of this invention, a chromosomal rearrangement of PSG, an aberrant modification of PSG (such as of the methylation pattern of the genomic DNA), or allelic loss of a PSG. Methods to detect such lesions in the PSG of this invention are known to those having ordinary skill in the art following the teachings of the specification.

Methods of Detecting Noncancerous Prostate Diseases

The invention also provides a method for determining the expression levels and/or structural alterations of one or more PSNAs and/or PSPs in a sample from a patient suspected of having or known to have a noncancerous prostate disease. In general, the method comprises the steps of obtaining a sample from the patient, determining the expression level or structural alterations of a PSNA and/or PSP, comparing the expression level or structural alteration of the PSNA or PSP to a normal prostate control, and then ascertaining whether the patient has a noncancerous prostate disease. In general, if high expression relative to a control of a PSNA or PSP is indicative of a particular noncancerous prostate disease, a diagnostic assay is considered positive if the level of expression of the PSNA or PSP is at least two times higher, and more preferably are at least five times higher, even more preferably at least ten times higher, than in preferably the same cells, tissues or bodily fluid of a normal human control. In contrast, if low expression relative to a control of a PSNA or PSP is indicative of a noncancerous prostate disease, a diagnostic assay is considered positive if the level of expression of the PSNA or PSP is at least two times lower, more preferably are at least five times lower, even more preferably at least ten times lower than in preferably the same cells, tissues or bodily fluid of a normal human control. The normal human control may be from a different patient or from uninvolved tissue of the same patient.

One having ordinary skill in the art may determine whether a PSNA and/or PSP is associated with a particular noncancerous prostate disease by obtaining prostate tissue from a patient having a noncancerous prostate disease of interest and determining which PSNAs and/or PSPs are expressed in the tissue at either a higher or a lower level than in normal prostate tissue. In another embodiment, one may determine whether a PSNA or PSP exhibits structural alterations in a particular noncancerous prostate disease state by obtaining prostate tissue from a patient having a noncancerous prostate disease of interest and determining the structural alterations in one or more PSNAs and/or PSPs relative to normal prostate tissue.

Methods for Identifying Prostate Tissue

In another aspect, the invention provides methods for identifying prostate tissue. These methods are particularly useful in, e.g., forensic science, prostate cell differentiation and development, and in tissue engineering.

In one embodiment, the invention provides a method for determining whether a sample is prostate tissue or has prostate tissue-like characteristics. The method comprises the steps of providing a sample suspected of comprising prostate tissue or having prostate tissue-like characteristics, determining whether the sample expresses one or more PSNAs and/or PSPs, and, if the sample expresses one or more PSNAs and/or PSPs, concluding that the sample comprises prostate tissue. In a preferred embodiment, the PSNA encodes a polypeptide having an amino acid sequence selected from SEQ ID NO: 136 through 240, or a homolog, allelic variant or fragment thereof. In a more preferred embodiment, the PSNA has a nucleotide sequence selected from SEQ ID NO: 1 through 135, or a hybridizing nucleic acid, an allelic variant or a part thereof. Determining whether a sample expresses a PSNA can be accomplished by any method known in the art. Preferred methods include hybridization to microarrays, Northern blot hybridization, and quantitative or qualitative RT-PCR. In another preferred embodiment, the method can be practiced by determining whether a PSP is expressed. Determining whether a sample expresses a PSP can be accomplished by any method known in the art. Preferred methods include Western blot, ELISA, RIA and 2D PAGE. In one embodiment, the PSP has an amino acid sequence selected from SEQ ID NO: 136 through 240, or a homolog, allelic variant or fragment thereof. In another preferred embodiment, the expression of at least two PSNAs and/or PSPs is determined. In a more preferred embodiment, the expression of at least three, more preferably four and even more preferably five PSNAs and/or PSPs are determined.

In one embodiment, the method can be used to determine whether an unknown tissue is prostate tissue. This is particularly useful in forensic science, in which small, damaged pieces of tissues that are not identifiable by microscopic or other means are recovered from a crime or accident scene. In another embodiment, the method can be used to determine whether a tissue is differentiating or developing into prostate tissue. This is important in monitoring the effects of the addition of various agents to cell or tissue culture, e.g., in producing new prostate tissue by tissue engineering. These agents include, e.g., growth and differentiation factors, extracellular matrix proteins and culture medium. Other factors that may be measured for effects on tissue development and differentiation include gene transfer into the cells or tissues, alterations in pH, aqueous:air interface and various other culture conditions.

Methods for Producing and Modifying Prostate Tissue

In another aspect, the invention provides methods for producing engineered prostate tissue or cells. In one embodiment, the method comprises the steps of providing cells, introducing a PSNA or a PSG into the cells, and growing the cells under conditions in which they exhibit one or more properties of prostate tissue cells. In a preferred embodiment, the cells are pluripotent. As is well-known in the art, normal prostate tissue comprises a large number of different cell types. Thus, in one embodiment, the engineered prostate tissue or cells comprises one of these cell types. In another embodiment, the engineered prostate tissue or cells comprises more than one prostate cell type. Further, the culture conditions of the cells or tissue may require manipulation in order to achieve full differentiation and development of the prostate cell tissue. Methods for manipulating culture conditions are well-known in the art.

Nucleic acid molecules encoding one or more PSPs are introduced into cells, preferably pluripotent cells. In a preferred embodiment, the nucleic acid molecules encode PSPs having amino acid sequences selected from SEQ ID NO: 136 through 240, or homologous proteins, analogs, allelic variants or fragments thereof. In a more preferred embodiment, the nucleic acid molecules have a nucleotide sequence selected from SEQ ID NO: 1 through 135, or hybridizing nucleic acids, allelic variants or parts thereof In another highly preferred embodiment, a PSG is introduced into the cells. Expression vectors and methods of introducing nucleic acid molecules into cells are well-known in the art and are described in detail, supra.

Artificial prostate tissue may be used to treat patients who have lost some or all of their prostate function.

Pharmaceutical Compositions

In another aspect, the invention provides pharmaceutical compositions comprising the nucleic acid molecules, polypeptides, antibodies, antibody derivatives, antibody fragments, agonists, antagonists, and inhibitors of the present invention. In a preferred embodiment, the pharmaceutical composition comprises a PSNA or part thereof. In a more preferred embodiment, the PSNA has a nucleotide sequence selected from the group consisting of SEQ ID NO: 1 through 135, a nucleic acid that hybridizes thereto, an allelic variant thereof, or a nucleic acid that has substantial sequence identity thereto. In another preferred embodiment, the pharmaceutical composition comprises a PSP or fragment thereof. In a more preferred embodiment, the PSP having an amino acid sequence that is selected from the group consisting of SEQ ID NO: 136 through 240, a polypeptide that is homologous thereto, a fusion protein comprising all or a portion of the polypeptide, or an analog or derivative thereof. In another preferred embodiment, the pharmaceutical composition comprises an anti-PSP antibody, preferably an antibody that specifically binds to a PSP having an amino acid that is selected from the group consisting of SEQ ID NO: 136 through 240, or an antibody that binds to a polypeptide that is homologous thereto, a fusion protein comprising all or a portion of the polypeptide, or an analog or derivative thereof.

Such a composition typically contains from about 0.1 to 90% by weight of a therapeutic agent of the invention formulated in and/or with a pharmaceutically acceptable carrier or excipient.

Pharmaceutical formulation is a well-established art, and is further described in Gennaro (ed.), Remington: The Science and Practice of Pharmacy, 20^{th} ed., Lippincott, Williams & Wilkins (2000); Ansel et al., Pharmaceutical Dosage Forms and Drug Delivery Systems, 7^{th} ed., Lippincott Williams & Wilkins (1999); and Kibbe (ed.), Handbook of Pharmaceutical Excipients American Pharmaceutical Association, 3^{rd} ed. (2000), the disclosures of which are incorporated herein by reference in their entireties, and thus need not be described in detail herein.

Briefly, formulation of the pharmaceutical compositions of the present invention will depend upon the route chosen for administration. The pharmaceutical compositions utilized in this invention can be administered by various routes including both enteral and parenteral routes, including oral, intravenous, intramuscular, subcutaneous, inhalation, topical, sublingual, rectal, intra-arterial, intramedullary, intrathecal, intraventricular, transmucosal, transdermal, intranasal, intraperitoneal, intrapulmonary, and intrauterine.

Oral dosage forms can be formulated as tablets, pills, dragees, capsules, liquids, gels, syrups, slurries, suspensions, and the like, for ingestion by the patient.

Solid formulations of the compositions for oral administration can contain suitable carriers or excipients, such as carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; and other agents such as acacia and alginic acid.

Agents that facilitate disintegration and/or solubilization can be added, such as the cross-linked polyvinyl pyrrolidone, agar, alginic acid, or a salt thereof, such as sodium alginate, microcrystalline cellulose, corn starch, sodium starch glycolate, and alginic acid.

Tablet binders that can be used include acacia, methylcellulose, sodium carboxymethylcellulose, polyvinylpyrrolidone (Povidone™), hydroxypropyl methylcellulose, sucrose, starch and ethylcellulose.

Lubricants that can be used include magnesium stearates, stearic acid, silicone fluid, talc, waxes, oils, and colloidal silica.

Fillers, agents that facilitate disintegration and/or solubilization, tablet binders and lubricants, including the aforementioned, can be used singly or in combination.

Solid oral dosage forms need not be uniform throughout. For example, dragee cores can be used in conjunction with suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures.

Oral dosage forms of the present invention include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Additionally, dyestuffs or pigments can be added to the tablets or dragee coatings for product identification or to characterize the quantity of active compound, i.e., dosage.

Liquid formulations of the pharmaceutical compositions for oral (enteral) administration are prepared in water or other aqueous vehicles and can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents.

The pharmaceutical compositions of the present invention can also be formulated for parenteral administration. Formulations for parenteral administration can be in the form of aqueous or non-aqueous isotonic sterile injection solutions or suspensions.

For intravenous injection, water soluble versions of the compounds of the present invention are formulated in, or if provided as a lyophilate, mixed with, a physiologically acceptable fluid vehicle, such as 5% dextrose (“D5”), physiologically buffered saline, 0.9% saline, Hanks' solution, or Ringer's solution. Intravenous formulations may include carriers, excipients or stabilizers including, without limitation, calcium, human serum albumin, citrate, acetate, calcium chloride, carbonate, and other salts.

Intramuscular preparations, e.g. a sterile formulation of a suitable soluble salt form of the compounds of the present invention, can be dissolved and administered in a pharmaceutical excipient such as Water-for-Injection, 0.9% saline, or 5% glucose solution. Alternatively, a suitable insoluble form of the compound can be prepared and administered as a suspension in an aqueous base or a pharmaceutically acceptable oil base, such as an ester of a long chain fatty acid (e.g., ethyl oleate), fatty oils such as sesame oil, triglycerides, or liposomes.

Parenteral formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like).

Aqueous injection suspensions can also contain substances that increase the viscosity of the suspension, such as sodium carboxymethyl cellulose, sorbitol, or dextran. Non-lipid polycationic amino polymers can also be used for delivery. Optionally, the suspension can also contain suitable stabilizers or agents that increase the solubility of the compounds to allow for the preparation of highly concentrated solutions.

Pharmaceutical compositions of the present invention can also be formulated to permit injectable, long-term, deposition. Injectable depot forms may be made by forming microencapsulated matrices of the compound in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in microemulsions that are compatible with body tissues.

The pharmaceutical compositions of the present invention can be administered topically.

For topical use the compounds of the present invention can also be prepared in suitable forms to be applied to the skin, or mucus membranes of the nose and throat, and can take the form of lotions, creams, ointments, liquid sprays or inhalants, drops, tinctures, lozenges, or throat paints. Such topical formulations further can include chemical compounds such as dimethylsulfoxide (DMSO) to facilitate surface penetration of the active ingredient. In other transdermal formulations, typically in patch-delivered formulations, the pharmaceutically active compound is formulated with one or more skin penetrants, such as 2-N-methyl-pyrrolidone (NMP) or Azone. A topical semi-solid ointment formulation typically contains a concentration of the active ingredient from about 1 to 20%, e.g., 5 to 10%, in a carrier such as a pharmaceutical cream base.

For application to the eyes or ears, the compounds of the present invention can be presented in liquid or semi-liquid form formulated in hydrophobic or hydrophilic bases as ointments, creams, lotions, paints or powders.

For rectal administration the compounds of the present invention can be administered in the form of suppositories admixed with conventional carriers such as cocoa butter, wax or other glyceride.

Inhalation formulations can also readily be formulated. For inhalation, various powder and liquid formulations can be prepared. For aerosol preparations, a sterile formulation of the compound or salt form of the compound may be used in inhalers, such as metered dose inhalers, and nebulizers. Aerosolized forms may be especially useful for treating respiratory disorders.

Alternatively, the compounds of the present invention can be in powder form for reconstitution in the appropriate pharmaceutically acceptable carrier at the time of delivery.

The pharmaceutically active compound in the pharmaceutical compositions of the present invention can be provided as the salt of a variety of acids, including but not limited to hydrochloric, sulfuric, acetic, lactic, tartaric, malic, and succinic acid. Salts tend to be more soluble in aqueous or other protonic solvents than are the corresponding free base forms.

After pharmaceutical compositions have been prepared, they are packaged in an appropriate container and labeled for treatment of an indicated condition.

The active compound will be present in an amount effective to achieve the intended purpose. The determination of an effective dose is well within the capability of those skilled in the art.

A “therapeutically effective dose” refers to that amount of active ingredient, for example PSP polypeptide, fusion protein, or fragments thereof, antibodies specific for PSP, agonists, antagonists or inhibitors of PSP, which ameliorates the signs or symptoms of the disease or prevents progression thereof, as would be understood in the medical arts, cure, although desired, is not required.

The therapeutically effective dose of the pharmaceutical agents of the present invention can be estimated initially by in vitro tests, such as cell culture assays, followed by assay in model animals, usually mice, rats, rabbits, dogs, or pigs. The animal model can also be used to determine an initial preferred concentration range and route of administration.

For example, the ED50 (the dose therapeutically effective in 50% of the population) and LD50 (the dose lethal to 50% of the population) can be determined in one or more cell culture of animal model systems. The dose ratio of toxic to therapeutic effects is the therapeutic index, which can be expressed as LD50/ED50. Pharmaceutical compositions that exhibit large therapeutic indices are preferred.

The data obtained from cell culture assays and animal studies are used in formulating an initial dosage range for human use, and preferably provide a range of circulating concentrations that includes the ED50 with little or no toxicity. After administration, or between successive administrations, the circulating concentration of active agent varies within this range depending upon pharmacokinetic factors well-known in the art, such as the dosage form employed, sensitivity of the patient, and the route of administration.

The exact dosage will be determined by the practitioner, in light of factors specific to the subject requiring treatment. Factors that can be taken into account by the practitioner include the severity of the disease state, general health of the subject, age, weight, gender of the subject, diet, time and frequency of administration, drug combination(s), reaction sensitivities, and tolerance/response to therapy. Long-acting pharmaceutical compositions can be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Where the therapeutic agent is a protein or antibody of the present invention, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose.

Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Conventional methods, known to those of ordinary skill in the art of medicine, can be used to administer the pharmaceutical formulation(s) of the present invention to the patient. The pharmaceutical compositions of the present invention can be administered alone, or in combination with other therapeutic agents or interventions.

Therapeutic Methods

The present invention further provides methods of treating subjects having defects in a gene of the invention, e.g., in expression, activity, distribution, localization, and/or solubility, which can manifest as a disorder of prostate function. As used herein, “treating” includes all medically-acceptable types of therapeutic intervention, including palliation and prophylaxis (prevention) of disease. The term “treating” encompasses any improvement of a disease, including minor improvements. These methods are discussed below.

Gene Therapy and Vaccines

The isolated nucleic acids of the present invention can also be used to drive in vivo expression of the polypeptides of the present invention. In vivo expression can be driven from a vector, typically a viral vector, often a vector based upon a replication incompetent retrovirus, an adenovirus, or an adeno-associated virus (AAV), for purpose of gene therapy. In vivo expression can also be driven from signals endogenous to the nucleic acid or from a vector, often a plasmid vector, such as pVAX1 (Invitrogen, Carlsbad, Calif., USA), for purpose of “naked” nucleic acid vaccination, as further described in U.S. Pat. Nos. 5,589,466; 5,679,647; 5,804,566; 5,830,877; 5,843,913; 5,880,104; 5,958,891; 5,985,847; 6,017,897; 6,110,898; and 6,204,250, the disclosures of which are incorporated herein by reference in their entireties. For cancer therapy, it is preferred that the vector also be tumor-selective. See, e.g., Doronin et al., J. Virol. 75: 3314-24 (2001).

In another embodiment of the therapeutic methods of the present invention, a therapeutically effective amount of a pharmaceutical composition comprising a nucleic acid of the present invention is administered. The nucleic acid can be delivered in a vector that drives expression of a PSP, fusion protein, or fragment thereof, or without such vector. Nucleic acid compositions that can drive expression of a PSP are administered, for example, to complement a deficiency in the native PSP, or as DNA vaccines. Expression vectors derived from virus, replication deficient retroviruses, adenovirus, adeno-associated (AAV) virus, herpes virus, or vaccinia virus can be used as can plasmids. See, e.g., Cid-Arregui, supra. In a preferred embodiment, the nucleic acid molecule encodes a PSP having the amino acid sequence of SEQ ID NO: 136 through 240, or a fragment, fusion protein, allelic variant or homolog thereof.

In still other therapeutic methods of the present invention, pharmaceutical compositions comprising host cells that express a PSP, fusions, or fragments thereof can be administered. In such cases, the cells are typically autologous, so as to circumvent xenogeneic or allotypic rejection, and are administered to complement defects in PSP production or activity. In a preferred embodiment, the nucleic acid molecules in the cells encode a PSP having the amino acid sequence of SEQ ID NO: 136 through 240, or a fragment, fusion protein, allelic variant or homolog thereof.

Antisense Administration

Antisense nucleic acid compositions, or vectors that drive expression of a PSG antisense nucleic acid, are administered to downregulate transcription and/or translation of a PSG in circumstances in which excessive production, or production of aberrant protein, is the pathophysiologic basis of disease.

Antisense compositions useful in therapy can have a sequence that is complementary to coding or to noncoding regions of a PSG. For example, oligonucleotides derived from the transcription initiation site, e.g., between positions −10 and +10 from the start site, are preferred.

Catalytic antisense compositions, such as ribozymes, that are capable of sequence-specific hybridization to PSG transcripts, are also useful in therapy. See, e.g., Phylactou, Adv. Drug Deliv. Rev. 44(2-3): 97-108 (2000); Phylactou et al., Hum. Mol. Genet. 7(10): 1649-53 (1998); Rossi, Ciba Found. Symp. 209: 195-204 (1997); and Sigurdsson et al., Trends Biotechnol. 13(8): 286-9 (1995), the disclosures of which are incorporated herein by reference in their entireties.

Other nucleic acids useful in the therapeutic methods of the present invention are those that are capable of triplex helix formation in or near the PSG genomic locus. Such triplexing oligonucleotides are able to inhibit transcription. See, e.g., Intody et al., Nucleic Acids Res. 28(21): 4283-90 (2000); McGuffie et al, Cancer Res. 60(14): 3790-9 (2000), the disclosures of which are incorporated herein by reference. Pharmaceutical compositions comprising such triplex forming oligos (TFOs) are administered in circumstances in which excessive production, or production of aberrant protein, is a pathophysiologic basis of disease.

In a preferred embodiment, the antisense molecule is derived from a nucleic acid molecule encoding a PSP, preferably a PSP comprising an amino acid sequence of SEQ ID NO: 136 through 240, or a fragment, allelic variant or homolog thereof. In a more preferred embodiment, the antisense molecule is derived from a nucleic acid molecule having a nucleotide sequence of SEQ ID NO: 1 through 135, or a part, allelic variant, substantially similar or hybridizing nucleic acid thereof.

Polypeptide Administration

In one embodiment of the therapeutic methods of the present invention, a therapeutically effective amount of a pharmaceutical composition comprising a PSP, a fusion protein, fragment, analog or derivative thereof is administered to a subject with a clinically-significant PSP defect.

Protein compositions are administered, for example, to complement a deficiency in native PSP. In other embodiments, protein compositions are administered as a vaccine to elicit a humoral and/or cellular immune response to PSP. The immune response can be used to modulate activity of PSP or, depending on the immunogen, to immunize against aberrant or aberrantly expressed forms, such as mutant or inappropriately expressed isoforms. In yet other embodiments, protein fusions having a toxic moiety are administered to ablate cells that aberrantly accumulate PSP.

In a preferred embodiment, the polypeptide is a PSP comprising an amino acid sequence of SEQ ID NO: 136 through 240, or a fusion protein, allelic variant, homolog, analog or derivative thereof. In a more preferred embodiment, the polypeptide is encoded by a nucleic acid molecule having a nucleotide sequence of SEQ ID NO: 1 through 135, or a part, allelic variant, substantially similar or hybridizing nucleic acid thereof.

Antibody, Agonist and Antagonist Administration

In another embodiment of the therapeutic methods of the present invention, a therapeutically effective amount of a pharmaceutical composition comprising an antibody (including fragment or derivative thereof) of the present invention is administered. As is well-known, antibody compositions are administered, for example, to antagonize activity of PSP, or to target therapeutic agents to sites of PSP presence and/or accumulation. In a preferred embodiment, the antibody specifically binds to a PSP comprising an amino acid sequence of SEQ ID NO: 136 through 240, or a fusion protein, allelic variant, homolog, analog or derivative thereof. In a more preferred embodiment, the antibody specifically binds to a PSP encoded by a nucleic acid molecule having a nucleotide sequence of SEQ ID NO: 1 through 135, or a part, allelic variant, substantially similar or hybridizing nucleic acid thereof.

The present invention also provides methods for identifying modulators which bind to a PSP or have a modulatory effect on the expression or activity of a PSP. Modulators which decrease the expression or activity of PSP (antagonists) are believed to be useful in treating prostate cancer. Such screening assays are known to those of skill in the art and include, without limitation, cell-based assays and cell-free assays. Small molecules predicted via computer imaging to specifically bind to regions of a PSP can also be designed, synthesized and tested for use in the imaging and treatment of prostate cancer. Further, libraries of molecules can be screened for potential anticancer agents by assessing the ability of the molecule to bind to the PSPs identified herein. Molecules identified in the library as being capable of binding to a PSP are key candidates for further evaluation for use in the treatment of prostate cancer. In a preferred embodiment, these molecules will downregulate expression and/or activity of a PSP in cells.

In another embodiment of the therapeutic methods of the present invention, a pharmaceutical composition comprising a non-antibody antagonist of PSP is administered. Antagonists of PSP can be produced using methods generally known in the art. In particular, purified PSP can be used to screen libraries of pharmaceutical agents, often combinatorial libraries of small molecules, to identify those that specifically bind and antagonize at least one activity of a PSP.

In other embodiments a pharmaceutical composition comprising an agonist of a PSP is administered. Agonists can be identified using methods analogous to those used to identify antagonists.

In a preferred embodiment, the antagonist or agonist specifically binds to and antagonizes or agonizes, respectively, a PSP comprising an amino acid sequence of SEQ ID NO: 136 through 240, or a fusion protein, allelic variant, homolog, analog or derivative thereof. In a more preferred embodiment, the antagonist or agonist specifically binds to and antagonizes or agonizes, respectively, a PSP encoded by a nucleic acid molecule having a nucleotide sequence of SEQ ID NO: 1 through 135, or a part, allelic variant, substantially similar or hybridizing nucleic acid thereof.

Targeting Prostate Tissue

The invention also provides a method in which a polypeptide of the invention, or an antibody thereto, is linked to a therapeutic agent such that it can be delivered to the prostate or to specific cells in the prostate. In a preferred embodiment, an anti-PSP antibody is linked to a therapeutic agent and is administered to a patient in need of such therapeutic agent. The therapeutic agent may be a toxin, if prostate tissue needs to be selectively destroyed. This would be useful for targeting and killing prostate cancer cells. In another embodiment, the therapeutic agent may be a growth or differentiation factor, which would be useful for promoting prostate cell function.

In another embodiment, an anti-PSP antibody may be linked to an imaging agent that can be detected using, e.g., magnetic resonance imaging, CT or PET. This would be useful for determining and monitoring prostate function, identifying prostate cancer tumors, and identifying noncancerous prostate diseases.

### EXAMPLES

### Example 1

### Gene Expression analysis

PSGs were identified by a systematic analysis of gene expression data in the LIFESEQ® Gold database available from Incyte Genomics Inc (Palo Alto, Calif.) using the data mining software package CLASP™ (Candidate Lead Automatic Search Program). CLASP™ is a set of algorithms that interrogate Incyte's database to identify genes that are both specific to particular tissue types as well as differentially expressed in tissues from patients with cancer. LifeSeq® Gold contains information about which genes are expressed in various tissues in the body and about the dynamics of expression in both normal and diseased states. CLASP™ first sorts the LifeSeq® Gold database into defined tissue types, such as breast, ovary and prostate. CLASP™ categorizes each tissue sample by disease state. Disease states include “healthy,” “cancer,” “associated with cancer,” “other disease” and “other.” Categorizing the disease states improves our ability to identify tissue and cancer-specific molecular targets. CLASP™ then performs a simultaneous parallel search for genes that are expressed both (1) selectively in the defined tissue type compared to other tissue types and (2) differentially in the “cancer” disease state compared to the other disease states affecting the same, or different, tissues. This sorting is accomplished by using mathematical and statistical filters that specify the minimum change in expression levels and the minimum frequency that the differential expression pattern must be observed across the tissue samples for the gene to be considered statistically significant. The CLASP™ algorithm quantifies the relative abundance of a particular gene in each tissue type and in each disease state.

To find the PSGs of this invention, the following specific CLASP™ profiles were utilized: tissue-specific expression (CLASP 1), detectable expression only in cancer tissue (CLASP 2), highest differential expression for a given cancer (CLASP 4); differential expression in cancer tissue (CLASP 5), and. cDNA libraries were divided into 60 unique tissue types (early versions of LifeSeq® had 48 tissue types). Genes or ESTs were grouped into “gene bins,” where each bin is a cluster of sequences grouped together where they share a common contig. The expression level for each gene bin was calculated for each tissue type. Differential expression significance was calculated with rigorous statistical significant testing taking into account variations in sample size and relative gene abundance in different libraries and within each library (for the equations used to determine statistically significant expression see Audic and Claverie “The significance of digital gene expression profiles,” Genome Res 7(10): 986-995 (1997), including Equation 1 on page 987 and Equation 2 on page 988, the contents of which are incorporated by reference). Differentially expressed tissue-specific genes were selected based on the percentage abundance level in the targeted tissue versus all the other tissues (tissue-specificity). The expression levels for each gene in libraries of normal tissues or non-tumor tissues from cancer patients were compared with the expression levels in tissue libraries associated with tumor or disease (cancer-specificity). The results were analyzed for statistical significance.

The selection of the target genes meeting the rigorous CLASP™ profile criteria were as follows:

(a) CLASP 1: tissue-specific expression: To qualify as a CLASP 1 candidate, a gene must exhibit statistically significant expression in the tissue of interest compared to all other tissues. Only if the gene exhibits such differential expression with a 90% of confidence level is it selected as a CLASP 1 candidate.

(b) CLASP 2: detectable expression only in cancer tissue: To qualify as a CLASP 2 candidate, a gene must exhibit detectable expression in tumor tissues and undetectable expression in libraries from normal individuals and libraries from normal tissue obtained from diseased patients. In addition, such a gene must also exhibit further specificity for the tumor tissues of interest.

(c) CLASP 5: differential expression in cancer tissue: To qualify as a CLASP 5 candidate, a gene must be differentially expressed in tumor libraries in the tissue of interest compared to normal libraries for all tissues. Only if the gene exhibits such differential expression with a 90% of confidence level is it selected as a CLASP 5 candidate.

The CLASP™ scores for SEQ ID NO: 1-135 are listed below:

| SEQ ID NO: 1 | DEX0265_1 CLASP2 | |
|---|---|---|
| SEQ ID NO: 2 | DEX0265_2 CLASP2 | |
| SEQ ID NO: 3 | DEX0265_3 CLASP2 | |
| SEQ ID NO: 4 | DEX0265_4 CLASP2 | |
| SEQ ID NO: 5 | DEX0265_5 CLASP2 | |
| SEQ ID NO: 6 | DEX0265_6 CLASP2 | |
| SEQ ID NO: 7 | DEX0265_7 CLASP2 | |
| SEQ ID NO: 8 | DEX0265_8 CLASP2 | |
| SEQ ID NO: 9 | DEX0265_9 CLASP2 | |
| SEQ ID NO: 10 | DEX0265_10 CLASP2 | |
| SEQ ID NO: 11 | DEX0265_11 CLASP2 | |
| SEQ ID NO: 12 | DEX0265_12 CLASP2 | |
| SEQ ID NO: 13 | DEX0265_13 CLASP2 | |
| SEQ ID NO: 14 | DEX0265_14 CLASP5 CLASP1 | |
| SEQ ID NO: 15 | DEX0265_15 CLASP5 CLASP1 | |
| SEQ ID NO: 16 | DEX0265_16 CLASP2 | |
| SEQ ID NO: 17 | DEX0265_17 CLASP2 | |
| SEQ ID NO: 18 | DEX0265_18 CLASP2 | |
| SEQ ID NO: 20 | DEX0265_20 CLASP2 | |
| SEQ ID NO: 21 | DEX0265_21 CLASP2 | |
| SEQ ID NO: 22 | DEX0265_22 CLASP2 | |
| SEQ ID NO: 23 | DEX0265_23 CLASP2 | |
| SEQ ID NO: 24 | DEX0265_24 CLASP2 | |
| SEQ ID NO: 25 | DEX0265_25 CLASP2 | |
| SEQ ID NO: 26 | DEX0265_26 CLASP2 | |
| SEQ ID NO: 27 | DEX0265_27 CLASP2 | |
| SEQ ID NO: 28 | DEX0265_28 CLASP2 | |
| SEQ ID NO: 29 | DEX0265_29 CLASP2 | |
| SEQ ID NO: 30 | DEX0265_30 CLASP5 CLASP1 | |
| SEQ ID NO: 31 | DEX0265_31 CLASP2 CLASP1 | |
| SEQ ID NO: 32 | DEX0265_32 CLASP2 CLASP1 | |
| SEQ ID NO: 33 | DEX0265_33 CLASP2 | |
| SEQ ID NO: 34 | DEX0265_34 CLASP2 | |
| SEQ ID NO: 35 | DEX0265_35 CLASP2 | |
| SEQ ID NO: 36 | DEX0265_36 CLASP2 | |
| SEQ ID NO: 37 | DEX0265_37 CLASP2 | |
| SEQ ID NO: 38 | DEX0265_38 CLASP1 | |
| SEQ ID NO: 39 | DEX0265_39 CLASP2 | |
| SEQ ID NO: 40 | DEX0265_40 CLASP2 | |
| SEQ ID NO: 41 | DEX0265_41 CLASP2 | |
| SEQ ID NO: 42 | DEX0265_42 CLASP1 | |
| SEQ ID NO: 43 | DEX0265_43 CLASP1 | |
| SEQ ID NO: 44 | DEX0265_44 CLASP5 CLASP1 | |
| SEQ ID NO: 45 | DEX0265_45 CLASP5 CLASP1 | |
| SEQ ID NO: 46 | DEX0265_46 CLASP5 CLASP1 | |
| SEQ ID NO: 47 | DEX0265_47 CLASP5 CLASP1 | |
| SEQ ID NO: 48 | DEX0265_48 CLASP2 | |
| SEQ ID NO: 49 | DEX0265_49 CLASP2 | |
| SEQ ID NO: 50 | DEX0265_50 CLASP2 CLASP1 | |
| SEQ ID NO: 51 | DEX0265_51 CLASP2 CLASP1 | |
| SEQ ID NO: 52 | DEX0265_52 CLASP2 | |
| SEQ ID NO: 53 | DEX0265_53 CLASP2 | |
| SEQ ID NO: 54 | DEX0265_54 CLASP2 | |
| SEQ ID NO: 55 | DEX0265_55 CLASP2 | |
| SEQ ID NO: 56 | DEX0265_56 CLASP2 | |
| SEQ ID NO: 57 | DEX0265_57 CLASP2 | |
| SEQ ID NO: 58 | DEX0265_58 CLASP2 | |
| SEQ ID NO: 59 | DEX0265_59 CLASP2 | |
| SEQ ID NO: 60 | DEX0265_60 CLASP5 CLASP1 | |
| SEQ ID NO: 61 | DEX0265_61 CLASP5 CLASP1 | |
| SEQ ID NO: 62 | DEX0265_62 CLASP2 | |
| SEQ ID NO: 63 | DEX0265_63 CLASP2 | |
| SEQ ID NO: 64 | DEX0265_64 CLASP2 | |
| SEQ ID NO: 65 | DEX0265_65 CLASP2 | |
| SEQ ID NO: 66 | DEX0265_66 CLASP2 | |
| SEQ ID NO: 67 | DEX0265_67 CLASP5 CLASP1 | |
| SEQ ID NO: 68 | DEX0265_68 CLASP5 CLASP1 | |
| SEQ ID NO: 69 | DEX0265_69 CLASP2 | |
| SEQ ID NO: 70 | DEX0265_70 CLASP2 | |
| SEQ ID NO: 71 | DEX0265_71 CLASP2 | |
| SEQ ID NO: 72 | DEX0265_72 CLASP2 | |
| SEQ ID NO: 73 | DEX0265_73 CLASP2 | |
| SEQ ID NO: 74 | DEX0265_74 CLASP2 | |
| SEQ ID NO: 75 | DEX0265_75 CLASP2 | |
| SEQ ID NO: 76 | DEX0265_76 CLASP2 | |
| SEQ ID NO: 77 | DEX0265_77 CLASP2 | |
| SEQ ID NO: 78 | DEX0265_78 CLASP2 | |
| SEQ ID NO: 79 | DEX0265_79 CLASP2 CLASP1 | |
| SEQ ID NO: 81 | DEX0265_81 CLASP5 CLASP1 | |
| SEQ ID NO: 82 | DEX0265_82 CLASP2 | |
| SEQ ID NO: 83 | DEX0265_83 CLASP2 | |
| SEQ ID NO: 84 | DEX0265_84 CLASP2 | |
| SEQ ID NO: 85 | DEX0265_85 CLASP5 CLASP1 | |
| SEQ ID NO: 86 | DEX0265_86 CLASP5 CLASP1 | |
| SEQ ID NO: 87 | DEX0265_87 CLASP2 | |
| SEQ ID NO: 88 | DEX0265_88 CLASP2 | |
| SEQ ID NO: 89 | DEX0265_89 CLASP2 | |
| SEQ ID NO: 90 | DEX0265_90 CLASP5 CLASP1 | |
| SEQ ID NO: 91 | DEX0265_91 CLASP5 CLASP1 | |
| SEQ ID NO: 92 | DEX0265_92 CLASP5 CLASP1 | |
| SEQ ID NO: 93 | DEX0265_93 CLASP5 CLASP1 | |
| SEQ ID NO: 94 | DEX0265_94 CLASP2 | |
| SEQ ID NO: 95 | DEX0265_95 CLASP5 CLASP1 | |
| SEQ ID NO: 96 | DEX0265_96 CLASP2 | |
| SEQ ID NO: 97 | DEX0265_97 CLASP5 CLASP1 | |
| SEQ ID NO: 98 | DEX0265_98 CLASP5 CLASP1 | |
| SEQ ID NO: 99 | DEX0265_99 CLASP2 | |
| SEQ ID NO: 100 | DEX0265_100 | CLASP2 |
| SEQ ID NO: 101 | DEX0265_101 | CLASP1 |
| SEQ ID NO: 102 | DEX0265_102 | CLASP1 |
| SEQ ID NO: 103 | DEX0265_103 | CLASP1 |
| SEQ ID NO: 104 | DEX0265_104 | CLASP2 |
| SEQ ID NO: 105 | DEX0265_105 | CLASP2 |
| SEQ ID NO: 106 | DEX0265_106 | CLASP5 CLASP1 |
| SEQ ID NO: 107 | DEX0265_107 | CLASP2 CLASP1 |
| SEQ ID NO: 108 | DEX0265_108 | CLASP2 CLASP1 |
| SEQ ID NO: 109 | DEX0265_109 | CLASP2 |
| SEQ ID NO: 110 | DEX0265_110 | CLASP2 |
| SEQ ID NO: 111 | DEX0265_111 | CLASP2 CLASP1 |
| SEQ ID NO: 112 | DEX0265_112 | CLASP2 CLASP1 |
| SEQ ID NO: 113 | DEX0265_113 | CLASP2 |
| SEQ ID NO: 114 | DEX0265_114 | CLASP2 |
| SEQ ID NO: 115 | DEX0265_115 | CLASP2 |
| SEQ ID NO: 116 | DEX0265_116 | CLASP2 CLASP1 |
| SEQ ID NO: 117 | DEX0265_117 | CLASP2 CLASP1 |
| SEQ ID NO: 118 | DEX0265_118 | CLASP5 CLASP1 |
| SEQ ID NO: 119 | DEX0265_119 | CLASP5 CLASP1 |
| SEQ ID NO: 120 | DEX0265_120 | CLASP5 CLASP1 |
| SEQ ID NO: 121 | DEX0265_121 | CLASP5 CLASP1 |
| SEQ ID NO: 122 | DEX0265_122 | CLASP2 CLASP1 |
| SEQ ID NO: 123 | DEX0265_123 | CLASP2 CLASP1 |
| SEQ ID NO: 124 | DEX0265_124 | CLASP2 |
| SEQ ID NO: 125 | DEX0265_125 | CLASP2 |
| SEQ ID NO: 126 | DEX0265_126 | CLASP2 |
| SEQ ID NO: 127 | DEX0265_127 | CLASP5 CLASP1 |
| SEQ ID NO: 128 | DEX0265_128 | CLASP5 CLASP1 |
| SEQ ID NO: 129 | DEX0265_129 | CLASP5 CLASP1 |
| SEQ ID NO: 130 | DEX0265_130 | CLASP5 CLASP1 |
| SEQ ID NO: 131 | DEX0265_131 | CLASP1 |
| SEQ ID NO: 132 | DEX0265_132 | CLASP1 |
| SEQ ID NO: 133 | DEX0265_133 | CLASP2 |
| SEQ ID NO: 134 | DEX0265_134 | CLASP5 CLASP1 |
| SEQ ID NO: 135 | DEX0265_135 | CLASP5 CLASP1 |

DEX0265 CLASP expression Level

| | | | |
|---|---|---|---|
| SEQ ID NO: 1 | PRO .0013 | | |
| SEQ ID NO: 2 | PRO .0013 | | |
| SEQ ID NO: 3 | PRO .0044 | | |
| SEQ ID NO: 4 | PRO. 0038 | | |
| SEQ ID NO: 5 | PRO .0038 | | |
| SEQ ID NO: 6 | PRO .0038 | | |
| SEQ ID NO: 7 | PRO .0038 | | |
| SEQ ID NO: 8 | PRO .0038 | | |
| SEQ ID NO: 9 | PRO .0038 | | |
| SEQ ID NO: 10 | PRO .0038 | | |
| SEQ ID NO: 11 | PRO .0038 | | |
| SEQ ID NO: 12 | PRO .0038 | | |
| SEQ ID NO: 13 | PRO .0038 | | |
| SEQ ID NO: 14 | PRO .0017 | | |
| SEQ ID NO: 15 | PRO .0017 | | |
| SEQ ID NO: 16 | PRO .0038 | | |
| SEQ ID NO: 17 | PRO .0038 | | |
| SEQ ID NO: 18 | PRO .0038 | | |
| SEQ ID NO: 20 | PRO .0038 | | |
| SEQ ID NO: 21 | PRO .0013 | | |
| SEQ ID NO: 22 | PRO .0013 | | |
| SEQ ID NO: 23 | PRO .0017 | | |
| SEQ ID NO: 24 | PRO .0044 | | |
| SEQ ID NO: 25 | PRO .0044 | CON .0017 | |
| SEQ ID NO: 26 | PRO .0013 | ADR .0024 | |
| SEQ ID NO: 27 | PRO .002 | PAN .0043 | |
| SEQ ID NO: 28 | PRO .0044 | | |
| SEQ ID NO: 29 | PRO .0044 | | |
| SEQ ID NO: 30 | PRO .0017 | BRN .0001 | BLO .0003 |
| SEQ ID NO: 31 | PRO .002 | | |
| SEQ ID NO: 32 | PRO .002 | | |
| SEQ ID NO: 33 | PRO .0013 | | |
| SEQ ID NO: 34 | PRO .0013 | | |
| SEQ ID NO: 35 | PRO .0038 | | |
| SEQ ID NO: 36 | PRO .0065 | | |
| SEQ ID NO: 37 | PRO .0065 | | |
| SEQ ID NO: 38 | PRO .0014 | MAM .0004 | NRV .0009 |
| SEQ ID NO: 39 | PRO .0038 | | |
| SEQ ID NO: 40 | PRO .0032 | | |
| SEQ ID NO: 41 | PRO .0032 | | |

| | | | | | |
|---|---|---|---|---|---|
| SEQ ID NO: 42 | PRO .0014 | BLO .0003 | UTR .0004 | MSL .002 | EYE .0101 |
| SEQ ID NO: 43 | PRO .0014 | BLO .0003 | UTR .0004 | MSL .002 | EYE .0101 |

| | | | |
|---|---|---|---|
| SEQ ID NO: 44 | PRO .0023 | UTR .0004 | INS .001 |

| | | | | | |
|---|---|---|---|---|---|
| SEQ ID NO: 45 | PRO .0565 | UTR .0013 | SKN .0015 | BLD .0016 | BLD .0016 |
| SEQ ID NO: 46 | PRO .0565 | UTR .0013 | SKN .0015 | BLD .0016 | BLD .0016 |
| SEQ ID NO: 47 | PRO .0565 | UTR .0013 | SKN .0015 | BLD .0016 | BLD .0016 |

| | |
|---|---|
| SEQ ID NO: 48 | PRO .0065 |
| SEQ ID NO: 49 | PRO .0065 |

| | | | | | |
|---|---|---|---|---|---|
| SEQ ID NO: 50 | PRO .0052 | UTR .0004 | FTS .0004 | NRV .0009 | KID .0019 |
| SEQ ID NO: 51 | PRO .0052 | UTR .0004 | FTS .0004 | NRV .0009 | KID .0019 |

| | |
|---|---|
| SEQ ID NO: 52 | PRO .0013 |
| SEQ ID NO: 53 | PRO .0013 |
| SEQ ID NO: 54 | PRO .0044 |
| SEQ ID NO: 55 | PRO .0044 |
| SEQ ID NO: 56 | PRO .0044 |
| SEQ ID NO: 57 | PRO .0044 |
| SEQ ID NO: 58 | PRO .0044 |
| SEQ ID NO: 59 | PRO .0044 |

| | | | | | |
|---|---|---|---|---|---|
| SEQ ID NO: 60 | PRO .0017 | BRN .0003 | UTR .0004 | KID .0006 | FTS .0006 |
| SEQ ID NO: 61 | PRO .0017 | BRN .0003 | UTR .0004 | KID .0006 | FTS .0006 |

| | | |
|---|---|---|
| SEQ ID NO: 62 | PRO .0044 | |
| SEQ ID NO: 63 | PRO .0044 | |
| SEQ ID NO: 64 | PRO .0044 | |
| SEQ ID NO: 65 | PRO .0044 | |
| SEQ ID NO: 66 | PRO .0013 | MAM .0011 |

| | | | | | |
|---|---|---|---|---|---|
| SEQ ID NO: 67 | PRO .0025 | FTS .0001 | MAM .0004 | LNG .0004 | INL .0004 |
| SEQ ID NO: 68 | PRO .0025 | FTS .0001 | MAM .0004 | LNG .0004 | INL .0004 |

| | | | | |
|---|---|---|---|---|
| SEQ ID NO: 69 | PRO .0029 | | | |
| SEQ ID NO: 70 | PRO .0044 | | | |
| SEQ ID NO: 71 | PRO .0044 | | | |
| SEQ ID NO: 72 | PRO .0013 | | | |
| SEQ ID NO: 73 | PRO .0013 | | | |
| SEQ ID NO: 74 | PRO .0038 | | | |
| SEQ ID NO: 75 | PRO .002 | | | |
| SEQ ID NO: 76 | PRO .0038 | | | |
| SEQ ID NO: 77 | PRO .0017 | | | |
| SEQ ID NO: 78 | PRO .0039 | | | |
| SEQ ID NO: 79 | PRO .0045 | SYN .0026 | | |
| SEQ ID NO: 81 | PRO .0023 | FTS .0004 | CON .0007 | THY .0019 |
| SEQ ID NO: 82 | PRO .002 | | | |
| SEQ ID NO: 83 | PRO .002 | | | |
| SEQ ID NO: 84 | PRO .002 | | | |
| SEQ ID NO: 85 | PRO .012 | | | |
| SEQ ID NO: 86 | PRO .012 | | | |
| SEQ ID NO: 87 | PRO .0013 | | | |
| SEQ ID NO: 88 | PRO .0038 | LNG .0015 | | |
| SEQ ID NO: 89 | PRO .0021 | | | |
| SEQ ID NO: 90 | PRO .0018 | BRN .0001 | BRN .0002 | MAM .0004 |
| SEQ ID NO: 91 | PRO .0018 | BRN .0001 | BRN .0002 | MAM .0004 |
| SEQ ID NO: 92 | PRO .0018 | BRN .0001 | BRN .0002 | MAM .0004 |
| SEQ ID NO: 93 | PRO .0018 | BRN .0001 | BRN .0002 | MAM .0004 |
| SEQ ID NO: 94 | PRO .0043 | | | |
| SEQ ID NO: 95 | PRO .0017 | LNG .0004 | BMR .0017 | |
| SEQ ID NO: 96 | PRO .002 | MAM .0007 | | |
| SEQ ID NO: 97 | PRO .0017 | BRN .0003 | MAM .0008 | |
| SEQ ID NO: 98 | PRO .0017 | BRN .0003 | MAM .0008 | |
| SEQ ID NO: 99 | PRO .002 | LNG .0015 | | |
| SEQ ID NO: 100 | PRO .002 | LNG .0015 | | |

| | | | | | |
|---|---|---|---|---|---|
| SEQ ID NO: 101 | PRO .0017 | FTS .0001 | INL .0004 | INL .0006 | BRN .0007 |
| SEQ ID NO: 102 | PRO .0017 | FTS .0001 | INL .0004 | INL .0006 | BRN .0007 |
| SEQ ID NO: 103 | PRO .0017 | FTS .0001 | INL .0004 | INL .0006 | BRN .0007 |

| | |
|---|---|
| SEQ ID NO: 104 | PRO .0013 |
| SEQ ID NO: 105 | PRO .0013 |

| | | | | | |
|---|---|---|---|---|---|
| SEQ ID NO: 106 | PRO .0017 | FTS .0004 | BRN .0006 | LNG .0007 | OVR .0014 |

| | | | | |
|---|---|---|---|---|
| SEQ ID NO: 107 | PRO .0054 | FTS .0001 | MAM .0004 | MAM .0018 |
| SEQ ID NO: 108 | PRO .0054 | FTS .0001 | MAM .0004 | MAM .0018 |
| SEQ ID NO: 109 | PRO .0038 | CON .0013 | BRN .0015 | |
| SEQ ID NO: 110 | PRO .0038 | CON .0013 | BRN .0015 | |
| SEQ ID NO: 111 | PRO .002 | | | |
| SEQ ID NO: 112 | PRO .002 | | | |
| SEQ ID NO: 113 | PRO .0013 | | | |
| SEQ ID NO: 114 | PRO .0029 | PAN .0043 | | |
| SEQ ID NO: 115 | PRO .0029 | PAN .0043 | | |
| SEQ ID NO: 116 | PRO .0094 | | | |
| SEQ ID NO: 117 | PRO .0094 | | | |
| SEQ ID NO: 118 | PRO .0017 | BRN .0003 | UTR .0008 | INS .001 |
| SEQ ID NO: 119 | PRO .0017 | BRN .0003 | UTR .0008 | INS .001 |
| SEQ ID NO: 120 | PRO .0017 | BRN .0003 | UTR .0008 | INS .001 |
| SEQ ID NO: 121 | PRO .0017 | BRN .0003 | UTR .0008 | INS .001 |
| SEQ ID NO: 122 | PRO .0031 | | | |
| SEQ ID NO: 123 | PRO .0031 | | | |
| SEQ ID NO: 124 | PRO .0038 | | | |
| SEQ ID NO: 125 | PRO .0038 | | | |
| SEQ ID NO: 126 | PRO .0038 | | | |
| SEQ ID NO: 127 | PRO .0062 | BLO .0003 | LNG .0004 | |
| SEQ ID NO: 128 | PRO .0062 | BLO .0003 | LNG .0004 | |
| SEQ ID NO: 129 | PRO .0062 | BLO .0003 | LNG .0004 | |
| SEQ ID NO: 130 | PRO .0062 | BLO .0003 | LNG .0004 | |
| SEQ ID NO: 131 | PRO .0014 | BRN .0004 | UTR .0004 | MAM .0008 |
| SEQ ID NO: 132 | PRO .0014 | BRN .0004 | UTR .0004 | MAM .0008 |
| SEQ ID NO: 133 | PRO .002 | | | |
| SEQ ID NO: 134 | PRO .0017 | UTR .0004 | | |
| SEQ ID NO: 135 | PRO .0017 | UTR .0004 | | |

Abbreviation for tissues:

BLO Blood; BRN Brain; CON Connective Tissue; CRD Heart; FTS Fetus; INL Intestine, Large; INS Intestine, Small; KID Kidney; LIV Liver; LNG Lung; MAM Breast; MSL Muscles; NRV Nervous Tissue; OVR Ovary; PRO Prostate; STO Stomach; THR Thyroid Gland; TNS Tonsil/Adenoids; UTR Uterus

The chromosomal locations were determined for several of the sequences. Specifically:

DEX0265_{—}1 chromosome 1

DEX0265_{—}9 chromosome X

DEX0265_{—}42 chromosome 3

DEX0265_{—}43 chromosome 3

DEX0265_{—}49 chromosome 1

DEX0265_{—}71 chromosome 19

DEX0265_{—}83 chromosome 13

DEX0265_{—}98 chromosome 10

### Example 2

### Relative Quantitation of Gene Expression

Real-Time quantitative PCR with fluorescent Taqman probes is a quantitation detection system utilizing the 5′-3′ nuclease activity of Taq DNA polymerase. The method uses an internal fluorescent oligonucleotide probe (Taqman) labeled with a 5′ reporter dye and a downstream, 3′ quencher dye. During PCR, the 5′-3′ nuclease activity of Taq DNA polymerase releases the reporter, whose fluorescence can then be detected by the laser detector of the Model 7700 Sequence Detection System (PE Applied Biosystems, Foster City, Calif., USA). Amplification of an endogenous control is used to standardize the amount of sample RNA added to the reaction and normalize for Reverse Transcriptase (RT) efficiency. Either cyclophilin, glyceraldehyde-3-phosphate dehydrogenase (GAPDH), ATPase, or 18S ribosomal RNA (rRNA) is used as this endogenous control. To calculate relative quantitation between all the samples studied, the target RNA levels for one sample were used as the basis for comparative results (calibrator). Quantitation relative to the “calibrator” can be obtained using the standard curve method or the comparative method (User Bulletin #2: ABI PRISM 7700 Sequence Detection System).

The tissue distribution and the level of the target gene are evaluated for every sample in normal and cancer tissues. Total RNA is extracted from normal tissues, cancer tissues, and from cancers and the corresponding matched adjacent tissues. Subsequently, first strand cDNA is prepared with reverse transcriptase and the polymerase chain reaction is done using primers and Taqman probes specific to each target gene. The results are analyzed using the ABI PRISM 7700 Sequence Detector. The absolute numbers are relative levels of expression of the target gene in a particular tissue compared to the calibrator tissue.

One of ordinary skill can design appropriate primers. The relative levels of expression of the PSNA versus normal tissues and other cancer tissues can then be determined. All the values are compared to normal thymus (calibrator). These RNA samples are commercially available pools, originated by pooling samples of a particular tissue from different individuals.

The relative levels of expression of the PSNA in pairs of matching samples and 1 cancer and 1 normal/normal adjacent of tissue may also be determined. All the values are compared to normal thymus (calibrator). A matching pair is formed by mRNA from the cancer sample for a particular tissue and mRNA from the normal adjacent sample for that same tissue from the same individual.

In the analysis of matching samples, the PSNAs that show a high degree of tissue specificity for the tissue of interest. These results confirm the tissue specificity results obtained with normal pooled samples.

Further, the level of mRNA expression in cancer samples and the isogenic normal adjacent tissue from the same individual are compared. This comparison provides an indication of specificity for the cancer stage (e.g. higher levels of mRNA expression in the cancer sample compared to the normal adjacent). Altogether, the high level of tissue specificity, plus the mRNA overexpression in matching samples tested are indicative of SEQ ID NO: 1 through 135 being a diagnostic marker for cancer.

| | | | QPCR |
|---|---|---|---|
| | | | prostate |
| Sequences | Sequence ID NO | Gene ID | code |
| DEX0101_47 | DEX0265_65(SEQ ID NO:65) | 408410 | Pro144 |
| DEX0101_72 | DEX0265_97(SEQ ID NO:97) | 66398 | Pro148 |
| | DEX0265_98(SEQ ID NO:98) | | |

DEX0265_{—}65(SEQ ID NO:65); Pro144

The relative levels of expression of Pro 144 in 24 normal different tissues were determined. All the values are compared to normal brain (calibrator). These RNA samples are commercially pools, originated by pooling samples of a particular tissue from different individuals.

| Tissue | NORMAL |
|---|---|
| Adrenal Gland | 0.00 |
| Bladder | 0.00 |
| Brain | 1.00 |
| Cervix | 0.00 |
| Colon | 0.23 |
| Endometrium | 0.42 |
| Esophagus | 0.00 |
| Heart | 0.00 |
| Kidney | 1.34 |
| Liver | 0.00 |
| Lung | 5.66 |
| Mammary Gland | 0.58 |
| Muscle | 0.00 |
| Ovary | 6.45 |
| Pancreas | 1.21 |
| Prostate | 6.92 |
| Rectum | 8.54 |
| Small Intestine | 0.92 |
| Spleen | 0.78 |
| Stomach | 0.00 |
| Testis | 0.00 |
| Thymus | 1.45 |
| Trachea | 5.37 |
| Uterus | 0.00 |

0 = negative

The relative levels of expression in the table above show that Pro144 mRNA expression is high in prostate.

The absolute numbers in the table above were obtained analyzing pools of samples of a particular tissue from different individuals. They can not be compared to the absolute numbers originated from RNA obtained from tissue samples of a single individual in the table below.

The relative levels of expression of Pro144 in 16 pairs of matching samples and prostate normal, and 6 prostatitis & Benign Prostatic Hyperplasia (BPH) samples and 1 cancer ovary and 1 normal ovary, 1 cancer mammary and 1 normal mammary. All the values are compared to normal brain (calibrator). A matching pair is formed by mRNA from the cancer sample for a particular tissue and mRNA from the normal adjacent sample for that same tissue from the same individual.

| | | | PROSTATISIS & | MATCHING | |
|---|---|---|---|---|---|
| | | | (BPH) BENIGH | NORMAL | |
| Sample ID | Tissue | CANCER | HYPERPLACIA | ADJACENT | NORMAL |
| Pro77P | Prostate 1 | | | | 0.37 |
| Pro12B | Prostate 2 | 32.79 | | 0.31 | |
| Pro101XB | Prostate 3 | 44.48 | | 4.66 | |
| Pro91X | Prostate 4 | 36.5 | | 2.48 | |
| Pro125XB | Prostate 5 | 6.15 | | 9.45 | |
| Pro23B | Prostate 6 | 7.84 | | 2.78 | |
| Pro65XB | Prostate 7 | 41.21 | | 42.52 | |
| Pro90XB | Prostate 8 | 6.21 | | 2.16 | |
| Pro69XB | Prostate 9 | 3.49 | | 2.44 | |
| Pro10P | Prostate 10 | | 0.57 | | |
| | (BPH) | | | | |
| Pro13P | Prostate 11 | | 0.79 | | |
| | (BPH) | | | | |
| Pro34P | Prostate 12 | | 0.07 | | |
| | (BPH) | | | | |
| Pro277 | Prostate 13 | | 2.96 | | |
| | (BPH) | | | | |
| Pro267A | Prostate 14 | | 0.38 | | |
| | (BPH) | | | | |
| Pro271A | Prostate 15 | | 0 | | |
| | (BPH) | | | | |
| Bld46XK | Bladder 1 | 0.02 | | 0 | |
| Bld46K | Bladder 2 | 0 | | 0 | |
| BldTR14 | Bladder 3 | 0.55 | | 0.11 | |
| ClnSG33 | Colon 1 | 0.09 | | 1.20 | |
| Liv 15XA | Liver 1 | 0.06 | | 0.28 | |
| Mam12B | Mammary1 | 0.16 | | | |
| MamA04 | Mammary2 | | | | 0.02 |
| Ovr32RA | Ovary1 | | | | 0.73 |
| Ovr1461 | Ovary2 | 0.10 | | | |
| Tst647T | Testis 1 | 0.21 | | 0.04 | |
| Utr135XO | Uterus 1 | 0.26 | | 0.35 | |

0.00 = Negative

We compared the level of mRNA expression in cancer samples and the isogenic normal adjacent tissue from the same individual. This comparison provides an indication of specificity for the cancer stage (e.g. higher levels of mRNA expression in the cancer sample compared to the normal adjacent). The table above shows overexpression of Pro144 in 63% of the prostate matching samples tested (5 out of total of 8 prostate matching samples).

Altogether, the tissue specificity, plus the mRNA differential expression in the prostate matching samples tested are believed to make Pro 144 a good marker for diagnosing, monitoring, staging, imaging and treating prostate cancer.

Primers Used for QPCR Expression Analysis

In DEX0265_{—}65(SEQ ID NO:65)

| Primer Probe | Start | | | |
|---|---|---|---|---|
| Oligo | From | End To | QueryLength | sbjctDescript |
| Pr0144For | 294 | 317 | 24 | DEX0101_47 |
| Pro144Rev | 419 | 401 | 19 | DEX0101_47 |
| Pro144Probe | 320 | 346 | 27 | DEX0101_47 |

DEX0265_{—}97(SEQ ID NO:97) & DEX0265_{—}98(SEQ ID NO:98); Pro148 Which may be Found in Chromosome 10

Table 1. The relative levels of expression of Pro 148 in 24 normal different tissues were determined. All the values are compared to normal rectum (calibrator). These RNA samples are commercially pools, originated by pooling samples of a particular tissue from different individuals.

| Tissue | NORMAL |
|---|---|
| Adrenal Gland | 0.46 |
| Bladder | 0.34 |
| Brain | 1.00 |
| Cervix | 0.51 |
| Colon | 0.12 |
| Endometrium | 3.08 |
| Esophagus | 0.07 |
| Heart | 0.01 |
| Kidney | 1.08 |
| Liver | 0.18 |
| Lung | 1.51 |
| Mammary Gland | 0.52 |
| Muscle | 0.00 |
| Ovary | 1.71 |
| Pancreas | 0.91 |
| Prostate | 3.42 |
| Rectum | 1.00 |
| Small Intestine | 0.56 |
| Spleen | 1.00 |
| Stomach | 0.99 |
| Testis | 7.89 |
| Thymus | 1.02 |
| Trachea | 2.38 |
| Uterus | 0.19 |

0 = negative

The relative levels of expression in the table above show that Pro148 mRNA expression is highest in prostate.

The absolute numbers in the table above were obtained analyzing pools of samples of a particular tissue from different individuals. They cannot be compared to the absolute numbers originated from RNA obtained from tissue samples of a single individual in the table below.

The relative levels of expression of Pro148 in 15 pairs of matching samples and I prostate normal, and 6 Benign Prostatic Hyperplasia (BPH) samples, and 1 cancer ovary and 1 normal ovary, and 1 mammary normal and 1 mammary cancer. All the values are compared to normal rectum (calibrator). A matching pair is formed by mRNA from the cancer sample for a particular tissue and mRNA from the normal adjacent sample for that same tissue from the same individual.

| | | | PROSTATISIS & | MATCHING | |
|---|---|---|---|---|---|
| | | | (BPH) BENIGH | NORMAL | |
| Sample ID | Tissue | CANCER | HYPERPLACIA | ADJACENT | NORMAL |
| Pro77P | Prostate 1 | | | | 2.87 |
| Pro12B | Prostate 2 | 18.32 | | 3.69 | |
| Pro101XB | Prostate 3 | 7.44 | | 9.48 | |
| Pro91X | Prostate 4 | 5.46 | | 3.82 | |
| Pro125XB | Prostate 5 | 2.15 | | 10.09 | |
| Pro23B | Prostate 6 | 10.30 | | 11.27 | |
| Pro65XB | Prostate 7 | 8.54 | | 25.19 | |
| Pro90XB | Prostate 8 | 9.68 | | 4.16 | |
| Pro69XB | Prostate 9 | 13.69 | | 15.83 | |
| Pro10P | Prostate 10 | | 2.23 | | |
| | (BPH) | | | | |
| Pro13P | Prostate 11 | | 1.36 | | |
| | (BPH) | | | | |
| Pro34P | Prostate 12 | | 7.24 | | |
| | (BPH) | | | | |
| Pro277 | Prostate 13 | | 1.08 | | |
| | (BPH) | | | | |
| Pro267A | Prostate 14 | | 0.37 | | |
| | (BPH) | | | | |
| Pro271A | Prostate 15 | | 0.64 | | |
| | (BPH) | | | | |
| Bld46XK | Bladder 1 | 0.14 | | 0.74 | |
| Bld46K | Bladder 2 | 0.00 | | 0.27 | |
| BldTR14 | Bladder 3 | 5.76 | | 2.55 | |
| ClnSG33 | Colon 1 | 6.59 | | 8.22 | |
| Liv 15XA | Liver 1 | 1.01 | | 0.80 | |
| Mam12B | Mammary1 | 0.84 | | | |
| MamAO4 | Mammary2 | | | | 4.07 |
| Ovr32RA | Ovary1 | | | | 2.21 |
| Ovr1461 | Ovary2 | 3.63 | | | |
| Tst647T | Testis 1 | 1.75 | | 1.52 | |
| Utr135XO | Uterus 1 | 1.04 | | 1.66 | |

We compared the level of mRNA expression in cancer samples and the isogenic normal adjacent tissue from the same individual. This comparison provides an indication of specificity for the cancer stage (e.g. higher levels of mRNA expression in the cancer sample compared to the normal adjacent). Table 2 shows overexpression of Pro148 in 25% of the prostate matching samples tested (2 out of total of 8 prostate matching samples).

Altogether, the tissue specificity, plus the mRNA differential expression in the prostate matching samples tested are believed to make Pro148 a good marker for diagnosing, monitoring, staging, imaging and treating prostate cancer.

Primers Used for QPCR Expression Analysis

In DEX0265_{—}97(SEQ ID NO:97)

| Primer Probe | Start | | | |
|---|---|---|---|---|
| Oligo | From | End To | QueryLength | sbjctDescript |
| Pr0148For | 537 | 558 | 22 | DEX0101_72 |
| Pro148Rev | 672 | 650 | 23 | DEX0101_72 |
| Pro148Probe | 619 | 596 | 24 | DEX0101_72 |

In DEX0265_{—}98(SEQ ID NO:98)

| Primer Probe | Start | | | |
|---|---|---|---|---|
| Oligo | From | End To | QueryLength | sbjctDescript |
| Pro148For | 537 | 558 | 22 | flexsednt |
| | | | | DEX0101_72 |
| Pro148Rev | 672 | 650 | 23 | flexsednt |
| | | | | DEX0101_72 |
| Pro148Probe | 619 | 596 | 24 | flexsednt |
| | | | | DEX0101_72 |

### Example 3

### Protein Expression

The PSNA is amplified by polymerase chain reaction (PCR) and the amplified DNA fragment encoding the PSNA is subcloned in pET-21d for expression in E. coli. In addition to the PSNA coding sequence, codons for two amino acids, Met-Ala, flanking the NH₂-terminus of the coding sequence of PSNA, and six histidines, flanking the COOH-terminus of the coding sequence of PSNA, are incorporated to serve as initiating Met/restriction site and purification tag, respectively.

An over-expressed protein band of the appropriate molecular weight may be observed on a Coomassie blue stained polyacrylamide gel. This protein band is confirmed by Western blot analysis using monoclonal antibody against 6× Histidine tag.

Large-scale purification of PSP was achieved using cell paste generated from 6-liter bacterial cultures, and purified using immobilized metal affinity chromatography (IMAC). Soluble fractions that had been separated from total cell lysate were incubated with a nickle chelating resin. The column was packed and washed with five column volumes of wash buffer. PSP was eluted stepwise with various concentration imidazole buffers.

### Example 4

### Protein Fusions

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector. For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and a polynucleotide of the present invention, isolated by the PCR protocol described in Example 2, is ligated into this BamHI site. Note that the polynucleotide is cloned without a stop codon, otherwise a fusion protein will not be produced. If the naturally occurring signal sequence is used to produce the secreted protein, pC4 does not need a second signal peptide. Alternatively, if the naturally occurring signal sequence is not used, the vector can be modified to include a heterologous signal sequence. See, e.g., WO 96/34891.

### Example 5

### Production of an Antibody from a Polypeptide

In general, such procedures involve immunizing an animal (preferably a mouse) with polypeptide or, more preferably, with a secreted polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100, μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al., Gastroenterology 80: 225-232 (1981).

The hybridoma cells obtained through such a selection are then assayed to identify clones which secrete antibodies capable of binding the polypeptide. Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the protein-specific antibody can be blocked by the polypeptide. Such antibodies comprise anti-idiotypic antibodies to the protein specific antibody and can be used to immunize an animal to induce formation of further protein-specific antibodies. Using the Jameson-Wolf methods the following epitopes were predicted. (Jameson and Wolf, CABIOS, 4(1), 181-186, 1988, the contents of which are incorporated by reference).

The following peptide sequences were predicted based on the nucleotide sequence.

DEX0265_{—}136 is SEQ ID NO:136, etc.

| | |
|---|---|
| >DEX0265_136 | LOAA DEX0101_1 |
| >DEX0265_137 | LOAA DEX0101_2 |
| >DEX0265_138 | LOAA DEX0101_3 |
| >DEX0265_139 | LOAA DEX0101_4 |
| >DEX0265_140 | LOAA DEX0101_5 |
| >DEX0265_141 | LOAA DEX0101_6 |
| >DEX0265_142 | LOAA DEX0101_7 |
| >DEX0265_143 | LOAA DEX0101_8 |
| >DEX0265_144 | LOAA DEX0101_9 |
| >DEX0265_145 | LOAA DEX0101_10 |
| >DEX0265_146 | LOAA DEX0101_11 |
| >DEX0265_147 | LOAA DEX0101_12 |
| >DEX0265_148 | LOAA DEX0101_14 |
| >DEX0265_149 | LOAA DEX0101_15 |
| >DEX0265_150 | LOAA DEX0101_16 |
| >DEX0265_151 | LOAA DEX0101_17 |
| >DEX0265_152 | LOAA DEX0101_18 |
| >DEX0265_153 | LOAA DEX0101_19 |
| >DEX0265_154 | LOAA DEX0101_20 |
| >DEX0265_155 | LOAA DEX0101_21 |
| >DEX0265_156 | flexsedAA DEX0101_21 |
| >DEX0265_157 | LOAA DEX0101_22 |
| >DEX0265_158 | LOAA DEX0101_24 |
| >DEX0265_159 | LOAA DEX0101_25 |
| >DEX0265_160 | flexsedAA DEX0101_25 |
| >DEX0265_161 | LOAA DEX0101_27 |
| >DEX0265_162 | LOAA DEX0101_28 |
| >DEX0265_163 | LOAA DEX0101_29 |
| >DEX0265_164 | LOAA DEX0101_30 |
| >DEX0265_165 | flexsedAA DEX0101_30 |
| >DEX0265_166 | LOAA DEX0101_31 |
| >DEX0265_167 | flexsedAA DEX0101_31 |
| >DEX0265_168 | LOAA DEX0101_32 |
| >DEX0265_169 | LOAA DEX0101_34 |
| >DEX0265_170 | LOAA DEX0101_35 |
| >DEX0265_171 | flexsedAA DEX0101_35 |
| >DEX0265_172 | LOAA DEX0101_36 |
| >DEX0265_173 | flexsedAA DEX0101_36 |
| >DEX0265_174 | LOAA DEX0101_37 |
| >DEX0265_175 | LOAA DEX0101_38 |
| >DEX0265_176 | LOAA DEX0101_39 |
| >DEX0265_177 | LOAA DEX0101_41 |
| >DEX0265_178 | flexsedAA DEX0101_41 |
| >DEX0265_179 | LOAA DEX0101_42 |
| >DEX0265_180 | flexsedAA DEX0101_42 |
| >DEX0265_181 | LOAA DEX0101_44 |
| >DEX0265_182 | LOAA DEX0101_45 |
| >DEX0265_183 | LOAA DEX0101_46 |
| >DEX0265_184 | LOAA DEX0101_47 |
| >DEX0265_185 | LOAA DEX0101_48 |
| >DEX0265_186 | LOAA DEX0101_49 |
| >DEX0265_187 | flexsedAA DEX0101_49 |
| >DEX0265_188 | LOAA DEX0101_50 |
| >DEX0265_189 | LOAA DEX0101_51 |
| >DEX0265_190 | flexsedAA DEX0101_51 |
| >DEX0265_191 | LOAA DEX0101_52 |
| >DEX0265_192 | LOAA DEX0101_53 |
| >DEX0265_193 | LOAA DEX0101_54 |
| >DEX0265_194 | LOAA DEX0101_57 |
| >DEX0265_195 | LOAA DEX0101_58 |
| >DEX0265_196 | LOAA DEX0101_59 |
| >DEX0265_197 | LOAA DEX0101_60 |
| >DEX0265_198 | LOAA DEX0101_61 |
| >DEX0265_199 | LOAA DEX0101_62 |
| >DEX0265_200 | LOAA DEX0101_63 |
| >DEX0265_201 | LOAA DEX0101_64 |
| >DEX0265_202 | LOAA DEX0101_65 |
| >DEX0265_203 | LOAA DEX0101_66 |
| >DEX0265_204 | LOAA DEX0101_67 |
| >DEX0265_205 | LOAA DEX0101_68 |
| >DEX0265_206 | LOAA DEX0101_69 |
| >DEX0265_207 | LOAA DEX0101_70 |
| >DEX0265_208 | LOAA DEX0101_71 |
| >DEX0265_209 | LOAA DEX0101_72 |
| >DEX0265_210 | flexsedAA DEX0101_72 |
| >DEX0265_211 | LOAA DEX0101_73 |
| >DEX0265_212 | LOAA DEX0101_74 |
| >DEX0265_213 | LOAA DEX0101_75 |
| >DEX0265_214 | flexsedAA DEX0101_75 |
| >DEX0265_215 | LOAA DEX0101_76 |
| >DEX0265_216 | flexsedAA DEX0101_76 |
| >DEX0265_217 | LOAA DEX0101_77 |
| >DEX0265_218 | LOAA DEX0101_78 |
| >DEX0265_219 | flexsedAA DEX0101_78 |
| >DEX0265_220 | LOAA DEX0101_79 |
| >DEX0265_221 | flexsedAA DEX0101_79 |
| >DEX0265_222 | LOAA DEX0101_80 |
| >DEX0265_223 | LOAA DEX0101_81 |
| >DEX0265_224 | LOAA DEX0101_82 |
| >DEX0265_225 | flexsedAA DEX0101_82 |
| >DEX0265_226 | LOAA DEX0101_83 |
| >DEX0265_227 | LOAA DEX0101_84 |
| >DEX0265_228 | LOAA DEX0101_85 |
| >DEX0265_229 | LOAA DEX0101_86 |
| >DEX0265_230 | LOAA DEX0101_87 |
| >DEX0265_231 | LOAA DEX0101_88 |
| >DEX0265_232 | LOAA DEX0101_89 |
| >DEX0265_233 | flexsedAA DEX0101_89 |
| >DEX0265_234 | LOAA DEX0101_90 |
| >DEX0265_235 | LOAA DEX0101_91 |
| >DEX0265_236 | flexsedAA DEX0101_91 |
| >DEX0265_237 | LOAA DEX0101_93 |
| >DEX0265_238 | flexsedAA DEX0101_93 |
| >DEX0265_239 | LOAA DEX0101_94 |
| >DEX0265_240 | LOAA DEX0101_95 |

| | Antigenicity Index(Jameson-Wolf) | |
|---|---|---|
| positions | AI | avg length |

DEX0265_138

| | | |
|---|---|---|
| 25-38 | 1.06 | 14 |
| 43-60 | 1.03 | 18 |

DEX0265_140

| | | |
|---|---|---|
| 10-23 | 1.11 | 14 |

DEX0265_141

| | | |
|---|---|---|
| 19-45 | 1.02 | 27 |

DEX0265_147

| | | |
|---|---|---|
| 5-14 | 1.19 | 10 |
| 52-72 | 1.09 | 21 |

DEX0265_154

| | | |
|---|---|---|
| 21-32 | 1.05 | 12 |

DEX0265_155

| | | |
|---|---|---|
| 33-44 | 1.12 | 12 |

DEX0265_164

| | | |
|---|---|---|
| 33-43 | 1.09 | 11 |
| 128-145 | 1.06 | 18 |

DEX0265_167

| | | |
|---|---|---|
| 229-251 | 1.00 | 23 |

DEX0265_169

| | | |
|---|---|---|
| 13-25 | 1.20 | 13 |

DEX0265_172

| | | |
|---|---|---|
| 18-48 | 1.09 | 31 |

DEX0265_174

| | | |
|---|---|---|
| 88-123 | 1.00 | 36 |

DEX0265_176

| | | |
|---|---|---|
| 27-47 | 1.18 | 21 |

DEX0265_178

| | | |
|---|---|---|
| 17-26 | 1.21 | 10 |
| 50-59 | 1.11 | 10 |
| 501-521 | 1.10 | 21 |
| 326-336 | 1.03 | 11 |
| 371-395 | 1.03 | 25 |

DEX0265_180

| | | |
|---|---|---|
| 118-134 | 1.19 | 17 |
| 63-77 | 1.02 | 15 |
| 158-167 | 1.02 | 10 |

DEX0265_184

| | | |
|---|---|---|
| 75-94 | 1.08 | 20 |

DEX0265_187

| | | |
|---|---|---|
| 379-414 | 1.06 | 36 |
| 47-69 | 1.03 | 23 |

DEX0265_190

| | | |
|---|---|---|
| 21-30 | 1.02 | 10 |
| 32-61 | 1.01 | 30 |

DEX0265_195

| | | |
|---|---|---|
| 17-32 | 1.02 | 16 |

DEX0265_215

| | | |
|---|---|---|
| 8-17 | 1.21 | 10 |

DEX0265_216

| | | |
|---|---|---|
| 49-61 | 1.20 | 13 |
| 24-42 | 1.11 | 19 |

DEX0265_219

| | | |
|---|---|---|
| 69-81 | 1.12 | 13 |

DEX0265_221

| | | |
|---|---|---|
| 273-290 | 1.12 | 18 |
| 21-70 | 1.02 | 50 |

DEX0265_224

| | | |
|---|---|---|
| 69-90 | 1.12 | 22 |
| 18-31 | 1.12 | 14 |
| 35-48 | 1.12 | 14 |

DEX0265_232

| | | |
|---|---|---|
| 3-41 | 1.13 | 39 |

DEX0265_233

| | | |
|---|---|---|
| 287-312 | 1.26 | 26 |
| 350-395 | 1.18 | 46 |
| 693-717 | 1.14 | 25 |
| 531-557 | 1.10 | 27 |
| 168-189 | 1.10 | 22 |
| 34-63 | 1.09 | 30 |
| 397-438 | 1.08 | 42 |
| 749-762 | 1.06 | 14 |
| 459-469 | 1.03 | 11 |
| 80-95 | 1.02 | 16 |
| 267-285 | 1.01 | 19 |
| 471-526 | 1.01 | 56 |

DEX0265_238

| | | |
|---|---|---|
| 98-109 | 1.42 | 12 |
| 12-34 | 1.11 | 23 |
| 59-96 | 1.07 | 38 |
| 128-164 | 1.07 | 37 |
| 249-272 | 1.02 | 24 |

Examples of post-translational modifications (PTMs) of the PSPs of this invention are listed below. In addition, antibodies that specifically bind such post-translational modifications may be useful as a diagnostic or as therapeutic. Using the ProSite database (Bairoch et al., Nucleic Acids Res. 25(1):217-221 (1997), the contents of which are incorporated by reference), the following PTMs were predicted for the PSPs of the invention (http://npsa-pbil.ibcp.fr/cgi-bin/npsa automat.pl?page=npsa_prosite.html most recently accessed Oct. 23, 2001). For full definitions of the PTMs see http://www.expasy.org/cgi-bin/prosite-list.pl most recently accessed Oct. 23, 2001.

| | |
|---|---|
| DEX0265_136 | Pkc_Phospho_Site 25-27; |
| DEX0265_138 | Camp_Phospho_Site 32-35; Myristyl 41-46; |
| DEX0265_140 | Asn_Glycosylation 12-15; Camp_Phospho_Site 19-22; Myristyl |
| | 46-51; Pkc_Phospho_Site 14-16; |
| DEX0265_141 | Myristyl 14-19;37-42; |
| DEX0265_142 | Ck2_Phospho_Site 5-8; Myristyl 13-18 Pkc_Phospho_Site 30-32; |
| DEX0265_143 | Myristyl 17-22; Pkc_Phospho_Site 7-9; |
| DEX0265_145 | Ck2_Phospho_Site 56-59; Pkc_Phospho_Site 21-23; |
| DEX0265_146 | Ck2_Phospho_Site 29-32; Myristyl 4-9; Pkc_Phospho_Site 10-12; |
| DEX0265_147 | Myristyl 58-63;62-67;83-88 Pkc_Phospho_Site 19-21; |
| DEX0265_150 | Ck2_Phospho_Site 28-31; Pkc_Phospho_Site 7-9;12-14;28-30;54- |
| | 56;77-79; |
| DEX0265_153 | Ck2_Phospho_Site 34-37; Myristyl 27-32; Pkc_Phospho_Site 24- |
| | 26;34-36; |
| DEX0265_154 | Asn_Glycosylation 63-66; |
| DEX0265_155 | Ck2_Phospho_Site 10-13; Myristyl 6-11;43-48; |
| DEX0265_156 | Myristyl 35-40;62-67; |
| DEX0265_158 | Prokar_Lipoprotein 4-14; |
| DEX0265_159 | Myristyl 6-11; |
| DEX0265_160 | Myristyl 4-9;18-23; |
| DEX0265_162 | Myristyl 19-24; |
| DEX0265_163 | Myristyl 45-50; |
| DEX0265_164 | Ck2_Phospho_Site 89-92; Myristyl 30-35; Pkc_Phospho_Site 16- |
| | 18;89-91;139-141; |
| DEX0265_165 | Ck2_Phospho_Site 9-12; Pkc_Phospho_Site 9-11;59-61; |
| DEX0265_167 | Asn_Glycosylation 41-44;221-224;233-236; Ck2_Phospho_Site |
| | 27-30;43-46;56-59;304-307;359-362;432-435;458-461; Myristyl |
| | 39-44;71-76;194-199;210-215;292-297;297-302;427-432;452-457; |
| | Pkc_Phospho_Site 11-13;49-51;289-291;382-384; |
| | Tyr_Phospho_Site 130-138; |
| DEX0265_169 | Ck2_Phospho_Site 14-17; Pkc_Phospho_Site 37-39; |
| DEX0265_170 | Camp_Phospho_Site 126-129; Myristyl 116-121; |
| | Pkc_Phospho_Site 97-99;120-122;124-126; Tyr_Phospho_Site 58- |
| | 65;59-65; |
| DEX0265_171 | Myristyl 116-121;118-123 Pkc_Phospho_Site 97-99; |
| | Tyr_Phospho_Site 58-65;59-65; |
| DEX0265_173 | Myristyl 19-24;44-49; |
| DEX0265_174 | Asn_Glycosylation 28-31; Myristyl 57-62;109-114; |
| | Pkc_Phospho_Site 19-21;53-55;90-92;97-99; |
| DEX0265_175 | Amidation 47-50; Myristyl 8-13;88-93;120-125; |
| | Pkc _Phospho_Site 89-91 |
| DEX0265_176 | Amidation 46-49; Myristyl 3-8;5-10;29-34; Pkc_Phospho_Site 38- |
| | 40; |
| DEX0265_178 | Amidation 513-516;555-558 Asn_Glycosylation 144-147;202- |
| | 205;214-217;374-377;459-462;464-467; Camp_{—Phospho}_Site |
| | 241-244;392-395;530-533;557-560; Ck2_Phospho_Site 8-11;19- |
| | 22;265-268;560-563; Myristyl 78-83;91-96;116-121;154-159;198- |
| | 203;200-205;285-290;412-417;441-446;462-467;469-474; |
| | Pkc_Phospho_Site 56-58;81-83;166-168;236-238;390-392;391- |
| | 393;528-530;Zinc_Finger_C2h2 520-542;550-572;580-600; |
| DEX0265_179 | Myristyl 19-24; |
| DEX0265_180 | Ck2_Phospho_Site 16-19;55-58;84-87;108-111;125-128;142-145; |
| | Myristyl 75-80;79-84; Pkc_Phospho_Site 16-18;55-57;71-73;84- |
| | 86;125-127; |
| DEX0265_181 | Ck2_Phospho_Site 56-59;Myristyl 40-45; Prokar_Lipoprotein 71- |
| | 81; |
| DEX0265_182 | Camp_Phospho_Site 6-9 Pkc_Phospho_Site 9-11;27-29;28-30; |
| DEX0265_183 | Asn_Glycosylation 10-13; Ck2_Phospho_Site 42-45; Myristyl 14- |
| | 19;17-22;21-26; |
| DEX0265_184 | Ck2_Phospho_Site 87-90;92-95; Myristyl 15-20;40-45;45-50; |
| | Pkc_Phospho_Site 79-81; |
| DEX0265_185 | Asn_Glycosylation 15-18;19-22; Myristyl 12-17; |
| | Pkc_Phospho_Site 16-18; |
| DEX0265_186 | Ck2_Phospho_Site 37-40; Myristyl 73-78; |
| DEX0265_187 | Amidation 96-99; Asn_Glycosylation 135-138;229-232;455-458; |
| | Camp_Phospho_Site 175-178;193-196; Ck2_Phospho_Site 109- |
| | 112;122-125;137-140;214-217;459-462;465-468; Myristyl 30- |
| | 35;79-84;283-288;439-444; Pkc_Phospho_Site 109-111;173- |
| | 175;191-193;196-198;232-234;271-273;392-394;405-407; |
| | Tyr_Phospho_Site 241-248;307-313;393-400; |
| DEX0265_188 | Pkc_Phospho_Site 5-7; |
| DEX0265_189 | Asn_Glycosylation 48-51; Ck2_Phospho_Site 39-42;50-53; |
| | Myristyl 4-9; Pkc_Phospho_Site 36-38; |
| DEX0265_190 | Myristyl 18-23;19-24;55-60 Pkc_Phospho_Site 25-27;52-54; |
| DEX0265_191 | Pkc_Phospho_Site 31-33;36-38; |
| DEX0265_192 | Myristyl 16-21; Pkc_Phospho_Site 20-22;26-28; |
| DEX0265_193 | Asn_Glycosylation 38-41;Ck2_Phospho_Site 4-7; |
| | Pkc_Phospho_Site 8-10;13-15;40-42; |
| DEX0265_195 | Ck2_Phospho_Site 9-12; |
| DEX0265_201 | Asn_Glycosylation 11-14; |
| DEX0265_202 | Pkc_Phospho_Site 9-11;18-20; |
| DEX0265_203 | Asn_Glycosylation 32-35; Ck2_Phospho_Site 10-13; |
| | Leucine_Zipper 15-36; Pkc_Phospho_Site 6-8; |
| DEX0265_204 | Ck2_Phospho_Site 3-6;10-13; |
| DEX0265_205 | Ck2_Phospho_Site 4-7; |
| DEX0265_206 | Asn_Glycosylation 37-40; Ck2_Phospho_Site 17-20; |
| DEX0265_207 | Asn_Glycosylation 63-66; Ck2_Phospho_Site 84-87; |
| | Pkc_Phospho_Site 31-33; |
| DEX0265_208 | Asn_Glycosylation 11-14;36-39; Ck2_Phospho_Site 24-27;43-46; |
| | Pkc_Phospho_Site 10-12;43-45;47-49; |
| DEX0265_210 | Pkc_Phospho_Site 47-49; |
| DEX0265_211 | Pkc_Phospho_Site 21-23; |
| DEX0265_212 | Ck2_Phospho_Site 133-136; Myristyl 129-134; |
| DEX0265_213 | Asn_Glycosylation 93-96; Camp_Phospho_Site 337-340; |
| | Ck2_Phospho_Site 28-31;86-89;95-98 G_Protein_Receptor 138- |
| | 154; Myristyl 7-12;21-26;26-31;40-45;136-141;178-183;332-337; |
| | Pkc_Phospho_Site 3-5;60-62;64-66;116-118;316-318;336-338; |
| DEX0265_214 | Asn_Glycosylation 2-5; Camp_Phospho_Site 245-248; |
| | Ck2_Phospho_Site 4-7; G_Protein_Receptor 47-63; Myristyl 45- |
| | 50;86-91;240-245; Pkc_Phospho_Site 25-27;74-76;224-226;244- |
| | 246; |
| DEX0265_215 | Asn_Glycosylation 3-6; Ck2_Phospho_Site 12-15; |
| | Pkc_Phospho_Site 12-14; |
| DEX0265_216 | Asn_Glycosylation 29-32; Camp_Phospho_Site 41-44; |
| | Ck2_Phospho_Site 81-84; Myristyl 61-66;96-101;99-104;100-105; |
| DEX0265_217 | Pkc_Phospho_Site 19-21; |
| DEX0265_218 | Myristyl 11-16; |
| DEX0265_219 | Camp_Phospho_Site 75-78; Ck2_Phospho_Site 51-54;86-89; |
| | Pkc_Phospho_Site 51-53;60-62; |
| DEX0265_220 | Asn_Glycosylation 5-8; Ck2_Phospho_Site 21-24;30-33; |
| DEX0265_221 | Asn_Glycosylation 244-247;359-362; Camp_Phospho_Site 239- |
| | 242; Ck2_Phospho_Site 46-49;150-153;201-204;311-314; |
| | Myristyl 296-301; Pkc_Phospho_Site 46-48;65-67;78-80;155- |
| | 157;227-229;248-250;289-291;334-336;351-353; |
| | Tyr_Phospho_Site 89-96;90-96; |
| DEX0265_224 | Ck2_Phospho_Site 50-53; Myristyl 9-14;43-48;76-81;84-89;87- |
| | 92; Pkc_Phospho_Site 16-18;69-71;77-79; |
| DEX0265_225 | Ck2_Phospho_Site 30-33;104-107;115-118;139-142; |
| | Glycosaminoglycan 31-34; Myristyl 71-76;72-77;122-127; |
| | Pkc_Phospho_Site 76-78;104-106;151-153; |
| DEX0265_226 | Myristyl 58-63; Prokar_Lipoprotein 51-61; |
| DEX0265_227 | Ck2_Phospho_Site 14-17; Myristyl 64-69; |
| DEX0265_229 | Pkc_Phospho_Site 3-5; |
| DEX0265_230 | Amidation 47-50; |
| DEX0265_231 | Pkc_Phospho_Site 2-4;20-22; |
| DEX0265_232 | Myristyl 47-52;74-79; Prokar_Lipoprotein 79-89; |
| DEX0265_233 | Amidation 60-63; Camp_Phospho_Site 305-308;355-358; |
| | Ck2_Phospho_Site 50-53;81-84;126-129;153-156;203-206;215- |
| | 218;230-233;511-514;556-559;581-584;656-659;775-778;808- |
| | 811; Glycosaminoglycan 466-469; Myristyl 40-45;150-155;178- |
| | 183;244-249;256-261;264-269;269-274;342-347;346-351;411- |
| | 416;436-441;475-480;476-481;479-484;529-534;537-542;773- |
| | 778; PkcPhospho_Site 44-46;226-228;303-305;304-306;373- |
| | 375;380-382;427-429;445-447;456-458;511-513;550-552;565- |
| | 567;595-597;653-655;661-663;731-733;803-805;849-851; |
| | Tyr_Phospho_Site 510-518;752-758; |
| DEX0265_234 | Pkc_Phospho_Site 55-57; |
| DEX0265_235 | Ck2_Phospho_Site 24-27; Pkc_Phospho_Site 21-23; |
| DEX0265_236 | Camp_Phospho_Site 30-33; Ck2_Phospho_Site 39-42;72-75; |
| | Myristyl 13-18; |
| DEX0265_237 | Pkc_Phospho_Site 16-18; |
| DEX0265_238 | Asn_Glycosylation 391-394; Camp_Phospho Site 72-75; |
| | Ck2_Phospho_Site 57-60;75-78;117-120;143-146;174-177;304- |
| | 307;393-396;401-404;408-411; Glycosaminoglycan 249-252; |
| | Myristyl 27-32;100-105;159-164;184-189;213-218;217-222;246- |
| | 251;266-271;362-367;433-438; Pkc_Phospho_Site 96-98;131- |
| | 133;143-145;262-264;320-322;337-339;393-395;425-427; |
| | Tnfr_Ngfr_1 97-137;137-178;139-178; |
| DEX0265_239 | Myristyl 4-9; Pkc_Phospho_Site 8-10; |
| DEX0265_240 | Ck2_Phospho_Site 12-15; |

### Example 6

### Method of Determining Alterations in a Gene Corresponding to a Polynucleotide

RNA is isolated from individual patients or from a family of individuals that have a phenotype of interest. cDNA is then generated from these RNA samples using protocols known in the art. See, Sambrook (2001), supra. The cDNA is then used as a template for PCR, employing primers surrounding regions of interest in SEQ ID NO: 1 through 135. Suggested PCR conditions consist of 35 cycles at 95° C. for 30 seconds; 60-120 seconds at 52-58° C.; and 60-120 seconds at 70° C., using buffer solutions described in Sidransky et al., Science 252(5006): 706-9 (1991). See also Sidransky et al., Science 278(5340): 1054-9 (1997).

PCR products are then sequenced using primers labeled at their 5′ end with T4 polynucleotide kinase, employing SequiTherm Polymerase. (Epicentre Technologies). The intron-exon borders of selected exons is also determined and genomic PCR products analyzed to confirm the results. PCR products harboring suspected mutations are then cloned and sequenced to validate the results of the direct sequencing. PCR products is cloned into T-tailed vectors as described in Holton et al., Nucleic Acids Res., 19: 1156 (1991) and sequenced with T7 polymerase (United States Biochemical). Affected individuals are identified by mutations not present in unaffected individuals.

Genomic rearrangements may also be determined. Genomic clones are nick-translated with digoxigenin deoxyuridine 5′ triphosphate (Boehringer Manheim), and FISH is performed as described in Johnson et al., Methods Cell Biol. 35: 73-99 (1991). Hybridization with the labeled probe is carried out using a vast excess of human cot-1 DNA for specific hybridization to the corresponding genomic locus.

Chromosomes are counterstained with 4,6-diamino-2-phenylidole and propidium iodide, producing a combination of C-and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. Id. Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 7

### Method of Detecting Abnormal Levels of a Polypeptide in a Biological Sample

Antibody-sandwich ELISAs are used to detect polypeptides in a sample, preferably a biological sample. Wells of a microtiter plate are coated with specific antibodies, at a final concentration of 0.2 to 10 μg/ml. The antibodies are either monoclonal or polyclonal and are produced by the method described above. The wells are blocked so that non-specific binding of the polypeptide to the well is reduced. The coated wells are then incubated for >2 hours at RT with a sample containing the polypeptide. Preferably, serial dilutions of the sample should be used to validate results. The plates are then washed three times with deionized or distilled water to remove unbound polypeptide. Next, 50 μl of specific antibody-alkaline phosphatase conjugate, at a concentration of 25-400 ng, is added and incubated for 2 hours at room temperature. The plates are again washed three times with deionized or distilled water to remove unbound conjugate. 75 μl of 4-methylumbelliferyl phosphate (MUP) or p-nitrophenyl phosphate (NPP) substrate solution are added to each well and incubated 1 hour at room temperature.

The reaction is measured by a microtiter plate reader. A standard curve is prepared, using serial dilutions of a control sample, and polypeptide concentrations are plotted on the X-axis (log scale) and fluorescence or absorbance on the Y-axis (linear scale). The concentration of the polypeptide in the sample is calculated using the standard curve.

### Example 8

### Formulating a Polypeptide

The secreted polypeptide composition will be formulated and dosed in a fashion consistent with good medical practice, taking into account the clinical condition of the individual patient (especially the side effects of treatment with the secreted polypeptide alone), the site of delivery, the method of administration, the scheduling of administration, and other factors known to practitioners. The “effective amount” for purposes herein is thus determined by such considerations.

As a general proposition, the total pharmaceutically effective amount of secreted polypeptide administered parenterally per dose will be in the range of about 1, μtg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the secreted polypeptide is typically administered at a dose rate of about 1 μg/kg/hour to about 50 mg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the secreted protein of the invention are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

The secreted polypeptide is also suitably administered by sustained-release systems. Suitable examples of sustained-release compositions include semipermeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22: 547-556 (1983)), poly (2-hydroxyethyl methacrylate) (R. Langer et al., J. Biomed. Mater. Res. 15: 167-277 (1981), and R. Langer, Chem. Tech. 12: 98-105 (1982)), ethylene vinyl acetate (R. Langer et al.) or poly-D- (−)-3-hydroxybutyric acid (EP 133,988). Sustained-release compositions also include liposomally entrapped polypeptides. Liposomes containing the secreted polypeptide are prepared by methods known per se: DE Epstein et al., Proc. Natl. Acad. Sci. USA 82: 3688-3692 (1985); Hwang et al., Proc. Natl. Acad. Sci. USA 77: 4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, the secreted polypeptide is formulated generally by mixing it at the desired degree of purity, in a unit dosage injectable form (solution, suspension, or emulsion), with a pharmaceutically acceptable carrier, I. e., one that is non-toxic to recipients at the dosages and concentrations employed and is compatible with other ingredients of the formulation.

For example, the formulation preferably does not include oxidizing agents and other compounds that are known to be deleterious to polypeptides. Generally, the formulations are prepared by contacting the polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The secreted polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any polypeptide to be used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

Polypeptides ordinarily will be stored in unit or multi-dose containers, for example, sealed ampules or vials, as an aqueous solution or as a lyophilized formulation for reconstitution. As an example of a lyophilized formulation, 10-ml vials are filled with 5 ml of sterile-filtered 1% (w/v) aqueous polypeptide solution, and the resulting mixture is lyophilized. The infusion solution is prepared by reconstituting the lyophilized polypeptide using bacteriostatic Water-for-Injection.

The invention also provides a pharmaceutical pack or kit comprising one or more containers filled with one or more of the ingredients of the pharmaceutical compositions of the invention. Associated with such container (s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

### Example 9

### Method of Treating Decreased Levels of the Polypeptide

It will be appreciated that conditions caused by a decrease in the standard or normal expression level of a secreted protein in an individual can be treated by administering the polypeptide of the present invention, preferably in the secreted form. Thus, the invention also provides a method of treatment of an individual in need of an increased level of the polypeptide comprising administering to such an individual a pharmaceutical composition comprising an amount of the polypeptide to increase the activity level of the polypeptide in such an individual.

For example, a patient with decreased levels of a polypeptide receives a daily dose 0.1-100 μg/kg of the polypeptide for six consecutive days. Preferably, the polypeptide is in the secreted form. The exact details of the dosing scheme, based on administration and formulation, are provided above.

### Example 10

### Method of Treating Increased Levels of the Polypeptide

Antisense technology is used to inhibit production of a polypeptide of the present invention. This technology is one example of a method of decreasing levels of a polypeptide, preferably a secreted form, due to a variety of etiologies, such as cancer.

For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided above.

### Example 11

### Method of Treatment Using Gene Therapy

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37° C. for approximately one week.

At this time, fresh media is added and subsequently changed every several days. After an additional two weeks in culture, a monolayer of fibroblasts emerge. The monolayer is trypsinized and scaled into larger flasks. pMV-7 (Kirschmeier, P. T. et al., DNA, 7: 219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in Example 1. Preferably, the 5primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB 101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media.

If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 12

### Method of Treatment Using Gene Therapy-In Vivo

Another aspect of the present invention is using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide.

The polynucleotide of the present invention may be operatively linked to a promoter or any other genetic elements necessary for the expression of the polypeptide by the target tissue. Such gene therapy and delivery techniques and methods are known in the art, see, for example, WO 90/11092, WO 98/11779; U.S. Pat. No. 5,693,622; 5,705,151; 5,580,859; Tabata H. et al. (1997) Cardiovasc. Res. 35 (3): 470-479, Chao J et al. (1997) Pharmacol. Res. 35 (6): 517-522, Wolff J. A. (1997) Neuromuscul. Disord. 7 (5): 314-318, Schwartz B. et al. (1996) Gene Ther. 3 (5): 405-411, Tsurumi Y. et al. (1996) Circulation 94 (12): 3281-3290 (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner P. L. et al. (1995) Ann. NY Acad. Sci. 772: 126-139 and Abdallah B. et al. (1995) Biol. Cell 85 (1): 1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 μg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice.

The results of the above experimentation in mice can be use to extrapolate proper dosages and other treatment parameters in humans and other animals using naked DNA.

### Example 13

### Transgenic Animals

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40: 691-698 (1994); Carver et al., Biotechnology (NY) 11: 1263-1270 (1993); Wright et al., Biotechnology (NY) 9: 830-834 (1991); and Hoppe et al., U.S. Pat. No. 4,873,191 (1989)); retrovirus mediated gene transfer into germ lines (Van der Putten et al., Proc. Natl. Acad. Sci., USA 82: 6148-6152 (1985)), blastocysts or embryos; gene targeting in embryonic stem cells (Thompson et al., Cell 56: 313-321 (1989)); electroporation of cells or embryos (Lo, 1983, Mol Cell. Biol. 3: 1803-1814 (1983)); introduction of the polynucleotides of the invention using a gene gun (see, e.g., Ulmer et al., Science 259: 1745 (1993); introducing nucleic acid constructs into embryonic pleuripotent stem cells and transferring the stem cells back into the blastocyst; and sperm mediated gene transfer (Lavitrano et al., Cell 57: 717-723 (1989); etc. For a review of such techniques, see Gordon, “Transgenic Animals,” Intl. Rev. Cytol. 115: 171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380: 64-66 (1996); Wilmut et al., Nature 385: 810813 (1997)).

The present invention provides for transgenic animals that carry the transgene in all their cells, as well as animals which carry the transgene in some, but not all their cells, I. e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89: 6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265: 103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

### Example 14

### Knock-Out Animals

Endogenous gene expression can also be reduced by inactivating or “knocking out” the gene and/or its promoter using targeted homologous recombination. (E. g., see Smithies et al., Nature 317: 230-234 (1985); Thomas & Capecchi, Cell 51: 503512 (1987); Thompson et al., Cell 5: 313-321(1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (I. e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc.

The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

We claim:

1. An isolated nucleic acid molecule comprising
(a) a nucleic acid molecule comprising a nucleic acid sequence that encodes an amino acid sequence of SEQ ID NO: 136 through 240;
(b) a nucleic acid molecule comprising a nucleic acid sequence of SEQ ID NO: 1 through 135;
(c) a nucleic acid molecule that selectively hybridizes to the nucleic acid molecule of (a) or (b); or (d) a nucleic acid molecule having at least 60% sequence identity to the nucleic acid molecule of (a) or (b).

2. The nucleic acid molecule according to claim 1, wherein the nucleic acid molecule is a cDNA.

3. The nucleic acid molecule according to claim 1, wherein the nucleic acid molecule is genomic DNA.

4. The nucleic acid molecule according to claim 1, wherein the nucleic acid molecule is a mammalian nucleic acid molecule.

5. The nucleic acid molecule according to claim 4, wherein the nucleic acid molecule is a human nucleic acid molecule.

6. A method for determining the presence of a prostate specific nucleic acid (PSNA) in a sample, comprising the steps of:
(a) contacting the sample with the nucleic acid molecule according to claim 1 under conditions in which the nucleic acid molecule will selectively hybridize to a prostate specific nucleic acid; and
(b) detecting hybridization of the nucleic acid molecule to a PSNA in the sample, wherein the detection of the hybridization indicates the presence of a PSNA in the sample.

7. A vector comprising the nucleic acid molecule of claim 1.

8. A host cell comprising the vector according to claim 7.

9. A method for producing a polypeptide encoded by the nucleic acid molecule according to claim 1, comprising the steps of (a) providing a host cell comprising the nucleic acid molecule operably linked to one or more expression control sequences, and (b) incubating the host cell under conditions in which the polypeptide is produced.

10. A polypeptide encoded by the nucleic acid molecule according to claim 1.

11. An isolated polypeptide selected from the group consisting of:
(a) a polypeptide comprising an amino acid sequence with at least 60% sequence identity to of SEQ ID NO: 136 through 240; or
(b) a polypeptide comprising an amino acid sequence encoded by a nucleic acid molecule comprising a nucleic acid sequence of SEQ ID NO: 1 through 135.

12. An antibody or fragment thereof that specifically binds to the polypeptide according to claim 11.

13. A method for determining the presence of a prostate specific protein in a sample, comprising the steps of:
(a) contacting the sample with the antibody according to claim 12 under conditions in which the antibody will selectively bind to the prostate specific protein; and
(b) detecting binding of the antibody to a prostate specific protein in the sample, wherein the detection of binding indicates the presence of a prostate specific protein in the sample.

14. A method for diagnosing and monitoring the presence and metastases of prostate cancer in a patient, comprising the steps of:
(a) determining an amount of the nucleic acid molecule of claim 1 or a polypeptide of claim 6 in a sample of a patient; and
(b) comparing the amount of the determined nucleic acid molecule or the polypeptide in the sample of the patient to the amount of the prostate specific marker in a normal control; wherein a difference in the amount of the nucleic acid molecule or the polypeptide in the sample compared to the amount of the nucleic acid molecule or the polypeptide in the normal control is associated with the presence of prostate cancer.

15. A kit for detecting a risk of cancer or presence of cancer in a patient, said kit comprising a means for determining the presence the nucleic acid molecule of claim 1 or a polypeptide of claim 6 in a sample of a patient.

16. A method of treating a patient with prostate cancer, comprising the step of administering a composition according to claim 12 to a patient in need thereof, wherein said administration induces an immune response against the prostate cancer cell expressing the nucleic acid molecule or polypeptide.

17. A vaccine comprising the polypeptide or the nucleic acid encoding the polypeptide of claim 11.


SEQUENCE LISTING (240 sequences)

SEQ ID NO: 1 - DNA, 199 bp, Homo sapiens
aaatgcaagcctcgcactgggatccaaaggtaaattaagatgatggtccctgcctgcagaagctctcagcctagtgcagggaacaaacatgtgaatacatgcccttctatttggtaaaatgaatggttgagagagcaagaggggaagagatgactctgcccatgaagatggagaaggtatcaggaaaaggacataattg

SEQ ID NO: 2 - DNA, 515 bp, Homo sapiens
gcatttgttctggtgtgcaagagagccggtcacacagcgagagagtgagccccactggccactgactcctcactgtgaacacagctttggtgcttctgttcttcatccccgtgaaataatgagcacttctcaatggaagcacttagtgagttcatgaaggttgcgagatccattccttgaaactgttgagagactcttgttttcagaggctatttcagagaaacagaggctactctgtttcacacatacttactgaaaccctaaatgcaagcctcgcactgggatccaaaggtaaattaagatgatggtccctgcctgcagaagctctcagcctagtgcagggaacaaacatgtgaatacatgcccttctatttggtaaaatgaatggttgagagagcaagaggggaagagatgactctgcccatgaagatggagaaggtatcaggaaaaggacataattgggctgggcatagtggctcacgtgtacaatcccaacactttgggaggcctagtag

SEQ ID NO: 3 - DNA, 297 bp, Homo sapiens
gaattcgctggttggaggagtggcttttatctgaagagaagacacataaaagagaaaaatggtacatttactggtagtaagatttaaaaaagtagtttatgttctcatgtacctttgccctgaacttctcccccagctttctctttaagctcttccagtctgttttaattaccaggttttgttttggcttttctggggggatggggcctgtttagggcagggaaatttgtttgaaaagggcattacaagcaagaaatttttaaaatgggtgtttttttaaaaaagattaaaattacc

SEQ ID NO: 4 - DNA, 387 bp, Homo sapiens
ttctatgactgtgctactgctcatggtgttgttttctttccttagctttcacacagcaactctagagttgtgtgactgagacagtgataaaatgacataaaatgcagaggcatggcactactataagagagcataactattagatttaagatttagagcacatataaatgttcactattcatcctttctacattcatttatgcattcatgtgttgaactcctgctatgcctgctgtataggcaatggggatagagtaaggaaaagacactcaagtcttggctgtcatggagttccatttggcaagggctggggcatgaggggaaggacagagaacaaagggataactaaatatgtaatttgtcaggtggggataagtcccatggagg

SEQ ID NO: 5 - DNA, 318 bp, Homo sapiens
tttaaggtggctggtcactggaatagttaggaatgacactgcaaaaagggtccttagatatacaggatgatcagcccataacatgaggctcagtcttaggaaagttctaagacatggctttggttcttggacccagaacagtgtacagtctatacaatggctgcccaaaaccctctccaaaatacaacctctttaaaggaaaagggcttctcgtttgatgtgcagcgtaatacatcactgccatttataaactgtagctgtaactttgggtaaagacttcatgaatggataaaacgtaagattccgagtactgtagag

SEQ ID NO: 6 - DNA, 466 bp, Homo sapiens
gaagtgaagggttggctatctgctctagaggttcaggtaaagcttccctcctcaggaactgagggtaagtaggaattagccagtaaaaagagtgagggtagatgagtaaaggcacttcaggcaatgagaacaatccatgtaaaagtccttaggcaagaaagtgattggctggttcagggaactgaaagatgagtgtgggtggggcccagggatgatggggtattttgaggtaggctggagccaggccaagcagggtctctagagagagcaaattgataaatgtgcattttattttaagtgcaatgtagagccatgaagtacttttttttgcttgcttgcttgttttgttttttaatcgaagaagtaacctgatcagattcatgttttgaaaagactcctggctcctgtgtggagaaaggattggaggagaggaagaagagatagagaagaattagaaggtgataca

SEQ ID NO: 7 - DNA, 460 bp, Homo sapiens
gctcttaaaaattattgaggatcccaaagaacttatgtttgtatggatcatatctataaatagttgccatcttagaaattaaaatgaaggaatttaataacgcttattttaaaacaataatggacctgttacctgttaatgtaaataacgtttgtatggaaaattactatgtttcaaaaaatagtgagcagagtggcacccttgagaacagccactgtacacttacagtgcctagtacgcgccaggaaagactctgttgaatgagtgaataggagggtgaaagattttagaccagcctgaagggtcagatgcaggtgcatggataggtggggatctgagatggggccgccttttcccctagatggccctggggcccactcccctcctccccactcatccaccccagcctcactcagtcctttctcttcctttccccaacaatcttctctccacagcagtg

SEQ ID NO: 8 - DNA, 1200 bp, Homo sapiens
actattgatattttgggcctgaaaatcttcattgtggggctgtgctatgcatcgtaggatgctagcagcatccctaaccactaccccgtaggttctagtgctgcccccatccccaggagactccccagtcaatagccctcatcataatcaccagcatttctcatctactgtgtttctctaagctacacagcaagttacagctgcctcgtgaggtagtgagttccccgtcactggaatgaggtgttcaaagagaggctggaggatgtttgctggcagagattctgctttcaggagttactgtacgaacagatcgttctcaggcttctgggttccagaaccttctttctctcttaaaaattattgaggatcccaaagaacttatgtttgtatggatcatatctataaatagttgccatcttagaaattaaaatgaaggaatttaataacgcttattttaaaacaataatggacctgttacctgttaatgtaaataacgtttgtatggaaaattactatgtttcaaaaaatagtgagcagagtggcacccttgagaacagccactgtacacttacagtgcctagtacgcgccaggaaagactctgttgaatgagtgaataggagggtgaaagattttagaccagcctgaagggtcagatgcaggtgcatggataggtggggatctgagatggggccgccttttcccctagatggccctggggcccactcccctcctccccactcatccaccccagcctcactcagtcctttctcttcctttccccaacaatcttctctccacagcagtgcaaagaatagaacaagaaatcattttgatgtcgtcagaaatcaatatgtaagctggatatatcttttaaataaactcatttctataacaaaataatcattgtatgaatttcagtccttcaaagggggttcctttggagggtttcccttgccaaatgaatattgaggcccggttttgccaggatgtatttattcccgtctatattcatgttgagaaagctaactgggtgttatattatcttccttattacaatactgaaacctaacaaactgggtgtcatattcttttccttattacaataccgaaacctaagttcttcccctttctaaggagaagctgcctcctccccctttattttttcctctggctagaggaaattcacagagccaagatta

SEQ ID NO: 9 - DNA, 502 bp, Homo sapiens
  misc_feature (391)..(391): n= a, c, g, or t
gggacagttcttatattctccttcctctctcaactgatgtcttttatgactgtttgctgaactgcacgaagtagccctgccatcatgacagtccctcccaccctgagtaaacaagtcttcataggtatattgtttcatttgtatcaaggaagacagtcttctggaattctgtaggtttgtgctgtttctctgatgtgcctcttctctggtctcttgttatgctgactgatccctgcctcctaaatggaaatcttgaaaattacaccctttaacataactctgctctgaaaagaaccccagacttgactataatctgggcttcataggatattctaacatcaaaattcatattattcctctcagctgctctccaccatgcaccccactaacntctgcnctaatcctctntntcccacattctttagtcactgctctgtcccttcagtgtagcctagaaaaactactgtttacgtttngcttcgacatgttatggctttgaacg

SEQ ID NO: 10 - DNA, 870 bp, Homo sapiens
  misc_feature (29)..(29): n= a, c, g, or t
cttatacttcctccagcagtgtgtaaganagtccattttccatacctttatattacttcttgagtaggaatatctgaaaattattgcttatctattgccatgtaacaaaccatcctcaaacaaccattatattatctctcttgagttcatagcgttattagactcagctgggaagttctgctccttgtggtattggcaggggttgcagctatctgggggactcaattgggctggaatcactgagctggcttgttcacatggctgccaatggtgctgctgtctcctccatgtggcttgacagaagagcagctgggttcctggagaggtaagcaggagctgccagtactcttaaggcctgacctctgaagtaatcccagatatcattttccccatatggtattgggtcaaagcactcccagggccatcccaggttggaggggagggcaaaaagactcagaaatggttggtgttcatctttggagactatttacacagtgattgtatattaaaaaaaacagcaattatatctttgaatttgagtaaagttaaagaaatggtatctgtgaaacttcgaataaacaagtggagcttaaaatatttgattcttgcatattaggttaatgtgctctatgctaatcaggttggagaaatattaagctctgctaggaattctattggactgaaggtaagtcatcctgccagtaactacctaagtcaaggtgtgaacaagctggaatttcttcccattctaatttttttcaagtatacccgtagaaaacaaaatgaagaatcgggctcagaatcatttccagctacctatcacatctttggaagcttcaaggttagatataatggatagcatgcaaaaaa

SEQ ID NO: 11 - DNA, 901 bp, Homo sapiens
cttatacttcctccagcagtgtgtaagagagtccattttccatacctttatattacttcttgagtaggaatatctgaaaattattgcttatctattgccatgtaacaaaccatcctcaaacaaccattatattatctctcttgagttcatagcgttattagactcagctgggaagttctgctccttgtggtattggcaggggttgcagctatctgggggactcaattgggctggaatcactgagctggcttgttcacatggctgccaatggtgctgctgtctcctccatgtggcttgacagaagagcagctgggttcctggagaggtaagcaggagctgccagtactcttaaggcctgacctctgaagtaatcccagatatcattttccccatatggtattgggtcaaagcactcccagggccatcccaggttggaggggagggcaaaaagactcagaaatggttggtgttcatctttggagactatttacacagtgattgtatattaaaaaaaacagcaattatatctttgaatttgagtaaagttaaagaaatggtatctgtgaaacttcgaataaacaagtggagcttaaaatatttgattcttgcatattaggttaatgtgctctatgctaatcaggttggagaaatattaagctctgctaggaattctattggactgaaggtaagtcatcctgccagtaactacctaagtcaaggtgtgaacaagctggaatttcttcccattctaatttttttcaagtatacccgtagaaaacaaaatgaagaatcgggctcagaatcatttccagctacctatcacatctttggaagcttcaaggttagatataatggatagcatgcaaaaaaaaaaaaaaagtcgacgcggccgcgaatttag

SEQ ID NO: 12 - DNA, 353 bp, Homo sapiens
gccttaattggaatccaggtcgttatccaagcaggttttccacaggggtgttctaactgggagatttgggggatgcatgagggccaaaggtcaccgtgtgactgagaaagggtcactgctgcatagaccagcccagtccctgagagggtggggtctttaaggcaagtcaagtgggttgtatccaactataccttgggaagggagtccccgggaggcgataatgtaaggcagacaatctggattgaccatcttgaagaaatgggaggaggtgaagaacaggaaatgatgtcaaggatgactaagccctgtttctggtatgagaaagctaaacctatattcaaaatgaatgctga

SEQ ID NO: 13 - DNA, 363 bp, Homo sapiens
gcgggattaaagccttaattggaatccaggtcgttatccaagcaggttttccacagggtgttctaactgggagatttgggggatgcatgagggccaaaggtcaccgtgtgactgagaaagggtcactgctgcatagaccagcccagtccctgagagggtggggtctttaaggcaagtcaagtgggttgtatccaactataccttgggaagggagtccccgggaggcgataatgtaaggcagacaatctggattgaccatcttgaagaaatgggaggaggtgaagaacaggaaatgatgtcaaggatgactaagccctgtttctggtatgagaaagctaaacctatattcaaaatgaatgctga

SEQ ID NO: 14 - DNA, 837 bp, Homo sapiens
gaaagtgtttagaatagcatgtccaaggttatccagctagaaagtggtagagctggaatttaaattcaaaagttctgacctgggagtctacaatattaattctgtgccaaattgcgttgaggacctcttgggaaaagttaagctctgattgatataggtctaacacacataaaataactggaaaagacttaagacttggaataatattctttaggtaaagtcataaaagtttgaggtgcacaagacagatccagcttctgcctgttatcagatgtcatgtccgacttagtatgtttcttagattcttcatttatttcactgaaatatcgttattaatgacacattgaaataattaaatgtagtagtctttacttgatagttcagagctctgccacgatctggcatatgagatgcatgggaagcatccacaattctcatgctttacataattaaatttgaaagatagtccaaactaacctggttctcttttcctgatgctatctagactcagagaaggacgatatgcagagcgatggctgataaaaaggacacacactagaaaaccggccatcagacacaactaattcctttttttggttctcagttagtggtatttgacagtgctgcctttgatggctacatggtggactgggtaatggattgtgaaaatcacaaggatgtcagactgccagtggtagcttcagtgaggaatgtggagaaatacaaattaataggtgttaaatatgtgcaggaaattgagactgaggcatcctgctttatagagaatggagatttgtctaaatgttcttgttatgatggttttgttgtttattgaat

SEQ ID NO: 15 - DNA, 1309 bp, Homo sapiens
gaaagtgtttagaatagcatgtccaaggttatccagctagaaagtggtagagctggaatttaaattcaaaagttctgacctgggagtctacaatattaattctgtgccaaattgcgttgaggacctcttgggaaaagttaagctctgattgatataggtctaacacacataaaataactggaaaagacttaagacttggaataatattctttaggtaaagtcataaaagtttgaggtgcacaagacagatccagcttctgcctgttatcagatgtcatgtccgacttagtatgtttcttagattcttcatttatttcactgaaatatcgttattaatgacacattgaaataattaaatgtagtagtctttacttgatagttcagagctctgccacgatctggcatatgagatgcatgggaagcatccacaattctcatgctttacataattaaatttgaaagatagtccaaactaacctggttctcttttcctgatgctatctagactcagagaaggacgatatgcagagcgatggctgataaaaaggacacacactagaaaaccggccatcagacacaactaattcctttttttggttctcagttagtggtatttgacagtgctgcctttgatggctacatggtggactgggtaatggattgtgaaaatcacaaggatgtcagactgccagtggtagcttcaaaaatgaaaggaattgaaattaagagaagggagagattgaagtgtggcaccaagattgaaaggagaaagaggtttagggatagtgagggaagttggagaagagagaaaaacaggccacttaccagatttgaaattggcctctgtgctctttcccctgtggacaataactaccatttcatcctggacacccattgtttgcctggaccacaccttgttatgaccataaccctcagtcagactcaaccttccttcaagatctgcaggtcagtcagctcccccagcaccagtcctgttttcagggattctttaaaacacctgcctaagaaaagcttgcttcacaagctctctgtcaaatttacttccaattaccctgtttcctcaaggcagttatttaaacttttggaacttgtaagccaaactactggtcttgtcttggccccgatgttctgtgtgtttgctggccactattacccacgtgtcctggacctttataccttctgggaaaagccaactaacattaacattaccacagaccttaagtctgataagaaatatttacaatctattctctccgaagcctgctacctggagacttcaactgcatga

SEQ ID NO: 16 - DNA, 406 bp, Homo sapiens
  misc_feature (272)..(272): n= a, c, g, or t
gcctaagctgctgagggaagagtgtcaaatcttagcttgccaagggttcagaagaagacccattgcagctagaggaaaagataaagaaaaacctcaaaacaccatctctatgccttgaggggcagaaaatcataccaggttattagaagtctcctaccactggaggagggcagtatctctgaaaaacctcaaaacctgaaactgaggtcacagagtactgaggctgaaccagacagtagagaatgcttttccttgactccccaacccacatncccctcaccatcaggctaggaagcacatcaagcaacaagtaaccacagcctaccactagagaggaagcaagagcatagactctgaggtgaacatgcacagtgaaggcctaaagcttgggatggaacaataccat

SEQ ID NO: 17 - DNA, 916 bp, Homo sapiens
  misc_feature (664)..(664): n= a, c, g, or t
tattaatggaatcataactttttaaagactcatcagaaagctgagcttacaggcagccaacagcttaaaatctaaggaaaagtatgtcaagaagagatgagatagggacactggttcacctgaacattggagggagacaggaggactgccatgtaagtgggtaagaaggattcaactaacagattgctaaagacgtggtgtgacccgtaactgtcacctagcattcacagacacaagggaagttgctcacacttaaaggatcttctttatggaccttggcgagggcaggacattagaagatgtcctggttcagaattggattaaaggggtgtgttctctgctgtgggaaaagtatgaagtcttgcctgcactgtttttttttaagtatcagcagcctaagctgctgagggaagagtgtcaaatcttagcttgccaagggttcagaagaagacccattgcagctagaggaaaagataaagaaaaacctcaaaacaccatctctatgccttgaggggcagaaaatcatccaggttattagaagtctcctaccactggaggagggcagtatctctgaaaaacctcaaaacctgaaactgaggtcacagagtactgaggctgaaccagacagtagagaatgcttttccttgactccccaacccacatncccctcaccatcaggctaggaagcacatcaagcaacaagtaaccacagcctaccactagagaggaagcaagagcatagactctgaggtgaacatgcacagtgaaggcctaaagcttgggatggaacaataccattgaggaaagtactcgagcaaaactgagtaacctatgaattatagtggaactcaaaaggaaagttaaaacatattttgtattgaacgaagatgaaacatggcacacgtggaatacagct

SEQ ID NO: 18 - DNA, 402 bp, Homo sapiens
aatggaaacactggggtttgagagagatgagatggcttgcccaaggtcacatagctcggggcacaaacccaggtggctgctgtggtgctggcttctcccctgccctgtgctgcctccctcttggcggaaggcggcactgtcagacgctctgctgtgagtagcctgaggtgtccagagacactcagcgtttaacactcttctcctgactccccagagcctgccgagccgacctgtcagggaggcagcctggtcacattgtccttggctgccgggacatgtccaggtcatccgtcctgagtcagctgcattctgagccctggcagctgggagaggaaaaggaagccagaggcacagaggacttggagtttaaaagaaggaggagggatgaggccaggcacagtg

SEQ ID NO: 19 - DNA, 58 bp, Homo sapiens
  misc_feature (36)..(36): n= a, c, g, or t
atttaaattttttttcatttttttgagaggtttttnttgttttgttttgttgttgttg

SEQ ID NO: 20 - DNA, 370 bp, Homo sapiens
  misc_feature (276)..(303): n= a, c, g, or t
aagacaaccaaatgatgacatgtgacaataaagccataatagctacttattttcctggttagggaagggatattactagttctaggagtaactgctagctctttcttgtactttttttattgtggaaatataaaaatatacaaaaataaaatgttataattgacttcagtgtcccataaaccagcttcaacagttaccaatttatgtctaatctttcctatctataactctgtgcccctctgttattttgaagctcatcagacatcaagtcattcnnnnnnnnnnnnnnnnnnnnnnnnnnnnatacacatcatgtgtgcacatgtgtgtatgcatgtatacacatttgtatacaagtgtgcactcatgt

SEQ ID NO: 21 - DNA, 188 bp, Homo sapiens
caacatctaatttcatacaacatctaatttcatgatgagctagatttggtattaccaataaacatatccactccatatcttagtttctgacatcctactcagtgaccactgtttatttttctagttcactctttttaataactccagttcaacaattattcaaccatagtttctctatgttctagaaa

SEQ ID NO: 22 - DNA, 212 bp, Homo sapiens
aaggactgggattacacatgtggccaacatctaatttcatacaacatctaatttcatgatgagctagatttggtattaccaataaacatatccactccatatcttagtttctgacatcctactcagtgaccactgtttatttttctagttcactctttttaataactccagttcaacaattattcaaccatagtttctctatgttctagaaa

SEQ ID NO: 23 - DNA, 864 bp, Homo sapiens
aaccactcagaggcaagagttggcatttttactgctttttaggttattttgcccattttgattgacatgcttgatatagtatgaatatgcatggccagtactttttgattcaaaatacgagaatcctttgttttccttggtggtggtttttgtgatacgttcgtggagaaataatgttcagaaagggatttgccaaaaaacattttcattgttacggcaagtttctgttgtagactaaagtctacttaaaataatgtccttatccggcaatacaaatgatttctagcttttgaaaacatagaattatggtaaaatgttacaggcttaacagcctattgttgtgttttttactgaagaggaaatacattttttggatagtaaaaatatggagcaaacagatgttttgcaaatgcttagagctttttccgaaaaaggagaatatttgataggacatgggccatggctggctctctataccctggacaggctctttgcatgacaaaaagtattctgcggccctagcctggacctgctgattcctccatgatgagaacctggatgacacagggcagactctcatttgaaaataccgttgagagtgctggacgtgcccaaaccctggtgcgttagcatcttaaacattttattttttggcccagagtttttctgactcacatcctatactttattctctaaaaccagtccagttttcaaggaggtagtttctttccttggtcgtgaacaaaccactcaagatctttctcggggatatataccaggtaaaaagactccagctgtccttcttatggaacacatactcttggaaattacaaaagctacgtgctgattttaaaaggaattatt

SEQ ID NO: 24 - DNA, 285 bp, Homo sapiens
aaataaatgcacctcaggctttattcaatagaaatttcatgtgaatcactggaccatcattgtccaatagaaattttatgtgaaccacaaatgtaagtagtaggtataatttaaaataaaaaagtaaaactaggtcaaatgaattaagcaaaatctcaggctgcacttattcataataggtgaattttctcaaactcagaaatcaatgcatgaagaattatatttctgagtgtaaaaatataaatatagttattcttgtctagtcagtcatttaataggtattaa

SEQ ID NO: 25 - DNA, 216 bp, Homo sapiens
gtgtttacagagatcacctatcaatatactttagaataatgatgttagaaaaacctagggattaaggaagacagtttgagattagagatttgtagagtgaagatttagtagcacaaatcaagtttgatggaacaggctgttcatagtcttgtgaatgtgtgaatttgttggatatccagtttttttgctgtgaataataaactgtttctctgatgc

SEQ ID NO: 26 - DNA, 1104 bp, Homo sapiens
attgtaccttaataagagtctgcaggttgaatgcagaaccctgtgagttatttacggtttcgcctgggagctttgaaactgacccttgaatgggtgttctcctgcggggaggggcagggtgaattaccttttcttaactgattttaaaaagcctcattgtccctaaaaatcatttccttgcacgagttgcaatttatgtgtggaactgaatcatcaattccgtgaagagaaagatattcgtaaagtacatctctctaaagttaagaaagtaagaacgtttggctagttaaaaacaaacactactgccattgtaaaaaacacgccagtgacatacttggtgagatattttggccttttgagagtcggggcacacctgttcctgtccctgccaagttgtgtgtgctttccctgccattgttaatctggtcgtgtacacaagatgaaacggtaaaacttgctatctgtataattattaactagtttgtcagttaccctttcagagaataatcaataacttcagtcttaacttttaaaactccagaaattttactttttaagtctggaaaatgtttcaaagagagattaaatgaaacaaacaaacagcaacaacaaacaacaaaaccagaaaatcaacctacaactttttagtttctgagtagccagctacaaggcaaagaaatgatgaatatatatagtatgtactgaacaagcagtgcactggattttaaaatgatatttttattgttaccattgctgttaaaatacaaacagctcacactatttgaaatccttgagattcacggattgaaaaaaaaccgaaaaggacacctctgagtgagggagtagccctgctgagatctagcaccagaatgccatttgccaacagttcactaacatcatgaatcatttttgaaagcaaagttataccctttgcacttaacgctttccctaaatggaaaaaatttaaagagtttacagcaatggtagaatggcagccccagttgtcatttttttaccactgtcatttccatagagaggggctgtcatcataatgtatgtgcatgcagcctgggtccctctgttaatctatcagggaaaagcagc

SEQ ID NO: 27 - DNA, 937 bp, Homo sapiens
  misc_feature (579)..(579): n= a, c, g, or t
ggctgagtgagcattgtgttatagacccttaacatgaggcaggtaggtacttttatactgttgttcacttaccaactgggtgacttgggtaagtcacttctggagcttcctcaataatatgatagtaccttcatggtaaatagaattgaccaaggctcttactatagtatgacaagttatagatgattaatacatgaaaatgtagagttaaaaatttgtagatgaaattagaaaagagaagtaccaagcatggatttattataaagtatttaataccattttgggtaaaataatctgaaatatgttctttaaatattttgttatgaaaagtttctaatatacacaatagtagagtgaatggcataacaaatgcttatgtacctaatcacccaccttccacattgccattctttaaatgaaggtttaggataatccatttcaggttaccgcttatgacccaaaatgggaacctgtgaagccttattacttgccagagtgttggcatggtgctcaatttaataccattttgaatatcattaggatgctgttgaaaacaaaatcaaaacttgattaaagccntatatgtctttgcagctaaatataatgaacgtagaaaggactaataaaatgaaaaggaaaatttagatttattatctctaaaatcatgaatgaaatggcctggtagacgaaattatttacattaatttagaatgcagaggtaaagaagagtcttatcaaaatctattaaaatcatagtctaatattgtcttacgatatttttataactttttgaaacactattttaggataattgtgaaatatgagtcttcagtttacacattgattttcaaaatcctggtttgttagtttggggtgatctcagttgtacaagtctaatttggaggccgggcgcagtttctcatacctataatcctga

SEQ ID NO: 28 - DNA, 364 bp, Homo sapiens
  misc_feature (64)..(64): n= a, c, g, or t
gtttgtcttagccctttttattgctgcagtaacatgaatactgtaagactgggcgacttaggangaatagaaatttttctcacagttggaggccttgaagtccaagggtaaggcacatccatcctgctgatagatgcatcccccaaggggaagatccctgtgtcctcccagggcagaagaggaaagagcaacccccaggcagcctctcctccaagggcctttgatccctttcaggggaggagttctcacagcctagtccggtcttcatactaacacactgaagacacttacattttggagaagacaccttcacaccatagcatgtctattggtatagttgtaaagtttctttataaagttaagg

SEQ ID NO: 29 - DNA, 1276 bp, Homo sapiens
gaagctgcggcgacgtcctgagtcggtcaggacgacgaggagcagtgcctgactgcgcaggtcctggatgcctcatcccttagtttcaacaccagattgaaatggtttgccatctgcttcgtatgtggcgttttcttttctattcttggaactggattgctgtggcttccgggcggcataaagctttttgcagtgttttataccctcggcaatcttgctgcgttacgcaggtacgtttggcttctcaaaatatcttgtttgctcttgctaccaaatttacctttctaataagcactttcccagtaggatgtttatgtagttctgtacatactgtgaattcagccacaggatttacattcattaaataaatatgtattgactgctgctgtatgccgtctcccataacagacggtattagtgctaggaatagccagcctttgtggggcacttaatgtgtgtctgggactgttctaagtgttttgtatgtattatgacgagcttgtgaggtaggcagtattatcatccccgtttataactggagataatagcacagagcaggggagtcagtgctgcccatggcgatgaacctaaatcatggctgaggtgggaatcctgcaggcagtgggaatccggaggtgggatcccaccatggctgaggtgggaatccggagcccaggcttggagctgctgcactctaggctttgttcttgtggagcttacattttaatgaagaagatgttcacatgataagccagaaaaagatgggatggcattggtgctgtgcagagaattaaaacagggtgtggcagtagtgacagagggctactttatattgggagtatgagaatccctctttgagaatctttgtcgcatctttgggttgttgtttgtcttagccctttttattgctgcagtaacatgaatactgtaagactgggcgacttaggaagaatagaaatttttctcacagttggaggccttgaagtccaagggtaaggcacatccatcctgctgatagatgcatcccccaaggggaagatccctgtgtcctcccagggcagaagaggaaagagcaacccccaggcagcctctcctccaagggcctttgatccctttcaaagggaggagttctcacagcctagtccggtcttcatactaacacactgaagacacttacattttggagaagacaccttcacaccatagcatgtctattggtatagttgtaaagtttctttataaagttaaggaaaaaaaaaaaaaaaaaatgagcggc

SEQ ID NO: 30 - DNA, 373 bp, Homo sapiens
gctcagggggagatgaacaccttccccaaaaacacagcaagtaagcagcagagacaggatttgaacccaggccccgaatcccaatatctggcctctgaaccaccactactgtcctgctgactttcgcacatgttttcatatctgtagaaaaagtctggaagattagacaccaaaatgtaactggtggtgtccttggagtcctccaagtgggataatcatggggactttcattttcttgtttcccaaatattctacgatgaccagctattacctgtccatgctgagaaaaaagctgcagcttagcagaaggccttggtagtgactcatcccctcaagatctctatcatgtcccgactctaaagggtctggttga

SEQ ID NO: 31 - DNA, 60 bp, Homo sapiens
cttagagggaaaaataaaacaactaactcctgaatatgaaggagctaaattacacttcat

SEQ ID NO: 32 - DNA, 70 bp, Homo sapiens
cttagagggaaaaataaaacaactaactcctgaatatgaaggagctaaattacacttcatctttcctact

SEQ ID NO: 33 - DNA, 275 bp, Homo sapiens
  misc_feature (47)..(47): n= a, c, g, or t
caactcttagcatgcagtacagtggttttccacaaatgaaacatacncatgaaatcagcccccagatgaagaacagaaccccagaagcctgtttcctctgcacacacagccttccccaggggtgacccaactgactccaacatcacagattaagtgctcctgtttttatacttcgtataattganatcatacaatgtgtactccttcatgtcttggagtggctgttttgttttgtttgactaacacctttaaaaaaaaaaaggcttatgntattt

SEQ ID NO: 34 - DNA, 849 bp, Homo sapiens
aaaacaaataaaccccccggatttttttttacaaacaccccggaaaatccccaatccgggggcccccacttatctttttacccggtaagcgcgtttaatggccccccaggtcttttcccaaactggaggatcggaaaatatccggagggctcaactgaagaagtgtgttttttcccccatttgggtgtcaaccacactgttcacttttcccccacaatttttaaccccaggtcccttatcctcaatttagaggtttgaatttggaacttattgacttatttaccagtatcttaaatacaattttttaagatcttttaattaaaaccttttttaccaaaaagggcacaactcttagcatgcagtacagtggttttccacaaatgaaacatacccatgaaatcagcccccagatgaagaacagaaccccagaagcctgtttcctctgcacacacagccttccccaggggtgacccaactgactccaacatcacagattaagtgctcctgtttttatacttcgtataattgaaatcatacaatgtgtactccttcatgtcttggagtggctgttttgttttgtttgactaacacctttaaaaaaaaaaaagcttatgcatatctggcatgttgtaggggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctgcgcaattactactatgacgtggtgaactaaccgctcccgcacctgtcggtggctgccttg

SEQ ID NO: 35 - DNA, 215 bp, Homo sapiens
  misc_feature (68)..(164): n= a, c, g, or t
cctctttctagtcacgcccatcagctgcaacatgtgtttaattatgatttagttttgtatatactcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaagtcccataagagggtgtctgcaaactgaggagtaagaaagccagtccc

SEQ ID NO: 36 - DNA, 276 bp, Homo sapiens
  misc_feature (136)..(136): n= a, c, g, or t
ctcatgagcttcacatacaagatctattgaatctctacaattctgagatgcagagaggacaggcattggacagaaaaagttctgaagggtaaattattgcccaagcccacatagcaggtaagagatagagccaggntccaaacccagaaccctgggctccagataaaagggtccctgtatattaaaaactggtccaggttgaaaatacaattcaactggtggatgactgaaggatgattagaaaccacgaattttcaaggctcccaccaaaaaaaa

SEQ ID NO: 37 - DNA, 301 bp, Homo sapiens
ctcatgagcttcacatacaagatctattgaatctctacaattctgagatgcagagaggacaggcattggacagaaaaagttctgaagggtaaattattgcccaagcccacatagcaggtaagagatagagccaggatccaaacccagaaccctgggctccagataaaagggtccctgtatattaaaaactggtccaggttgaaaatacaattcaactggtggatgactgaaggatgattagaaaccacgaattttcaaggctcccaccagaaaaaaaaaaaaaaaaaaaaagtcgtatcga

SEQ ID NO: 38 - DNA, 573 bp, Homo sapiens
  misc_feature (48)..(48): n= a, c, g, or t
ttctcttaagagcttgtccacaggctggcctctgaggtgacctgggtnaacccactcatgtggctgtgtcacccagtgtctggggacaaggagtgtggccacgctggcagccagccagcagccttctgccctgtagaaagacagcccccaaactgtctgccacaccatcacattcatcacctccacacctggggaaaagggccccgataaggagggcttttcctgaggaataagccccagaagagaaacctctcacttccccctgactgttccattgtgaataatagtaaggttgataatagtaaggctttttccgtttttgatataagtttagaaactgtcacattgtcatctattcctttaaaaatgtgtttactgaagtgtgttaatcattttgactggacatttcagactgggaaagatttgtttaaatggattgaatgagtatttccaaaaaattgtacacagtttactgaggtcaatttgatgtactggaatatagataatttgttttctatattacactcataactattcttttctttntataaagtatatgcttttccttaaaaa

SEQ ID NO: 39 - DNA, 407 bp, Homo sapiens
  misc_feature (258)..(258): n= a, c, g, or t
aaacaaagattacttgaacacaagcactgcaataccgccacagttgatatggcaactgagatggctgctaagtgattaacaggcagtgaacacatacggtgtggatatgctggacaaggtgatggatgattcacccctgggcaggacagagcaggatggcatgagatttcatcatgatactcagagtggcacatgatttaaaacttacaagttatttatttctagaaattttccatattatatctttggactgcagtnaaccacaagtnactganactgcagaaagtgaanccncagataagaggggncagctctataatgcnatatcaatcantatagcagagaaaaagaaagtatncctgtttattttataaagttgncaaaatcttgatnacnnaaagctgaca

SEQ ID NO: 40 - DNA, 663 bp, Homo sapiens
tgaaaacctgaaatgctctgaaaacctgaaatggcgttttggactgctgcaagggaaactgtgatcttaacaacatcagtgcggctcagacatgaccaccatgtgagggcaaaggtggggctggttggctgccagaaccaagggcatccgcggcgttccttggcattggcgttaacagcagatggattttccgcagcggccaggcggaagatgcctagcattggtccgccgtctcactgtactgaatttcaagaatcatggcctactgccagccagttgctggatttgtgcgactcggtgaaggatgatgcccggagggtgatctcgacctttaacattccacacacctacctccacgcaccaatcgccggaatctccaacccgcgggccgcgtgggctttctaccctgcaccgctgcagccgcggccacgggaagaggcgcgctcccggcggcccaagctgggagccaagttctaacgggtgtggcgggaagtgtggtggcccgccagcatgctgccacgacgctcgctccaccgacgcccagagctgtggccgaggccgcggggctggcacccgctgggccgccactctcggggattttggtggcaaaagcggaggtcccgccgaggctggatgacggtagacgcgggatggtctgtctaa

SEQ ID NO: 41 - DNA, 1234 bp, Homo sapiens
atattgaaatctgcctcatttaattcctacctctggccctacgttctgtttttggcaccagatagataaaacccatactctattttacactcattcaaatcattgtatttcccccaccggtgaatatattaccaaagttggttgagtttcataatgcaaacccaaagggaaaattatcttgaataatcatatgacaatatgtggaaagtttaggggctgtgtacgccacctcatggttttgaactgtgtggttaaagacgttgagagttgacagattagtcagggatttttcaagagtattaattacaaactttgaaatcttggtcagtgaaagttcattttagtgcatcttgcaaatacgatcaaaggtcttttcagtactgatttgatgatgcttaaccacagtgtgtgaagaatgaagtttttcattagaaattactgatgtaatagagcacattttcagggaagatgtattgttggactattacaattcatgctcttcccccaccatatccctaccctctatattccccatcccctatttcccccaccttcctccttgggacctcactgaaaacctgaaatgctctgaaaacctgaaatggcgttttggactgctgcaagggaaactgtgatcttaacaacatcagtgcggctcagacatgaccaccatgtgagggcaaaggtggggctggttggctgccagaaccaagggcatccgcggcgttccttggcattggcgttaacagcagatggattttccgcagcggccaggcggaagatgcctagcattggtccgccgtctcactgtactgaatttcaagaatcatggcctactgccagccagttgctggatttgtgcgactcggtgaaggatgatgcccggagggtgatctcgacctttaacattccacacacctacctccacgcaccaatcgccggaatctccaacccgcgggccgcgtgggctttctaccctgcaccgctgcagccgcggccacgggaagaggcgcgctcccggcggcccaagctgggagccaagttctaacgggtgtggcgggaagtgtggtggcccgccagcatgctgccacgacgctcgctccaccgacgcccagagctgtggccgaggccgcggggctggcacccgctgggccgccactctcggggattttggtggcaaaagcggaggtcccgccgaggctggatgacggtagacgcgggatggtctgtctaa

SEQ ID NO: 42 - DNA, 1191 bp, Homo sapiens
caaagggtcaagcagtcggccctttaacaaaaggtcaagcagtcggccctttaaatgaccttaaattaaagccttgcagacaagcagatttaccagcattatagataagtatttcaggaagtatttactttactaaatatgactgtatatttgtcctataaaaagtaaatcttcaaaagattttaaaaattacagtattttggttgataggagataagaattttattaaaactttcttaaaacatttggtaatacattaatgaaatgaccacaactttaaacgaccacttatcctcataaatatggatttttgccttcaccaggtgattacattataaagctgtgctttcacagaagacaaaataaagttgtatgatttatattgaagtcaaatgtgttgtgagattacatttggaaagattatattggaaaaattctaatagtttttcagactacgtgagagacttaagtgaccatgtgtaatagaaaagttccactgaaagttaaaatctgtaacagcatgacccagcctgtacatttgggtcaagtatcttttgagatagctgcagagtttcctttacaatgctgtctttcagttttctatagctttatgcgtattgatcatcctcagtaagggcccttttctttcccccatcccttccacccctttatagtgttttatttaggggattaatttaggcaaaactatgcccacaaatctatgggtttcctttcctatgggttactggcaactaaaaatataaatgtacattcttatcagacagaaaatgttattaatgatattattcagtgattaacatagatgcccaattgaaagctttctggtaaaatttaataaatttaacaggatatttttaatgaagtgatttttgccattattaaatcttcctacctccagttttcttccgtgcccatatacaggtgagcaatagggaccactgctgcataggatacattgttattcggttacataaagcacataataaaatttaattcttgccattatgttgtctattttcaaaattgggggtagtagagacagatgaagatttagatatagaaatggaattctgtcttaagcacttaaatgatcggtatggattgttactgatccagccacccatgctataagtctagaaactcagccaggcatggtggctcatgcctgta

SEQ ID NO: 43 - DNA, 4714 bp, Homo sapiens
gaagtataagtgggattttcaccaaaaccctatccttccccagactccagtgacagtttaacggtcagctgcatcaaggcttgtgtactgtgcagtcttaaaaggcaccactcctgtccatctcggtgaaattgatggtggagcaaaagggagtttaggtggttccggtctggggagaagacgtgcagggtttcagatgcgaaagtcgcacccaaccgcttcactcgggctctaggccatccgcaggggccctgcttccttccacctgcgagctttttctgcagaatgcgggaagccgccgccgccgccacagaggagggggcggaggcagaggcggaggcggcacccaggggccggggcaggggaggccgggaccatcgcagtgacaatttattttcctgcagcagcggcagcagggacggttgctgcaggttcggggtcggccggcctgcgcgtgggcttgcgaggacgctgttcgtcccctgcgctggggtgtccgacagcgaggaggagaacgacgcacggagcccgcgcgactggaaccagcaaagctccatctgtcggcagaggagaagggggaggaggcacggccgaggcaaacgagcggacgcctcgtcgccgggtgccggtatcaccccgctgcaacgccttccagcaaaagccaccgcggcccgggttgcagcagccggacggatgccaaggccacacggcagccacgggggcagccgtcgcagtcgccgtcccacacgggctgcggacaccaagggttgctaatgaagtgattgagaagaaacagtgaacatcctcatttcacagataagacaacatggatcagccttttactgtgaattctctgaaaaagttagctgctatgcctgaccatacagatgtttccctaagcccagaagagcgagtccgtgccctaagcaagcttggttgtaatatcaccatcagtgaagacatcactccacgacgttactttaggtctggagtagagatggagaggatggcgtctgtgtatttggaagaaggaaatttggaaaatgcctttgttctttataataaatttataaccttatttgtagaaaagcttcctaaccatcgagattaccagcaatgtgcagtacctgaaaagcaggatattatgaagaaactgaaggagattgcattcccaaggacagatgaattgaaaaacgaccttttaaagaaatataacgtagaataccaagaatatttgcaaagcaaaaacaaatataaagctgaaattctcaaaaaattggagcatcagagattgatagaggcagaaaggaagcggattgctcagatgcgccagcagcagctagaatcggagcagtttctgtttttcgaagatcaactcaagaagcaagagttagcccgaggtcaaatgcgaagtcagcaaacctcagggctgtcagagcagattgatgggagcgctttgtcctgcttttccacacaccagaacaattccttgctgaatgtatttgcagatcaacctaataaaagtgatgcaaccaattatgctagccactctcctcctgtaaacagggccttaacgccagctgctactctaagtgctgttcagaatttagtggttgaaggactgcgatgtgtagttttgccagaagatctttgccacaaatttctgcaactggcagaatctaatacagtgagaggaatagaaacctgtggaatactctgtggaaaactgacacataatgaatttactattacccatgtaattgtgccaaagcagtctgcgggaccagactattgtgacatggagaatgtagaggaattattcaatgttcaggatcaacatgatctcctcactctaggatggatccatacacatcccactcaaactgcatttttatccagcgttgatcttcacactcactgttcctatcaactcatgttgccagaggccattgccattgtttgctcaccaaagcataaagacactggcatcttcaggctcaccaatgctggcatgcttgaggtttctgcttgtaaaaaaaagggctttcatccacacaccaaggagcccaggctgttcagtattcagaaattcctttctgggataatttctggcactgctttggagatggagcccctgaaaattggctatggaccaaatggattcccactcttggggatctctaggtcatcatcaccatctgaacagctctgagagagacaagaagtggaatggagaaggtgattaggtaatccaagaattgccaggcgaacaattgtgaaaatctttcatagagttcccgatgaaaacaactctttcttgttagccattatctctgcctttcctttctaaagaaaaacacgtcatttcatcttgattatcagtttctcaagtactcatggatgtgaggccaggtaccctgtcaccttggctctgtttgggatgcaatgttgtgtttgtcaccacttcccattccccaaaagcatctttaacatagaccattcattcttcagattcaaaatgcttaagctgaactaacctcaaagggccctcattaatttacagacatttgaataacacccatattcatttctgctactgaaccaaagccttttccaaaaaaatcatctagcaaactgtttttgccaggccaaatattaacaggtatatatatttgcatacataaatatttacctctatgtcttgtgtgtttttgttatgagacagagtctcactctgtcgcccaggctggagtgcagtggcacagtctcagctcactgcaacctccacctcccaggttcaagcgattctcatgcctcagcctcccgagtagcgaggactacaggcgtgcaccaccacgcccagctaatttttgtatttttagtagagatggggttttaccctgttggccagggtggtctcgaattcctgacctctgatgatctgcccgcctggacctctcaaagtgctgggattacagacatgagccactgtgccctgcctctgttttgtgtatttacataaatacatgtctccagagctttaaattcttcttaaattttaaaactagaactaaaccagacattgagaggacccagaaatataccctgactaacgtgggtggaatttcatttggcttgactctttggtgaaaaaaaaaaaaatcaggctctcaaggactcaatttggtgaaccaatgccaaatagaaaaatgttcttgacattttccccaaaatagatgtctaaagattttgcacttcaaattggtactttatgtattaaaagtgatattgaaactaaattcctacccaattagttgaaaaatatgaaaaaagcaattgctagagaatgcaatttattaaatgttggtggtcttttaaaaaatgaattcctcttgtagccagtgagatcagttatttatttatttattttaatagtaacaaggtctcactatattgtccaggctgatcttgaactcctggcctcaagctgtcctcccatctcggcctcccaaaatgctgggattacaggcatgagccaccatgcctggctgagtttctagacttatagcatgggtggctggatcagtaacaatccataccgatcatttaagtgcttaagacagaattccatttctatatctaaatcttcatctgtctctactacccccaattttgaaaatagacaacataatggcaagaattaaattttattatgtgctttatgtaaccgaataacaatgtatcctatgcagcagtggtccctattgctcacctgtatatgggcacggaagaaaactggaggtaggaagatttaataatggcaaaaatcacttcattaaaaatatcctgttaaatttattaaattttaccagaaagctttcaattgggcatctatgttaatcactgaataatatcattaataacattttctgtctgataagaatgtacatttatatttttagttgccagtaacccataggaaaggaaacccatagatttgtgggcatagttttgcctaaattaatcccctaaataaaacactataaaggggtggaagggatgggggaaagaaaagggcccttactgaggatgatcaatacgcataaagctatagaaaactgaaagacagcattgtaaaggaaactctgcagctatctcaaaagatacttgacccaaatgtacaggctgggtcatgctgttacagattttaactttcagtggaacttttctattacacatggtcacttaagtctctcacgtagtctgaaaaactattagaatttttccaatataatctttccaaatgtaatctcacaacacatttgacttcaatataaatcatacaactttattttgtcttctgtgaaagcacagctttataatgtaatcacctggtgaaggcaaaaatccatatttatgaggataagtggtcgtttaaagttgtggtcatttcattaatgtattaccaaatgttttaagaaagttttaataaaattcttatctcctatcaaccaaaatactgtaatttttaaaatcttttgaagatttactttttataggacaaatatacagtcatatttagtaaagtaaatacttcctgaaatacttatctataatgctggtaaatctgcttgtctgcaaggctttaatttaaggtcatttaaagggccgactgcttgaccttttgttaaagggccgactgcttgaccctttg

SEQ ID NO: 44 - DNA, 521 bp, Homo sapiens
  misc_feature (15)..(15): n= a, c, g, or t
gaaattatacatggncntaccacatgcccatcacatggttagcattttgataccacagctgtgcattattacatgtcaaagactgcatatactatggtggtccccatacaattataatgtattttctactgtaattaggtacacaaatacccttgtgttacattgccgacannnnnnnnnnnnnnnnnnnnnnnnnnnnnnnnnnnnnnnnnnnnnnnnnnnnnnnnnnnnnnnnnnnnnnnnnnnnnnnnnnnnnnnnctaggtttaagtgcagtctagggtggggaacattttaatgttgatccacatgccaggtgtttgtgagacaagattatgctttaatcaaaatattttggaattgtctagaaaatagtgctggctgcttgtctcaaaaaanaaananaaagtcacaactccctctggtgaataagaaagttggtgtaaaagttgggaacgttttcaatgtttattacactataagtgtaataaatattatacaaaattataaattcacatttaa

SEQ ID NO: 45 - DNA, 2675 bp, Homo sapiens
gtgtgagctaacgagcacggcctattatcttgtactttctaactgagccctctattttctttattttaataatatttctccccacttgagaatcacttgttagttcttggtaggaattcagttgggcaatgataacttttatgggcaaaaacattctattatagtgaacaaatgaaaataacagcgtattttcaatattttcttattccttaaattccactcttttaacactatgcttaaccacttaatgtgatgaaatattcctaaaagttaaatgactattaaagcatatattgttgcatgtatatattaagtagccgatactctaaataaaaataccactgttacagataaatggggcctttaaaaatatgaaaaacaaacttgtgaaaatgtataaaagatgcatctgttgtttcaaatggcactatcttcttttcagtactacaaaaacagaataattttgaagttttagaataaatgtaatatatttactataattctaaatgtttaaatgcttttctaaaaatgcaaaactatgatgtttagttgctttattttacctctatgtgattatttttcttaattgttattttttataatcattatttttctgaaccattcttctggcctcagaagtaggactgaattctactattgctaggtgtgagaaagtggtggtgagaaccttagagcagtggagatttgctacctggtctgtgttttgagaagtgccccttagaaagttaaaagaatgtagaaaagatactcagtcttaatcctatgcaaaaaaaaaaaatcaagtaattgttttcctatgaggaaaataaccatgagctgtatcatgctacttagcttttatgtaaatatttcttatgtctcctctattaagagtatttaaaatcatatttaaatatgaatctattcatgctaacattatttttcaaaacatacatggaaatttagcccagattgtctacatataaggtttttatttgaattgtaaaatatttaaaagtatgaataaaatatatttataggtatttatcagagatgattattttgtgctacatacaggttggctaatgagctctagtgttaaactacctgattaatttcttataaagcagcataaccttggcttgattaaggaattctactttcaaaaattaatctgataatagtaacaaggtatattatactttcattacaatcaaattatagaaattacttgtgtaaaagggcttcaagaatatatccaatttttaaatattttaatatatctcctatctgataacttaattcttctaaattaccacttgccattaagctatttcataataaattctgtacagtttcccccaaaaaaagagatttatttatgaaatatttaaagtttctaatgtggtattttaaataaagtatcataaatgtaataagtaaatatttatttaggaatactgtgaacactgaactaattattcctgtgtcagtctatgaaatccctgttttgaaataagtaaacagctaaaatgtgttgaaattattttgtaaatccatgacttaaaacaagatacatacatagtataacacacctcacagtgttaagatttatattgtgaaatgagacaccctaccttcaattgttcatcagtgggtaaaacaaattctgatgtacattcaggacaaatgattagccctaaatgaaactgtaataatttcagtggaaactcaatctgtttttacctttaaacagtgaattttacatgaatgaatgggttcttcactttttttttagtatgagaaaattatacagtgcttaattttcagagattctttccatatgttactaaaaaatgttttgttcagcctaacatactgagttttttttaactttctaaattattgaatttccatcatgcattcatccaaaattaaggcagactgtttggattcttccagtggccagatgagctaaattaaatcacaaaagcagatgcttttgtatgatctccaaattgccaactttaaggaaatattctcttgaaattgtctttaaagatcttttgcagctttgcagatacccagactgagctggaactggaatttgtcttcctattgactctacttctttaaaagcgggctgcccattacattcctcagctgtccttgcagttaggtgtacatgtgactgagtgttggccagtgagatgaagtctcctcaaaggaaggcagcatgtgtcctttttcatcccttcatcttgctgctgggattgtggatataacaggagccctggcagctgtctccagaggatcaaagccacacccaaagagtaaggcagattagagaccagaagaccttgactacttccctacttccactgctttttcctgcatttaagccattgtaaatctgggtgtgttacatgaagtgaaaattaattctttctgcccttcagttctttatcctgataccatttaacactgtctgaattaactagactgcaataattctttcttttgaaagcttttaaaggataatgtgcaattcacattaaaattgattttccattgtcaattagttatactcattttcctgccttgatctttcattagatatttgtatctgcttggaatatattatcttctttttacctgtgtaatggtattactaa

SEQ ID NO: 46 - DNA, 2848 bp, Homo sapiens
gtgtgagctaacgagcacggcctattatcttgtactttctaactgagccctctattttctttattttaataatatttctccccacttgagaatcacttgttagttcttggtaggaattcagttgggcaatgataacttttatgggcaaaaacattctattatagtgaacaaatgaaaataacagcgtattttcaatattttcttattccttaaattccactcttttaacactatgcttaaccacttaatgtgatgaaatattcctaaaagttaaatgactattaaagcatatattgttgcatgtatatattaagtagccgatactctaaataaaaataccactgttacagataaatggggcctttaaaaatatgaaaaacaaacttgtgaaaatgtataaaagatgcatctgttgtttcaaatggcactatcttcttttcagtactacaaaaacagaataattttgaagttttagaataaatgtaatatatttactataattctaaatgtttaaatgcttttctaaaaatgcaaaactatgatgtttagttgctttattttacctctatgtgattatttttcttaattgttattttttataatcattatttttctgaaccattcttctggcctcagaagtaggactgaattctactattgctaggtgtgagaaagtggtggtgagaaccttagagcagtggagatttgctacctggtctgtgttttgagaagtgccccttagaaagttaaaagaatgtagaaaagatactcagtcttaatcctatgcaaaaaaaaaaaatcaagtaattgttttcctatgaggaaaataaccatgagctgtatcatgctacttagcttttatgtaaatatttcttatgtctcctctattaagagtatttaaaatcatatttaaatatgaatctattcatgctaacattatttttcaaaacatacatggaaatttagcccagattgtctacatataaggtttttatttgaattgtaaaatatttaaaagtatgaataaaatatatttataggtatttatcagagatgattattttgtgctacatacaggttggctaatgagctctagtgttaaactacctgattaatttcttataaagcagcataaccttggcttgattaaggaattctactttcaaaaattaatctgataatagtaacaaggtatattatactttcattacaatcaaattatagaaattacttgtgtaaaagggcttcaagaatatatccaatttttaaatattttaatatatctcctatctgataacttaattcttctaaattaccacttgccattaagctatttcataataaattctgtacagtttcccccaaaaaaagagatttatttatgaaatatttaaagtttctaatgtggtattttaaataaagtatcataaatgtaataagtaaatatttatttaggaatactgtgaacactgaactaattattcctgtgtcagtctatgaaatccctgttttgaaataagtaaacagctaaaatgtgttgaaattattttgtaaatccatgacttaaaacaagatacatacatagtataacacacctcacagtgttaagatttatattgtgaaatgagacaccctaccttcaattgttcatcagtgggtaaaacaaattctgatgtacattcaggacaaatgattagccctaaatgaaactgtaataatttcagtggaaactcaatctgtttttacctttaaacagtgaattttacatgaatgaatgggttcttcactttttttttagtatgagaaaattatacagtgcttaattttcagagattctttccatatgttactaaaaaatgttttgttcagcctaacatactgagttttttttaactttctaaattattgaatttccatcatgcattcatccaaaattaaggcagactgtttggattcttccagtggccagatgagctaaattaaatcacaaaagcagatgcttttgtatgatctccaaattgccaactttaaggaaatattctcttgaaattgtctttaaagatcttttgcagctttgcagatacccagactgagctggaactggaatttgtcttcctattgactctacttctttaaaagcggctgcccattacattcctcagctgtccttgcagttaggtgtacatgtgactgagtgttggccagtgagatgaagtctcctcaaaggaaggcagcatgtgtcctttttcatcccttcatcttgctgctgggattgtggatataacaggagccctggcagctgtctccagaggatcaaagccacacccaaagagtaaggcagattagagaccagaaagaccttgactacttccctacttccactgctttttcctgcatttaagccattgtaaatctgggtgtgttacatgaagtgaaaattaattctttctgcccttcagttctttatcctgataccatttaacactgtctgaattaactagactgcaataattctttcttttgaaagcttttaaaggataatgtgcaattcacattaaaattgattttccattgtcaattagttatactcattttcctgccttgatctttcattagatattttgtatctgcttggaatatattatcttctttttaactgtgtaattggtaattactaaaactctgtaatctccaaaatattgctatcaaattacacaccatgttttctatcattctcatagatctgccttataaacatttaaataaaaagtactatttaatgatttaacttctgttttgaaatgttgtatacacgtggatttttttctcattaaataataattctagt

SEQ ID NO: 47 - DNA, 434 bp, Homo sapiens
acattcttcagagtgggttgggtgcatccaaaaatgtatatctcctttggcataatgagccttggcttactttccctcctggcagtcacttctatcccttcagtgagcaatgctttaaactggagagaattcagttttattcagtctacacttggatatgtcgctctgctcataagtactttccatgttttaatttatggatggaaacgagcttttgaggaagagtactacagattttatacaccaccaaactttgttcttgctcttgttttgccctcaattgtaattctgggtaagattattttattccttccatgtataagccgaaagctaaaacgaattaaaaaaggctgggaaaagagccaatttctggaagaaggtctgggagggacaattcgcatgtcgccccggagagggtcacagtaatgggatga

SEQ ID NO: 48 - DNA, 425 bp, Homo sapiens
caactatacttttcttccagatggctgcccagttatggcagtgtccttcatttagttcagctttgccctgctgattgaaatgttgtctataggtgtacttagttcctacagtttgaggttattactggacttttattctgtttcattgatcagattacctatttatacctaatgactctattccttggttatcatcttccctcattctacttcccagagttttcctgggaagttttctttgtttatttttctatatggcttagaattagctaaccaagtcctgctcctctgtcctgttgacatttttattggtattgtattatatttatagattaatttagagataattgacatcgttttgaggttcaaaaacagggtctatattcatttctttatgttcaagaacagtatatgtggccaggcat

SEQ ID NO: 49 - DNA, 2620 bp, Homo sapiens
ttttttttttggtagagatggggatcttgctgtgttgcccagggtggtggtctccagctcctggcctcaagcaatcctactgcctcagcctcccacactgctgggattacaggtgtaagccaccacacctggccactgatccctttcagtctaaatttggtttctcttaccttccccaaaatgtcaggaaaaattacctccacccatctccatctcagtaaacaatattcaatattcttcccacatcaaagctggccttaagggacaggttcgctgactctcaagattaagccttcaacccctagttcccaccttcaacaaggcatggattcttagcttctttgggaaaggtcaaatgggaaggaagctagcagcttggaggtaagctctgcctcaaggcaatcagattatgtcacaataccaaccaattctatgctgataacaggcagggaagatgaaacactttcaggccatcgagtagggggattaggagaccaagcactggtccccagagggatggcaacagaatacatatcgatttcatcacagctttctagtatgtcgtagacacaaagaaaaaaacaatagggctaattctacgcttgatttatcaggtatggtgttgttttttttgtttgtttgttttgtttttttgacacggagtcttactctgtcaccaggctggagtgcagtggcgcgatcttggcttactgcaacctctgcctcccgggttcaagcaattctctccctgcctcagcctcagcctcccaagtagctggtattacaggtgcctgccaccacacccagcttatttttatattatttagtagggatagggttttaccatattggccagccttgaactcctgacctcaggtgatttgcccgccttggcctcccaaactgctgggattacaggcgtgagccaccgcacccagcccacacccagctaattttttgtattttactaaaacagggtttcaccatgttgtccaggatggtcttaatctcctcaccttgtgatccacctacctcggcctcccaaagtgctgggattacaggcgtgagccactgcgcctggccagatatgttaacatagctccacacctacaggtcctcccctctgggccaagtttgaagaaacagattctcaccttggcttaaaggaccctagtggtttctacaaagatagtatttctgggtggacttggctcattttctgttcaaaagacccaagtctccccaagggcagacatgaaacagctaagcaaccaaacatctgggggctggttttctggattttggttgatctttttggacagaaaagcaccaacagcaaaagaaaaggcaagggggaataggtgaaggtgagccactgtcactacaacaactgagttcatccatgatgactaaggaatatcaggacattctagtttttgagaattcttcctttcagagtaaatgcttcttatatccttctggagaacagaactaaatccagaagctgtagtatcattaactttgcctagaaatctagaaatctcaacctcatatctgttcataactccttccctttctccaaagagaatacggaaggcaaagtccattttctgaatatggcatttttccttttctatttttttgagacagagttttgctcttgttgcccagactggagtgcagtggcacaatcttggctcactgcaacctccgcctcccaggttcaacaattctcccgcctcagcctcccgagtagctgggaccacaggcacctgccaccaagaacggctaagttttatatttttagcagagatggggtttccccatgttggtcagactggtctcaagctcctggcctcaagtgatccgcctgcctcagcatcccaaagtgttgggattaaaggcgtgagcccccatgcctggccacatatactgttcttgaacataaagaaatgaatatagaccctgtttttgaacctcaaaacgatgtcaattatctctaaattaatctataaatataatacaataccaataaaaatgtcaacaggacagaggagcaggacttggttagctaattctaagccatatagaaaaataaacaaagaaaacttcccaggaaaactctgggaagtagaatgagggaagatgataaccaaggaatagagtcattaggtataaataggtaatctgatcaatgaaacagaataaaagtccagtaataacctcaaactgtaggaactaagtacacctatagacaacatttcaatcagcagggcaaagctgaactaaatgaaggacactgccataactgggcagccatctggaagaaaagtatagttggattcccacctcgacactttatttaacttacattttgtgtgggtaagtgcatttaacttaaaaataaaattacagaagtacctggagaaaacacaagaatatttaatttgaattagaaataataataaaataggaaaggcctttcttatgacacaaaacctagactctgaaaagactgataaatttcacatcaaaacatttaaaacatttaatttctgatgaaaaaatagaccataatcaaagtcaaaacgcaaatgaaaaactgtgaaaaatatctgca

SEQ ID NO: 50 - DNA, 1439 bp, Homo sapiens
  misc_feature (565)..(565): n= a, c, g, or t
cggacgccgtcttcggtgcttccaacaccctcgggtccctctcgcggcccggccttcctacgctgctacgttgtgcattacccacaacagcaaaaatgttccactggtggtttcaagattcctaaaaaaatagattcaaaacatctttaatactctctgaagaagcaatacatcacaggaaaagctgtaacctttcctgttgctgatactatccagactgcgggctgatgaggacggtaactgcagccaattcgtattctgcgtggctgccgctggaggtgcacaggagctggtccacccaccctccacactcccaaccaaggaagctgctgaatttctgagtgtttgcctaatgcccttttatataacctgaaaagagtcacagtacttgaaggcagtattttccaagaaacagccttcccctattaacgcaacatccctgaaaataatactgctgtttctacggcagtgacaggccctgcatggaacgcatttttccttccttttttccccatcacagagccacgggcaagaagctgcggcggcagagcctcgtcaaagccancnggacagagggatggctacttgcantgntaagtangagcgcttcacagtgaccatgggccagcctggaangccttcccccangacggggggacaggcgtgagtgggtgtgaagcacagggaagcttctctgcctggtgatcccaagagggcctgcaacaggtgccctgcctggctgaaggaggaggctgccgggaaatcagacttggatttgccaggttgtttgaaggttaatgttatgggtaatcaaagggaggagacaaaagacagaaaaatggaaacccttcagaacagtgaaaggtttcatttacctttggtaaataaaagtgtgttcatattttcacaaggattcttctatttcaccagcttacaactcaaaccgggctgggcaaacacgctgcgatacggcggtcacggctgtaatccagagagaatgctcagaaatgggaaacaaaaacacaaggcaggatgcccctttgtgctggaagtcagaatctccatgtgttcttctcgctgcccgccgaggtctgagcgggtccctgctgtgaatgaccccgggaacggtgccagagtcacacagacttggtgcagacaacagagctacctttcaggggctctgtgtccttggacaggtcactagacttctctgaacccgttcgctcatttccagaaagggataagatctctcttacagttcatggggtgcgcaagaatgatgggtgccaagcccatgaccccaggttctggcaaggaatcggcctctccaaaaatgatggtttttattattctcactattctagaaaatgtctttgtgaattgtttgcttgattgaaacggtttctttttaataaaatcttgattttaaaaagccaaaa

SEQ ID NO: 51 - DNA, 1612 bp, Homo sapiens
cggacgccgtcttcggtgcttccaacaccctcgggtccctctcgcggcccggccttcctacgctgctacgttgtgcattacccacaacagcaaaaatgttccactggtggtttcaagattcctaaaaaaatagattcaaaacatctttaatactctctgaagaagcaatacatcacaggaaaagctgtaacctttcctgttgctgatactatccagactgcgggctgatgaggacggtaactgcagccaattcgtattctgcgtggctgccgctggaggtgcacaggagctggtccacccaccctccacactcccaaccaaggaagctgctgaatttctgagtgtttgcctaatgcccttttatataacctgaaaagagtcacagtacttgaaggcagtattttccaagaaacagccttcccctattaacgcaacatccctgaaaataatactgctgtttctacggcagtgacaggccctgcatggaacgcatttttccttccttttttccccatcacagagccacgggcaagaagctgcggcggcagagcctcgtcaaagccagctggacagagggatggctacttgcattgttaagtaggagcgcttcacagtgaccatgggccagcctggaactgccttcccccagggacggggggacaggcgtgagtgggtgtgaagcacagggaagcttctctgcctggtgatcccaagagggccttcaacaggtgccctgcctggctgaaggaggaggctgccgcgaaatcagacttggatttgccaggttgtttgaaggttaatgttatgggtaatcaaagggaggagacaaaagacagaaaaatggaaacccttcagaacagtgaaaggtttcatttacctttggtaaataaaagtgtgttcatattttcacaaggattcttctatttcaccagcttacaactcaaaccgggctgggcaaacacgctgcgatacggcggtcacggctgtaatccagagagaatgctcagaaatgggaaacaaaaacacaaggcaggatgcccctttgtgctggaagtcagaatctccatgtgttcttctcgctgcccgccgaggtctgagcgggtccctgctgtgaatgaccccgggaacggtgccagagtcacacagacttggtgcagacaacagagctacctttcaggggctctgtgtccttggacaggtcactagacttctctgaacccgttcgctcatttccagaaagggataagatctctcttacagttcatggggtgcgcaagaatgatgggtgccaagcccatgaccccaggttctggcaaggaatcggcctctccaaaaatgatggtttttattattctcactattctagaaaatgtctttgtgaattgtttgcttgattgaaacggtttctttttaataaaatcttgattttaaaaagcccgaagtcacctctgttttctttattcagcagaataggatgcggggcgaggacaacatttagagacttggtaaatgtttttttctttaacttacattgccgtgtgtgtgcgtgtttctcctttcatctttccctgctccccgaatttaagaaaggttctcgtgccgaatgttctgg

SEQ ID NO: 52 - DNA, 480 bp, Homo sapiens
  misc_feature (115)..(115): n= a, c, g, or t
ggttgctattttgagacctcacagcctcatatccactggggaggataatatgcaaaccaagcccaggctgggaatgaggggtgccatggccgatcttcatcccaaaacacatganggtctcaggaacagagacctgccacgtattttacagggacactttgattgcttaaaatcatccctggttttatttggaaaaaaaaacatgtttttaatccattccacagattctgcctttgcagtttgctgggcatggacccagcccctaggagtccagtctgactcattacagaccgcgtgccagtgtttgcaaggcagtcaccgggcaggtgccatctgtcgatgctgtgtgcaccctgctccacacctccccgagactgaccctgccgactctgtcccattttccctgggctccaggttagccagaattgtgcccaccccttcctgttttgttttgttttgtttttctgagtttaaatttta

SEQ ID NO: 53 - DNA, 732 bp, Homo sapiens
  misc_feature (663)..(663): n= a, c, g, or t
ctcaggcatgactctttggaggatgttggcactgagctttgaagaacatgttaagtggagagcaaaagaagggcattccagaaagataaatgggtaaagataaagcacaagggcaagaacaggtagaactgatgagagaaagggaccctgttttgaaaaggtcagaaataaggttccttatgtaggataggtccacattatggcaggccttggacagacagaagggtttatattttatttaaaaccatgctgtaggattattaatctgcaagcagtatccaggggtaattggaaaggagagagcctggattcaaggaggccatggtgggatgggattggaaggaaattgtttcagtcttagaagccttagatttccagataaccaggttctgtgcccatagactgtgatgcctccaaagagcaggtttctagcaaaggtaatttagaaaacaacttggtaacagatggggaagacagctggggcctggatagatactgccaggagagcaggagtgggggccatgcacagggaaccttatggccactatggagttgttgtatgaggttgctgatggtttggtcacgagggctcaggcatcagagaaactggacagtcagtgtttttcctccgcatgttattcagaatatttctgatgttggctnttgaaactaaaacacagggcccaggataatgttacaataatccncattcgatgatcaagcntnaagtgc

SEQ ID NO: 54 - DNA, 766 bp, Homo sapiens
  misc_feature (473)..(654): n= a, c, g, or t
gctgtgtgtcatcttgaccaacttgcttaatctctctggcatctcagtttcttatctgtgaaatggcttgatcttgctgggctaagaatgtcaagtgagatgcttatgggcatagagcacaaaaccatgacctggcttatcctaggtctgcagcagatgttggttattgaccctgccctccctggtcagggaccacatcctaagttttgaagtcttagtgctaggctgttaataactccttaataactaatttatgggttgagatattggactattatttgaaaaacaggaactaaggaaactatcatattaaatagagttaaacttcctggtttttcatttaccccaaaagacttctcttgtgtttaggttcaacattttttatctatatctgtgatttccagtcactttgaaggagacatgttcaaatccattctattctgttccatgtagatgttaaaggtacattaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacagtctccttggatggttcctccccaatatctccccgctcttcagggcatataactatagatgacatacacagtagactgcagattgcctaatcttttgaagatttgttag

SEQ ID NO: 55 - DNA, 916 bp, Homo sapiens
  misc_feature (473)..(654): n= a, c, g, or t
gctgtgtgtcatcttgaccaacttgcttaatctctctggcatctcagtttcttatctgtgaaatggcttgatcttgctgggctaagaatgtcaagtgagatgcttatgggcatagagcacaaaaccatgacctggcttatcctaggtctgcagcagatgttggttattgaccctgccctccctggtcagggaccacatcctaagttttgaagtcttagtgctaggctgttaataactccttaataactaatttatgggttgagatattggactattatttgaaaaacaggaactaaggaaactatcatattaaatagagttaaacttcctggtttttcatttaccccaaaagacttctcttgtgtttaggttcaacattttttatctatatctgtgatttccagtcactttgaaggagacatgttcaaatccattctattctgttccatgtagatgttaaaggtacattaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacagtctccttggatggttcctccccaatatctccccgctcttcagggcatataactatagatgacatacacagtagactgcagattgcctaatcttttgaagatttgttagtcttacagcatgtctacaagccatatcgggtcttttttacatctaatgtcttgatcattgcatttggagagttttgctggaaagggcattgtgtctggttagatgagaaatcggcagagagaaatgaggaaggaggatcctgtagctgat

SEQ ID NO: 56 - DNA, 333 bp, Homo sapiens
ctgcattttagcaagggcctcgcgtggacagaattgcacgcttgagtttgagaagtactgcttggcctctgccccacttacccttaccttcttgagatgaggtaaagcacttggtaggcactaggcgggtaggtttatctttgctcccaagtctcttccgagaacttgggaaggctgcctagcttagtgagggaatccattctggcttcagaccagtctgggtttgaaacctacacatcccactaacttaggcagggtaccgaaactccctgagcttcatgtcctcatcagtaaaacgaggttaatcacacctacaccttagcgcgatggctt

SEQ ID NO: 57 - DNA, 4285 bp, Homo sapiens
gaggatttctctgatggacaagttttttttgtttgttgtttgtttttttttaagccatcgcgctaaggtgtaggtgtgattaacctcgttttactgatgaggacatgaagctcagggagtttcggtaccctgcctaagttagtgggatgtgtaggtttcaaacccagactggtctgaagccagaatggattccctcactaagctaggcagccttcccaagttctcggaagagacttgggagcaaagataaacctacccgcctagtgcctaccaagtgctttacctcatctcaagaaggtaagggtaagtggggcagaggccaagcagtacttctcaaactcaagcgtgcaattctgtccacgcgaggcccttgctaaaatgcagattctaggcacgccccgtgaagtaccgagtcggattaggagaggcggccaggggagcctgcctttagaaagagcttcggatgattcagatgcacacgtttgggaccgtgggctatgggggtcgccgcaataaccatgtgaggattaacgggggcttaaccgattgtgaaaatccccttagaagtgtcgcgtaaacaaccccaactcccactatggtgccggaccccagtgcctcactgtacaaagtcgcccctccccaggcgacaacgagagagggactaaataaaaggttgatggctttccactcaccaggcatggggttgagtggcagccggctactgccgaagagcaggtaaaagctcctgccacacctgggcgagactggacttagggcgcaatgtatatgctatttctggggcatttgtgggcctcaccctaaggacttctgattggcttcagggcagatctatcattcatcctctgggttagaggtgggtggaaggtcaacctcgaccaatacaaaagcactcttcggctaagcccctccccttccagcttcctgttgctgtacatcaaccacccagcgggcgggactttgcctccctccaattggctgatgcacatgtcactcattattcaattggctttcggttggtgcgctctaccgtccatcagtgacattgcacagacctattggtcaaatcaacgtcgctctagacgcctctggttgattggttaatagtgctgtcggtccccaatgttccaagcgcttattggtgaaggctgccgtcgctcgggcggtggcgggctccgggattggcggttgcttggcgggcggtgtcaggctctcggtggcggcggaggcggcggaggccagggaggaagatgtcgtaatgagcgatccacagaccagcatggctgccactgctgctgtgagtcccagtgactacctgcagcctgccgcctccaccacccaggactcccagccatctcccttagccctgcttgctgcaacatgtagcaaaattggccctccagcagttgaagctgctgtgacacctcctgctcccccacagcccacaccgcggaaacttgtccctatcaaacctgcccctctccctctcagccccggcaagaatagctttggaatcttgtcctccaaaggaaatatacttcagattcaggggtcacaactgagcgcctcctatcctggagggcagctggtgttcgctatccagaatcccaccatgatcaacaaagggacccgatcaaatgccaatatccagtaccaggcggtccctcagattcaggcaagcaattcccaaaccatccaagtacagcccaatctcaccaaccagatccagatcatccctggcaccaaccaagccatcatcaccccctcaccgtccagtcacaagcctgtccccatcaagccagcccccatccagaagtcgagtacgaccaccacccccgtgcagagcggggccaatgtggtgaagctgacaggtgggggcggcaatgtgacgctcactctgcccgtcaacaaccttgtgaacgccagtgacaccggggcccctactcagctcctcactgaaagccccccaaccccgctgtctaagactaacaagaaagcaaggaagaagagccttcctgcctcccagccccctgtggctgtggctgagcaggtggagacggtgctgatcgagaccaccgcggacaacatcatccaggcaggaaataacctgctcattgttcagagccctggtgggggccagccagctgtggtccagcaggtccaggtggtgccccccaaggccgagcagcagcaggtggtacagatcccccagcaggctctgcgggtggtgcaggcggcatctgccaccctccccactgtaccccagaagccctcccagaactttcagatccaggcagctgagccgacacctactcaggtctacatccgcacgccttccggtgaggtgcagacagtccttgtccaggacagccccccagcaacagctgcagccacctctaacaccacctgtagcagccctgcatcccgtgctccccatctgagtgggaccagcaaaaagcactcagctgcaattctccgaaaagagcgtcccctgccaaagattgccccagccgggagcatcatcagcctgaatgcagcccagttggcggcagctgcccaggcaatgcagaccatcaacatcaatggtgtccaggtccagggcgtgcctgtcaccatcaccaacacaggcgggcagcagcagctgacagtgcagaatgtttctgggaacaacctgaccatcagtgggctgagccccacccagatccagctgcaaatggaacaagccctggccggagagacccagcccggggagaagcggcgccgcatggcctgcacgtgtcccaactgcaaggatggggagaagaggtctggagagcagggcaagaagaagcacgtttgccacatccccgactgtggcaagacgttccgtaagacgtccttgctgcgtgcccatgtgcgcctgcacactggcgagcggccctttgtctgcaactggttcttctgtgggaagaggttcacacggagtgacgagctccaacggcatgctcgcacccacacaggggacaaacgcttcgagtgcgcccagtgtcagaagcgcttcatgaggagtgaccacctcaccaagcattacaagacccacctggtcacgaagaacttgtaaggccaactgcggcgggaggccctgaagatgcagtcccccacctgtgtcctccctgggcccctggtggaaaggagccctgtggctgccttgggcctgccctcagccccactcctgttctgcaactgtccccacaggaaggggctctgttccctgtattgtcctccttctgaagccccttggctctgccttggcccttcccctcaccacgagctcccggcctgcccagactgtggacactggccgtgcccaatgagacgttctaaaccaggacgcgtgggaacccttatttccaaaggaaaaacatgcatttcactccgtcgaggagcaaagtgagcccctaccccccaccccgatccccgctcccaacactgccggagtcgcgtcatgccatgccccctctcctgcacctccctggccctgccggccactgtggacgccctggggcttggcacccacctctggagaaactcggggccacctccactccatgtgcccagccccgccacaacctctcctccagcacattccagctctatttaaaaagtaaagacacccaccgactcctgatccccctctttttctatggagaacgttgccttatactctacttcagatgatgaacactgtgtactgtgtgtgctttaaagaagttttatttaattgctcccttcttcctttccttgttattcacctccctgatgcctgctttcagttgagggttgggggcaatgatgagcatatgaattttttctcactctagcaattcccttttctaaatgacacagcatttaaactcaaatctggattcagataacagcacctgcacatcctgcacctcctccctctcccttcacctcacccctgcccggcccaagctctacttgtgtacagtgtatattgtataatagacaattgtgtctactacatgtttaaaaacacattgcttgttatttttgaggcttttaaattaaacaaaaatccaactttatttttagttgtaactgcttgaggtatgttttatgaattaagtgacagatttgttatcctttattaacgtactttgttggtcagcactgggctgacaaaaaattttttcttgctaataaatttaagttcctgaggcaaaatcttcaaacttggcagttcggctttcctttgtcttttccc

SEQ ID NO: 58 - DNA, 129 bp, Homo sapiens
gtttggactcagcgactgcaaagcaacaaagtgaaagaagcgtggattcctaatgcaggaggcactgaaccaatcaatacccactgccctccagacctccacatgaaagaaaaaaactgctgtcctgtg

SEQ ID NO: 59 - DNA, 1961 bp, Homo sapiens
gaaacaggagacattacaactaatgccagagaaataaaaagcaagaccataagggactattatgaacaaatttgacaacctagaagaagtggataaattcctaaaaacatacaacctataaagacagaattaagaagaaatagaaaacctgaaaagaccaataacaaataaggagattgagtcagtaataaaaaaccaacccaaacaggaaagcctacgaccaggtggcttcacaggttaattttactgtacatttaaagaagaattaatatcctttcttcctaaaggcttgtaaaaagtagaagagtaaatacttccatactcattttatgaggccagcatcacactgttaccaaatccagacaaagacaccacaaacaaagaaaatgacaggccaatatccctgatgaatatggatgcaaaaactttcaatgaaatattagcaaaccaaattcaacagtacattaaaaagatcattcaccatgatcaacgttccctggggcctagccaaccgcgggcagggctgggcaaggcgggaggagcgcggacccaagatgcggctacgctgcgtctgctcaggctgcgagttcccggctctggggactcaccttgcggagcttacccaggcggactctccgcagcccctgatgggtgtgtgtgtcagaccgtttactaaacaccaggactgttgtaggcgactgtaagaaataagaatattgcatggttacaattcttgaatgcttaccttgtgtcttgttccttactctacagaaattttctaatcccgtggcgaacccccgtacttcaacgtatgacattctgtaaatctacaaaacagatgggtccctgcctttgagaagtttttacaatttagtgggtactggacacggacctaatttaattctcacatgccttccctttctaataagctgcatgttatgtatccctattctacagatgtggggaatccgaaatttttagcttttactgggacacacagctagcaagcaactaaagcgagaattccaacctgagctccaatccgaactattttcataaaaggcaggatcccaggctgagggagttcgaggttgggagacagtttagcagtcgctgagtccaaacatgttgttttcctagaaaagaaaactgagatccaggaaagggaagtgattttccaaagacacacctgtgaaaggtaataatttcactgaacggatcttaagtggcaggtgcactaagattgtctgctatcttcacaacaaccccgaagtaaataagtgctcttatcttggtcttacaggtgaggagaacagcccagtgaggctgaggccacacaattaaccccagtctttgcactacaactctaccagggatcctggtttggctcattttctttcttctgcaccatgattgttcaccttttcattaccagcaacatacccaaagacctagctatagactcgttggaatctgttacttagcagctgtgtgaatcaagtgttcaataggaataataagatgtattcatcttaccttttcagaacattatgaagaacaaatgtggtaagacatatgaaaatgctttgtaaagtgttagatgcttaatacacagaagacattgcttccagatctgtttcaacgtaccccaagaatggaataagtggaatatgctggtttcatgcactgtggcaacgggggtctcatcttggctagacttgaataagtggatttgttgcctggcctctgtggatggctctttctctgggcttttgtgtatttgattttttatttgctccagcagccgtgttgaaccatgaaaggacctcaggaatgaacccacacacaagcaaagcaacaaagtgaaagaagcgtggattcctaatgcaggaggcactgaaccaatcaatacccactgccctccagacctccacatgaaagaaaaaaactgctgtcctgtg

SEQ ID NO: 60 - DNA, 541 bp, Homo sapiens
  misc_feature (493)..(493): n= a, c, g, or t
cagaggtctcctaagcttgactcctcttcggcatgagactaaggaagctccccaagttccagtcagaggtctgaatttgtgaatttgctgaggctgataggacagtgtgggctgtccctgaaagttggaggaggtgacaacaaggttgggggccttccatatgggtgcctgctgtaccacatatccagtaaaaacagaacatttacgaatgcaggcttatggaagagtcttgccctaacagaaaaacagggatgaggctgggaagaggataaagttatagcaggataaagttataagaagataacaaagcaggatgacatcacatgggaccaagaagggggctgcatcatccctggacctgctcctgggggctgtcggattgtgggcgtggccgctttggatgcccaggtgagccctgacacagaggtgcttcctcacattacagtgaggagaggatggggccagtacaaagactgaagaaccagcactcctnctgggtccgtctttgctaagttgagtcatcccagccnaggctcagatg

SEQ ID NO: 61 - DNA, 1008 bp, Homo sapiens
  misc_feature (960)..(960): n= a, c, g, or t
ggaacgtggtagtgtcaggagaggaccacgtaaagagctggccagcagatggaagctgagtgctgctggagagtaggaggctggattagtaacgtgatgtgaactgaggaagttaaaggtttgtgtcctccatcagattactgcatttcagctttgtagaaggaagtggcaggttgggcagtgccagtggcagggatgatggtggcctggagacccttgaggggcagaaacagtggcttactcctgtgcttctgtggtctgtttcctcactttgccccatccatcaagtgttgaggctcggtggagttgagcttccttttccctccatcactcacatcccacttcccatcacctgcttccaagcaagagaactccaggttactggcctccggcagcagtccagctgttaccagtaaactggaagctgacgtcactaacaagcattcagggagcaccaagaagcagaccagaggtctcctaagcttgactcctcttcggcatgagactaaggaagctccccaagttccagtcagaggtctgaatttgtgaatttgctgaggctgataggacagtgtgggctgtccctgaaagttggaggaggtgacaacaaggttgggggccttccatatgggtgcctgctgtaccacatatccagtaaaaacagaacatttacgaatgcaggcttatggaagagtcttgccctaacagaaaaacagggatgaggctgggaagaggataaagttatagcaggataaagttataagaagataacaaagcaggatgacatcacatgggaccaagaagggggctgcatcatccctggacctgctcctgggggctgtcggattgtgggcgtggccgctttggatgcccaggtgagccctgacacagaggtgcttcctcacattacagtgaggagaggatggggccagtacaaagactgaagaaccagcactcctnctgggtccgtctttgctaagttgagtcatcccagccnaggctcagatg

SEQ ID NO: 62 - DNA, 476 bp, Homo sapiens
cagatggcaggagatgaaaatggcagcctgatgtaggaatgaggtaggggtgggggtctctcacctcagaaccacccggaactggttgaacacctccttgatctgtctaaggctgattatctggagggaagacagcaaggcagaggttagaccaagggcacctcgagggaccagggggcatctatgttagtagagaaatcaagaccccttggctgacccctggaccatctgtctcgagaagaggacgtgtgcacacagcctctcagcgaagtggcagccaagccctcctccagagcagagaaacacctgattaataattttaaaactaagtatttgctgaggctataaaaaccaccttttcgaagtggagacctctataaaatgaaatgattctataccttgaaaataccttctaaaggttttttttttatttttactacacatgtacttctcagctttctcttacttccaaaaaa

SEQ ID NO: 63 - DNA, 498 bp, Homo sapiens
gatgctcttggaggcgagtgcccagatggcaggagatgaaaatggcagcctgatgtaggaatgaggtaggggtgggggtctctcacctcagaaccacccggaactggttgaacacctccttgatctgtctaaggctgattatctggagggaagacagcaaggcagaggttagaccaagggcacctcgagggaccagggggcatctatgttagtagagaaatcaagaccccttggctgacccctggaccatctgtctcgagaagaggacgtgtgcacacagcctctcagcgaagtggcagccaagccctcctccagagcagagaaacacctgattaataattttaaaactaagtatttgctgaggctataaaaaccaccttttcgaagtggagacctctataaaatgaaatgattctataccttgaaaataccttctaaaggttttttttttatttttactacacatgtacttctcagctttctcttacttccaaaaaa

SEQ ID NO: 64 - DNA, 413 bp, Homo sapiens
  misc_feature (327)..(327): n= a, c, g, or t
gacagtgtggagttggggggcttacaggaagcatgaggatgcctctggttaggggcagggatctgggagcctccctggaggacagtttgcaggaggaccatttgcatgggacctcatcaaatggctagagcttaatggaaaggcattccaaggcaaagcacaacctgagcagaggcttggtggggagcttcgatgggcagtggcagggggagtgtaggtgggtggcagaagtgccctcgtgcccagggcctctcatatcactggaggacttggggtggagggtgctgggtgctcagcctctgccggtccaactggcccaaccacttntctctggcactgcccactgtccctgctcacagctctgccaanagttgttggcaaaccctgggtgaagtctgggtcaggggcctagg

SEQ ID NO: 65 - DNA, 661 bp, Homo sapiens
tggagagtgggttagccaccataaattgttttgggacttcggatacccccttctgcaaatgctatctagagatgctttaatttcatttcaattggttattgcatccctgatattttgaaactgttatgagtagaaaacattctggtttatgaacaatctgcttttcactatgaaatagatgggaattttaattattcctgcgctttgtcttagagaagttacatgcatgcatcaagtgttgtgtgatcttgtgcagttcttcttagattgagtttttggctcctcgtgtgcaggccttctaaaatcacaatgtccctgagttgggaaggaacatcaggggctatctggttcattatcctaccccccaccgcctcccacgccccacatttctggcagcctacgagtctttggtccctccccagtgctacctggccagctctcagccttgggaggccacctgcccgtgtttgacctgggttaatgaccgccagttcctttcccttaatacgactcatctgctctcctgcatgctcgctccttgggtgagagctgccctctgcaacctcataacggaaaatggcttgacttctgccacctcctcctccttcccatgaaagaatctggtcttttctatctctctattatttttaaaaccagatta

SEQ ID NO: 66 - DNA, 847 bp, Homo sapiens
accgaaacctggcctctgggagagtcctgtgtgtcacatcctgtgaacaccctactctgtcctcatatgtgtgtggctctgcactgtaagatcagaacacaggagactgaggaagtagcaatgcagagtttgagtgagggagaaacaaagggacaggtttctggaactattttggactcccgtagtaacttttaactctgctacaatgcccatgtacttggtcttttaagctctctctggcttttgatctctttttttttttcctcctacatgaaacagaagacagagttacgggaaatgaagggtataggaggaaagaaagagctgtagttatcgtagatgccagaggggtaagaggcagctcttctttcaggggcttagctgagggccagagtagtacagctcgtctccacggggggtggagaggggtgcctcaaaacaaaagtgaacatttctctcttctaagtcggcttctaatggccctggtttttgtctgttttaacctaaaaggttattgcactattaacaggtaaaaaattctcttctgaccaaatacatgtatttctagcataatatattctctctctctctctggataaaatttattagcatatcagcataaacataatttttgtaaggtcagacaagagatttgaagtgtaattcaaggggaatttgtattaacgtaatgaaacattctgctgtgaagtttactagcaatgcctcactctgggttatcatttctgactcctcccatttcctttctcacccgtcacctggccctcattcattctctgtcctagaagagaacctctaaaaccagtgacagtccatccagcctgctgtt

SEQ ID NO: 67 - DNA, 1773 bp, Homo sapiens
  misc_feature (1081)..(1081): n= a, c, g, or t
tgcattgtcagaagtgaggccagtcacctggtcaagcaacccctcctgtcgctctgtgaccctccccgggcgtccgcggtgcttctttacttgcatcttgtgggaatgacatctgcgcaggcagcccctttttagcaccgctcctcgcacgcgagcccagcccttaaaaacccagcccagccccgggtttgcagatactcagggcaggggaaggcgcctttccgcatttggtgtagtcccgcatgctgccatttgatgtcgcgggagaacgcgcctacagggatagggcggggcaaggaggcgaggaaggaaaaaaagcgacaagtgaagactgacatccattaaggtcaatggtagctcaatcttcggttctctgactcccaagcttcccattctcttaggaggctgcatttgtggcgctaaaaaaagggatgggcgtgggagattccaggacatgtgctcgctgaagatacaactgttagtccaaggtaactcccactcgtagtggctcaaccctctttgtcaattcaccttcctctaagttaactatggctgctccacctgtctttagggaaacctactccccatcgcgctgggtgttttggagtttttaaaagcgtcgcggggtaaagtcagtgttgtgcttttccttttctcagcaggggtgaccctggagatcattaccaaaatttaaaaatttcaccactgggcacacatgttacccttctcgtgatggcatggcaaagcttgatttgcaagagtttcttttggattgtccccagtgattccctttaaaaataggagctccttcaagctcctgtatcagcaagtgcttgcacctcataggtgatgatcatgagggggacaaggagcctctagacttaagagacaagttgccagcattctccagatggagtgtttccctccgcagcaccccctgctggtcagtttgacaagcaggtgaacagaagttctcaaatacacagtaagcagataaccccaacatcaccagaaatatcaacttgtgatggcattttgtcaagttgtcttgttatcggcatcatctgttgctttctgaggnaagtattttgagttctcctactgagactgtggatttgcctatttctttttgtatgtcagcttttgccatatgtattttgtagttgtgttgtgaggaagattcatgacttatcttgtgggagaattctgtatttagcagattttcgtcttaagttctgttttgcttagtattgatgttattactttgctttttcagtttcaaaatcatttagtatatctttttcctttcttttaggctttgtgtttttatttcagatgtgctttcttatgtactgacctgtgatccccaaattcatatgttgaagtcccaactccaaatgtatttggaggtgggcctttcagacgtaattgagtttagatgacgtcatgagggtggagcccccatgatgacactaagtccttctaagaaaaggaagagagtctgagccctcctctacaccatgtgagggcacagagagagagcagccatcaacaagctgagagaggaggactgagaatgaaacctacctcgccagaaccttgtcatatcccttcccggtttnnnnnnnnnnnnnnnnnnnnnnnnnnnnnnnnnnnnnnnnnnnnnnnnnnnnnnnnnnnatggcagccagagcagattaatgcatgcttcttggactttgtttttaactcaatatgaaaaacatatgttttaataaaaaaattaccccattc

SEQ ID NO: 68 - DNA, 5378 bp, Homo sapiens
  misc_feature (741)..(741): n= a, c, g, or t
gtccttaggggatggtaaatttgacagggctgtaagtgagcacttgttgtctgctgaacactgggtggtcaagagacccactgagattggttagttggaacagaacatgaagagtggataccttaatctgcttgggctgctataacaaaataccatagactgggtggcttaaacaatggaagtttatttctcatagtactgaaagctgcgaagtccaaaatcaatgtactggcagattaagttcttggtgaggaccctcttcctggctcgtaaatggctgacttctcactgtatccttacatgacagggagagaaacagctcaggtgtctcttcttccaagggcactaattccatcatgggggctccaccctcatgacctcatctaaactcaattacgtctgaaaggcccacctccaaatacatttggagttgggacttcaacatatgaatttggggatcacaggtcagtacataagaaagcacatctgaaataaaaacacaaagcctaaaagaaaggaaaaagatatactaaatgattttgaaactgaaaaagcaaagtaataacatcaatactaagcaaaacagaacttaagacgaaaatctgctaaatacagaattctcccacaagataagtcatgaatcttcctcacaacacaactacaaaatacatatggcaaaagctgacatacaaaaagaaataggcaaatccacagtctcagtaggagaactcaaaatacttncctcagaaagcaacagatgatgccgataacaagacaacttgacaaaatgccatcacaagttgatatttctggtgatgttggggttatctgcttactgtgtatttgagaacttctgttcacctgcttgtcaaactgaccagcagggggtgctgcggagggaaacactccatctggagaatgctggcaacttgtctcttaagtctagaggctccttgtccccctcatgatcatcacctatgaggtgcaagcactgctgatacaggagcttgaaggagctcctatttttaaagggaatcactggggacaatccaaaagaaactcttgcaaatcaagctttgccatgccatcacgagaagggtaacatgtgtgcccagtggtgaaatttttaaattttggtaatgatctccagggtcacccctgctgagaaaaggaaaagcacaacactgactttaccccgcgacgcttttaaaaactccaaaacacccagcgcgatggggatgggattgcatcaaagctctcagagcacagctgcaaagggtaaagcaaagctagagctctactctggacctatcactctagacagtttctctgtgaggggcagttttatacacatttcaggtggcagaataaattcaatccttgtttctccatcttatcgagtagtagaagttagttacattctctttgaactcatcatgaattccatgaagactgaagaaaacaagtcatttagcgctatggaagatgaccagaggactagacctgaagtttcaaaggatactgtcatgaagcagacacatgctgacacacctgttgatcattgtctatctggcataagaaagtgtagcagcacctttaagcttaaaagtgaagtcaacaagcatgaaacagcccttgaaatgcggaatccaaatttgaacaataaagaatgttgtttcacctttacgttgaatggaaactccagaaaattagaccgtagtgtgtttacagcatatggtaaacccagcgagagtatctactcagccctgagtgctaatgactatttcagtgaaaggataaagaatcagtttaataagaacattattgtttatgaagaaaagacaatagatggacatataaatttaggaatgcctctcaagtgcctgcctagtgattctcattttaaaattacatttggtcaaagaaagagtagcaaagaagatggacacatattacgccaatgtgaaaatccaaacatggaatgcattctttttcatgttgttgctataggaaggacaagaaagaagattgttaagatcaacgaacttcatgaaaaaggaagtaaactttgtatttatgccttgaagggtgagactattgaaggagccttatgcaaggatggccgttttcggtctgacataggtgaatttgaatggaaactaaaggaaggtcataagaaaatttatggaaaacagtccatggtggatgaagtatctggaaaagtcttagaaatggacatttcaaaaaaaaaagcattacaacagaaagatatccataaaaaaattaaacagaatgaaagtgccactgatgaaattaatcaccagagtctgatacagtctaagaaaaaagtccacaaaccaaagaaagatggagagaccaaagatgtagaacacagcagagagcaaattctcccacctcaggatctaagccattatattaaagataaaactcgccagacaattcccaggattagaaattattacttttgtagtttgccccgaaaatataggcaaataaactcacaagttagacggaggccgcatctgggtaggcggtatgctattaatctggatgtccaaaaggaggcaattaatctcttaaagaattatcaaacgttgaatgaagccataatgcatcagtatccgaattttaaagaggaggcacagtgggtaagaaaatattttcgggaagaacaaaagagaatgaatctttcaccagctaagcaattcaacatatataaaaaggacttcggaaaaatgactgcaaattctgtttcagttgcaacctgcgaacagcttacatattatagcaagtcagttgggttcatgcaatgggacaataatggaaacacaggtaatgctacttgctttgtcttcaatggtggttatattttcacctgtcgacatgttgtacatcttatggtgggtaaaaacacacatccaagtttgtggccagatataattagcaaatgtgcgaaggtaaccttcacttatacagagttctgccctactcctgacaattggttttccattgagccatggcttaaagtgtccaatgaaaatctagattatgccattttaaaactaaaagaaaatggaaatgcgtttcctccaggactatggcgacagatttctcctcaaccatctactggtttgatttatttaattggtcatcctgaaggccagatcaagaaaatagatggttgtactgtgattcctctaaacgaacgattgaaaaaatatccaaacgattgtcaagatgggttggtagatctctatgataccaccagtaatgtatactgtatgtttacccaaagaagtttcctatcagaggtttggaacacacacacgcttagttatgatacttgtttctctgatgggtcctcaggctccccagtgtttaatgcatctggcaaattggttgctttgcatacctttgggcttttttatcaacgaggatttaatgtgcatgcccttattgaatttggttattctatggattctattctttgtgatattaaaaagacaaatgagagcttgtataaatcattaaatgatgagaaacttgagacctacgatgaagagaaagcccggcccaggccagcctaccggcgactaggatgctttcgctttcgctctcgctttccaatactcgggactggggaaaccgggagaatagaagcaggcaaggaccgccgtgggcacggggtcagtgagacagggtcctgctcgcggcgtcaaggaggagcgctgtgggtgtccccagcgcagccaatcggcttccgaagtagctggagctctggagcctttgcttcctcaaatacgagcgggaactgcgttgagcgctggattccaggccgagtgctggcgaggcgcgcagctgtcagaaaagagatagaaactcaccaaggccaagaaatgcttgtgcgtggcacagaaggaatcaaagagtacataaaccttggaatgcccctcagttgtttccctgaaggtggccaggtggtcattacattttcccaaagtaaaagtaagcagaaggaagataaccacatatttggcaggcaggacaaagcatcgactgaatgtgtcaaattttacattcatgcaattggaattgggaagtgtaaaagaaggattgttaaatgtgggaagcttcacaaaaaggggcgcaaactctgtgtttatgctttcaaaggagaaaccatcaaggatgcactgtgcaaggatggcagatttctttcctttctggagaatgatgattggaaactcattgaaaacaatgacaccattttagaaagcacccagccagttgatgaattagaaggcagatactttcaggttgaggttgagaaaagaatggtccccagtgcagcagcttctcagaatcctgagtcagagaaaagaaacacctgtgtgttgagagaacaaatcgtggctcagtaccccagtttgaaaagagaaagtgaaaaaatcattgaaaacttcaagaaaaaaatgaaagtaaaaaatggggaaacattatttgaattgcatagaacaacgtttgggaaagtaacaaaaaattcttcttcgattaaagtagtgaaacttcttgtacgtctcagtgactcagttgggtacttattctgggacagtgcaactacgggttacgccacctgctttgtttttaaaggattgttcattttaacttgtcggcatgtaatagatagcattgtgggagacggaatagagccaagtaagtgggcaaccataattggtcaatgtgtaagggtgacatttggttatgaagagctaaaagacaaggaaacaaactacttttttgttgaaccttggtttgagatacataatgaagagcttgactatgctgtcctgaaactgaaggaaaatggacaacaagtacctatggaactatataatggaattactcctgtgccacttagtgggttgatacatattattggccatccatatggagaaaaaaagcagattgatgcttgtgctgtgatccctcagggtcagcgagcaaagaaatgtcaggaacgtgttcagtctaaaaaagcagaaagtccagagtatgtccatatgtatactcaaagaagtttccagaaaatagttcacaaccctgatgtgattacctatgacactgaatttttctttggggcttccggctcccctgtgtttgattcaaaaggttcattggtggccatgcatgctgctggctttgcttatacttaccaaaatgagactcgtagtatcattgagtttggctctaccatggaatccatcctccttgatattaagcaaagacataaaccatggtatgaagaagtatttgtaaatcagcaggatgtagaaatgatgagtgatgaggacttgtga

SEQ ID NO: 69 - DNA, 818 bp, Homo sapiens
gtggatctagagttgggagaaggctgcccagaagctctgagactttagtcttggaaagataggtaccattttcatgggtatctaaactattcaaacttaacatgttattttattctggcttgcatgtttatttcgttttcttttgtttttggctttcttctcaattagagaagttcttttaaagcaaggttcatgtcttatgcctctgtattttccatatattactagcacagtgttatgtacgtagtaaattataaatagtttgtgattaaccacttaaagataaaatttatttatgaaaacaattacatcagccttttccaaagtacaccttactaagcagtattctcttaattgttcttatggaagctatattttccctttcattatattcatcaactttcaagtaggagtttaatgtgtgttttttctactagactgttaagacccatgaggacaaggactgtgtgtgtcctgtataccactgaatcctgggtgcttgcaatgtgtgcatatagtagttgttcaataaatgttcattgaataaatgaaataggtctctcaaggaacccgtttaaaaaagccttccttgacctagggagtttatgaaatgctgcatggatattatgccatcctcttgaatattcacagttgcatgtcggcagatgaaatctccaaaaaccttgcagcaaagaaatctgtttaaagtgggttatacagtttattaaattaaactgaactgatgaactaccctcctcctttatttcccttaggagaatttgttaatattctgtgaaatcactgatgagcagacactc

SEQ ID NO: 70 - DNA, 390 bp, Homo sapiens
gtgcatgaagagttcccatgtacccctcccacgcccagtttctcacattatttatattttgcattcgtgtggtgtgttggttgcacctgactcaactctacgttggtactgttactaacagccatagtttccattagggttcttggtgttgcacattctttgagtttggacaaaagtataatgaatgtgtctgccgttggagtgtcacagagtggtgtcactgccctaaaattcctccatgctccccctgtcatccctccatgacctttggcagccacagctcttctcactgcctccacagttctgtcttttccaggctgtctgtagttggagtcccagtgttgcaggcttttcagactggcttcttccacttcacaaccagcatttaag

SEQ ID NO: 71 - DNA, 1645 bp, Homo sapiens
  misc_feature (1489)..(1489): n= a, c, g, or t
atggttccagcaggatgggtcggcaggagtggggaaagaaagtctgaatcaccaagatttcagtttgccctcatctcccgaaccccagtgtgggaccactgctgtgtcccttcagtgggctgtggtgaggaagagaaaaagttgacaatccaagtacaaaatgaaaaccccatgcacgctgccagctgccagctgcacttgctacagttcaggacagatgaagggaaccccagcctttggccccctggactcagcctgctctcagaagggcaccgaggcgagtgcctggtggatctgaggctgtgggcaaagggagaggagcgtgttctggagaacgatccggccctgaatgtcagcagagccaggttgagaaatccagctctcagtgaagacaatcacatccctttgcaaacagtcgccccaggaagcacgtttattgccatgacgtggatttggctcaagcctgttttggaagccggcgtgggcaccaccctcgagcggcttagaaccgctggagaaaactggcctcgccagcccccgctgctgaggccccaagcaggaaccttaaatgctggttgtgaagtggaagaagccagtctgaaaagcctgcaacactgggactccaactacagacagcctggaaaagacagaactgtggaggcagtgagaagagctgtggctgccaaaggtcatggagggatgaacagggggagcatggaggaattttagggcagtgacaccactctgtgacactccaacggcagacacattcattatacttttgtccaaactcaaagcaatgtgcaacaccaagaaccctaatggaaactatggctgttagtaacagtaccaacgtagagttgagtcaggtgcaaccaacacaccacacgaatgcaaaatataaataatgtgagaaactgggcgtgggaggggtacatgggaactctctgcactatctgctcggtatttttataaacctaaaactgctctaaaacaaccaatctgtttttttgttttttttttaagcggagctgggtacagtggttcacgcctataatcccagcattttgggaggccaaggtgggaggatcattggcatccaggagttcgagaccagcctggggaatacagtgagacccccatctctacaaaaaataaaataaaaatgaaaaaaaggcagatcccacacccagaggtgcagacccctgaggccacacgctgcccctggccctcaccaggtggcgatggttgcgttgttctgagacatgtggatgcaccttgtcctacaatccctgcaatgatgggtgctgtgtggtcacctctgtagccctgtggccacaagactcacccacagtggcacatcgagggctcctgtggggtttggctgatcagggcaatgaacagtgggcagagggcatgaggcataagagcctgggttggtcccttctctgctgcctctacagtgaaactaggcagccccctccccganaaatcttcgccatccccttaacccccaggcctgccgtacgccttgctggcttccttccaccctgctggtccccctagaaatgtcccgttgtgaagcactcctcaaactggtttgaatacaccagccttttctttcaggcaccttgagaatgaatgg

SEQ ID NO: 72 - DNA, 129 bp, Homo sapiens
tacacgatggtatggcatatgaggaaggggaggtggaaggtatatggggaaggggatgggactgcctcttttcactgtccagtggggccaaaaatcacctataaaataaacacacaaaaagtaagggtg

SEQ ID NO: 73 - DNA, 552 bp, Homo sapiens
  misc_feature (125)..(387): n= a, c, g, or t
tacacgatggtatggcatatgaggaaggggaggtggaaggtatatggggaaggggatgggactgcctcttttcactgtccagtggggccaaaaatcacctataaaataaacacacaaaaagtaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtttcccatgcactcatttagtttgaaccttcacagcaacccaatgaggtaatactcccatttcacatataatactgagagatgagttgcacaagattatacactgttaagtagcagagccagaatggacttcagaatcccaactacaatacaaatgtttattta

SEQ ID NO: 74 - DNA, 591 bp, Homo sapiens
tacaggtggtgttgtactactttagcttattgttcttcaaaaatgtagtcatttggaagagtaatgtgtttcttctgtcttttctaagtgttaaaaaaaaagtctacataagagtctagaagtttgttgccaaatccttatttattttacagattgaaaggcttaactactgtgttccttatagcacatggggattcaagataattgaaatactttaaatataaatttaagtttgacataaagtgttacatcataagcattattgtgaatcttcctttcattctaagagaatattttgaagatgtattttcatggtaactatagtgttctactgttttataaaacgtagggtaggcatcaaaagcaagagaaattgaaacattaaatagaaatgcagtgttagcaatctgtttttataaatcttgtctcttagatctgctcagtctttcagacaacactaagcctaattgaaaccagcttttcttcagaggtcctaggccaattttcattcagaagacacaagcatcatccatatccagattggtatacaacaggattcagcagatttgttctgtaaagatccagctaata

SEQ ID NO: 75 - DNA, 272 bp, Homo sapiens
cgacttgcctgaagattccctagtaagtgagagccacgggggcccccaggcctccctgccatatccagggtgctttttagtctatcagctttcccagctcttcagtgagatgttcaggcacatttcaataaagggtctatagccagctctttgtggggagtggaaagagtgaagcagcttctttagtgtagggcttctcagagtttctagtgtactagggtgcattgcagatctcagaagtggagtagatggatttaaaaaaagacagtaat

SEQ ID NO: 76 - DNA, 338 bp, Homo sapiens
  misc_feature (111)..(111): n= a, c, g, or t
tgcatataccctttgaccaggcaattccatttctggtaatttatcctaaaaataagcctgcacaagtgaaaaatgattatatagaaggtttttatggcagcaatatttacnaaaacaaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatggtgtctattaaaatgaaaatacctcaagatgtattgttaagtgaataagagatgggttaaaaataagagtatgtaaaattaagactataaattgctaagaanatttaaaaaatnaacaa

SEQ ID NO: 77 - DNA, 89 bp, Homo sapiens
  misc_feature (85)..(85): n= a, c, g, or t
gatttcactgggcatcctgtattttcatttgctcaataaataagtaaatatattcactcaaaaaatagaaaataaacttgctcantaaa

SEQ ID NO: 78 - DNA, 67 bp, Homo sapiens
atttaaacaagatttcaggagaactcattcactatggtgagaacagtaccatgctatgagggatatt

SEQ ID NO: 79 - DNA, 731 bp, Homo sapiens
  misc_feature (212)..(300): n= a, c, g, or t
gttagctgttgcatccattccattatgtggaggtaccttaatttatctaataagtcctgtgattagtcttatagtttgttttcaaaattttttttgtcacgataggccctattcagttgactttgtgatcagctctgaacatataatctggtgattgccttaagataaattgaataataaaagtaacaatagtagctaccatcggttgaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctctgccagctagttgtgggcttcctttgaggcacaggggagctgaatgggagggtctacataggacagggcgtgtctgagtccaggaaggacagctggacttgggccagtctcacaggtgggaaggctgtctttctgtgaggatgggtgacttgttgacctttgatcctcagagccctgatgcagcaggagtgaaatgattcccagagccacagaaaacaagagtatctttgtcttggattttagatcctggagctatcaggtggtaagaaggctgtctgacccagattaagtgatgtcatggtatcatgttggggtgtgggccttcctgttgtaaattatttgggatttcatagagatcgaagtcatgtgtggatgacatgacttcaggatgtcccattagaagaggaacgtgggggtcaggcgtggt

SEQ ID NO: 80 - DNA, 76 bp, Homo sapiens
ccaccacgcctggccaatcttataacattccattttatgcgttttaatcctttaaaaaaatctccctggccgggcg

SEQ ID NO: 81 - DNA, 713 bp, Homo sapiens
cggctcgaggggtagcagagcgcgcggcaaggaggccgggtccggaggtgcccacgtgctacgtatcctcgtgtgaaactttccgtaattcacttatccgagtcctttaggcgccgtacactagtataggtggtagctagggtaggcgtcggctagtgccctgcccctgcgcctgctagaatacgcctccctcggctaacgggtttgggatagtactgggagtacagctgcctcgtgctcttaggctagcctcgcctccctgggcgccctttgcccttctctaggacctatataacgtagggtgtcctatgggtaggctctgtcaagcctgtgagtccatatatctatttgtaaccttggtaacgaactaggcataagtgtttagccatctagtatttatccgttgtagaagctggcaaagacctcatagatatagccgggcctagtcctaagtgaagacagtggagagtggaaacaggagcaaacgaaatctgtaaactggttgatgattccatgaacttttatgaaatccccttgtattggcttccttccctcttctgtcttacttctctactccctacaagtgttttctgggatcacctccaaataaactacttgcaatctaatcctgtctcaagtctgctgctgtgggaacccaaactaagacacctgcaaaaaatatccttccaattactgaaaaatatgttcacttg

SEQ ID NO: 82 - DNA, 176 bp, Homo sapiens
cattgccacagagttaaatcagtgaaaaattattgaaaatctagaaaacattaccagtatatgtggaaagttagcacttgctaaaaatggcattttaattgagtaaaagaaggttcctaataagcagagttgctatctggttggaaaaaataattattctaatattgtatgcaaaa

SEQ ID NO: 83 - DNA, 628 bp, Homo sapiens
tttttaagtgcagttctgagaggtaaagtgatttatccgatatcacatagcttagagagaggaagagaaaggatttgaacccaggtttgtccagagcctggaccctagaccactgcactccagcctggctgactgtgtgagactccatctcgaaatacataaaaaaaaaaaaaaagataaaaaataccaaaagaccaaaaataaacaacaaaatatcctggtagaagatgtaatatacaaatatctattttttatagatattttagatagagatttaatacaattctggcaagaaaaccaagctttaaataaatgacaaagattattttaaaattaatttggaaggaaaaatatgcgaagattgctaatctttttttaagttgaatgagaggagactttcttcaccagatattaaaactgaccataaagctataataatcaaaatggcatggcattgccacagagttaaatcagtgaaaaattattgaaaatctagaaaacattaccagtatatgtggaaagttagcacttgctaaaaatggcattttaattgagtaaaagaaggttcctaataagcagagttgctatctggttggaaaaaataattattctaatattgtatgcaaaa

SEQ ID NO: 84 - DNA, 106 bp, Homo sapiens
agaacctgcagcattcattatctggaaagatcaatcatttcctaatgaaatccagttttgtatgttagggatcattatctgataatacagtatgtgtcctgctttc

SEQ ID NO: 85 - DNA, 1416 bp, Homo sapiens
  misc_feature (1122)..(1208): n= a, c, g, or t
gtaatctttttattgttcaacttcccaaataaaatataaacttttaacaaacagggaccttggctttcttgttgaattctgtatccttagtgtttcacatggggactgacacaaagtaggtttcaagaaatagttgttgaagtaaatatttccttaggacctactctgctgagattactagttgccttgtatcatagtaggtcatacttatcccttctttttaagtaacataattctcattttaacctggaaggccatactaagtggccaaaagacaatccagctcctgtgtcattaagaataactgtgtaaatctgattcatgagatgtaaacagaaatgttgtgtttcacttccatgaagtcttttactccatcctcctccttagatcatgcatgtgatggctggaactcagtcattttggaccatgaagaccagacccacacgctggggcctaagaagcagaagtatgaaagccacctggatccctgaagaccatggaattgttagaacaattctactctatctgtagacttgttttatttgaaagataagtagatttctgtcttattcaattcactgattttggttctttgttatttgcaatcaaatctaattccagtagatacaactcctaagtgcagagcactgttctagggtattgtggggcatgctgacatgggtctcatgcctgcatagcactcatagcctgcagaagaacagcagtgctgccatatctgatggtcatttgacacactgaactccagccatatcaattttcttttcagtccccaaatggcagtcaccacaaaagaagaaaagataaagctcttagtgggatctgaatcaagagataccacttacagtggaaggaaaaaagatcattttcatggaggaactgcccttgaatttcgccttgaatattgatgagtattggaatctgcagagactgggataaggttgggatgaggtcgaacactacaggaacagaaaatatggaacatgtttgggagcaggccagggattctgtcatataaagtgcatgaaaaagcatatcatgtaatatttatgattattgctctggagttagactgtttgggtttgaatcccagatccagtgttnnnnnnnnnnnnnnnnnnnnnnnnnnnnnnnnnnnnnnnnnnnnnnnnnnnnnnnnnnnnnnnnnnnnnnnnnnnnnnnnnnnnnnntcttggcttgttaccagaattaaatgagtttttatgtgtggagggctcatgagagtggctgtcccaaataagcattctctaaatgttagatatgactgtcatccccttaaaactgcaagagttagttgaaaccatagcaagccgagccatgaatgccatgttaatgcatgttaatgccattattataaaggtaccaaaaagctgctga

SEQ ID NO: 86 - DNA, 1435 bp, Homo sapiens
  misc_feature (1121)..(1207): n= a, c, g, or t
gtaatctttttattgttcaacttcccaaataaaatataaacttttaacaaacagggaccttggctttcttgttgaattctgtatccttagtgtttcacatggggactgacacaaagtaggtttcaagaaatagttgttgaagtaaatatttccttaggacctactctgctgagattactagttgccttgtatcatagtaggtcatacttatcccttctttttaagtaacataattctcattttaacctggaaggccatactaagtggccaaaagacaatccagctcctgtgtcattaagaataactgtgtaaatctgattcatgagatgtaaacagaaatgttgtgtttcacttccatgaagtcttttactccatcctcctccttagatcatgcatgtgatggctggaactcagtcattttggaccatgaagaccagacccacacgctggggcctaagaagcagaagtatgaaagccacctggatccctgaagaccatggaattgttagaacaattctactctatctgtagacttgttttatttgaaagataagtagatttctgtcttattcaattcactgattttggttctttgttatttgcaatcaaatctaattccagtagatacaactcctaagtgcagagcactgttctagggattgtggggcatgctgacatgggtctcatgcctgcatagcactcatagcctgcagaagaacagcagtgctgccatatctgatggtcatttgacacactgaactccagccatatcaattttcttttcagtccccaaatggcagtcaccacaaaagaagaaaagataaagctcttagtgggatctgaatcaagagataccacttacagtggaaggaaaaaagatcattttcatggaggaactgcccttgaatttcgccttgaatattgatgagtattggaatctgcagagactgggataaggttgggatgaggtcgaacactacaggaacagaaaatatggaacatgtttgggagcaggccagggattctgtcatataaagtgcatgaaaaagcatatcatgtaatatttatgattattgctctggagttagactgtttgggtttgaatcccagatccagtgttnnnnnnnnnnnnnnnnnnnnnnnnnnnnnnnnnnnnnnnnnnnnnnnnnnnnnnnnnnnnnnnnnnnnnnnnnnnnnnnnnnnnnnntcttggcttgttaccagaattaaatgagtttttatgtgtggagggctcatgagagtggctgtcccaaataagcattctctaaatgttagatatgactgtcatccccttaaaactgcaagagttagttgaaaccatagcaagccgagccatgaatgccatgttaatgcatgttaatgccattattataaaggtaccaaaaagctgctgacagtttgtgagcaaagttgt

SEQ ID NO: 87 - DNA, 321 bp, Homo sapiens
ggtcgacggagaattgagcagagaaattggtaggtgacattcataaatctaataaggcatgactatgacaataatagtgatgatagttaatgggagtggcgggcaaaataagttagaaacaattactcataatccactacaatattcaaagacgggacactgaaaggtcatagagggagaaataatgtgtatagagaaagtgggggaaaagtggagacacagcaaaagacctgtttattagagcaatgcaattttcactccaatagctaccttcaccagaatgcttctcaaaatgatgcaaaagactgcgacaacaggccg

SEQ ID NO: 88 - DNA, 531 bp, Homo sapiens
taggtaatacacagtttttttgttgtgatggtcattaatacttagaagatatttttcttggatatttatatgttgttacagagttttccaatttttgaaaagtagtctttgctttgttctatttttgactatttttaagccttcgcattttatgtcagcttcaaaattaagattctccaacaagctcaagcttgaaactcagagctctagaatatctttagaaagctgtctgcaagccacagttgtagtctattcttagagcagtacctaacggcagcggagttactgcattagagctacctgggagtgtctgtgaagaatagattcccctgtgaggccctatacccactgaggagtattttggggggtgggccctgggaatctgcctcttaatttagctccacagctgatgctgtgcatactaagttttcagaactactgttgccttttggtatgtgtcagagactttgtctgagagcatcgttctctaaaatgccatcaagtgttttggtcctcaaaaaatagggtcga

SEQ ID NO: 89 - DNA, 463 bp, Homo sapiens
  misc_feature (90)..(90): n= a, c, g, or t
ggaagtcgggtgttttttttctcacttaaattatttaaaacccagaaaagaaatgatatcttctggtttttaaaggagaccatgaagttntgcatagctatcattgatgtgtagttcatactgcatttttagaagtggaaaatagttatttggaggaagataacaaatctggaaccttaggtgcaaggagaaaaagaatagatgaaagggaaagatgtttgtaaattataaaaatttcaattagctattggttttctgcactttatattttaactgcagaatttttcaaaatcagttaatcttggtggaattagcaggatgttaataggagtgactcagaaaaaaacattttgtgactgtctaagtttggaaagtattggattaaatacaattgaggnttctttactatggaactcctcagaacttataatangctgatatccttgattccccagatgagggg

SEQ ID NO: 90 - DNA, 240 bp, Homo sapiens
tctttttttttctttttcagtgaagttagagacaaggtcatcagccaagactggggagaaagggagatgaaaaagcctggagaaagtttaactcatctaagaggctgttaagtgaatcaacaaaagatgccattttctattaagtcatctattttattaaagagttaaatgcaactgttgctgtatatcatgaatttatgaaagatttaggagtgggagttctttttttctcaaaaaaaa

SEQ ID NO: 91 - DNA, 631 bp, Homo sapiens
tcatctgtgcttagacaaaattacaaagtgttcttggttttgtctcacttcactctggttacagagtggccttgtctgatgttggtgttcaataaaatcgtttggtgggtatgtttgcttcagagagcaggcaggactccttctcatccacgaccttatggaggatacactggtataggagtagaaatgttcagttgggataagggtagatgaagaaatagatgacaggtaaaatcatctctctctctctttttcttttctttttttttctttttcagtgaagttagagacaaggtcatcagccaagactggggagaaagggagatgaaaaagcctggagaaagtttaactcatctaagaggctgttaagtgaatcaacaaaagatgccattttctattaagtcatctattttattaaagagttaaatgcaactgttgctgtatatcatgaatttatgaaagatttaggagtgggagttctttttttctcacaaaaaaaatgagacaggttaccatctttttatataggagtggtacaaacctctttatatgaaatacccattctagggtaaatattcatttgatccacataattggccctttgttttggcttttggactgtggctctcgg

SEQ ID NO: 92 - DNA, 672 bp, Homo sapiens
  misc_feature (531)..(531): n= a, c, g, or t
attatttcaagcattgcagaagctgcttccatgtccttaaggtgacaaagcatatgaggactttgcaagtacttggagtaaaggaagagaagagaattcacagagtgaaaagaggagaaagagtgctctaaaatatcaccaatggactgcaacatgtattgaagttagagacaaggtcatcagccaagactggggagaaagggagatgaaaaagcctggagaaagtttaactcatctaagaggctgttaagtgaatcaacaaaagatgccattttctattaagtcatctattttattaaagagttaaatgcaactgttgctgtatatcatgaatttatgaaagatttaggagtgggagttctttttttctcaaaaaaaatgagacaggttaccatctttttatataggagtggtacaaacctcttatttgaaatacccatcctagggtaaatattcattgatacaaataattggtaacctttgttttggcttttggactgtgtctctcaaaggaaaaaaaaaaatcttgcngttttcaaaagttaaatagaacatcaaccagggaccatggccatccagaagactgagttccataaaaatggactccccactcccactggaatcctctgtttttgtgtatctaaataaatagacgccttgctcattcctggt

SEQ ID NO: 93 - DNA, 1526 bp, Homo sapiens
attatttcaagcattgcagaagctgcttccatgtccttaaggtgacaaagcatatgaggactttgcaagtacttggagtaaaggaagagaagagaattcacagagtgaaaagaggagaaagagtgctctaaaatatcaccaatggactgcaacatgtattgaagttagagacaaggtcatcagccaagactggggagaaagggagatgaaaaagcctggagaaagtttaactcatctaagaggctgttaagtgaatcaacaaaagatgccattttctattaagtcatctattttattaaagagttaaatgcaactgttgctgtatatcatgaatttatgaaagatttaggagtgggagttctttttttctcaaaaaaaatgagacagggttaccatctttttatataggagtggtacaaacctcttatttgaaatacccatcctagggtaaatattcattgatacaaataattggtaacctttgttttggcttttggactgtgtctctcaaaggaaaaaaaaaaatcttgctgttttcaaaagttaaatagaacatcaaccagggaccatggccatccagaagactgagttccataaaaatggactccccactcccactggaatcctctgtttttgtgtatctaaataaatagacgccttgctcattcctggtattaggtgtgattgagagataactcacttagcggcagcagcagcagcagtttctgtgcatgctatagcccatgctcttgtgagcatcaaaaaggagatgaagcctgattaaccattttgaaatctatcggatgagaagtccttacaaaacaacttttatacgctttaattaaaaacgtgttccggcagctcagagctgaatgggcttcagggggagcagggctgagcccgttgctatgacgacaccaaatcttttcacgcctggcccttcctcagcacactctgtctagctctctccagctattaatttgcctctgtcccagctctcactccctcagtcccctgggctgtgagtctatacatgacctaataccttttgctgcttctatggtacttccgtttctctcatcttttcatcacttatatttccttgggaatctctgttttctgttcctcttgaaaacaagttacccgcttgagttaaaaacacttaaaaatgttatttcaaagcatggtaatcattgtttattatccctaatgataaattcaattgtaagggtctgcatatagttgagaagaatcttactatgttctacatttttttatatttcaggttgaacgttggctactacactactctttctgttgctgagggtaagatgaaaataggaaggagtctgttatactctgggtaagttaatttgtgcttagtgttcaaatctcaggttattccacatacagcatcacctggtaacaactgtaagtgaacataagacttttaaatatgtgtttactacaaatcctgaaaaaaaaagcagactttactttatttaagaaggctctttggatatttt

SEQ ID NO: 94 - DNA, 441 bp, Homo sapiens
tgcaaggtttatataatctctacagatgaagaaaaaatttaaagtaagttttgtgaatgtctcaacgtcatgcaacctgaaccatagcagagctggcattccaacccagttaatctgacagcaaaacctgttctcttttcttcacataatgatactggttcccagagtcataatcagtgtctttagaatggcctatttatttttgtgtgtttttttcctgcatggaattttatattaagtctctctgtactaaagcatatcagcagattgggggaaccacttgttgttttcaatttaataaacacttattgagctgctacaggaactgtttataatgcttttccaaaaaagtgaccttctcaacatgatagaaaacatttccgaaaaactgttagctaaaactatacgcagtggagaaagattgaaggctttccccataag

SEQ ID NO: 95 - DNA, 357 bp, Homo sapiens
  misc_feature (353)..(353): n= a, c, g, or t
tgtttccgaagaaggtcaacattctgaggtgccattgtcaggtatgaggtaaacacactgagaataccacattttggcaactcagacaagactattttccaaacatgtaaccctgggaagaaggtaacctatcatagtgtgtgttaagaagtcaagcaaccaagaaatgttaattgaaggcccgttttgtacaaggttgcatggaaaagacacactagcgtaagacatggacagtggccctgagtgacgtgcagccggtttaggcgaattaagatttacatacttgaaaagggacaaacaagtctggacgtggtgactcatacctgttattcccaacactgtgggaggtggangcag

SEQ ID NO: 96 - DNA, 618 bp, Homo sapiens
cagcagtccttttaagggtagggaattataccttatttctctttccacccctcatgtggcccaattctgtgtcacagtcaacaaataaatctttgtcatttgaatggcattacaatccagtctcagagccagaatctaaacaattaataatattgtataattgtacagaattgggtaacagtgcaaaagaaagctacaaggcagtcacacataattgagtgacatggataatgattaccatatttagagggtggaagagatgattggatggattagtgctatatggaatgacgtgggataagctagaactttttggaagggtctagacagatgtacaggacatggatagatgaagagaaaggaggaaggaaattctaaggggaaagattgaggtgagcaaggcacgtttgggaggtggtgagtagctgtgttggcctcagggaagggtttgagtgctcacaggaggcaggctctgcagggccaggggaaggagtttggagtttgtcatgtgaaccgctatatctggcagcatgggatctagattctgtaggggtcacctaagctaggatgtgcctataaagtgtatttttaaaacctcatgggttattctttttcccc

SEQ ID NO: 97 - DNA, 905 bp, Homo sapiens
gtagcaagtaagtcttcaagaaacctttggaagagagaaggcctgtatatgaaatgccagaaggaaggggcaaaaggttcagaactaggactctgggtatggagttcagacacctgtgaattgggcattctgaaccacttgtacaatgtgcaaagctgattttttcttaccatagcacagggttgctttctattacagatgtacctatctggaaacatgttcattcaacagtttgcagtcctgcagttattttcaaaacagtttttgcttccctctttttgtagtaaacacggtctttttccagtgtgctagataaagtctggctctgggcagtaaagggacatggctgctgcttatttcaggaacttaggggcaagtgtcttcatgcctgtggaaacaggagccagctagtactatttccagcaagaaatttaagagaaaggagagatttttattatgattttgatttctttactacaacattgcatgtgtctggagtatagccattacactttatgaaaaaggcaaaatggtcatttggggtgttttaggaagtttgccaaaaggctcctttgtcattataatccttcctaagctgccatccacgggtttaggtcatggatatgaaaagtgaaagggtttagagatgaagtagtgtcccctgagtgcttaccaacctgttaatctttttgagatgttaattttttcatatagagccccctaaaatcttgatggctctagatcagtcaagcctaagagaagacgtatttatggaaaaaaacaaaaaacaaaaaaaccttgctggattgctagtaatatctacttcttggaaattaatacttcatattttttaaaaaaattattgatgcattaggaatattttttgcttagcagttacaaatttt

SEQ ID NO: 98 - DNA, 1275 bp, Homo sapiens
gtagcaagtaagtcttcaagaaacctttggaagagagaaggcctgtatatgaaatgccagaaggaaggggcaaaaggttcagaactaggactctgggtatggagttcagacacctgtgaattgggcattctgaaccacttgtacaatgtgcaaagctgattttttcttaccatagcacagggttgctttctattacagatgtacctatctggaaacatgttcattcaacagtttgcagtcctgcagttattttcaaaacagtttttgcttccctctttttgtagtaaacacggtctttttccagtgtgctagataaagtctggctctgggcagtaaagggacatggctgctgcttatttcaggaacttaggggcaagtgtcttcatgcctgtggaaacaggagccagctagtactatttccagcaagaaatttaagagaaaggagagatttttattatgattttgatttctttactacaacattgcatgtgtctggagtatagccattacactttatgaaaaaggcaaaatggtcatttggggtgttttaggaagtttgccaaaaggctcctttgtcattataatccttcctaagctgccatccacgggtttaggtcatggatatgaaaagtgaaagggtttagagatgaagtagtgtcccctgagtgcttaccaacctgttaatctttttgagatgttaattttttcatatagagccccctaaaatcttgatggctctagatcagtcaagcctaagagaagacgtatttatggaaaaaaacaaaaaacaaaaaaaccttgctggattgctagtaatatctacttcttggaaattaatacttcatattttttaaaaaaattattgatgcattaggaatattttttgcttagcagttacaaattttaagaggcacatatacaccacggaatactatgcagccataaaaaaggatgagttcatgtcctttgtagggacatggatgaagctggaaaccatcattctcaacaaactatcgccaggacaaacaaccaaacaccgcatgttctcactcacaggtgggaactgaacagtgagaacacttggacacgggaaggggaacatcacacactggggcctgtcgtggggtggggggagcggggagggatagcattaggagatatacctaatgtaaatgatgagttaatgggtgcagcacaccaacatggcacaggtatacatatgtaacaaacctgcacattgtgcacatgtacactagaacttaaagtataatttaa

SEQ ID NO: 99 - DNA, 483 bp, Homo sapiens
attctttcctagttatgcaaagctgagttctgtaattccatgattttcagtaagaaggaaaaaagcagacctaagaagcttaaaacttacatggaaaataaaagaacacaaaatttaataaattgaagatggagcagtttgtatccacaagggatatatacctaaaacaaaactcatgattgaaatattaggatggaaagagacataccaggctaataccaacaaaaagaaagctacataaataacaaataaaatagaatttaagggaaaaagtataaataggacaagaaacactacataataaaggaatgaaccaccaaggtgtcatgaacttatatgtatttaacaatataccctcaaaatatattaagcaaaaacttacaaaattatgaaaagttggcccacaattacagtacgtatttttaactgcttctgatggatccggttgactaaagattagttaagtatatgggaagattttga

SEQ ID NO: 100 - DNA, 892 bp, Homo sapiens
caggagaaccatcaatggggtccttttaaaccagtaatcaattccaacatgttattcagaatagcagtgaaggctctccaatacctaggcgtaatcttcatagagaaccattccaaaaatggagcagggtttctacattaatggatccagacccagggccccttttccccagatttatggattcagtatattttgaatcagtatccaaatgggttttttaatttgagttatacccaaaggaatttaaatggtttaattataaagaccaaatgggtttttttcatgggaactcaaaaagttagcctgggtaacagagcaggactctgtctcaaaaaaaaaaaaaaaaaaaaaggaatctaattctttcctagttatgcaaagctgagttctgtaattccatgattttcagtaagaaggaaaaaagcagacctaagaagcttaaaacttacatggaaaataaaagaacacaaaatttaataaattgaagatggagcagtttgtatccacaagggatatatacctaaaacaaaactcatgattgaaatattaggatggaaagagacataccaggctaataccaacaaaaagaaagctacataaataacaaataaaatagaatttaagggaaaaagtataaataggacaagaaacactacataataaaggaatgaaccaccaaggtgtcatgaacttatatgtatttaacaatataccctcaaaatatattaagcaaaaacttacaaaattatgaaaagttggcccacaattacagtacgtatttttaactgcttctgatggatcaggttgactaaagattagttaagtatatggaagatttgaacaatgacagtaataagcttggtgtagtatagaacaaatagaagcaacatat

SEQ ID NO: 101 - DNA, 1732 bp, Homo sapiens
  misc_feature (1187)..(1241): n= a, c, g, or t
gcagtaaggaaagtaagtggacaaaaggattccacacctgtcacaggcgctgccccactccctgagtcaggtgagatgagctccggaaggcaggtgggtaatggatgatgctcacctagtgttccttgggccatgaagatcaaatatttcagccccataggatgtgtaagcttgatttctggtcatctctccttaaggaatcatggaccattcattattttgtttaaaaggacacataagaacgtacatgtctcaatagagttactggtcacaggactgaactgtggtcatgaaagccaagcaacttactttctggcctccatattgctttgtaggagaaatgatactgacaatgatgtcacacgaggagggaagccaggcacgaatctggtggaggtgcggtcagttgtgaccagctttgcaaagggagcggtgggcgaggctgtggtctctcccaggtgacctccatcgccatgcagagctgctctcacttctcctcgggaaaggccagcgtcaggtattcctaggaggaagcccaggtgtgtccagagaacagtagtcttgttagaactggaaaaatgtcttatggccaggtggcccctggtctgaaggaaaaataggagctgagtgtgaacttttatcactcagaagatataatccacctctcccctcctgcccttcgctcctgggttggttgtgaggacagtgctttgtgacagcagtggagccctgtctgtccttccgggctagatttcttctttctcttattcctcctgtttcgttgtatccacgatttgtcgagttgcagggctcaccctttcctgaagaaaccatctcttttcccagctccgcacctttgtccgagatggaggcgtgtcctcccgcccatccccagtggggggtgttctgcccattctccagagcccagcactgctccatctcttttcctccctctccttccccttgctgttacctgctgccttcttgtgctcccactgcccttggccatccctctgtgtcattgtgcgctgtggtgcacctgtcttctctactacaccttaagagcaggactctgcctccttcctaccagcacctcatgcagcaagtgctgccaatagcaggcacccagcagatagcgaatgcacgactccaaaacccagctttgcctcctggctgtagttcagacnnnnnnnnnnnnnnnnnnnnnnnnnnnnnnnnnnnnnnnnnnnnnnnnnnnnnnntgaagactgggatgctatgtagctcagaggaaacatacacctgaaggtgctgccccggggggtgcaatgacctgttactgaggctggatggaggatggcatccatcatcagctacaggtgcctcctctatctacaggttcctgctccatctatactagccacctcctggacaagggctccagtgtcctccatgacaccagctttctccagagcctgtgcaggatttccttcctctaccctgaatcagggtgattcttaaaggacagtttcaggaacatataggggcacttgggtaatcttggtcagtaactgacctttaactatcatccatgtgaacatctacagttagggattttcttggtgatgtttggcaaaaagtaaagaattccccaagtgtgaagcctcattcattcactgattcaagcaaatagttattgagcacactactatgtgttaggccctgggccaggtgctgggaatacagcagagac

SEQ ID NO: 102 - DNA, 2490 bp, Homo sapiens
  misc_feature (1305)..(1359): n= a, c, g, or t
cttttctacattacggcttcatgtgaccaaattatggccatagtatttcagatttattcatccactaagtatttattaagtacctattctgtgctaggtatcaggtgctggggctatagcagtaaggaaagtaagtggacaaaaggattccacacctgtcacaggcgctgccccactccctgagtcaggtgagatgagctccggaaggcaggtgggtaatggatgatgctcacctagtgttccttgggccatgaagatcaaatatttcagccccataggatgtgtaagcttgatttctggtcatctctccttaaggaatcatggaccattcattattttgtttaaaaggacacataagaacgtacatgtctcaatagagttactggtcacaggactgaactgtggtcatgaaagccaagcaacttactttctggcctccatattgctttgtaggagaaatgatactgacaatgatgtcacacgaggagggaagccaggcacgaatctggtggaggtgcggtcagttgtgaccagctttgcaaagggagcggtgggcgaggctgtggtctctcccaggtgacctccatcgccatgcagagctgctctcacttctcctcgggaaaggccagcgtcaggtattcctaggaggaagcccaggtgtgtccagagaacagtagtcttgttagaactggaaaaatgtcttatggccaggtggcccctggtctgaaggaaaaataggagctgagtgtgaacttttatcactcagaagatataatccacctctcccctcctgcccttcgctcctgggttggttgtgaggacagtgctttgtgacagcagtggagccctgtctgtccttccgggctagatttcttctttctcttattcctcctgtttcgttgtatccacgatttgtcgagttgcagggctcaccctttcctgaagaaaccatctcttttcccagctccgcacctttgtccgagatggaggcgtgtcctcccgcccatccccagtggggggtgttctgcccattctccagagcccagcactgctccatctcttttcctccctctccttccccttgctgttacctgctgccttcttgtgctcccactgcccttggccatccctctgtgtcattgtgcgctgtggtgcacctgtcttctctactacaccttaagagcaggactctgcctccttcctaccagcacctcatgcagcaagtgctgccaatagcaggcacccagcagatagcgaatgcacgactccaaaacccagctttgcctcctggctgtagttcagacnnnnnnnnnnnnnnnnnnnnnnnnnnnnnnnnnnnnnnnnnnnnnnnnnnnnnnntgaagactgggatgctatgtagctcagaggaaacatacacctgaaggtgctgccccggggggtgcaatgacctgttactgaggctggatggaggatggcatccatcatcagctacaggtgcctcctctatctacaggttcctgctccatctatactagccacctcctggacaagggctccagtgtcctccatgacaccagctttctccagagcctgtgcaggatttccttcctctaccctgaatcagggtgattcttaaaggacagtttcaggaacatataggggcacttgggtaatcttggtcagtaactgacctttaactatcatccatgtgaacatctacagttagggattttcttggtgatgtttggcaaaaagtaaagaattccccaagtgtgaagcctcattcattcacttattcaacaaatagttattgagcacctactatgtgttaggccctgggccaggtgctgggaatacagcgatgagcaaatcgatcacagttcttactcacatggagcttacactctacccctcaccctgcaaactctgctttctctccgaacaagcctgaacaagcctcgttgtagcccatctaagtcctgcgtcaggggaaatggggaatggggagtggcagggagatcgacctttagcatccaaagaggccttgcagcatcagacatcaaccagtcagggaagctggaccgatctttgccaatgtccccaggaattgaagtgtgtgagcgtgtctgccctgtgatgagaaggataccccaaaccaagacagatgtctcacaaaacacaagcagagatgtcagtcttcaggcctcgcatggagaagatgaaagagaggggtggcttttccaaagcccagcgtcagcacgaagcttccacgagattattttccctcttattttgaagtcctgtttggtatatgctatggtccccactttgtcatcaacgctcctgaactcatcttatgcccgatgtacccgaaagcacttcataaactgtaaagtgctgaacaaatggagcagggacatcttcatcatcatcattatcaaaattgccatcatcctcagcatcatcagcatcagcatcatccaaaatggctttattaaaagcaactaa

SEQ ID NO: 103 - DNA, 1450 bp, Homo sapiens
aggccattcacaaatctctctgtccctctcgtttgccatccagacacacgatagtgtgcttttcccccttaacaataggtgtggtcctgtgccttcctgttgccattgtgttggccactgaaatgtaggtggaggcaacacagccacttctgagtagaagctttaagagtcagtgtaggctttgttactttcccttttctttttgtcatggtgaccagcagtgttcccaatggtagctgttttgtcatcctgagtcccagaggggtgaagaccatgatgcggagcagaagccccaccagcctctggtggacctgcatcaggagcaagaaacaaaccttaatggctttaagcctctcaaggtttgaggtcatgaccacagcacaacctagcctatgccagctgacaggggcagagggcagcagtagcagtgggatgctttcctggagagcaaacctcctctcagcagagttaggacggcagctttcacctctgcattcctcaggctgtaaatgatggggttcagcgagggcgtcacaaccccgtagaacaatgcgacagtcttatccacgttgggatccttggccttgggtttgaagtacatgaaggagattgtcccataaaaaaccaccaccactgtgcggtgggctgagcaggtggagaaggctttgcaccggcctgcagcagagggtaccctaaggatggcagacaggatgaaaaggtaagacaggcagatgagcaagaggggggccagtgtcaggacggctgtggccaccattaatgccagcgcattgagggagatgtccccacaggccagttttagcactgccaagatctcatagaagtagttgatgacgtggccacagaaggggaggtgccagacaaggatggactgtagcagtgagttggcaaagcctgtcccccagctcagcgctgccatctgcatgcaggtctgcccactcatgagctctgggtacctaagcggctggcagatagccacataacggtcatatgccatcacagccagcagcaggcactccgttgatcccagcgccagggtcaggtacatctgcagggcacagccagggaaggaaatggtcctctgggtttccaggaaattgtctagcatgagaggcacaaaggaggaggtgccgcagatgtccatgagggagaggttgtttctggcctccatattgctttgtaggagaaatgatactgacaatgatgtcacacgaggagggaagccaggcacgaatctggtggaggtgcggtcagttgtgaccagctttgcaaagggagcggtgggcgaggctgtggtctctcccaggtgacctccatcgccatgcagagctgctctcacttctcctcgggaaaggccagcgtcaggtattcctgcctcttgctcccctgccacatagacagtctgcttgccccctgctttcagggacctcat

SEQ ID NO: 104 - DNA, 236 bp, Homo sapiens
ctgaaaggaagtcaccaccttgtttttaacttgtattttgtgacttgttaccacttcagtgctttagacacataggctcagtgctgcaatattgtgtctaggtttgattatgctaaatttaacctttcttatgtatcaccggagtagtaaggatttcaaactaccctgcctggtagctttcgctttccttagaaaagcttatcacttcttgactgggccccgtggctcccgcccat

SEQ ID NO: 105 - DNA, 948 bp, Homo sapiens
ttattttttcttcttcttcttcttccttcttcttcttccttcttcttctcttctcttcttcttcttcttcttcttcttcttcttcttcttcttcttcttcttcttcttcttcttttcttcttcttcttcttcttcttcttcttcttcttcttcttcttcttcttcttcttcttcttcttcttcttcatcattattattattattattattttttgagacagagttttgctcttgttgcccagactggagtgcaacagcgcaatctcagctcaccgcaacctccgcctcccgggttcaagcgattctcctgcctcagcctcccaaggagctgggattacaggcatgcgccaccccacggcttgtatttttgtagacggagtttctccatgttggtgaggctggtctcaaactcccgacctcaggtgatccgccagccttggccttggcctcccaaagtgctgggattatgggcgggagccacggggcccagtcaagaagtgataagcttttctaaggaaagcgaaagctaccaggcagggtagtttgaaatccttactactccggtgatacataagaaaggttaaatttagcataatcaaacctagacacaatattgcagcactgagcctatgtgtctaaagcactgaagtggtaacaagtcacaaaatacaagttaaaaacaaggtggtgacttcgctttcagctgcaacatgtaatgagaaaaagctgggaaaattcaaaatcaatggctcttttgggacgatcagagaactgagctaacagggcaaactgtgctggaaacctggaaagacacagaggagatccgcttacctggagcagaaactgccagagccataagctggtgctggatgctcggtgtggactagtgagagtgagaaactcggggaagcagtctcaggaggtgcgagacccacacttcgtgggcag

SEQ ID NO: 106 - DNA, 609 bp, Homo sapiens
  misc_feature (267)..(335): n= a, c, g, or t
aaaaattaatgaacctgcatttcccagattccctgcagtcataagtggtcctctaagttttggccgaaagagacctgagaagtgattgcaacttcagaatcactatttttttttttttgaaatttatacaaacttttttttgttggagtatacatataggaaagtacacaaatggtaagcttagtgaattttcacatgtggacacactcatgcatgggaacatatgccatccagatcaacaaataaaacaattgcaccattgcatcnnnnnnnnnnnnnnnnnnnnnnnnnnnnnnnnnnnnnnnnnnnnnnnnnnnnnnnnnnnnnnnnnnnnntttccagatctcagaagtcttctcttgtcactatcccttacaaaggcaacctgacttttaataccatagattaattttgtctgtttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtagttgtggttcattcattcttgttgtagcgtagtattctattgtgtgactatgctgcactatttctacctgtggatcatgtttttgtgaga

SEQ ID NO: 107 - DNA, 395 bp, Homo sapiens
  misc_feature (331)..(331): n= a, c, g, or t
tcctgtttttactgatttttattgattttgttcaaagatatctggggattggattggggaatgcttaatctgactattgaaagaaacatggatccctggagtatttcagatacggggttaccgccccagactgcctcccgcccagcccacattagttctcattttccctcatgatggtgaaaaacagtggagagctagtaaagggtttatgcagaaaacaaggagaagagaatgtgaagtttgaagtgcctatggaacatccagctgataatcttgcctagtaagagcaaaagaagccaagagaacaccaacgtttaaggagcaggtaganaaaacccagcaaaaggacagaggaatnagaggaccaaagaacaannttaagaancaaggagaga

SEQ ID NO: 108 - DNA, 1012 bp, Homo sapiens
atgggaaacgttggactcaacctagaggagagtgactggggaaagattcgcacacaggtgacaagggcgtttgtggtatgctggaccccgggcctggtggttctgctcctcgacggcctgaactgcaggcagtgtggcgtgcagcatgtgaaaaggtggttcctgctgctggcgctgctcaactccgtcgtgaaccccatcatctactcctacaaggacgaggacatgtatggcaccatgaagaagatgatctgctgcttctctcaggagaacccagagaggcgtccctctcgcatcccctccacagtcctcagcaggagtgacacaggcagccagtacatagaggatagtattagccaagatatctggggattggattggggaatgcttaatctgactattgaaagaaacatggatccctggagtatttcagatacggggttaccgccccagactgcctcccgccagcccacattagttctcattttccctcatgatggtgaaaaacagtggagagctagtaaagggtttatgcagaaaacaaggagaagagaatgtgaagtttgaagtgcctatggaacatccagctgataatcttgcctagtaagagcaaaagaagccaagagaacaccaacgtttaaggagcaggtagagaaaacccagcaaaaggacagaggaataagaggaccaaagaacaatgttaagaatcaaggagagaggtcaggcacgctggctcatgcctgtaatcccagcactttgggaggctgaggcaggtggatcacttgaggccaggagtatgagaccagcctgggcaatgtggtgaaattgcatctttactaaaaatacaaaaactagccaggcatggtggcacatgcctgtaataccaggtacttgggaagctgaggcataagaatcacttgaacccgggaggtggaggttgcagagtgagccaagatcgcagcactgcactctagtctgggtgacagagcaagactctgtcccaaaaaaaaaa

SEQ ID NO: 109 - DNA, 325 bp, Homo sapiens
caggaatcgttttacagatgagaaaaccaaattgagaaatgagacagaggcttgccggagttttgactccgcctgtcatattgtcttcagtctcactgttacgaatctgatgcaggcactgaatctctcagctctgtcacctctagcaaatttcatatcctctctgagctctacaattgatatcaaggttgttgtcagtctaaatgagattgaaaagcttaaatgagattaagtttacgtgttataaggtagtatagtgattcagtaagtgggttctgtatccaatttgccaggtgaccttaagcaagtcacatatctatgccta

SEQ ID NO: 110 - DNA, 1885 bp, Homo sapiens
atgacaggatcaaattcacacataacaatattaaccttaaatgtaaatgggttaaatgctccaattaaaagacagactggcaaattggatacagagtcaagacccatcagtgtactgtactcaggagacccatctcacatgcagagacacacatgggctcaaaataaagggatggaggaagatctaccaagcaagtggaaaacaaaaatagcaggggttgcaatcctagtctctgataaaacagacgttaaacaaacaaagatcaaaagagacaaagaaggccattacatagtggtaaagggatcaattcaacaagaagagctaactatcctgaatatatatgcacccaatacaggagcacccagattcataaagcaagtccttagagacctacaaagagacttaaactcccacacaataatagtgggagactttaacaccctactgtcaacattagacagatcaatgagacagaaagttaacaaggatatccaggaattgaactcagctctgcaccaagcggacctaatagacatctacagaactctccaccccaaatcaacagaatatacattcttctcagcaccacaccacacttattccaaaattgaccacatagttggaagtaaagcactcctcagcaaatgtaaaagaacagaaattataacaaactgtctctcagaccgcagtgcaatcaaactagaactcaggattaagaaactcactcaaaaccgctcaactacatggaaactgaacaacctgctcctgaatgactactgggtacataacgaaatgaaggcagaaataaagatgttctttgaaaccaacgagaacaaagacacaacataccagaatctctgggacacatttaaagcagtgtgtacagggaattttatagcactaaatgcccacaagagaaagcaggaaagatccaaaattgacaccctaacttcacaattaaaagaactagagaagcaagaacaaacacattcaaaagctagcagaaggcaagaaataactaagatcagagcaaaactgaaagagatggagacacaaaaaaaaccttcaaaaaatcaatgaatccaggagcgactactccccttccctcctggcgatctgctgcctggcgctggcggaccgcatgctgcgggtctcgcggcccgtggacttgcgactgggagaccacccggaggcggcgctggaggactgtatgggcaagttgcagctgctggtggccataaacagtacttccttgactcacatgctgcccgttcagatctgcgagaaagggttgcacttgctggtgaggcagatgtggggctgggaagctttggggaccacacaggcacacaagcagaagtgggggaaagccatatccaggcatgagaaatcccgtgttcaaatggagcatgttggaaaccaaggatttgactgtggtcacagccattgcaggttgagcccatttaccctgcaatcacgtggtccaactgttcatgctacgacatggactatagagaacacatctggagagccagcgcagtctgtacctcagtcacgttacaggaatcgttttacagatgagaaaaccaaattgagaaatgagacagaggcttgccggagttttgactccgcctgtcatattgtcttcagtctcactgttacgaatctgatgcaggcactgaatctctcagctctgtcacctctagcaaatttcatatcctctctgagctctacaattgatatcaaggttgttgtcagtctaaatgagattgaaaagcttaaatgagattaagtttacgtgttataaggtagtatagtgattcagtaagtgggttctgtatccaatttgccaggtgaccttaagcaagtcacatatctatgccta

SEQ ID NO: 111 - DNA, 335 bp, Homo sapiens
ctaagcttctcgtgaagctagatgaatagaaacacggagcatctataaactggattaaacataggggggtacaaacctcctggatatttttaattcttaggaagaaggagaatttagcaccagactcttgaaaagcagtggcatgaaagttagtttgatatattgtttgagtttgtattgccttaggccctgaatcaagaccaatggtttgctgtagctgttggtttcaaacaggagctaagagtgatgtcttccttgtggtctgttggctattcagtattccagtgcgaattgccaattcagttggaagaaacatagtctagaatgtaatgtca

SEQ ID NO: 112 - DNA, 934 bp, Homo sapiens
ctaagcttctcgtgaagctagatgaatagaaacacggagcatctataaactggattaaacataggggggtacaaacctcctggatatttttaattcttaggaagaaggagaatttagcaccagactcttgaaaagcagtggcatgaaagttagtttgatatattgtttgagtttgtattgccttaggccctgaatcaagaccaatggtttgctgtagctgttggtttcaaacaggagctaagagtgatgtcttccttgtggtctgttggctattcagtattccagtgcgaattgccaattcagttggaagaaacatagtctagaatgtaatgtcatttttttttttttttttttttttacagagcaaaactttgttggcttaaaaaccagggagtgggccgggggtgtgggggctcatggcctggtaatcccagcacttggggaggctgaggtggggccaatcactggtcgggagatcaaggccatccggggctaacatgggtgaaaccctggtctctactaaaaatacaaaggatggggtgggcgcggtgggggcacacgcccatatgtcccagctactgggagaggctagagcgcgggagaatcacttgaacccaggaggcggagtgttggcagtggagccgggatcacgtcactggcgaccccagccggggcgacagagcgagactctggtctcaaaaaaacaacaaaaaaaaacacccacgacgggcgagtaaagagtggcggggcgggggcgaaaaaaagaggcgatcagtgaacgcgacgcccagttagcccaatagcggtggtcccggtgcaccccagtttctttgtgccaccaggggggaaactcgcgcgggacccccaatatggggagggccgaataaattcggaaaagccgaggcacccgggatagagccgggcaaggcaagat

SEQ ID NO: 113 - DNA, 231 bp, Homo sapiens
cttttttcatttctcttggtacgtgtttgtgtaagtcttatttcttgtgggttttcattgatggacctagtgtctgtctttaaatcagtgtcacactgttttgattgctgtatcttttatagtaaatcttgaaatcaaattgtgtatgtcctccaattttgttgttcttttgtaatactgtagtgactatgttaggtcctttgcattttcatataaagtatggaatccacc

SEQ ID NO: 114 - DNA, 1139 bp, Homo sapiens
atcaatctggtgggctagaagagaaatcctaagaaaaccttcctgcttaatccatctgtacacctaatagataagcatcggtataccaatacaatgaagcgatcttcaaatgctttatcattcagcgctggggccccaggctgaggtgattaggagttagtagtagcagctggggccacttgcagagcggctcccattgtcagcttcagtgccctcagccctttctgcagctgcacagctgagcaagaagaaaccctggtgagaaggtgagaaagccccatgactcaacccgagggcaggtcccactcttaccagccctgcaagtttgagcctgaaaaacatcctttagtgccagggtggaggctccttggggtgccccacacccacctcctgtgacttaggagggccaagacagtggggggacgcactgcaaacaggaaacattccatcagggaacaaagggaatttctagcacccctagatcttgttctccctcccgctgggatggctgacggctgcctgtgtttccttgcaccaggaggataaagctttcccgtgatgaaagcattggcagcgccatctggaaggggctgattccacatcccttgcaatggggaaatctagccttggcctgaagatctaggcggctggctcatctccatccctttcttcctgcctttctgccctcttgcctagaatgtgttcccacaaaggggacaagccccgctgcttgcaattccaggagagctctaattgttaatactcttattacacttgccggattttattttctgatgttcattcagaaaaggtgatatgtataacccagtgtccagttgggtcaactgttttctctctttcacctccttgttagatagtgtctaataattgttccttaaaagcattttgagaaactagtctccatttcagaattttctaatacttgcccagtacccaacacccaataggtgtttgatgcacagttattgaaattgaattaaattgtgaaggatactaataagactcctgcatgtgctgtgttttaggaatattcaccccagcacagtggctcacgcctattcaggagggtaggggtttggggcggtggggaggatcacttgaagccaggagttccagac

SEQ ID NO: 115 - DNA, 3257 bp, Homo sapiens
gttttttttttggatgtggaagccgagacctaaagttggggggtgatctctgaggagatggatcggtacctgctgctggtgatctggggggaaggaaaattcccgtcggcggccagtagggaggcagaacatgggccagaggtgtcgtcgggtgagggtactgagaatcagccggacttcacagcagcaaatgtttatcacctcttgaaaagaagcattagtgcttcaattaatccagaagatagtactttccctgcctgttcagtgggaggtatacctggttccaagaagtggttctttgcagtgcaggcaatatatggattttatcagttttgtagttctgattggcaagagatacattttgatacagaaaaagataaaattgaagatgttcttcaaacgaatatcgaagaatgtttgggtgctgttgagtgttttgaagaagaagacagtaatagcagggaatcattatccttggctgagtatgcttatatggtttttgtattatcattaaaatacttaatattagacagttattttaatccatgagaatgaagattatatattttagcatctttactgaagaaactctagttaattgaaatttttgactctcaatttgggccttttatttgaataaaattctttaaaatgcatgtttcttaagcttacataatgtcaagaatcataaaaagtgatattttaataaacatgttcctttcttgaagataaattctgcctaatattttattttatttttgaaacagggtcttgctgtgtcacccaggctgcagtgcagtggtgcagtcacggcttgctacagcctagacctgggctcaagcgatcctccgacctcggcttccagagtagctggaactgcaggtgtgcaccaccacacccagctaatttttgtatttttttttgtagaaatatcatttcgccatgttgttcaggctgacctcgaattcctgagctcaagcaatctgcctgccttggcctcccaaactgctgggagtacagctgtgagccaccgtgcctggccaataatgtattttaaaagcttgaatagaaatgttatttaatgttaatagctagcattcataccaaagtatcattttcattttgctatttgaggctgaaatatgtgacttctttaatttatgtgtatttgcagcatttcaggtaccttttaaaatccatagagtttgtgaggattatatgtacatgattgagttcacttttaggtttttttcaattgtggtacaatatgtaagttgatccaggtgacataagttgtgataataatttttctcttgatgaaatatcccacctcttatagttaataccttttattttagtctttcataacatttcatgacaaaactgctcttattttagtattcacatattatatctgtaagtagctgtagacttaaaaattttcattttcctttttccagatcatatttgtttgtactgattaataaaaggagacctttccaaaccagagaaatatcattgaaccagggattagtagaggtaactttatttacacaatcttaggccagtcacgtaacttctctggtcgtttgttttctcatccataaagtattgaagtacttttagataatttcttaggtgctttcatttacccttaaaaactcttatcatcccatatgttttcctaccactgttaaagacaagtatagccaggcacagtggctcacgcctgtagtcccggcactttgggaggccgaggcgggtgggtcgcaaggtcaggagttcgagaccagcctggctaacatggtgaaaccccttttctactgaaaatagaaaaaaattagccgggcgtggtggcaggtgtctgtagtctcagctactcgggagcctgaggctggagaatcgcttgaacccgggaggcggaggttgcagtgagtctaagatgatgctactgtactccagcctgagcggcagagtgagacggtgtctctctcacacacacacacaaatgaaatccaagtgtctttaagacttaaaagcagtacatctctatgactccccacaggctggactttgctaatcaatctggtgggctagaagagaaatcctaagaaaaccttcctgcttaatccatctgtacacctaatagataagcatcggtataccaatacaatgaagcgatcttcaaatgctttatcattcagcgctggggccccaggctgaggtgattaggagttagtagtagcagctggggccacttgcagagcggctcccattgtcagcttcagtgccctcagccctttctgcagctgcacagctgagcaagaagaaaccctggtgagaaggtgagaaagccccatgactcaacccgagggcaggtcccactcttaccagccctgcaagtttgagcctgaaaaacatcctttagtgccagggtggaggctccttggggtgccccacacccacctcctgtgacttaggagggccaagacagtggggggacgcactgcaaacaggaaacattccatcagggaacaaagggaatttctagcacccctagatcttgttctccctcccgctgggatggctgacggctgcctgtgtttccttgcaccaggaggataaagctttcccgtgatgaaagcattggcagcgccatctggaaggggctgattccacatcccttgcaatggggaaatctagccttggcctgaagatctaggcggctggctcatctccatccctttcttcctgcctttctgccctcttgcctagaatgtgttcccacaaaggggacaagccccgctgcttgcaattccaggagagctctaattgttaatactcttattacacttgccggattttattttctgatgttcattcagaaaaggtgatatgtataacccagtgtccagttgggtcaactgttttctctctttcacctccttgttagatagtgtctaataattgttccttaaaagcattttgagaaactagtctccatttcagaattttctaatacttgcccagtacccaacacccaataggtgtttgatgcacagttattgaaattgaattaaattgtgaaggatactaataagactcctgcatgtgctgtgttttaggaatattcaccccagcacagtggctcacgcctattcaggagggtaggggtttggggcggtggggaggatcacttgaagccaggagttccagaccagccttggcaacatagcaagacaccatctgtaccaaaa

SEQ ID NO: 116 - DNA, 549 bp, Homo sapiens
  misc_feature (46)..(46): n= a, c, g, or t
gttactggcaccttgtcttttattggtgtcatgtttcttgcttttnaatagttcttatgtccttgcattgatgtctgtgcatctggtggaacagcacttcttccaaactttagagagtgcctttagtagggaaagactttgactctggaaggcatggcatagcattggcccagctaaaggttaggacccagaaagaaggtacagctgcaatttgggaaacggagccaatagggcaccattgcaacttaggccccaggacacgagggactgcatggtggggattctggacctggggatagtgggacatagaagtatcccagattctgtgaagccaggtgtgacagcagcaaggaccctggaatagcagagggcagatgtcacttgggccctggaaggaagggagcagtgctgcaatgattccattccttggggatgggagtatctcagcagctcatactctaatgggctagtccagttccaggaaagcagggtactacaattattcaacctgtagggtgaggtgacccagctcagtcagtgctctgttta

SEQ ID NO: 117 - DNA, 876 bp, Homo sapiens
gtgcaactgattgggtttggccatgacactgatttcctggaggcaaggtgctgcttccattcaggaatgggggtgcatgactgccctgagcagccaaggagccaattctttaggaggctgagtgccatttcagctcaagccttcacggggcagggccaaaagcaacttggaggggtgggtggagcatctccactgcagcttggccccaagaaataggatgtagcagcagctcagcttgtggatggtgcgcaacaatttgggggcggttcaacagcaacaaagctggtgggatggaagtgtactgtggctacctatccccagaacaggacacattccaacaatgtttctgatttcaaatgcagtagccacgtgggtcacagtggtgagtattttctcctcctctgtggggagtgtagctgtgtggattccagggagttccctcagctgggcttagtgcctgtgagaactgcaggggtcaccagaggtgaggactgtaggtgcctaaggtggtgatgggggctgctgtggtcctcttccttaccttttccctgcaaggagaatttccccctggttccaactgatcctgatgtagggatagggtggtggaggtgaggtgtttccttctgttgtctatgtggccatcttgggtttctgcacactacagagtttctaatacacctttaatgtattctaacatcctcccttagttattttcttctaaatgtagttgcttacttattgttttggctatctttgggagggtggtggtgagtattgggagcttctagttaggttatcttacatgatgatcataaaaagtggatttcaaatgctctcaccaaaaataatgacaaatatattaggtgatgaatatat

SEQ ID NO: 118 - DNA, 1076 bp, Homo sapiens
gcaaacattctctggatggtaagaaaagaacactgtccagtcattgataaaattagctacagaaccttacatttggagtgtaacccagcttggtttgttcaaaggagataggatacacatggataaagtcagcttacacacacaggaagagggtcagatgagaaactcatagactcttagagccatagctattccttaaagctggctaatgggtttatgggggttgattttactattcttttctcattttgaacatattgaaaattttctatattaaaacatttttttaagaaacaaatgtgcaggttttgattgagtggatgttggaaaggagaaagagtgtctttaggacaaaaggaagcacggttttgtacagtaagtcagacatttaattacacaaaaagctgatatatgctgataatatacattgattcatatagcacgtagctaaatttgtgaattacaggtacataacttcagagaaacaaggatggtttggatttattttgtctgtttccctcccctgagaaattcgtatgaccttgccttaaaacttttaaagaatacatttttaaatctatattcctgctaaagaaaacagtgagtcattaaaataccaccagataaaaaaaaaaggtttgatttttagttcaggtcggtaatggcctgtaagtaccttgaagatggtatcttcctatcacttatgccttgggacagtacccaagagtggctgaactttttacctaattaaaaaatgagtcatttatttgaaaaattccacttccatttattttatctgatttcttaaatatccggtcaactgctaatttgtcagatgtcataattcgtcagtcctacccttttctacaggctgaatttttaatttgaaacaactacacctctaccgagtgttactttcagacgtaaattactgcaggaagaccccccctctgccttggttaacatttaaatgagatgcctcctgtccaggacgccatgagacgaagagaataatcattttccctaaacgaaaatgtacacatttgtcattttggagtctaacataaccaggcgga

SEQ ID NO: 119 - DNA, 1137 bp, Homo sapiens
acatccaagatcccctttctcttctgtagtaaagtgcgtgattaaataactgagtcttgatgcaaacattctctggatggtaagaaaagaacactgtccagtcattgataaaattagctacagaaccttacatttggagtgtaacccagcttggtttgttcaaaggagataggatacacatggataaagtcagcttacacacacaggaagagggtcagatgagaaactcatagactcttagagccatagctattccttaaagctggctaatgggtttatgggggttgattttactattcttttctcattttgaacatattgaaaattttctatattaaaacatttttttaagaaacaaatgtgcaggttttgattgagtggatgttggaaaggagaaagagtgtctttaggacaaaaggaagcacggttttgtacagtaagtcagacatttaattacacaaaaagctgatatatgctgataatatacattgattcatatagcacgtagctaaatttgtgaattacaggtacataacttcagagaaacaaggatggtttggatttattttgtctgtttccctcccctgagaaattcgtatgaccttgccttaaaacttttaaagaatacatttttaaatctatattcctgctaaagaaaacagtgagtcattaaaataccaccagataaaaaaaaaaggtttgatttttagttcaggtcggtaatggcctgtaagtaccttgaagatggtatcttcctatcacttatgccttgggacagtacccaagagtggctgaactttttacctaattaaaaaatgagtcatttatttgaaaaattccacttccatttattttatctgatttcttaaatatccggtcaactgctaatttgtcagatgtcataattcgtcagtcctacccttttctacaggctgaatttttaatttgaaacaactacacctctaccgagtgttactttcagacgtaaattactgcaggaagaccccccctctgccttggttaacatttaaatgagatgcctcctgtccaggacgccatgagacgaagagaataatcattttccctaaacgaaaatgtacacatttgtcattttggagtctaacataaccaggcgga

SEQ ID NO: 120 - DNA, 184 bp, Homo sapiens
aatcaggctataagagaatatctccttacacttggtcttcagggcagaagtagcaacaaaaaagcacaagtcctctttgaaaccatatcttgatttagtgtcctcagcctaacaggtattgcagtgtggcccaacagagatgggtctgtgagatacttcaacacccggcagcttgctgggtcta

SEQ ID NO: 121 - DNA, 769 bp, Homo sapiens
aataaagttcaggagaagggtcaaataatgcagacttgtaagttcctgttaattcttgactatacaatgattccattttgaatgtttaacacacagttgctcagcagtctggatgttactgtattcccccacaaagacagctctgtaaatgaacataaatcaggctataagagaatatctccttacacttggtcttcagggcagaagtagcaacaaaaaagcacaagtcctctttgaaaccatatcttgatttagtgtcctcagcctaacaggtattgcagtgtggcccaacagagatgggtctgtgagatacttcaacacccggcagggctgctgggtctaacattggagtgttgggctgcagacactgactggggagaaatgttgtgaactttatttgcagttctgagagaaatgctgtgttgaaacaggagaggttttcagaagctgctctcagtagctggctccaggattggggtgatggtataatacttacaaaaggcagcacatcagatagagaatttgataggcagaaattaggtagctccggcaaggttatttttcaatggaagactaaggggtagatccgagtacattagcagaaaaagttctgtgatttaaatgggaaggttgagttagaaataattataaagcccagtcatgttgtgacttggaccagtgaaagaacgatgttctacaataattctggaaatggagttggtttagttaaaatcaaatcctttttttgatgatggggtaaagggttttg

SEQ ID NO: 122 - DNA, 395 bp, Homo sapiens
tgaggccattgccagactgcctctgctgcaggaaaacagtaccaggccagtgtctagtctttggttgcagctacagatatgaaatcttgtcaacacaagcacaatttgtcaggcagttgtgcaaggctgagtgacattcagtggtctaagccttttttccctccatgcagatataaacatggagggtaagcatggaggctgaaaagggaggaaggaggtctccaggcttaatccatgaaggtcccaggaaaacttgataaagcattcctgatcccacaaccgtgaatgcctctctgatcattagaagagtctggtcctctgttttcagggtcccttaccccttcctcagtcgtggataagcaccagtacattctgcctgacttctgtgtctcatg

SEQ ID NO: 123 - DNA, 435 bp, Homo sapiens
  misc_feature (54)..(54): n= a, c, g, or t
cctatatganctgtactttttataagtagtagttgctctcagaatagcctgtgncaattgtgaatgactggttttagttgacatcaacaagtaatactgtaattgtgtgttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnataattgtgttttaatactacccaaagtagcaatgtacctgatctcttgctagaacatagtgcctcatattcaatataata

SEQ ID NO: 124 - DNA, 450 bp, Homo sapiens
  misc_feature (95)..(95): n= a, c, g, or t
ggaaagagaaactgagggcaaggcagaaactttatcagggtcacctaaaaaactgacagcacagccagaactgagacccagggcttgggattccnagcccaagtcaaggtcagcatgtggcttgggccagcaccctaacctcatgtccttttggtatcaatgggaacaacagagatgctgtggtaggtaccagagtaaaccttctagattctcatggtgccagcctctagggaaaccaagtgggcacagatccccagtcccctggctacactggctcctgggcattccaggatcctggcaccctgccaagggtgaaaacagagctgcagactcctgggctgcattctgcccccttcttttgggtttgcagctcactgctgggtttgcaacaagcctaatctctgcatgtgcagacttagaggagccagctgagagggagcattgccagca

SEQ ID NO: 125 - DNA, 8210 bp, Homo sapiens
ggaaagagaaactgagggcaaggcagaaactttatcagggtcacctaaaaaactgacagcacagccagaactgagacccagggcttgggattcccagcccaagtcaaggtcagcatgtggcttgggccagcaccctaacctcatgtccttttggtatcaatgggaacaacagagatgctgtggtaggtaccagagtaaaccttctagattctcatggtgccagcctctagggaaaccaagtgggcacagatccccagtcccctggctacactggctcctgggcattccaggatcctggcaccctgccaagggtgaaaacagagctgcagactcctgggctgcattctgcccccttcttttgggtttgcagctcactgctgggtttgcaacaagcctaatctctgcatgtgcagacttagaggagccagctgagagggagcattgccagcagcttggaatcctccatgaagcctgccgaccccccttcccccaagacttttgctgggcagtcgccgccgccggctttggggcccaggacagaatgaccgagaacatgaaggagtgcttggcccagaccaatgcagccgtgggggatatggtgacggtggtgaagacggaggtctgctcaccactccgagaccaggagtatggccagccctgctctaggagaccggactcctcggccatggaagttgagcccaagaaactgaaggggaagcgcgacctcatcgtgcccaaaagcttccagcaagtggacttctggttctgtgagtcctgccaggagtacttcgtggatgaatgcccaaaccatggccccccggtgtttgtgtctgacacaccggtgcccgtgggcatcccagaccgggcggcgctcaccatcccacagggcatggaggtggtcaaggacactagtggagagagtgacgtgcgatgtgtaaacgaggtcatccccaagggccacatcttcggcccctatgaggggcagatctccacccaggacaaatcagctggcttcttctcctggctgattgtggacaagaacaaccgctataagtccatagatggctcagacgagaccaaagccaactggatgaggaatgtggcacacctggctgagcgtaagaggaagcccaagttctccaaggaggagctggacattcttgtcacagaggtgacccaccatgaagcagtgctctttgggagggagaccatgcggctgtcccatgctgacagggacaagattcgggaaggccatagccccggacaatcacgctccgtagccagggtgccccgatccggtcaacgacattaagcacagatgggcatgacctgaaacggaggaccaaggacaagctggccttcatgcagcagtccctgtcgggccctggggccgggggccgggcccccaccatcgtgctcacggcccacgagagggccatcaagtcggcgctgctcacggcccgtgcagggcgcggcttccccagggcggaactggatggcaccgacagcccttcgaccagctatgatgaagatgaggaggcgcctgggccctcaaggcagcctcttcgggtgcctctgcagcggtctccggaggaagaggcccacctggccaggcccgccctgctccgttcatcctcctcctcagaccagtctgagacggtgggccccaagccagaggccctgccccatccctcgccccaggcccaggctgcctgcaggacccctcggccgcaccccagcccacccaccacgggccttgactggcagctcctccacgtccatgcccagcagaccgaggtgttccggcagttctgccaggagctggtgaccgtgcaccgggacatggccaacagcatgcacgtcatcggccaggccatggccgagctgaccagccgtgtcggtcagatgtgccagacgctgacagagatccgggatggggttcaggcatctcagcgggggccagaagggggcagaccctacgggctccactccccaggccacccaggcccaagcccccctgccagagcccccaccagcttccccagcatcagcccccacacggactaccagggatagcgatgcttatatccgatgtgataagaacaccgatgtgttggggctaacagagaacacagacctaggacaagatgcaaattctggcggatctaagcaccgtaaatcttgcaagttctacacctccatggtggagcctgtggcttgcttctctcaaaaggtgttccctcatcccaccttttggaggtacgtggtcatctcccgggaggagagggagcagaacctgctggcgttccagcacagtgagcgcatctacttccgggcgtgcagggacatccggcctggggagtggctgcgggtctggtacagcgaggactacatgaagcgcctgcacagcatgtcccaggaaaccattcaccgcaacctggccagagtggtcttctccagagcacctgaagctgcatcatcctccatgagccccaagaccacaggggattgctcagagaagggagagaagaggttgcagagggagaagtctgagcaggttctggataacccagaagacctgaggggtcccattcatctctctgtgctgagacagggcaaaagtccctacaagcgtggctttgatgagggggatgtacacccccaagctaagaagaagaaaattgacctgattttcaaggatgttctggaggcctcactggaatctgcgaaggtggaagcccaccagttggccctgagcacctcactggtcatcaggaaagtccccaaataccaggatgacgcctacagtcagtgtgcaacaacaatgacccatggtgtgcagaatataggccagacccagggggagggggactggaaggtcccccagggggtctccaaggagccaggccaattggaggatgaagaagaggagccttcatcattcaaggccgacagtcctgccgaggcctcccttgcatctgaccctcatgaacttcccaccacctctttttgccctaactgtattcgcctaaagaagaaggttcgggagctccaggcagaattagacatgcttaagtctgggaaacttcctgagccccccgtattgccaccacaggtactggagctcccagagttctcggaccctgcagcctcagaaagcatggtctccggccccgccatcatggaggatgatgaccaggaagtcgattcagcagatgaatctgtctccaatgatatgatgacagcgacggatgagccctccaagatgtcatcggccaccgggcgccgaatccggcgctttaagcaggaatggctgaagaagttctggttcctgcggtactccccaaccctcaatgagatgtggtgccacgtctgccgccagtacacggtgcagtcctcacgcacctcggccttcatcattggctccaagcagtttaagattcacaccatcaaacttcacagccagagcaacctgcacaagaagtgcctgcaactgtacaagctccgcatgcacccggagaagacagaggagatgtgtcgcaacatgaccctgctcttcaacaccgcctaccacctggccttggagggcaggccctacctggacttccggcccctggcggagctgctgaggaagtgtgagctcaaggtggtggaccagtacatgaatgagggagactgccagatcctcatccatcacatcgcccgggccctgcgggaagacctggtggagcgcatccgccagtcaccttgcctcagcgtcatcctggatgggcagagcgacgacctgctggccgacacggtggctgtctatgttcagtacaccagcagtgatgggcccccggccacagagttcctgtccctgcaggagctgggattctctagcacagaaagctatctccaggcacttgaccgggccttctcggccttgggcatccggttgcaggatgaaaagccaactgttggcttgggtgtagacggagccaacatcacagccagcctccgtgccagcatgttcatgaccatccgcaagacgctgccctggctgctgtgcctgcccttcatggtgcaccggccccacctggagatcctggatgccatcagcgggaaggagctcccatgcctggaggagctggagaacaacctgaagcagctgctgagcttctaccgctactcaccgcgcctcatgtgcgagctgcggtccacggcggccaccctttgtgaggagacagagttcctgggcgatatccgggcagtgcggtggatcatcggcgagcagaacgtcctcaacgctctcatcaaggactacctggaggtggtggcccatctcaaggaggtcagcagccagacccagcgggcagacgcctcggccatcgcactggccctgctgcagttcctcatggactaccagtccatcaagctcatctacttcctgctggacgtgattgctgtgctctcgcgtctggcctacatcttccagggcgagtacctgctggtgtcccaggtggatgacaagatcgaggaggccatccaggagatcagccggctggctgactccccgggagaatacctgcaggagttcgaggagaatttccgagagagcttcaacgggatcgccatgaagaacctcagggtggctgaagccaagttccagtccatcagggagaagatctgccagaagacccaggtcatcctggctcagaggttcgactcccgcagccggatctttgtgaaggcctgccaggtgtttgacctggctgcctggcccaggagcagtgaggagctgatgagctatggcaaggaggatatggtgcaaatatttgatcacctggaggccatcccgaccttttcccgggatgtctgtagggaagggctggacccccggggtagtctgttgatggagtggcgagaactcaaggccgattactacaccaaaaatggcttcaaagacctgatcagccacatttgcaagtacaaacagaggtttccactcttgaacaagatcatccaggttcttaaagtcctccccacttccaccgcttgctgcgagaaaggccgcaatgccctccagcgagttcgcaaaaaccaccgctcccgcctgaccctggagcagcttagcgacctgttgacaatcgctgtaaacggaccgccaatcaccaactttgatgccaagcgagccctggacagctggtttgaggagaagtctgggaacagttacgcgctgtctgcagaagtcctcagtaggatgtctgcgctggagcagaagccagcactacagaccatggaccacgggacggagttttaccccgacatttagggagctggcgctgcagagttcactaagctgttgaatatttttttaatctatactcataagctttgatatattatataaatatatattatattatattatattatattatatatatatatatatataaactcacactgaaaatttttaaaaaccaaggtgacgcgtccaccagaagccactgggagatttcagaaaggaaaaatgttggaaactgactcttgtctacaaaatttggcagctgcaacatacatggcaactcattttcactcacagaagcacgtgctggggcctcctgtgttcccaccttactgtccaccaacagcataagctaaaatgacaggtctctgtcatcacctttaggtagctcattttgtttatgttttcatttgcgggtggcggggctctgggtttgggtttatgttcttgccttttcttttttcatttggttttatgatgggagggagctcctcagcctcctcattgacattctggtccggctgaatcagatctctgacttaagtcagggtgggttgtctgtctgcatttgggaggcaggggggttgacctttctccctccccacctgacttcagcttgagatcttttttttattcatttcctgatgagggttccttcactgtcctacaaacaaaagtgtcggtcaaactgtgacactgccacacctcacctctgttgcctcgtccatccctgggttgtggatcccttccttccagccccccctggaaactcacaatattacccattatactgaaggcaacattgcctcaccgctgagcttgaaatcctggggaagggagaaggggtaagcttttagcattcctgtttttacgaggtggaggataaaacaatataattccattccaatccagggcttttggggagatgaagagccaagaagtccagaccccaacaggggagtgatttttggctaaaaaacaaggaaaatgaaaagtacatattcgagttacatggattatttatactttttctttataatcatatcctgtgttgagggtattttttttcctttaataatcaagaaatgcctgctatagttcagtggcaggtagtgtcaatgcaaattgtgccctaagttatgcataactcaaatgagagtctgtagagatgtggtcctccttttgcaacaaggctgataacatgctacatggtcataggaaactggggaatgtgtctctgcctgtaaactcttccttttttgaacagggtagagatgtcctaaagaaatggagaaaagaagagaggactctcaaggcatcagcctacacagacacacacacacacacagacacacacacacacacacagacacacacacacacacacaatcacaatatacaatataagctttagaaatagccacttgcctattccctggggcaagtagtggtttaaactagaggagtctgatcaatgctctttcattcatttaactaccggtatacctcgcaagggagttttaaaaaatgtgcgtgagctgttaaaaacttctgttcatgttcctacatctgatttatgcatattttatatgcagagatcctatcacgtggatgcaggtcattttgggggagggaggaagatctgaattatatacatgtggtcagttctgctgagagcttcatctcttggttggcagagggcttgtgcagccctgacgggacccagaagcccatttgctcgcagtttcctctctctgtttttttcctcctggaggtaggagagagggcctgaccaggcaccagatgatggagcaagggcagctgcatgctccctctctccagaccagccttttgcttttggggtccgaaggggcatttgctcccatcctgagcctcctctgcccctgtcttgctcttccccaccatcctacaagtacctcagtctccagcaggcccacccctccacctgcagcccagggcgggtctgttctgccaatgcccacctccttgagccacagttagctgccaactgggtcttgggacaccctccagtacctggctcaagagagaccaggccgggccgagccttcttcccactgcagtggactagacccacggccaggggatgggcatccccaggtagcaatcccacaacgctgttccccctccctcacagaggccccgtggcccctaccccactcccgccgtaacaggcaggtttagttcacaaactccttcaaaaccctcccgcaccaggactgagcagcgtcagtggcaataggaaaggtccaaactggatcaagagctggtccaggaaagataccgcccctgccctgttagatgcttctgctgcccctagaggccaagcccctgaagtgcagccgtcctggcctccctcacttgctcaaccactgtcaggaggagaggatgtgggcagcatgagcatcgccaggcagcgctgcccaccatccacaggctttctggccagggcagggggcatcagctagcaggaaacggtgggagagacatatctgcacactcataaattcaatggctactccagtccagaagcagggctttggcccagcccgctccgcgcagcagctgctgctggctgtactcaggacaacgctgctgaaccattttcatgtcaatcacaaaggaaaaataagtggggatggggggaaatacctaggagtctattatcacatacatattaatatgttaatactttctttaaaaaaaacctcttgatgttattattttgcagactacgctttatagtacctgtgtgacgggacctagaacactggatacaaatagagctatgttggtttatcataatatgtacgcagaaactttctttttgtcatattatccttgtaatgtaagaagattgttaataaaagcatttaaattt

SEQ ID NO: 126 - DNA, 510 bp, Homo sapiens
caataaataggtgtcagataataaggactcggtaactcattatgtttccctttatgctttggctgccaggaggctcagaattgaatgaagaactcaatcactaacccttttacaacagattcccggcataaccaacacccctctttctccatgaggcctctaacactatctctggttgtttcttaactgtcctgtttagtatgtgtttgttttataaaacattgcaaatcttttgtgtggtgaaggtgggatgtgagagataggacataaatgcaaatggcccacagatgtctctactttgagatacaaagtaatactattgcctatcctccctccaaaccccttccgcatccctctgtgccctcctcgtttattcctaatggggaccacctcctcctggcaagtatcagtatttccattttgcaaaagaagacactaaggccctagaaagtgtaagtgacgtgccatggccacactgattatcctccccacactcatcgcccttaccac

SEQ ID NO: 127 - DNA, 206 bp, Homo sapiens
cctaacctcaagtgagccgcctgcctcgaagatgctttatttataagcgctttggacacactttaataaatgctctattgtatacagttgaactctatataacattttgaataatattgttaatgcttaccctaagtactactatcatctctgtattgtttaacaaaatatttccacatttgacaataaagtcagtatttgaaagg

SEQ ID NO: 128 - DNA, 439 bp, Homo sapiens
gtcgacttttttttttttttctttttttgagacagagtcccactctgtcacccaggctggagtgcagtggtgcaatcttggctcactgcaacctccacctcccaggttcaagcgattctcgtgtctcagcctccccagtagctgggactacaggtgcgccatgacgcctggctaatttttttgtattttcagtagagacggggtttcaccatgttggccagtctgctttcctcctaacctcaagtgagccgcctgcctcgaagatgctttatttataagcgctttggacacactttaataaatgctctattgtatacagttgaactctatataacattttgaataatattgttaatgcttaccctaagtactactatcatctctgtattgtttaacaaaatatttccacatttgacaataaagtcagtatttgaaagga

SEQ ID NO: 129 - DNA, 827 bp, Homo sapiens
  misc_feature (82)..(232): n= a, c, g, or t
aagagaaaagaatggacctttacgaaggtctcagagcaaggtaagtggtaaagccaaccaagtctatctcagctcatttagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccttatctggctttccattgcacttaaaataaaaactacactcatctaacatggcctaccagggcctgcctgatctcttcctcctgcctctttccctcatggttacacattgaagccaaactggccttctttctaattcctttcgaagaattgaaggctttaggaccttggtacatgctgttctcttctcttggaaaactccctcactctgtttgggtaactcctactattctttttttttgggcttaaatgtacctggtccatgcataaactatgtcctactggttactatgtcttatagtaccttgttctttttcttcctagtacttaggaccatctaaattttaaatttacttacgtgtttaatgattgtctcttccattaaattctccacatagtagggatcataatagtttcgttctccatggtacattcagaacatgtagcacagtgcctggcacatagtgtaagtactcaaaatacacttccaaagagaagagaaaagaatggacctttacgaaggtctcagagcaaggtaagtggtaaagccaaccaagtctatctcagctcatttagttacttcatttttttccc

SEQ ID NO: 130 - DNA, 1072 bp, Homo sapiens
  misc_feature (331)..(481): n= a, c, g, or t
taaatgtacctggtccatgcataaactatgtcctactggttactatgtcttatagtaccttgttctttttcttcctagtacttaggaccatctaaattttaaatttacttacgtgtttaatgattgtctcttccattaaattctccacatagtagggatcataatagtttcgttctccattgtacattcagaacatgtagcacagtgcctggcacatagtgtaagtactcaaaatacacttccaaagagaagagaaaagaatggacctttacgaaggtctcagagcaaggtaagtggtaaagccaaccaagtctatctcagctcatttagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccttatctggctttccattgcacttaaaataaaaactacactcatctaacatggcctaccagggcctgcctgatctcttcctcctgcctctttccctcatggttacacattgaagccaaactggccttctttctaattcctttcgaagaattgaaggctttaggaccttggtacatgctgttctcttctcttggaaaactccctcactctgtttgggtaactcctactattctttttttttgggcttaaatgtacctggtccatgcataaactatgtcctactggttactatgtcttatagtacctggttctttttcttcctagtacttaggaccatctaaattttaaatttacttacgtgtttaatgatggtctcttccattaaattctccacatagtagggatcataatagtttcgttctccatggtacattcagaacatgtagcacagggcctggcacatagtgtaagtactcaaaatacacttccaaagagaagagaaaagaatggacctttacgaaggtctcagagcaaggtaagtggtaaagccaaccaagtctatctcagctcatttagttacttcatttttt

SEQ ID NO: 131 - DNA, 538 bp, Homo sapiens
gagatataaccaccacacccaccttatatgtccttttatttcataaaaaacaattttagattatctgatatcacacatatcccataaatggtagtcattaatttagaagtgaatttataaagttaagagaagttaaaattgcacatgcattttgtttctaaaatctgagatgtctatgcatttccaggtattgtatatttgcaaatacattgggttagaaatcatctttaacattttgtgttagtgtaataacaatattatctaatattaatgctaccagaatccagttgctttttctgtgtatcaaaatttatcataactttttccacttaacactaggtaattaaacagaatctgcccaatttattctgcagtaaaattattttaaaatctatttttcctgctgactcttagaaattgcagaaagacaaaaaccagtttcatctccagtaatagtgtgaaacaatttccttccagtgggacagaaacctagacatactagggaaagatttaaatataaagaaaaatgccttggctg

SEQ ID NO: 132 - DNA, 6096 bp, Homo sapiens
  misc_feature (5057)..(5057): n= a, c, g, or t
ggcacgagcggagaaccccgcaatctctgcgcccacaaaatacaccgacgatgcccgatctactttaagggctgaaacccacgggcctgagagactataagagcgttccctaccgccatggaacaacggggacagaacgccccggccgcttcgggggcccggaaaaggcacggcccaggacccagggaggcgcggggagccaggcctgggctccgggtccccaagacccttgtgctcgttgtcgccgcggtcctgctgttggtctcagctgagtctgctctgatcacccaacaagacctagctccccagcagagagtggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctctctcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaagtgtgattctcttcaggaagtcagaccttccctggtttaccttttttctggaaaaagcccaactggactccagtcagtaggaaagtgccacaattgtcacatgaccggtactggaagaaactctcccatccaacatcacccagtggatggaacatcctgtaacttttcactgcacttggcattatttttataagctgaatgtgataataaggacactatggaaatgtctggatcattccgtttgtgcgtactttgagatttggtttgggatgtcattgttttcacagcacttttttatcctaatgtaaatgctttatttatttatttgggctacattgtaagatccatctacacagtcgttgtccgacttcacttgatactatatgatatgaaccttttttgggtggggggtgcggggcagttcactctgtctcccaggctggagtgcaatggtgcaatcttggctcactatagccttgacctctcaggctcaagcgattctcccacctcagccatccaaatagctgggaccacaggtgtgcaccaccacgcccggctaattttttgtattttgtctagatataggggctctctatgttgctcagggtggtctcgaattcctggactcaagcagtctgcccacctcagactcccaaagcggtggaattagaggcgtgagccccatgcttggccttacctttctacttttataattctgtatgttattattttatgaacatgaagaaactttagtaaatgtacttgtttacatagttatgtgaatagattagataaacataaaaggaggagacatacaatgggggaagaagaagaagtcccctgtaagatgtcactgtctgggttccagccctccctcagatgtactttggcttcaatgattggcaacttctacaggggccagtcttttgaactggacaaccttacaagtatatgagtattatttataggtagttgtttacatatgagtcgggaccaaagagaactggatccacgtgaagtcctgtgtgtggctggtccctacctgggcagtctcatttgcacccatagcccccatctatggacaggctgggacagaggcagatgggttagatcacacataacaatagggtctatgtcatatcccaagtgaacttgagccctgtttgggctcaggagatagaagacaaaatctgtctcccacgtctgccatggcatcaagggggaagagtagatggtgcttgagaatggtgtgaaatggttgccatctcaggagtagatggcccggctcacttctggttatctgtcaccctgagcccatgagctgccttttagggtacagattgcctacttgaggaccttggccgctctgtaagcatctgactcatctcagaaatgtcaattcttaaacactgtggcaacaggacctagaatggctgacgcattaaggttttcttcttgtgtcctgttctattattgttttaagacctcagtaaccatttcagcctctttccagcaaacccttctccatagtatwtcagtcatggaaggrtcatttatgcaggtagtcattccaggagtttttggtcttttctgtctcaaggcattgtgtgttttgttccgggactggtttgggtgggacaaagttagaattgcctgaagatcacacattcagactgttgtgtctgtggagttttaggagtggggggtgacctttctggtctttgcacttccatcctctcccacttccatctggcatcccacgcgttgtcccctgcacttctggaaggcacagggtgctgctgcctcctggtctttgcctttgctgggccttctgtgcaggacgctcagcctcagggctcagaaggtgccagtccggtcccaggtcccttgtcccttccacagaggccttcctagaagatgcatctagagtgtcagccttatcagtgtttaagatttgtcttttatttttaatttttttgagacagagtctcactctgtcgcccaggctggagtgcaatggtgcgatcttggctcaccgcaacctccgcctcctgggttcaagcaattctcctgcctcagcctcccaagtagctgggattacaggcacccgccaccacgcctggctaatttttgtatttttagtagagacggggtttcaccatgttggccaggctggtcttgaactcctgacctcaggtgatccacctgcctcggcctcccaaagtgctgggattacaggcatgagccaccatgcccggccccaaatgtcatgtttttaaataaaaacatagaaaatgatataaaggttcacagcatcatcaagaaaacagttcccccgtgtcgcggaggcggagatgtccatgccattcctacaccctctgggtctcaggtaatttccttcacggtcaagacctcttcgcggtagcctttcccttcctctggggtgaattcctcccagatgatctcgtcttccacctcatggttatcattcagtttgtctttgatctgtggaactggcgggaacagccgctgtatcctaaggaaccttttaaagaggaagccgaggacgatgccacagacaagggttcccacgattaggagcacataaatgtacacagagccgaggttcccgtcgtcagaaccaaattcaatggcttcactccaggagctccaattcaagatgcggacgtctgcagctctgatcttcacactgtgttttgctctgggctcagagcttggaaagttgtatctattttccaaatcaccagaaacattaatcagtaggttttccgtgccaggctgggtattctttctgtggacgtccagctggtactgaaagtccaggtacgacagcttctgataggtcctgggctgtttccaccgtacgaggcagtgcgtcgtgttgcaacgtacggtgacattgctgggagggttgaatcgttctattttctttgtgtccaaaagtgaatcaaagaattggatgccaatttctcggctggttccgttaaccagaaagtaattgcgagacgttaatcctgacaggttatccaggtgacatcccacatgggttcctgagtcttgtatgtaataaggacaccggatctccctccttctctttgagtttcgtatgtacaaaaaatactggacgtcacggggggccgtcggacccctcgcccaggtacagttcattaaatccgcattgtagatgaaacaggagaaattctgagcagcggtaccctcccttcctgaatttggataaagcagtttctgttgaaatcctctttgactagtattcacgtgaacctcaaatgtgactccttcatgcagacaaatttcacgaaatgtgcacgaacattcgttgttactgagcctgggttccacgactctgttcttcttgtcagttaagaaacacttgctgaaggttgtgttttcttggcagtcccagcttaaattcatcgtcctggagtcaaacctcacattgagactagaggctggtgccactgttcgcagatccgatttctctgggatcaggaggaatgctgggtgtggtaactcacagagcagaaggcttgtcaccaggagaagcatggtgctggtcagagagaagggaagagctngtcaaatgaggggtcatggccagctgggaaggtctgcagggccattattctgtctcccacatgggattaggacgtggacatctttggcatctcaagatggtcaatctagagtcaagcttccatggttcatgggtcatggctcagctgaccttcatgagggaacccatagctgacactaaaggcaggttctaacacacttcaaggaaattgcagtcactttcattcatttttcatagcttttactttctgttaataattagtggatttggggaaataagataaaggatggctcaggttttcattttcataacttttgttttgttttgctcttttgtactttttttttttttttttgagacggagtctcgctctgtcgcccaggctggagtgcagtggcgcatctcggctcactgcaagctccgcctcccgggctcaagcaatcctcctgccttggcctcccaaagtgttagggttagagatatgaaccaccacacccaccttatatgtccttttatttcataaaaaacaattttagattatctgatatcacacatatcccataaatggtagtcattaatttagaagtgaatttataaagttaagagaagttaaaattgcacatgcattttgtttctaaaatctgagatgtctatgcatttccaggtattgtatatttgcaaatacattgggttagaaatcatctttaacattttgtgttagtgtaataacaatattatctaatattaatgctaccagaatccagttgctttttctgtgtatcaaaatttatcataactttttccacttaacactaggtaattaaacagaatctgcccaatttattctgcagtaaaattattttaaaatctatttttcctgctgactcttagaaattgcagaaagacaaaaaccagtttcatctccagtaatagtgtgaaacaatttccttccagtgggacagaaacctagacatactagggaaagatttaaatataaagaaaaatgccttggctgaaaaaaaaaaaaaaaaaaggcggac

SEQ ID NO: 133 - DNA, 566 bp, Homo sapiens
gtactccctatttgaaattattttttggttgttaatatatgatattattaatgtttttaggtcacagaaagttctaagtggtaattttagatgtgtgggatctgagctaggactaaagcagagaatacccacgtaatcagaggtttctgggctccatagaggacgtagggcttttttttttctattggatttcttccagttttctcaggatcattagttctcttctgtagccaaaaattctggcctgttatgggattagagtctttaaggtttactcagactgtcattatgtgtagaaaaatgaattatgccctttggtaggacatgacacaaggctctgtttctagctgcaaatttaaattagattgtagagtgcttgggaaattggctttcaaaagaccaaagcttaatcttcactcctaaactgctggcttaattaaaatggatatttagaatttggtaaatgttgatttttctaataaaaggccttggtttaaaagggtgaccttaggattgtttctttcttaaaagcataattccagcccttctggcatggagcctggtcc

SEQ ID NO: 134 - DNA, 1072 bp, Homo sapiens
  misc_feature (603)..(846): n= a, c, g, or t
aagaagtacaggcttatctccgacttcagggtgtcatagtcatgggaatcatgcatacacacaaaccagaataaaccatttgaagcaaatgtgcttgaagtcagcacatctccaggaaggagagggtactgtgagctagatgaactgggtgcacagactctgtcttgacctttcactttatacccaattctttaggctaaatttgacattctaatggacttccagagttagtagttctcacactaaagactgtgtaattacaagttaaccatgttcaggttggtgatagaagcagaagtagatatttctgggctatgcagggctatggtaagggattggaatgaactaggtctgggcttaaatcttgtatctctagctatgaacttcagtttcctcatcagtctattggaatataactaccttttagggttgtggtgagacttagaatatctgtaacccctctagtgggttgtatggtaacagcaattgtgtcacattgttgttttgttgttagctgtaagaaaactcaggctgggctgagttgttccttgaaggcgtaattaattaattttttctgagacagagtctcatgctgtgtgatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaaggcataatttaaaagtcaccttctctatgaagcctccacaggttctaggcaagtataactcttccttccttgtgtgaccacagcacttaccatggcatgctctcatttgtctctttacatgcctcttcactacagagtctgggagcctctccagggcagggctgtatcttgattcacttttatgtccattatcaataccctgcacananctgatattaaata

SEQ ID NO: 135 - DNA, 1090 bp, Homo sapiens
  misc_feature (612)..(855): n= a, c, g, or t
taaagaagtacaaggcttattctccgacttcagggtgttcatagttcatgggaatcatgcatacacacaaaccagaataaaccatttgaagcaaatgtgcttgaagttcagcacatctccaggaaggagagggtactgtgagctagatgaactgggtgcacagactctgtcttgacctttcactttatacccaattctttaggctaaattttgacattctaattggacttccagagttagtagttctcacactaaagactgtgtaattacaagttaaccatgtacaggttggtgatagaagcagaagtagatatttctgggctatgcagggctatggtaagggattggaatgaaataggtctgggcttaaatcttgtatctctagctatgaacttcagtttcctcatcagtctattggaatataactaccttttagggttgtggtgagacttagaatatctgtaacccctctagtgggttgtatggtaacagcaattgtgtcacattgttgttttgttgttagctgtaagaaaactcaggctgggctgagttgttccttgaaggcgtaattaattaattttttctgagacagagtctcatgctgtgtgatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaaggcataatttaaaagtcaccttctctatgaagcctccacaggttctaggcaagtataactcttccttccttgtgtgaccacagcacttaccatggcatgctctcatttgtctctttacatgcctcttcactacagagtctgggagcctctccagggcagggctgtatcttgattcacttttatgtccattatcaataccctgcacaggacctgatattaaatattgaaata

SEQ ID NO: 136 - protein, 30 aa, Homo sapiens
MFVPCTRLRASAGRDHHLNLPLDPSARLAF

SEQ ID NO: 137 - protein, 38 aa, Homo sapiens
MFSCTFALNFSPSFLFKLFQSVLITRFCFGFSGGMGPV

SEQ ID NO: 138 - protein, 73 aa, Homo sapiens
MFTIHPFYIHLCIHVLNSCYACCIGNGDRVRKRHSSLGCHGVPFGKGWGMRGRTENKGITKYVICQVGISPME

SEQ ID NO: 139 - protein, 45 aa, Homo sapiens
MAVMYYAAHQTRSPFPLKRLYFGEGFGQPLYRLYTVLGPRTKAMS

SEQ ID NO: 140 - protein, 77 aa, Homo sapiens [sequence omitted]

SEQ ID NO: 141 - protein, 94 aa, Homo sapiens
MLWREDCWGKEEKGLSEAGVDEWGGGEWAPGPSRGKGGPISDPHLSMHLHLTLQAGLKSFTLLFTHSTESFLARTRHCKCTVAVLKGATLLTIF

SEQ ID NO: 142 - protein, 46 aa, Homo sapiens
MKQYTYEDLFTQGGRDCHDGRATSCSSANSHKRHQLREEGEYKNCP

SEQ ID NO: 143 - protein, 34 aa, Homo sapiens
MVLGQSTPRAIPGWRGGQKDSEMVGVHLWRLFTQ

SEQ ID NO: 144 - protein, 30 aa, Homo sapiens
MVNPDCLPYIIASRGLPSQGIVGYNPLDLP

SEQ ID NO: 145 - protein, 64 aa, Homo sapiens
MVDWVMDCENHKDVRLPVVASVRNVEKYKLIGVKYVQEIETEASCFIENGDLSKCSCYDGFVVY

SEQ ID NO: 146 - protein, 46 aa, Homo sapiens
MVRGMWVGESRKSILYCLVQPQYSVTSVSGFEVFQRYCPPPVVGDF

SEQ ID NO: 147 - protein, 96 aa, Homo sapiens
MTWTCPGSQGQCDQAASLTGRLGRLWGVRRRVLNAECLWTPQATHSRASDSAAFRQEGGSTGQGRSQHHSSHLGLCPELCDLGQAISSLSNPSVSI

SEQ ID NO: 148 - protein, 21 aa, Homo sapiens
MSAHLYTNVYTCIHTCAHMMC

SEQ ID NO: 149 - protein, 25 aa, Homo sapiens
MEWICLLVIPNLAHHEIRCCMKLDV

SEQ ID NO: 150 - protein, 87 aa, Homo sapiens
MRVCPVSSRFSSWRNQQVQARAAEYFLSCKEPVQGIESQPWPMSYQIFSFFGKSSKHLQNICLLHIFTIQKMYFLFSKKHNNRLLSL

SEQ ID NO: 151 - protein, 29 aa, Homo sapiens
MHLRLYSIEISCESLDHHCPIEILCEPQM

SEQ ID NO: 152 - protein, 12 aa, Homo sapiens
MCEFVGYPVFLL

SEQ ID NO: 153 - protein, 44 aa, Homo sapiens [sequence omitted]

SEQ ID NO: 154 - protein, 77 aa, Homo sapiens
MPFTLLLCILETFHNKIFKEHISDYFTQNGIKYFIINPCLVLLFSNFIYKFLTLHFHVLIIYNLSYYSKSLGQFYLP

SEQ ID NO: 155 - protein, 62 aa, Homo sapiens
MKTGLGCENSSPERDQRPLEERLPGGCSFLFCPGRTQGSSPWGMHLSAGWMCLTLGLQGLQL

SEQ ID NO: 156 - protein, 74 aa, Homo sapiens
GQDDEEQCLTAQVLDASSLSFNTRLKWFAICFVCGVFFSILGTGLLWLPGGIKLFAVFYTLGNLAALRRYVWLL

SEQ ID NO: 157 - protein, 46 aa, Homo sapiens
MKTCAKVSRTVVVVQRPDIGIRGLGSNPVSAAYLLCFWGRCSSPPE

SEQ ID NO: 158 - protein, 20 aa, Homo sapiens
MKCNLAPSYSGVSCFIFPSK

SEQ ID NO: 159 - protein, 47 aa, Homo sapiens
  MISC_FEATURE (33)..(33): X= any amino acid
MLESVGSPLGKAVCAEETGFWGSVLHLGADFMXMFHLWKTTVLHAKS

SEQ ID NO: 160 - protein, 62 aa, Homo sapiens
GMKGSEVTAMLEKGERMGCPAGCPREMYDLMNLCWTYDVENRPGFAAVELRLRNYYYDVVNN

SEQ ID NO: 161 - protein, 25 aa, Homo sapiens
MWAWAIIYPSELFLSNACPLCISEL

SEQ ID NO: 162 - protein, 59 aa, Homo sapiens
  MISC_FEATURE (44)..(44): X= any amino acid
MNVMVWQTVWGLSFYRAEGCWLAASVATLLVPRHWVTQPHEWVXPGHLRGQPVDKLLRE

SEQ ID NO: 163 - protein, 66 aa, Homo sapiens
  MISC_FEATURE (29)..(29): X= any amino acid
MILRVAHDLKLTSYLFLEIFHIISLDCSXPQVTXTAESEXXDKRGQLYNAISIXIAEKKKVXLFIL

SEQ ID NO: 164 - protein, 148 aa, Homo sapiens
MAFWTAARETVILTTSVRLRHDHHVRAKVGLVGCQNQGHPRRSLALALTADGFSAAARRKMPSIGPPSHCTEFQESWPTASQLLDLCDSVKDDARRVISTFNIPHTYLHAPIAGISNPRAAWAFYPAPLQPRPREEARSRRPKLGAKF

SEQ ID NO: 165 - protein, 64 aa, Homo sapiens
SQLLDLCDSVKDDARRVISTFNIPHTYLHAPIAGISNPRAAWAFYPAPLQPRPREEARSRRPKL

SEQ ID NO: 166 - protein, 52 aa, Homo sapiens
MCNRKVPLKVKICNSMTQPVHLGQVSFEIAAEFPLQCCLSVFYSFMRIDHPQ

SEQ ID NO: 167 - protein, 463 aa, Homo sapiens
DNMDQPFTVNSLKKLAAMPDHTDVSLSPEERVRALSKLGCNITISEDITPRRYFRSGVEMERMASVYLEEGNLENAFVLYNKFITLFVEKLPNHRDYQQCAVPEKQDIMKKLKEIAFPRTDELKNDLLKKYNVEYQEYLQSKNKYKAEILKKLEHQRLIEAERKRIAQMRQQQLESEQFLFFEDQLKKQELARGQMRSQQTSGLSEQIDGSALSCFSTHQNNSLLNVFADQPNKSDATNYASHSPPVNRALTPAATLSAVQNLVVEGLRCVVLPEDLCHKFLQLAESNTVRGIETCGILCGKLTHNEFTITHVIVPKQSAGPDYCDMENVEELFNVQDQHDLLTLGWIHTHPTQTAFLSSVDLHTHCSYQLMLPEAIAIVCSPKHKDTGIFRLTNAGMLEVSACKKKGFHPHTKEPRLFSIQKFLSGIISGTALEMEPLKIGYGPNGFPLLGISRSSSPSEQL

SEQ ID NO: 168 - protein, 62 aa, Homo sapiens
  MISC_FEATURE (2)..(3): X= any amino acid
MXXPHAHHMVSILIPQLCIITCQRLHILWWSPYNYNVFSTVIRYTNTLVLHCRXXXLGLSAV

SEQ ID NO: 169 - protein, 48 aa, Homo sapiens [sequence omitted]

SEQ ID NO: 170 - protein, 130 aa, Homo sapiens
MYISFGIMSLGLLSLLAVTSIPSVSNALNWREFSFIQSTLGYVALLISTFHVLIYGWKRAFEEEYYRFYTPPNFVLALVLPSIVILGKIILFLPCISRKLKRIKKGWEKSQFLEEGLGGTIRMSPRRGSQ

SEQ ID NO: 171 - protein, 132 aa, Homo sapiens
MYISFGIMSLGLLSLLAVTSIPSVSNALNWREFSFIQSTLGYVALLISTFHVLIYGWKRAFEEEYYRFYTPPNFVLALVLPSIVILGKIILFLPCISRKLKRIKKGWEKSQFLEEGLGGTISHVAPERVTVM

SEQ ID NO: 172 - protein, 50 aa, Homo sapiens
MSIISKLIYKYNTIPIKMSTGQRSRTWLANSKPYRKINKENFPGKLWEVE

SEQ ID NO: 173 - protein, 107 aa, Homo sapiens
FLFFADRVLLLLPRLECSGTILAHCNLRLPGSTNSPASASRVAGTTGTCHQERLSFIFLAEMGFPHVGQTGLKLLASSDPPASASQSVGIKGVSPHAWPHILFLNIK

SEQ ID NO: 174 - protein, 133 aa, Homo sapiens
MGNQREETKDRKMETLQNSERFHLPLVNKSVFIFSQGFFYFTSLQLKPGWANTLRYGGHGCNPERMLRNGKQKHKAGCPFVLEVRISMCSSRCPPRSERVPAVNDPGNGARVTQTWCRQQSYLSGALCPWTGH

SEQ ID NO: 175 - protein, 143 aa, Homo sapiens
  MISC_FEATURE (22)..(22): X= any amino acid
MQTKPRLGMRGAMADLHPKTHXGLRNRDLPRILQGHFDCLKSSLVLFGKKNMFLIHSTDSAFAVCWAWTQPLGVQSDSLQTACQCLQGSHRAGAICRCCVHPAPHLPETDPADSVPFSLGSRLARIVPTPSCFVLFCFSEFKF

SEQ ID NO: 176 - protein, 53 aa, Homo sapiens [sequence omitted]

SEQ ID NO: 177 - protein, 21 aa, Homo sapiens
MDSLTKLGSLPKFSEETWEQR

SEQ ID NO: 178 - protein, 612 aa, Homo sapiens
MAATAAVSPSDYLQPAASTTQDSQPSPLALLAATCSKIGPPAVEAAVTPPAPPQPTPRKLVPIKPAPLPLSPGKNSFGILSSKGNILQIQGSQLSASYPGGQLVFAIQNPTMINKGTRSNANIQYQAVPQIQASNSQTIQVQPNLTNQIQIIPGTNQAIITPSPSSHKPVPIKPAPIQKSSTTTTPVQSGANVVKLTGGGGNVTLTLPVNNLVNASDTGAPTQLLTESPPTPLSKTNKKARKKSLPASQPPVAVAEQVETVLIETTADNIIQAGNNLLIVQSPGGGQPAVVQQVQVVPPKAEQQQVVQIPQQALRVVQAASATLPTVPQKPSQNFQIQAAEPTPTQVYIRTPSGEVQTVLVQDSPPATAAATSNTTCSSPASRAPHLSGTSKKHSAAILRKERPLPKIAPAGSIISLNAAQLAAAAQAMQTININGVQVQGVPVTITNTGGQQQLTVQNVSGNNLTISGLSPTQIQLQMEQALAGETQPGEKRRRMACTCPNCKDGEKRSGEQGKKKHVCHIPDCGKTFRKTSLLRAHVRLHTGERPFVCNWFFCGKRFTRSDELQRHARTHTGDKRFECAQCQKRFMRSDHLTKHYKTHLVTKNLLVTKNL

SEQ ID NO: 179 - protein, 31 aa, Homo sapiens
MWRSGGQWVLIGSVPPALGIHASFTLLLCSR

SEQ ID NO: 180 - protein, 168 aa, Homo sapiens
ETGDITTNAREIKSKTIRDYMNKFDNLEEVDKFLKTYNLARQNAEEIENLKRPITNKEIESVIKNQPKQESLRPGGFTGAFYCTFKEELISFLPKGLAKVEEAILPYSFYEASITLLPNPDKDTTNKENDRPISLMNMDAKTFNEILANQIQQYIKKIIHHDQRSLGP

SEQ ID NO: 181 - protein, 95 aa, Homo sapiens
  MISC_FEATURE (10)..(10): X= any amino acid
MTQLSKDGPXRSAGSSVFVLAPSSPHCNVRKHLCVRAHLGIQSGHAHNPTAPRSRSRDDAAPFLVPCDVILLCYLLITLSCYNFILFPASSLFFC

SEQ ID NO: 182 - protein, 33 aa, Homo sapiens
MCSKNKKKTFRRYFQGIESFHFIEVSTSKRWFL

SEQ ID NO: 183 - protein, 85 aa, Homo sapiens
  MISC_FEATURE (65)..(65): X= any amino acid
MERHSKAKHNLSRGLVGSFDGQWQGECRWVAEVPSCPGPLISLEDLGWRVLGAQPLPVQLAQPLXSGTAHCPCSQLCQXLLANPG

SEQ ID NO: 184 - protein, 98 aa, Homo sapiens [sequence omitted]

SEQ ID NO: 185 - protein, 53 aa, Homo sapiens
MGIVAELKVTTGVQNSSRNLSLCFSLTQTLHCYFLSLLCSDLTVQSHTHMRTE

SEQ ID NO: 186 - protein, 85 aa, Homo sapiens
MSWNLPRPSLFLAPQMQPPKRMGSLGVREPKIELPLTLMDVSLHLSLFFLPRLLAPPYPCRRVLPRHQMAACGTTPNAERRLPLP

SEQ ID NO: 187 - protein, 496 aa, Homo sapiens
AAVRKEIETHQGQEMLVRGTEGIKEYINLGMPLSCFPEGGQVVITFSQSKSKQKEDNHIFGRQDKASTECVKFYIHAIGIGKCKRRIVKCGKLHKKGRKLCVYAFKGETIKDALCKDGRFLSFLENDDWKLIENNDTILESTQPVDELEGRYFQVEVEKRMVPSAAASQNPESEKRNTCVLREQIVAQYPSLKRESEKIIENFKKKMKVKNGETLFELHRTTFGKVTKNSSSIKVVKLLVRLSDSVGYLFWDSATTGYATCFVFKGLFILTCRHVIDSIVGDGIEPSKWATIIGQCVRVTFGYEELKDKETNYFFVEPWFEIHNEELDYAVLKLKENGQQVPMELYNGITPVPLSGLIHIIGHPYGEKKQIDACAVIPQGQRAKKCQERVQSKKAESPEYVHMYTQRSFQKIVHNPDVITYDTEFFFGASGSPVFDSKGSLVAMHAAGFAYTYQNETRSIIEFGSTMESILLDIKQRHKPWYEEVFVNQQDVEMMS

SEQ ID NO: 188 - protein, 41 aa, Homo sapiens
MCVFSTRLLRPMRTRTVCVLYTTESWVLAMCAYSSCSINVH

SEQ ID NO: 189 - protein, 68 aa, Homo sapiens
MTGGAWRNFRAVTPLCDTPTADTFIILLSKLKECATPRTLMETMAVSNSTNVELSQVQPTHHTNAKYK

SEQ ID NO: 190 - protein, 71 aa, Homo sapiens
WVQWFTPIIPAFWEAKVGGSLASRSSRPAWGIQADPHLYKKANKNEKKADPTPRGADPAGHTLPLALTRWR

SEQ ID NO: 191 - protein, 41 aa, Homo sapiens
MVWHMRKGRWKVYGEGDGTASFHCPVGPKITYKINTQKVRV

SEQ ID NO: 192 - protein, 31 aa, Homo sapiens
MKIGLGPLKKSWFQLGLVLSERLSRSKRQDL

SEQ ID NO: 193 - protein, 46 aa, Homo sapiens
MHPSTLETLRSPTLKKLLHSFHSPQRAGYRPFIEMCLNISLKSWES

SEQ ID NO: 194 - protein, 11 aa, Homo sapiens
MVRTVPCYEGY

SEQ ID NO: 195 - protein, 33 aa, Homo sapiens
MGHPEVMSSTHDFDLYEIPNNLQQEGPHPNMIP

SEQ ID NO: 196 - protein, 13 aa, Homo sapiens
MRFNPLKKSPWPG

SEQ ID NO: 197 - protein, 66 aa, Homo sapiens
MNFYEIPLYWLPSLFCLTSLLPTSVFWDHLQINYLQSNPVSSLLLWEPKLRHLQKISFQLLKNMFT

SEQ ID NO: 198 - protein, 24 aa, Homo sapiens
MPFLASANFPHILVMFSRFSIIFH

SEQ ID NO: 199 - protein, 14 aa, Homo sapiens
MIPNIQNWISLGND

SEQ ID NO: 200 - protein, 83 aa, Homo sapiens [sequence omitted]

SEQ ID NO: 201 - protein, 34 aa, Homo sapiens
MTMTIIVMIVNGSGGQNKLETITHNPLQYSKTGH

SEQ ID NO: 202 - protein, 35 aa, Homo sapiens
MSASKLRFSNKLKLETQSSRISLESCLQATVVVYS

SEQ ID NO: 203 - protein, 39 aa, Homo sapiens
  MISC_FEATURE (27)..(27): X= any amino acid
MLIGVTQKKTFCDCLSLESIGLNTIEXSLLWNSSELIIX

SEQ ID NO: 204 - protein, 30 aa, Homo sapiens
MASFVDSLNSLLDELNFLQAFSSPFLPSLG

SEQ ID NO: 205 - protein, 50 aa, Homo sapiens
  MISC_FEATURE (22)..(22): X= any amino acid
MELSLLDGHGPWLMFYLTFENXKIFFFPLRDTVQKPKQRLPIICINEYLP

SEQ ID NO: 206 - protein, 47 aa, Homo sapiens
MGKAFNLSPLRIVLANSFSEMFSIMLRRSLFWKSIINSSCSSSISVY

SEQ ID NO: 207 - protein, 91 aa, Homo sapiens
  MISC_FEATURE (2)..(2): X= any amino acid
MXPPPTVLGITGMSHHVQTCLSLFKYVNLNSPKPAARHSGPLSMSYASVSFPCNLVQNGPSINISWLLDFLTHTMIGYLLPRVTCLENSLV

SEQ ID NO: 208 - protein, 54 aa, Homo sapiens
MWPNSVSQSTNKSLSFEWHYNPVSEPESKQLIILYNCTELGNSAKESYKAVTHN

SEQ ID NO: 209 - protein, 74 aa, Homo sapiens
MNMFPDRYICNRKQPCAMVRKNQLCTLYKWFRMPNSQVSELHTQSPSSEPFAPSFWHFIYRPSLFQRFLEDLLA

SEQ ID NO: 210 - protein, 75 aa, Homo sapiens
HIYTTEYYAAIKKDEFMSFVGTWMKLETIILNKLSPGQNNQTPHVLTHRWELNSENTWTREGEHHTLGPVVGWGE

SEQ ID NO: 211 - protein, 46 aa, Homo sapiens
MTPWWFIPLLCSVSCPIYTFSLKFYFICYLCSFLFVGISLVCLFPS

SEQ ID NO: 212 - protein, 139 aa, Homo sapiens
  MISC_FEATURE (108)..(126): X= any amino acid
MEACPPAHPQWGVFCPFSRAQHCSISFPPSPSPCCYLLPSCAPTALGHPSVSLCAVVHLSSLLHLKSRTLPPSYQHLMQQVLPIAGTQQIANARLQNPALPPGCSSDXXXXXXXXXXXXXXXXXXXKTGMLCSSEETYT

SEQ ID NO: 213 - protein, 354 aa, Homo sapiens [sequence omitted]

SEQ ID NO: 214 - protein, 249 aa, Homo sapiens
NNLSLMDICGTSSFVPLMLDNFLETQRTISFPGCALQMYLTLALGSTECLLLAVMAYDRYVAICQPLRYPELMSGRPACRWQAELGTGFANSLLQSILVWHLPFCGHVINYFYEILAVLKLACGDISLNALALMVATAVLTLAPLLLICLSYLFILSAILRVPSAAGRCKAFSTCSAHRTVVVVFYGTISFMYFKPKAKDPNVDKTVALFYGVVTPSLNPIIYSLRNAEVKAAVLTLLRGGLLSRKASH

SEQ ID NO: 215 - protein, 42 aa, Homo sapiens
MLNLTFLMYHRSSKDFKLPCLVAFAFLRKAYHFLTGPRGSRP

SEQ ID NO: 216 - protein, 108 aa, Homo sapiens
LLLHHYYYYYYFLRQSFALVAQTGVQQRNLSSPQPPPPGFKRFSCLSLPRSWDYRHAPPHGLYFCRRSFSHVGEAGLKLPTSGDPPALALASQSAGIMGGSHGAQSRS

SEQ ID NO: 217 - protein, 28 aa, Homo sapiens
KQTKLIYGIKSQVAFVRDSDKRRLLRSG

SEQ ID NO: 218 - protein, 47 aa, Homo sapiens
MWAGREAVWGGNPVSEILQGSMFLSIVRLSIPQSNPQISLNKINKNQ

SEQ ID NO: 219 - protein, 101 aa, Homo sapiens
VTRAFVVCWTPGLVVLLLDGLNCRQCGVQHVKRWFLLLALLNSVVNPIIYSYKDEDMYGTMKKMICCFSQENPERRPSRIPSTVLSRSDTGSQYIEDSISQ

SEQ ID NO: 220 - protein, 38 aa, Homo sapiens
MQALNLSALSPLANFISSLSSTIDIKVVVSLNEIEKLK

SEQ ID NO: 221 - protein, 363 aa, Homo sapiens
MTGSNSHITILTLNVNGLNAPIKRRLANWIQSQDPSVYCTQETHLTCRDTHGLKIKGWRKIYQASGKQKAAGVAILVSDKTDVKQTKIKRDKEGHYIVVKGSIQQEELTILNIYAPNTGAPRFIKQVLRDLQRDLNSHTIIVGDFNTLLSTLDRSMRQKVNKDIQELNSALHQADLIDIYRTLHPKSTEYTFFSAPHHTYSKIDHIVGSKALLSKCKRTEIITNCLSDRSAIKLELRIKKLTQNRSTTWKLNNLLLNDYWVHNEMKAEIKMFFETNENKDTTYQNLWDTFKAVCTGNFIALNAHKRKQERSKIDTLTSQLKELEKQEQTHSKASRRQEITKIRAKLKEMETQKNLQKINESRS

SEQ ID NO: 222 - protein, 39 aa, Homo sapiens
MVCCSCWFQTGAKSDVFLVVCWLFSIPVRIANSVGRNIV

SEQ ID NO: 223 - protein, 23 aa, Homo sapiens
MDLVSVFKSVSHCFDCCIFYSKS

SEQ ID NO: 224 - protein, 97 aa, Homo sapiens
MWNQPLPDGAANAFITGKLYPPGARKHRQPSAIPAGGRTRSRGARNSLCSLMECFLFAVRPPTVLALLSHRRWVWGTPRSLHPGTKGCFSGSNLQGW

SEQ ID NO: 225 - protein, 163 aa, Homo sapiens
MDRYLLLVIWGEGKFPSAASREAEHGPEVSSGEGTENQPDFTAANVYHLLKRSISASINPEDSTFPACSVGGIPGSKKWFFAVQAIYGFYQFCSSDWQEIHFDTEKDKIEDVLQTNIEECLGAVECFEEEDSNSRESLSLAEYAYMVFVLSLKYLILDSYFNP

SEQ ID NO: 226 - protein, 80 aa, Homo sapiens
MESLQHCSLPSRAQVTSALCYSRVLAAVTPGFTESGILLCPTIPRSRIPTMQSLVSWGLSCNGALLAPFPKLQLYLLSGS

SEQ ID NO: 227 - protein, 70 aa, Homo sapiens
MVRNNLGAVQQQQSWWDGSVLWLPIPRTGHIPTMFLISNAVATWVTVVSIFSSSVGSVAVWIPGSSLSWA

SEQ ID NO: 228 - protein, 33 aa, Homo sapiens
MVWIYFVCFPPLRNSYDLALKLLKNTFLNLYSC

SEQ ID NO: 229 - protein, 15 aa, Homo sapiens
MVSKRTCAFLLLLLP

SEQ ID NO: 230 - protein, 51 aa, Homo sapiens [sequence omitted]

SEQ ID NO: 231 - protein, 28 aa, Homo sapiens
  MISC_FEATURE (11)..(11): X= any amino acid
MSTKTSHSQLXQAILRATTTYKKYXSYR

SEQ ID NO: 232 - protein, 102 aa, Homo sapiens
MSFWYQWEQQRCCGRYQSKPSRFSWCQPLGKPSGHRSPVPWLHWLLGIPGSWHPAKGENRAADSWAAFCPLLLGLQLTAGFATSLISACADLEEPAEREHCQ

SEQ ID NO: 233 - protein, 872 aa, Homo sapiens
QDRMTENMKECLAQTNAAVGDMVTVVKTEVCSPLRDQEYGQPCSRRPDSSAMEVEPKKLKGKRDLIVPKSFQQVDFWFCESCQEYFVDECPNHGPPVFVSDTPVPVGIPDRAALTIPQGMEVVKDTSGESDVRCVNEVIPKGHIFGPYEGQISTQDKSAGFFSWLIVDKNNRYKSIDGSDETKANWMRNVAHLAERKRKPKFSKEELDILVTEVTHHEAVLFGRETMRLSHADRDKIREGHSPGQSRSVARVPRSGQRHAAQMGMTANGGPRTSWPSCSSPCRALGPGAGPPPSCSRPTRGPSSRRCSRPVQGAASPGRNWMAPTALRPAMMKMRRRLGPQGSLFGCLCSGLRRKRPTWPGPPCSVHPPPQTSLRRWAPSQRPCPIPRPRPRLPAGPLGRTPAHPPRALTGSSSTSMPSRPRCSGSSARSWAPCTGTWPTACTSSARPWPSAPAVSVRCARRAQRSGMGFRHLSGGQKGADPTGSTPQATQAQAPLPEPPPASPASAPTRTTRDSDAYIRCDKNTDVLGLTENTDLGQDANSGGSKHRKSCKFYTSMVEPVACFSQKVFPHPTFWRYVVISREEREQNLLAFQHSERIYFRACRDIRPGEWLRVWYSEDYMKRLHSMSQETIHRNLARVVFSRAPEAASSSMSPKTTGDCSEKGEKRLQREKSEQVLDNPEDLRGPIHLSVLRQGKSPYKRGFDEGDVHPQAKKKKIDLIFKDVLEASLESAKVEAHQLALSTSLVIRKVPKYQDDAYSQCATTMTHGVQNIGQTQGEGDWKVPQGVSKEPGQLEDEEEEPSSFKADSPAEASLASDPHELPTTSFCPNCIRLKKKVRELQAELDMLKSGKLPEPPVLPPQVLELPEFSDPA

SEQ ID NO: 234 - protein, 58 aa, Homo sapiens
MQMAHRCLYFEIQSNTIAYPPSKPLPHPSVPSSFIPNGDHLLLASISISILQKKTLRP

SEQ ID NO: 235 - protein, 28 aa, Homo sapiens
MLTLSTTIISVLFNKIFPHLTIKSVFER

SEQ ID NO: 236 - protein, 79 aa, Homo sapiens
FFETESHSVTQAGVQWCNLGSLQPPPPRFKRFSCLSLPSSWDYRAPARLANFFVFSVETGFHHVGQSAFLLTSSEPPAS

SEQ ID NO: 237 - protein, 33 aa, Homo sapiens
MLKMISNPMYLQIYNTWKCIDISDFRNKMHVQF

SEQ ID NO: 238 - protein, 440 aa, Homo sapiens [sequence omitted]

SEQ ID NO: 239 - protein, 32 aa, Homo sapiens
MTQGSVSSCKFKLDCRVLGKLAFKRPKLNLHS

SEQ ID NO: 240 - protein, 36 aa, Homo sapiens
MRACHGKCCGHTRKEELYLPRTCGGFIEKVTFKLCL